(19) United States
(12) Patent Application Publication
(10) Pub. No.: US 2020/0306375 A1
(43) Pub. Date: Oct. 1, 2020

(54) COMPOSITIONS AND METHODS FOR TUMOR TRANSDUCTION

(71) Applicant: Aleta Biotherapeutics Inc., Natick, MA (US)

(72) Inventors: Roy Lobb, Wellesley, MA (US); Paul Rennert, Holliston, MA (US)

(21) Appl. No.: 16/487,752

(22) PCT Filed: Feb. 22, 2018
(86) PCT No.: PCT/US18/19266

§ 371 Date: Aug. 21, 2019

Related U.S. Application Data
(60) Provisional application No. 62/541,409, filed on Aug. 4, 2017.
(60) Provisional application No. 62/462,108, filed on Feb. 22, 2017.

(51) Int. Cl. A61K 39/395; A61K 48/00; A61K 38/17; A61K 35/17; A61P 35/00

(52) U.S. Cl. CPC A61K 39/39566; A61K 48/0016; A61P 35/00; A61K 35/17; A61K 38/1774

(57) ABSTRACT

The invention relates to cancer therapeutics, in particular, the system of making cancer cells more susceptible to effector cells by introduction of cellular therapy targets into the cancer cells.

### CROSS REFERENCE TO RELATED APPLICATIONS

This application claims priority to each of U.S. Provisional Patent Application Nos. 62/462,108 filed Feb. 22, 2017; and 62/541,409 filed Aug. 4, 2017, the entire contents of each of which are hereby incorporated by reference.

### BACKGROUND OF THE INVENTION

Recent examples of chimeric antigen receptor (CAR) T cell directed to solid tumors have been clinical (or even preclinical) failures. The invention described herein presents solutions for solid tumor and other oncology indications.

Cellular therapeutics that are introduced into cancer patients and traffic into tumor microenvironments encounter multiple barriers that limit their efficacy. These barriers include, but are not limited to: cellular anergy, intrinsic cell death, suboptimal cell trafficking, limited proliferation, limited effector function, poor “take” (survival, persistence and differentiation), active exclusion from tumors, persistent immunosuppression in the tumor microenvironment, and tumor-induced cell death. Almost all attempts to date to overcome these limitations require non-physiological manipulation of the cellular therapeutic both in vitro and in vivo. The efficacy of cellular therapeutics is therefore broadly hindered by both intrinsic and extrinsic factors.

Targeting solid tumors presents an additional set of challenges to overcome, for example, their overall lesser sensitivity to T cell mediated cytotoxicity, a microenvironment with differing immunosuppressive mechanisms between tumor types, and a lack of target antigens with favorable expression profiles. Additionally, solid tumors are heavily fortified against cellular therapeutic attack as they deploy an impenetrable extracellular matrix, hypoxia, and acidic pH. Despite the vast number of targets that have been investigated, only a small number are tumor-specific (e.g., expression is restricted to the tumor cell) and this finding, as such, illustrates the difficulty and the need to discover an effective solution for eliminating or reducing the tumor. Yet another problem for targeting tumors is the heterogeneity of the tumor cells which express different antigens and different levels of antigens. The invention described herein addresses these problems and provides additional benefits as well.

### SUMMARY OF THE INVENTION

The present invention is based, at least in part, upon the introduction of cellular therapy targets into cancer cells (e.g., tumor cells such as solid tumor cells) such that they are more susceptible to effector cells that can eliminate and/or reduce the cancer cells. As further detailed herein, cancer cells (e.g., tumors) can be transduced with fusion proteins. Such fusion proteins consist of a binding component fused to a target component. For example, the fusion protein can be (i) an antibody or antibody fragment which binds to one or more tumor-associated antigens (TAA) or tumor-specific antigens (TSA), fused to (ii) a target for cellular therapy, e.g. CD19; a cytokine-target fusion; a bi-specific T-cell engager (BiTE); or an anti-idiotype peptide or antibody or fragment thereof. They can also be transduced with CD19 variants (or mutants). These fusion proteins or variants (or mutants) can be secreted by the tumor cells and/or permeate the tumor microenvironment.

Accordingly, in one aspect, the invention provides for recombinant vectors encoding an antibody or antibody fragment to a tumor-associated antigens (TAA) or tumor-specific antigens (TSA) and a therapeutic agent, as a fusion protein. In other embodiments, the therapeutic agent is selected from the group consisting of a cytokines, peptides (e.g. anti-idiotype peptides), proteins, antibodies (e.g, anti-idiotype antibodies), antibody fragments, T-cellengager and NK-cell engager. In other embodiments, the tumor antigen is selected from the group consisting of glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1 , RU2 (AS), intestinal carboxyi esterase, mut hsp70-2, M-CSF, prostase, prostate-specific antigen (PSA), PAP, NY-ESO- 1, LAGE-la, p53, prostein, PSMA, Her2/neu, survivin and telomerase, prostate-carcinoma tumor antigen- 1 (PCTA-1), MAGE, ELF2M, neutrophil elastase, ephrinB2, CD22, insulin growth factor (IGF)-I, IGF-II, IGF-I receptor and mesothelin, EphA2, HER2, GD2, Glypican-3, 5T4, 8H9, αvβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-Al MAGE Al, HLA-A2, ILI IRa, IL13Ra2, KDR, Lambda, Lewis-Y, MCSP, Mesothelin, Mucl, MucI6, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, and VEGF receptors.

In other embodiments, the cytokine is capable of being secreted by a tumor cell comprising said vector. In other embodiments, the vector is a viral vector. In other embodiments, the vector integrates into the genome of a cancer cell. In other embodiments, the therapeutic agent is an antigen for a chimeric antigen receptor (CAR). In other embodiments, the therapeutic agent is an antigen for a T cell receptor (TCR). In other embodiments, the therapeutic agent is an antigen for an ADC antibody, ADCC antibody, and/or a radiotherapeutic antibody.

In some embodiments the therapeutic agent is an “anti-idiotype” peptide. In some embodiments, the therapeutic agent is an anti-idiotype” peptide that binds an antigen binding receptor of one or more additional cellular therapeutics (e.g., an scFv of a CAR-T cell). In some embodiments, an anti-idiotype peptide that binds an antigen binding receptor of one or more additional cellular therapeutics binds one or more CDRs of an antigen binding receptor (e.g., an scFv of a CAR-T cell).

In other embodiments, the therapeutic agent is an anti-idiotype antibody or fragment thereof. In some embodiments, the therapeutic agent is an anti-idiotype antibody or fragment that binds an antigen binding portion of one or more cellular therapeutics (e.g., an scFv of a CAR-T cell). For example, in some embodiments, the therapeutic agent is an anti-idiotype antibody or fragment (e.g., scFv) that binds to a B-cell specific marker antigen binding portion (e.g., a CAR that binds CD19, CD20, CD21, CD22, CD24, CD79a, CD79b, ROR1, or BCMA) of a CAR-T cell. Additionally, for example, in some embodiments, the therapeutic agent is an anti-idiotype antibody or fragment (e.g., scFv) that binds an anti-CD19 antibody or fragment (e.g., an anti-CD19 antibody (e.g., anti-CD19 scFv) expressed by a CAR-T cell). In other embodiments, the therapeutic agent is an immunostimulatory cytokine or molecule selected from the group consisting of TLR agonists, PAMP, DAMP and other stimulators. In other embodiments, the therapeutic agent is a peptide with immunomodulatory or anti-tumor properties.

In other embodiments, the TAA or TSA and therapeutic agent are expressed as a fusion protein. In other embodiments, the TAA or TSA is selected from the group consisting of a glioma-associated antigen, carcinoembryonic antigen (CEA), β-human chorionic gonadotropin, alphafetoprotein (AFP), lectin-reactive AFP, thyroglobulin, RAGE-1 , MN-CA IX, human telomerase reverse transcriptase, RU1 , RU2 (AS), intestinal carboxyi esterase, mut hsp70-2, M-CSF, prostase, prostate-specific antigen (PSA), PAP, NY-ESO- 1 , LAGE-la, p53, prostein, PSMA, Her2/neu, survivin and telomerase, prostate-carcinoma tumor antigen- 1 (PCTA-1), MAGE, ELF2M, neutrophil elastase, ephrinB2, CD22, insulin growth factor (IGF)-I, IGF-II, IGF-I receptor and mesothelin, EphA2, HER2, GD2, Glypican-3, 5T4, 8H9, αvβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-Al MAGE AI, HLA-A2, ILl IRa, IL13Ra2, KDR, Lambda, Lewis-Y, MCSP, Mesothelin, Mucl, Mucl6, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, and VEGF receptors

In other aspects, the invention provides for recombinant vectors encoding a cytokine to a tumor expressed cytokine receptor and a therapeutic agent as a fusion protein. In some embodiments, the cytokine receptor is a type I receptor or a type II receptor.

In other aspects, the invention provides for recombinant tumor cells comprising a vector as described above and herein. In some embodiments, the tumor cell expresses one or more fusion proteins.

In other aspects, the invention provides for methods of producing a recombinant tumor cell capable of expressing a fusion protein comprising an antibody or antibody fragment to a tumor-associated antigens (TAA) or tumor-specific antigens (TSA) and a therapeutic agent, said method comprising (a) introducing a vector as described above and herein into a tumor cell; and (b) culturing the tumor cell such that the vector is transduced into the tumor cell. In some embodiments, the vector and/or its components integrate in the tumor cells' genome. In some embodiments, the TAA or TSA is selected from the group consisting of glioma-associated antigen, carcinoembryonic antigen (CEA), β-human chorionic gonadotropin, alphafetoprotein (AFP), lectin-reactive AFP, thyroglubilin, RAGE-1 , MN-CA IX, human telomerase reverse transcriptase, RU1 , RU2 (AS), intestinal carboxyiesterase, mut hsp70-2, M-CSF, prostase, prostate-specific antigen (PSA), PAP, NY-ESO- 1 , LAGE-la, p53, prostein, PSMA, Her2/neu, survivin and telomerase, prostate-carcinoma tumor antigen- 1 (PCTA-1), MAGE, ELF2M, neutrophil elastase, ephrinB2, CD22, insulin growth factor (IGF)-I, IGF-II, IGF-I receptor and mesothelin, EphA2, HER2, GD2, Glypican-3, 5T4, 8H9, αvβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-Al MAGE AI, HLA-A2, ILl IRa, IL13Ra2, KDR, Lambda, Lewis-Y, MCSP, Mesothelin, Mucl, Mucl6, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, and VEGF receptors. In some embodiments, the TAA or TSA is an antigen for a chimeric antigen receptor (CAR). In some embodiments, the TAA or TSA is an antigen for a T cell receptor (TCR). In some embodiments, the therapeutic agent is an antigen for an ADC antibody, ADCC antibody, and/or a radiotherapeutic antibody. In other embodiments, the therapeutic agent is an anti-idiotype antibody or fragment thereof as described herein In some embodiments, the therapeutic agent is an immunostimulatory cytokine or molecule selected from the group consisting of TLR agonists, PAMP, DAMP and other stimulators. In some embodiments, the therapeutic agent is a peptide with immunomodulatory or anti-tumor properties.

In other aspects, the invention provides for methods of treating cancer in an individual in need thereof comprising administering a composition comprising a vector as described above and herein. In some embodiments, a fusion protein is expressed. In other embodiments, the cancer cell expresses a therapeutic agent that increases immune response against the cancer cell. In some embodiments, the cancer cell expresses one or more proteins or peptides that are capable of being recognized by or that recognize a CAR. In some embodiments, the cancer cell express one or more protein or peptide antigens that is capable of being recognized by a TCR. In some embodiments, the cancer cell express one or more fusion proteins containing therapeutic agents capable of being recognized by immune cells in the individual. In some embodiments, the method further comprises administration of CART cells to the individual.

### BRIEF DESCRIPTION OF THE DRAWINGS

FIG. 1 is a schematic showing a non-limiting example of the methods of introduction of a cellular therapy target into solid tumor cells. Step one corresponds to the transduction construct (e.g., tumor-selective transduction via e.g., viral transduction, RNA transduction, and gene insertion), and/or in vivo transduction and integration. Step two corresponds to the expression of a fusion protein (e.g., an antibody fragment to a tumor antigen fused to a CAR antigen, for example an scFv anti-tumor associated antigen (TAA) fused to CD19 or a fragment thereof). Step three corresponds to the antibody fragment fused to an antigen, e.g. CD19, which binds to tumor cells and presents the antigen, and all nearby tumor cells are coated with the antibody fragment fused to a protein, peptide, or other agent, for example, CD19, which is an antigen recognized by CAR T cells. Step four corresponds to the infusion in vivo of CAR T cells that recognize the CAR antigen (e.g., anti-CD19 CAR T cells). The response becomes cytotoxic to the CD19-coated tumor cell.

FIGS. 2A-2C demonstrate expression of fusion proteins from AAV transduced cells.

FIGS. 3A and 3B show summary results of an IFNγ ELISA measuring induction of IFNγ in CAR19 T- cells by expressed fusion proteins.

FIGS. 4A-4C show induction of cytotoxicity upon incubation of CAR19 T cells with AAV-1-expressed supernatant and BT474 cells.

FIG. 5 demonstrates secretion of anti- FMC63 (anti-Id) antibody from transfected 293T cells.

FIGS. 6A and 6B demonstrate expression of CAR19 (construct #140) with an FMC63 domain as detected by a Flag tag (6A) and detection of the CAR19 by anti- FMC63 antibody (86B).

FIGS. 7A-7C demonstrate Trastuzumab scFv/anti-ld scFv fusion proteins bind both FMC63 and Her2. FIG. 7A demonstrates binding of a Trastuzumab scFv/anti-ld scFv fusion protein to FMC63. FIG. 7B demonstrates binding of a Trastuzumab scFv/anti-ld scFv fusion protein to Her2. FIG. 7C demonstrates binding of a CD19 expressing construct (#42) with the FMC63 coated plate as a control.

FIGS. 8A and 8B demonstrate recognition of Her2 by Trastuzumab scFv/anti-ld scFv fusion proteins. FIG. 8A demonstrates Her2 expression on SKOV3 cells. FIG. 8B demonstrates binding to the SKOV3-Her2 cells by the Trastuzumab scFv/anti-ld scFv fusion protein.

FIG. 9 shows CAR19-mediated cytotoxicity redirected to HER2+SKOV3 cells by a Trastuzumab scFv/anti-ld scFv fusion protein.

FIGS. 10A and 10B summarize the calculated cytotoxicity of CAR19-mediated killing as redirected by a Trastuzumab scFv/anti-ld scFv fusion protein. FIG. 10A shows the calculated cytotoxicity. FIG. 1013 shows the calculated EC50 of construct #171.

FIG. 11 shows results of IFNγ ELISA for CAR19 killing redirected by construct #171.

FIGS. 12A and 12B demonstrate specificity of CAR19 redirected killing using Trastuzumab scFv/anti-ld scFv fusion proteins. FIG. 12A demonstrates results of CAR19-mediated cytotoxicity redirected to HER2+ SKOV3 cells by Trastuzumab scFv/anti-ld scFv construct #171 relative to a construct expressing an anti-Her2 protein (#16). FIG. 12B summaraizes the calculated cytotoxicity of CAR19-mediated killing as redirected by construct #171 or #16.

FIG. 13 demonstrates the lack of CAR19 redirected killing using Trastuzumab scFv/anti-ld scFv fusion proteins when the target cell (H929) lacks Her2.

### DETAILED DESCRIPTION OF THE INVENTION

The present invention is based, at least in part, upon the introduction of cellular therapy targets into solid tumor cells. Tumor cells can be transduced with fusion proteins such that the fusion proteins are secreted by the tumor cells and released to the tumor microenvironment to combat the tumor.

### I. Definitions

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are composed of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are fully human, or are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgG, IgE and IgM, anti-idiotype antibodies, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPsTM”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

As used herein, an “antibody fragment” includes a portion of an intact antibody, such as, for example, the antigen-binding or variable region of an antibody. Examples of antibody fragments include Fab, Fab′, F(ab′)₂, and Fv fragments; triabodies; tetrabodies; linear antibodies; single-chain antibody molecules; and multi-specific antibodies formed from antibody fragments. For example, antibody fragments include isolated fragments, “Fv” fragments (consisting of the variable regions of the heavy and light chains), recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)₂ fragment, scFv fragment, Fv fragment, dsFv diabody, dAb fragment, Fd′ fragment, Fd fragment, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids.

As used herein, “antigen” means a molecule that provokes an immune response; and/or an agent that binds to a T cell receptor (e.g., when presented by an MHC molecule) or to an antibody or antibody fragment. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source), or alternatively may exist on or in a cell. In some embodiments, an antigen is a recombinant antigen.

Idiotope: As used herein, the term “idiotope” refers to a unique antigenic determinant (epitope) of a variable region of an antibody, or antigen binding portion.

Idiotype: As used herein the term “idiotype” refers to a set of idiotopes of a particular antibody, or antigen binding portion.

The term, “autologous” refers to any material derived from the same individual to which it is later re-introduced into the individual.

The term “cell therapy antigen” as used herein is meant to refer to one or more antigens (that are genetically engineered or naturally occurring) that can be recognized by effector cells (e.g., genetically engineered CAR T cell or genetically engineered or naturally occurring TCR). An antigen that is expressed on tumors (i.e., “tumor-associated antigen” or TAA) is one type of cell therapy antigen. An antigen that is expressed selectively on tumors (i.e., “tumor-selective antigen” or TSA) is also a type of cell therapy antigen. An antigen can also be expressed on tumor cells through therapeutic intervention, for example, by transducing tumor cells, in vivo, with genetic materials of the present invention, as described herein.

As used herein, the term “fusion protein” generally refers to a polypeptide including at least two segments, each of which shows a high degree of amino acid identity to a peptide moiety that (1) occurs in nature, and/or (2) represents a functional domain of a polypeptide. Typically, a polypeptide containing at least two such segments is considered to be a fusion protein if the two segments are moieties that (1) are not included in nature in the same peptide, and/or (2) have not previously been linked to one another in a single polypeptide, and/or (3) have been linked to one another through action of the hand of man.

The term “promoter” refers to a region of a DNA sequence that directs expression of genes. The promoter is typically upstream from the start of transcription start site and is involved in recognition and binding of RNA polymerase and other transcription machinery (e.g., other proteins) to initiate transcription of a polynucleotide sequence.

The term “solid tumor” is meant as an abnormal mass that usually does not contain cysts or liquid areas. Solid tumors can be benign or malignant. Different types of solid tumors are named for the types of cells that form them (such as sarcomas, carcinomas, and lymphomas). Examples of solid tumors such as sarcomas and carcinoma, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

An “individual” or “subject” can be a vertebrate, a mammal, or a human. Mammals include, but are not limited to, farm animals, sport animals, pets, primates, mice and rats. In one aspect, a subject is a human. An “individual” or “subject” can be a “patient” (e.g., under the care of a physician) but in some cases, an individual or subject is not a patient.

“Pseudoviruses” or “papilloma pseudoviruses” or “papillomavirus gene transfer vectors” refer to one or more papillomavirus capsid proteins that assemble and package heterologous nucleic acids (e.g., DNA) with or without viral nucleic acids (e.g., DNA) into infectious particles. The methods used to produce papilloma pseudoviruses are known in the art and are described, for example, in U.S. Pat. Nos. 6,599,739, 7,205,126, and 6,416,945; and in Buck and Thomspon, Production of Papillomavirus-Based Gene Transfer Vectors. Current Protocols in Cell Biology 26.1.1-26.1.19, December 2007, all of which are incorporated by reference.

The term “T cell receptor” or “TCR” refers to a heterodimeric receptor found on the surface of T lymphocytes. TCRs are antigen-specific molecules that are responsible for recognizing antigenic peptides of the major histocompatibility complex (MHC) on the surface of antigen presenting cells (APCs), or any other nucleated cell. They are members of the immunoglobulin superfamily, and typically consist of two chains; alpha (α) and beta (β), while a small subset of TCRs are formed by variable gamma (γ) and delta (δ) chains. The chains pair on the surface of a T cell to form a heterodimeric receptor.

The term “transfected” or “transformed” or “transduced” is defined as a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny. In some embodiments, the host cell is a cancer cell, for example a tumor cell such as solid tumor cell.

The term “tropism” refers to the movement or targeting of a viral vector towards a receptor.

A “vector” is a composition which comprises an isolated nucleic acid, and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus and phage vectors (AAVP), retroviral vectors, human papilloma virus (HPV) pseudovirus vectors, and the like.

### II. Compositions

The invention provides for compositions that can be used for introduction of fusion proteins into cancer cells (e.g., tumor cells) that will make the cancer cells more susceptible for destruction by either an individual's immune system and/or additional therapeutic agents. Compositions can include, but are not limited to, vectors and various constructs described herein, host cells (including tumor cells) containing such vectors and/or constructs, host cells expressing or capable of expressing these vectors and/or constructs, kits containing the vectors, constructs, instructions, and/or reagents and the like.

Cancer cells (e.g., tumors) can be transduced with fusion proteins. As the fusion proteins are expressed (and/or secreted), the proteins can also permeate the tumor microenvironment. Non-limiting examples of fusion proteins contemplated by the invention include: antibody fusions so that the antibody or antibody fragment binds to one or more tumor-associated antigens (TAA) or tumor-specific antigens (TSA), cytokine target fusions, bi-specific T-cell engagers (BITES) or CD19 variants. In some embodiments, a fusion protein includes an antibody or fragment that binds a tumor antigen described herein, and an anti-idiotype antibody or fragment.

### Vector Design

The nucleic acid sequences coding the desired gene of interest can be cloned into a number of types of vectors. For example, the nucleic acid can be cloned into a plasmid, a phagemid, a phage derivative, an animal virus, and a cosmid. Other vectors can include expression vectors, replication vectors, probe generation vectors, sequencing vectors, and viral vectors. In other examples, the vector can be a foamy viral (FV) vector, a type of retroviral vector made from spumavirus. Viral vector design and technology is well known in the art as described in Sambrook et al, (Molecular Cloning: A Laboratory Manual, 2001), and in other virology and molecular biology manuals.

### Transduction Methods

Transfer of nucleic acid to a cell for gene-modification of the cell in order for the cell to express a gene of interest is widely performed via transduction (e.g., viral transduction). The nucleic acid sequence coding for the desired gene of interest or portion thereof (e.g., tumor associated antigen) can be obtained using recombinant methods known in the art. Exemplary methods include screening libraries from cells expressing the gene, deriving the gene from a vector, or isolating directly from cells and tissues. These methods are performed using standard techniques. In other embodiments, the gene of interest can be produced synthetically rather than cloned. Gene delivery methods are well-known in the art, for example, U.S. Pat. No. 5,399,346.

### Viral Transduction

Viruses are highly efficient at nucleic acid delivery to specific cell types, while often avoiding detection by the infected host immune system. These features make certain viruses attractive candidates as vehicles for introduction of cellular therapy targets into cancer cells, e.g., solid tumor cells. A number of viral based systems have been developed for gene transfer into mammalian cells. Examples of viral vectors include, but are not limited to, retroviruses, adenoviruses, adeno-associated viruses, herpes viruses, lentiviruses, poxviruses, herpes simplex 1 virus, herpes virus, oncoviruses (e.g., murine leukemia viruses), and the like. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Lentiviral and Retroviral transduction can be enhanced by the addition of polybrene (SantaCruz sc-134220; Millipore TR-1003-G; Sigma 107689), a cationic polymer (also known as hexamehtrine bromide) that is used to increase the efficiency of the retrovirus transduction.

For example, retroviruses provide a platform for gene delivery systems. Retroviruses are enveloped viruses that belong to the viral family Retroviridae. Once in a host's cell, the virus replicates by using a viral reverse transcriptase enzyme to transcribe its RNA into DNA. The retroviral DNA replicates as part of the host genome, and is referred to as a provirus. A selected gene can be inserted into a vector and packaged in retroviral particles using techniques known in the art. The recombinant virus can then be isolated and delivered to cells of the subject either in vivo or ex vivo. A number of retroviral systems are known in the art, for example See U.S. Pat Nos. 5,994,136, 6,165,782, and 6,428,953.

Retroviruses include the genus of Alpharetrovirus (e.g., avian leukosis virus), the genus of Betaretrovirus; (e.g., mouse mammary tumor virus) the genus of Deltaretrovirus (e.g., bovine leukemia virus and human T-lymphotropic virus), the genus of Epsilonretrovirus (e.g., Walleye dermal sarcoma virus), and the genus of Lentivirus.

In other embodiments, the retrovirus is a lentivirus a genus of viruses of the Retroviridae family, characterized by a long incubation period. Lentiviruses are unique among the retroviruses in being able to infect non- dividing cells; they can deliver a significant amount of genetic information into the DNA of the host cell, so they are one of the most efficient methods of a gene delivery vector. Lentiviral vectors have an advantage to other viral vectors in that they can transduce non-proliferating cells and show low immunogenicity. In some examples, the lentivirus includes, but is not limited to human immunodeficiency viruses (HIV-1 and HIV-2), simian immunodeficiency virus (S1V), feline immunodeficiency virus (FIV), equine infections anemia (EIA), and visna virus. Vectors derived from lentiviruses offer the means to achieve significant levels of gene transfer in vivo.

In embodiments, the vector is an adenovirus vector. Adenoviruses are a large family of viruses containing double stranded DNA. They replicate the DNA of the host cell, while using the host's cell machinery to synthesize viral RNA DNA and proteins. Adenoviruses are known in the art to affect both replicating and non-replicating cells, to accommodate large transgenes, and to code for proteins without integrating into the host cell genome.

In some embodiments, an AAVP vector is used. The AAVP vector is a hybrid of prokaryotic-eukaryotic vectors, which are chimeras of genetic cis-elements of recombinant adeno-associated virus and phage. An AAVP combines selected elements of both phage and AAV vector systems, providing a vector that is simple to produce in bacteria with no packaging limit, while allowing infection of mammalian cells combined with integration into the host chromosome. Vectors containing many of the appropriate elements are commercially available, and can be further modified by standard methodologies to include the necessary sequences. Among other things, AAVPs do not require helper viruses or trans-acting factors. In addition, the native tropism of AAV for mammalian cells is eliminated since there is not AAV capsid formation. Other methods and details are in U.S. Pat. No. 8,470,528 and Hajitou A. et al., Cell, 125: 358-398, both of which are incorporated herein by reference.

In other aspects, a human papilloma (HPV) pseudovirus is used. Recent studies have shown that DNA plasmids can be packaged into papillomavirus L1 and L2 capsid protein to generate pseudovirion that can efficiently deliver DNA. The encapsulation protects the DNA from nucleases and provides a targeted delivery with great stability. Many of the safety concerns associated with the use of viral vectors are mitigated with the HPV pseudoviros because its construct is different from the natural HPV viral genome. Other methods and examples are in Hung, C., et al., Plos One, 7:7(e40983); 2012, U.S. Pat. No. 8,394,411, and Kines, R., et al Int J of Cancer, 2015, all of which are incorporated herein by reference.

In some aspects, an oncolytic virus is used. Oncolytic virus therapy selectively replicates the virus in cancer cells, and subsequently spreads within a tumor without affecting normal tissue. Alternatively, the oncolytic virus preferentially infects and kills cells without causing damage to normal tissues. Oncolytic viruses are also effective at inducing immune responses to themselves as well as to the infected tumor cell. Typically, oncolytic viruses fall into two classes: (I) viruses that naturally replicate preferentially in cancer cells and are nonpathogenic in humans. Exemplary class (I) oncolytic viruses include autonomous parvoviruses, myxoma virus (poxvirus), Newcastle disease virus (NDV; paramyxovirus), reovirus, and Seneca valley virus (picornavirus). A second class (II), include viruses that are genetically manipulated for use as vaccine vectors, including measles virus (paramyxovirus), poliovirus (picornavirus), and vaccinia virus (poxvirus). Additionally, oncolytic viruses may include those genetically engineered with mutations/deletions in genes required for replication in normal but not in cancer cells including adenovirus, herpes simplex virus, and vesicular stomatitis virus. Oncolytic viruses can be used as a viral transduction method due to their low probability of genetic resistance because they can target multiple pathways and replicate in a tumor-selective method. The viral dose within the tumor can increase with time due to in situ viral amplification (as compared to small molecule therapies which decrease with time), and safety features can be built in (i.e., drug and immune sensitivity).

### Integration

In some aspects of the invention, the nucleic acids encoding cellular therapy target(s) or tumor-associated antigen(s) are integrated as part of the tumor cells' genetic makeup. Without being bound by theory, integration of the nucleic acid encoding cellular therapy target(s) can be helpful in that as the tumor cell replicates, progeny tumor cells would express the cellular therapy target(s) and, as such, be susceptible to the effector cells and other therapeutic agents (including combination therapy agents). It follows that in some indications, such as metastatic disease, the state of rapid tumor cell proliferation becomes useful in propagating the therapeutics of the present invention.

In one embodiment, integration can be achieved by using viruses that naturally integrate into the host cell. Integration is a crucial step in replication of retroviruses as it is a virus genome that has been integrated into the DNA of a host cell. Integration is not part of the viral replication cycle, but it can occasionally occur. The virus does not directly make new DNA copies of itself while integrated into the host genome; alternatively, it is passively replicated along with the host genome and passed on to the original cell's offspring. Integration of the viral DNA results in permanent insertion of the viral genome into the host chromosomal DNA, referred as a provirus in the case of retroviruses.

In other embodiments, integration can be achieved by commercially available kits, including the CompoZr® Targeted Integration Kit which is designed to integrate a gene of interest into the adeno-associated virus integration site 1 (AAVS1) on human chromosome 19.

In addition to the expression of TAA, the cancer cells (e.g., solid tumor cells) can be engineered so that they also express one or more fusion proteins of an antibody or antibody fragment (e.g., scFv) coupled to an antigen that an effector cell will recognize. The antibody or antibody fragment recognizes the tumor cell and binds and presents the antigen for an effector cell to recognize. In one non-limiting example, a solid tumor cell can be transduced with a fusion protein that includes an anti-tumor TAA scFv that binds to a tumor cell, and also part or all of the human CD19 protein. The scFv portion binds to the tumor cell and the CD19 is presented to effector cells for them to target the tumor cell and destroy the tumor cell.

In another embodiment, the cancer cells (e.g., solid tumor cells) can be engineered so that they also express one or more fusion proteins of an antibody or antibody fragment (e.g., scFv) coupled to an anti-idiotype antibody that will recognize an effector cell. The antibody or antibody fragment recognizes the tumor cell and binds and presents the anti-idiotype antibody that binds an antigen binding portion of one or more cellular therapeutics (e.g., and scFv of a CAR-T cell). In one non-limiting example, a solid tumor cell can be transduced with a fusion protein that is an “scFv/anti-idiotype scFv” fusion protein that includes (i) an scFv that binds a tumor antigen (as described herein) at the N-terminus and (ii) an anti-idiotype scFv that binds to an antigen binding portion at the C-terminus. In some embodiments, a fusion protein is an “anti-idiotype scFv/scFv” fusion protein that includes (i) an anti-idiotype scFv that binds to an antigen binding portion at the N-terminus, and (ii) an scFv that binds a tumor antigen (as described herein) at the C-terminus.

In some such embodiments, after being expressed, such fusion protein is secreted from a tumor and can bind on or near a tumor cell via its anti-tumor antibody or fragment (e.g., scFv). Upon subsequent treatment with an additional cellular therapeutic (e.g., CAR-T cell), the fusion protein (bound to a tumor antigen) binds to such additional cellular therapeutic via its idiotope-binding protein (e.g., via its anti-idiotype antibody that recognizes an antigen binding receptor of a CAR-T cell). In some embodiments the antigen binding receptor binds a B cell specific marker. In some embodiments, a B cell specific marker is a B cell antigen. In some embodiments, a B cell specific marker is a neoantigen and/or an antigen expressed by a B cell lineage cancer cell. For example, a fusion protein can include (i) an scFv that binds to a tumor antigen and (ii) an anti-idiotype antibody (e.g., anti-idiotype scFv) that binds to a B-cell antigen binding domain (e.g., a CAR that binds CD19, CD20, CD21, CD22, CD24, CD79a, CD79b, ROR1, or BCMA) of a CAR-T cell. Additionally, for example, a fusion protein can include (i) an scFv that binds to a tumor antigen and (ii) an anti-idiotype antibody (e.g., anti-idiotype scFv) that binds to an anti-CD19 scFv on a CD19 CAR-T cell.

### Expressed Gene Approach for Solid Tumor Targeting

Genes productively introduced into tumor cells will provide improved cellular therapeutic activity by addressing or circumventing critical barriers to efficacy. These genes will improve therapeutic efficacy by “propagating” the anti-tumor response, optimize cytokine support in the local environment, reverse local immunosuppression, improve cellular effector functions, and promote cellular access to tumors. Any gene can be included in an expressed construct as described herein, and the present disclosure is not limited to any particular gene. Exemplary, non-limiting types of genes that can be included as cellular therapy targets include, e.g., targets for additional cellular therapeutics, polypeptide antigens, antibodies, cytokines, agents targeting tumor microenvironment, and agents supporting immune cell growth/proliferation.

An expressed gene contains a promotor with its associated elements, and a gene sequence. An expressed gene contains three essential characteristics: 1) an optimal promoter for expression in the tumor cell, 2) an optimal expressed gene sequence, and 3) an optimized expression pattern with defined kinetics. A set of promoters is developed to drive diverse expression patterns. Examples include; rapid and sustained expression, measured in days, or rapid but reversible expression, or delayed expression. Also, the level of expression can be modified by selective use of regulatory and promoter elements. Such methods are well understood, for example, E.D. Papadakis, et al., Current Gene Therapy, 4: 89-113 (incorporated herein by reference).

### Ill. Methods for Treating Cancer

The invention provides for compositions and methods for treating cancer by engineering a system whereby the cancer cells (e.g., tumor cells or solid tumor cells) secrete fusion proteins that include TAA or TSA and a therapeutic agent. An individual having cancer or suspected of having cancer can be given a composition that allows for the in vivo transduction of their cancer cells. The administration can be by any means, including, but not limited to intravenously, systemically, intramuscularly, intraperitonally, or intra-tumoral injection.

### Targets for Additional Cellular Therapeutics

In some embodiments, the cell therapy antigen that is expressed on local tumor cells following transduction and secretion of fusion proteins is recognized by the effector cells, and can comprise a tumor associated antigen (TAA). In one embodiment, TAA expression can be restricted to the tumor cell population alone, expressed by all tumor cells, and expressed on the tumor cell surface. Other antigens are overexpressed on tumor cells, but may be found on normal cells at lower levels of expression and thus are tumor-selective antigens (TSA). In addition, some tumor antigens arise as “passenger mutations”, i.e. are non-essential antigens expressed by tumor cells that have defective control over DNA repair, thus accumulating mutations in diverse proteins. Some tumor antigens are proteins that are produced by tumor cells that elicit an immune response; particularly T-cell mediated immune responses. Tumor-specific molecules that may be targeted by cellular therapy targets may include tumor associated antigens well known in the art and can include, for example, a glioma-associated antigen, carcinoembryonic antigen (CEA), β-human chorionic gonadotropin, alphafetoprotein (AFP), lectin-reactive AFP, thyroglobulin, RAGE-1 , MN-CA IX, human telomerase reverse transcriptase, RU1 , RU2 (AS), intestinal carboxyi esterase, mut hsp70-2, M-CSF, prostase, prostate-specific antigen (PSA), PAP, NY-ESO- 1 , LAGE-la, p53, prostein, PSMA, Her2/neu, survivin and telomerase, prostate-carcinoma tumor antigen- 1 (PCTA-1), MAGE, ELF2M, neutrophil elastase, ephrinB2, CD22, insulin growth factor (IGF)-I, IGF-II, IGF-I receptor and mesothelin. The application of technologies, including next generation sequencing (NGS) of tumor genomes and exomes, and high-sensitivity mass spectrometry (protein sequencing) analysis of the tumor proteome, is continuing to identify novel TAA and TSA antigens of use for this invention.

NGS is a method of high-throughput sequencing that performs massively parallel sequencing, during which millions of fragments of DNA from a single sample are sequenced in unison. NGS facilitates high-throughput sequencing, which allows an entire genome to be sequenced in less than one day. The creation of NGS platforms has made sequencing accessible to more labs, rapidly increasing the amount of research and clinical diagnostics being performed with nucleic acid sequencing, and thus have revolutionized genomics and molecular biology. Some NGS technologies include Illumina (Solexa) sequencing, Roche 454 sequencing, Ion torrent: proton/PGM sequencing, and SOLiD sequencing.

An alternative method for identifying tumor specific antigens is direct protein sequencing. Protein sequencing of enzymatic digests using multidimensional MS techniques (MSn) including tandem mass spectrometry (MS/MS)) can also be used to identify antigens. Such proteomic approaches permit rapid, highly automated analysis (See, e.g., K. Gevaert and J. Vandekerckhove, Electrophoresis 21: 1145- 1154 (2000, incorporated herein by reference)). Furthermore, high-throughput methods for de novo sequencing of unknown proteins may be used to analyze the proteome of a patient's tumor to identify expressed antigens. For example, meta shotgun protein sequencing may be used to identify expressed antigens (See e.g., Guthals et al. (2012), Molecular and Cellular Proteomics 11(10): 1084-96, incorporated herein by reference).

Non-limiting examples of tumor antigens that can used include EphA2, HER2, GD2, Glypican-3, 5T4, 8H9, αvβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-Al MAGE Al, HLA-A2, ILl IRa, IL13Ra2, KDR, Lambda, Lewis-Y, MCSP, Mesothelin, Mucl, Mucl6, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, and VEGF receptors. Other exemplary antigens that can be used are antigens that are present with in the extracellular matrix of tumors, such as oncofetal variants of fibronectin, tenascin, or necrotic regions of tumors.

Additional tumor-selective molecules can be used include any membrane protein or biomarker that is expressed or overexpressed in tumor cells including, but not limited to, integrins (e.g., integrin αbβ3, α5β1), EGF Receptor Family (e.g., EGFR2, Erbb2/HER2/neu, Erbb3, Erbb4), proteoglycans (e.g., heparan sulfate proteoglycans), disialogangliosides (e.g., GD2, GD3), B7-H3 (aka CD276), cancer antigen 125 (CA- 125), epithelial cell adhesion molecule (EpCAM), vascular endothelial growth factor receptors 1 and 2 (VEGFR-1 , VEGFR-2), CD52, carcinoembryonic antigen (CEA), tumor associated glycoproteins (e.g., TAG-72), cluster of differentiation 19 (CD19), CD20, CD22, CD30, CD33, CD40, CD44, CD74, CD152, mucin 1 (M UC1), tumor necrosis factor receptors (e.g., TRAIL-R2), insulin-like growth factor receptors, folate receptor a, transmembrane glycoprotein NMB (GPNMB), C-C chemokine receptors (e.g., CCR4), prostate specific membrane antigen (PSMA), recepteur d'origine nantais (RON) receptor, cytotoxic T-lymphocyte antigen 4 (CTLA4), and other tumor specific receptors or antigens.

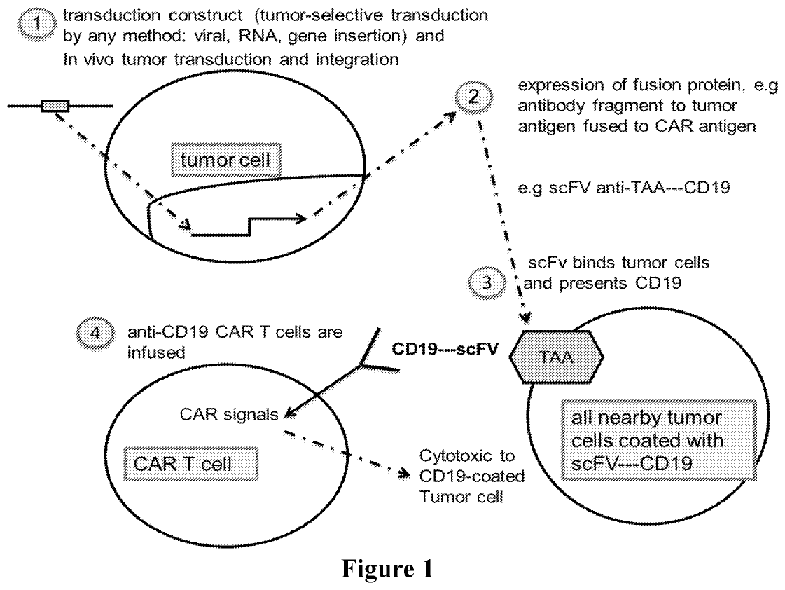

As exemplified by the non-limiting examples of TAA and TSA targets recited herein, there is a large and diverse selection of antigens to which one of skill in the art can direct the transduced and expressed therapeutics described herein. For example, the therapeutics might comprise a secreted fusion protein that contains a scFv antigen binding domain (e.g. anti-MUC16, anti-CEA, anti-PSMA) fused to the target antigen (e.g. CD19) that can be recognized by a CAR T cell. In this embodiment, the secreted fusion protein has two functional domains, the scFv that binds to the target tumor cell surface, and the CD19 protein domain that is presented as a target for the CAR T cell (FIG. 1). It will be immediately apparent that the scFv can be selected to target one of many diverse antigens, and that the protein domain to be recognized by the cellular therapeutic (also, the “effector cell” of the present invention), can also be diverse, e.g. recognized by a specific CAR T cell, or a TCR T cell, or a characterized TIL or an NK cell. It will be further recognized that modern antibody engineering allows the use of bispecific recognition to be built into the scFv portion of the therapeutic, such that effective binding to the tumor cells is accomplished only when both arms of a bispecific scFV can bind. The range of bispecific technologies available is broad and the molecular biology and protein chemistry tools and principles required for effective bispecific antibody engineering are well understood, see for example, Kontermann, R. MAbs 2012; 4(2) 182-97 (incorporated herein by reference).

Further, multiple genes can be encoded in a single CAR expression construct by using, for example, in frame or independent IRES initiation sites for individual elements. Alternatively, inducible methods have been described, whereby the application of a small molecule can induce or block expression of one or more CAR elements. A wide variety of such methods are disclosed, such as inhibitory CARs, costimulatory CARs, “cideCARs”, on switches and others, the use of which can further modify or alter the activity of CART cells (see Baas, T. SciBX 7(25); doi:10.1038/scibx.2014.725).

### Antibody-Drug Conjugate Targets

In other embodiments, the transduced tumor cells secrete fusion proteins that are targeted by antibody-drug conjugates that are known and include, e.g., brentuximab vedotin (ADCETRIS, Seattle Genetics); trastuzumab emtansine (Roche); Gemtuzumab ozogamicin (Pfizer); CMC-544; SAR3419; CDX-011; PSMA-ADC; BT-062; CD30, HER2, and IMGN901 (see, e.g., Sassoon et al., Methods Mol. Biol. 1045:1-27 (2013); Bouchard et al., Bioorganic Med. Chem. Lett. 24: 5357-5363 (2014)). In other embodiments, the transduced tumor cells secrete fusion proteins which can be targeted by antibodies having antibody dependent cellular cytotoxicity function such as those recognized by rituximab, ocrelizumab, ipilimumab, cituximab, erbitux and many others. Accordingly, in some embodiments, a nucleic acid encoding a polypeptide antigen as part of the fusion protein that binds to one or more of such known antibody-drug conjugates can be included in as a cellular therapy target described herein.

### Cytokine Fusion Proteins

In some embodiments, the tumor and/or tumor microenvironment is transduced with a cytokine fusion protein, e.g., a fusion protein of a cytokine (e.g., an anti-tumor cytokine) and a target for one or more additional cellular therapeutics described herein (e.g., a CAR-T target). Exemplary cytokines may bind to the tumors, and present the tumor with the cytokine. For example, some contemplated targets include CD19, CD20, CD21, CD22, CD24, and BCMA. Such a cellular therapy can provide both a target for one or more additional cellular therapeutics (e.g., a CAR-T target) and a stimulatory cytokine at a tumor surface. For example, an expressed and/or secreted construct can encode a cytokine-CD19 fusion protein, or a fusion of a cytokine and a CD19 fragment, e.g., a CD19 fragment to which a CD19- CAR-T cell binds. In some embodiments, a CD19 fragment is a CD19 IgC domain. Without wishing to be bound by theory, a single expressed construct encoding such a fusion protein advantageously allows a cellular therapeutic to be genetically engineered using a minimal (e.g., a single) transgene. An additional benefit of using cytokine fusion proteins is to utilize their tight binding to their receptors, in addition to the cytokine functional effect.

In some embodiments, one or more cytokines secreted as part of a cytokine fusion protein bind to cells at high affinity (e.g., KD of about 10⁻⁷, 10⁻⁸, 10⁻⁹, 10⁻¹⁰, 10⁻¹¹, or less) and/or have low internalization rates (e.g., less than about 10, 10², 10³, 10⁴, or 10⁵ cytokine molecules per cell per day). Binding affinity and internalization rates of various cytokines are known in the art and/or can be measured using known methods.

### Pro-Immune Response Agents

In some embodiments, the recombinant tumor described herein encodes, as a fusion protein as described herein, one or more pro-immune response agents, e.g., one or more cytokines used in cancer therapy. Non-limiting, exemplary cytokines that can be included include, e.g., IFNα, IFNβ, IFNγ, IL-1, IL-2, IL-7, IL-12, IL-15, IL-21, IL-36, TNF, LTα, GM-CSF, G-CSF, a TLR agonist, and an immune checkpoint antibody fragment.

Known problems associated with cytokine therapy include, e.g., high dose requirements, toxicity, and limited efficacy. Thus, in some embodiments, expressed constructs are used to deliver one or more cytokines at a specific site and/or at a specific dose (e.g., to reduce or eliminate one or more risks associated with cytokine therapy).

In some embodiments, expression of a cytokine (e.g., an immunostimulatory cytokine) at or near a surface of a tumor induces an immune response to the tumor. In some embodiments, an expressed cytokine fusion protein can be a target for one or more additional cellular therapeutics (e.g., one or more additional CAR-T cells). In some embodiments, secretion of a cytokine fusion protein near a surface of a tumor induces an immune response to the tumor and is also used as a target for one or more additional cellular therapeutics (e.g., one or more additional CAR-T cells). An example is an Interferon alpha cytokine fused to the CD19 protein domain recognized by a CAR T cell with CD19 reactivity. The IFNalpha molecule binds with high affinity to interferon receptors on or near the tumor cell, thus supporting cellular therapeutic activity directed to CD19. In another example, the interferon alpha cytokine is fused to a scFV that recognizes a TAA or a TSA on the same tumor cell type, thus binding back on to the cell and surrounding cells, and inducing or supporting an IFNalpha-driven immune response.

For example, release of IL-21 can be used to induce expansion and/or effector differentiation of CD8+ T cells and/or support NK cell activation and cytolytic activity. In one exemplary method, an expressed construct encodes IL-21. Upon binding of a scFv directed an antigen on a tumor cell, a cellular therapeutic described herein exhibits prolonged release of IL-21. Exemplary cellular therapeutics include, e.g., CAR-T cells, CAR-NK cells, TCR-T cells, TIL cells, allogenic NK cells, and autologous NK cells.

In another exemplary method, induced release of IL-15 fusion proteins can be used to support NK cell expansion and/or to recruit NK cells to promulgate an anti-tumor response and to support the survival and expansion of cellular therapeutics. Exemplary cellular therapeutics include, e.g., CAR-T cells, CAR-NK cells, TCR-T cells, TIL cells, allogenic NK cells, and autologous NK cells. In this example a scFv that recognizes a TAA or a TSA on the target tumor cell type binds, and presents IL-15 in the local environment. In a further exemplification, of relevance to all of the cytokine examples proposed, the secreted fusion protein is trifunctional, having a scFV that recognizes a TAA or a TSA on the target tumor cell type, a cytokine encoded into a beta loop or beta strand within the heavy or light chain variable region that is not engaged in antigen binding, and expressing a target antigen for the binding of a cellular therapeutic. The utilization of CDRs within the heavy and light variable domains of a scFv is readily determined from the sequence of the CDR as well as through the use of databases that indicate which residues are involved in antigen engagement and which are not involved but are otherwise solvent exposed, i.e. useful for expression of an encoded sequence such as a cytokine.

### Cell Recruiting Moieties

In some examples, the recombinant tumor cells can express, as part of a fusion protein, scFv or TCR that may be fused to cell recruiting moieties (e.g., anti-CD3, anti-CD16, or an anti-idiotype antibody or fragment). In other embodiments, bispecific T cell engager (BiTE®) technology is utilized to help engage the body's endogenous T cells and to target cancer cells that have been engineered to express one or more TAA's. The antibody constructs of the BiTE® technology are constructed by genetically linking the minimal binding domains of monoclonal antibodies for TAA or tumor-associated surface antigens and for the T cell receptor-associated molecule, onto a single polypeptide chain. One antibody is specific for a selected surface antigen on a targeted tumor cell, and the other antibody is specific for moiety (e.g., CD3), tied to the T-cell receptor complex on the surface of T cells. The BiTE® technology binds polyclonal cytotoxic T cells and targeted malignant cells.

### Polypeptide Antigens

In some embodiments, a target for one or more additional cellular therapeutics is or comprises a polypeptide antigen. The polypeptide antigen to be expressed by an expressed construct, and is not limited to any particular polypeptide or portion thereof, provided that an additional cellular therapeutic (e.g., CAR-T cell) can be engineered to recognize and bind to such polypeptide target. In some embodiments, the polypeptide target is a polypeptide that is not a tumor-associated antigen. In some embodiments, the target is a tumor antigen, e.g., BCMA, CD19, CD20, CD22, ROR1, mesothelin, CD33/IL3Ra, c-Met, PSMA, Glycolipid F77, EGFRvIII, GD-2, NY-ESO-1 TCR, or MAGE A3 TCR.

In some embodiments, the tumor and/or tumor microenvironment is transduced with a fusion protein comprising (i) an antibody or antigen-binding fragment thereof that binds to a tumor antigen described herein and (ii) an “anti-idiotype” peptide that binds an antigen binding receptor of one or more additional cellular therapeutics (e.g., an scFv of a CAR-T cell). In some embodiments, an anti-idiotype peptide that binds an antigen binding receptor of one or more additional cellular therapeutics binds one or more CDRs of an antigen binding receptor (e.g., an scFv of a CAR-T cell). In some embodiments, a fusion protein includes (i) an scFv that binds a tumor antigen (as described herein) at the N-terminus and (ii) an anti-idiotype peptide that binds to an antigen binding receptor (described herein) at the C-terminus. In some embodiments, a fusion protein includes (i) an anti-idiotype peptide that binds to an antigen binding receptor (described herein) at the N-terminus, and (ii) an scFv that binds a tumor antigen (as described herein) at the C-terminus.

One of skill in the art will recognize that several methods can be used to identify peptides that bind to antibodies or fragments thereof (e.g., scFvs or CDRs). Exemplary methods include screening or panning peptide libraries. For example, peptides that bind rituximab, an anti-CD20 antibody, have been identified (Klein et al. mABs 5:1, 22-33 January/February 2013; Philip et al. Blood. 2014 Aug 21;124(8):1277-87; Perosa et al. J Immunol 2007; 179: 7967-7974; Perosa et al. Blood. 2006 Feb 1;107(3):1070-7). In some embodiments, peptides that bind antibodies can be identified through the use of phage display libraries (see, e.g., Smith Science. 1985 Jun 14;228(4705):1315-7; Scott et al. Science. 1990 Jul 27;249(4967):386-90; Mintz et al. Nat Biotechnol. 2003 Jan;21(1):57-63; Spatola et al. Anal Chem. 2013; Rojas et al. MAbs. 2014;6(6):1368-76; Wang et al. Oncotarget. 2016 Nov 15;7(46):75293-75306; He et al. Virology Journal 2012, 9:217; Li et al. PLoS One. 2016 May 18;11(5):e0147361; de Oliveira-Junior et al. Biomed Res Int. 2015;2015:267989). In some embodiments, peptides that bind antibodies can be identified through screens of peptide libraries displayed on organisms other than phage (for example bacteria, see, e.g., US Pat. 9,309,510). In some embodiments, peptides that bind antibodies can be identified through other peptide libraries, for example, soluble peptide libraries (e.g., positional scanning libraries; see, for example, Pinilla et al. Biochem J. (1994) 301, 847-853), DNA-encoded cyclic libraries, etc. Any of such peptides can be used as an “anti-idiotype” peptide in methods and constructs described herein.

In some embodiments, after being expressed, such fusion protein is secreted from a transduced cell and can bind on or near a tumor cell via its anti-tumor antibody or fragment (e.g., scFv). Upon subsequent treatment with an additional cellular therapeutic (e.g., CAR-T cell), the fusion protein (bound to a tumor antigen) binds to such additional cellular therapeutic via its anti-idiotype peptide (e.g., that recognizes an antigen binding receptor of a CAR-T cell). For example, a fusion protein can include (i) an scFv that binds to a tumor antigen and (ii) an anti-idiotype peptide that binds to a B-cell specific marker binding domain (e.g., a CAR that binds CD19, CD20, CD21, CD22, CD24, CD79a, CD79b, ROR1, or BCMA) of a CAR-T cell. In some embodiments, a fusion protein can include (i) an scFv that binds to a tumor antigen and (ii) an anti-idiotype peptide that binds to an anti-CD19 scFv on a CD19 CAR-T cell.

### Antibody Fusion Proteins

In some embodiments, the tumor and/or tumor microenvironment is transduced with a fusion protein comprising (i) an antibody or antigen-binding fragment thereof that binds to a tumor antigen described herein and (ii) an anti-idiotype antibody or fragment that binds an antigen binding portion of one or more additional cellular therapeutics (e.g., an scFv of a CAR-T cell). In some embodiments, a fusion protein is an “scFv/anti-idiotype scFv” fusion protein that includes (i) an scFv that binds a tumor antigen (as described herein) at the N-terminus and (ii) an anti-idiotype scFv that binds to an antigen binding portion at the C-terminus. In some embodiments, a fusion protein is an “anti-idiotype scFv/scFv” fusion protein that includes (i) an anti-idiotype scFv that binds to an antigen binding portion at the N-terminus, and (ii) an scFv that binds a tumor antigen (as described herein) at the C-terminus.

In some such embodiments, after being expressed, such fusion protein is secreted from a tumor and can bind on or near a tumor cell via its anti-tumor antibody or fragment (e.g., scFv). Upon subsequent treatment with a cellular therapeutic (e.g., CAR-T cell), the fusion protein (bound to a tumor antigen) binds to such cellular therapeutic via its idiotope-binding protein (e.g., via its anti-idiotype antibody that recognizes an antigen binding portion of a CAR-T cell).₌In some embodiments the antigen binding receptor binds a B cell specific marker. In some embodiments, a B cell specific marker is a B cell antigen. In some embodiments, a B cell specific marker is a neoantigen and/or an antigen expressed by a B cell lineage cancer cell. For example, a fusion protein can include (i) an scFv that binds to a tumor antigen and (ii) an anti-idiotype antibody (e.g., anti-idiotype scFv) that binds to a B-cell specific marker binding domain (e.g., a CAR that binds CD19, CD20, CD21, CD22, CD24, CD79a, CD79b, ROR1, or BCMA) of a CAR-T cell. In some embodiments, a fusion protein can include (i) an scFv that binds to a tumor antigen and (ii) an anti-idiotype antibody (e.g., anti-idiotype scFv) that binds to an anti-CD19 scFv on a CD19 CAR-T cell.

Anti-idiotype antibodies are specific antibodies that can bind to the CDR sequences within a specific antibody of an antibody's scFv. Anti-idiotype antibodies can be characterized by their binding. Type 1 anti-idiotype antibodies bind to the CDRs of a target antibody variable domain in such a manner as to inhibit, disrupt or neutralize the activity of the target antibody, i.e., its ability to bind antigen. Type 2 anti-idiotype antibodies bind to the CDRs of the target antibody variable domains in such a manner as to be able to bind even when the antibody is bound to antigen. Thus Type 2 antibodies are not defined by their ability to inhibit or neutralize antigen binding. A Type 3 anti-idiotype antibody only binds a target antibody when is bound to antigen.

Anti-idiotype antibodies are known in the art, and any such antibody is useful in compositions and methods described herein. One example of a specific anti-idiotype antibody specific for an antibody scFv is antibody 136.20.1, which recognizes the scFv domain of the mouse anti-human antibody FMC63 (see, e.g., Jena B, et al. (2013) Chimeric Antigen Receptor (CAR)-Specific Monoclonal Antibody to Detect CD19-Specific T Cells in Clinical Trials. PLoS ONE 8(3): e57838; US 2016/0096902). The 136.20.1 antibody and its domains (e.g., the scFv domain), have been used to detect the FMC63 VH/VL pair, or scFv, e.g., as displayed on the surface of a CAR T cell. However, the 136.20.1 antibody has not previously been presented to an FMC63-based CAR T cell as a means of triggering CAR T activity. Indeed, in the scFv or similar monovalent format, 136.20.1 antibody triggering CART activity would not be expected. It has been shown that 136.20.1 binds to the antigen (CD19) recognition site of FMC63, since at concentrations above 5μg/mI136.20.1 inhibits binding of the FMC63 CART cell to CD19.

Another example is an anti-idiotype antibody that recognizes an anti-human CD22 scFv (as described in, e.g., Zhoa et al. 2014. Generation of Anti-Idiotype scFv for Pharmacokinetic Measurement in Lymphoma Patients Treated with Chimera Anti-CD22 Antibody SM03. PLoS ONE 9(5): e96697; US 2015/0175711). One such antibody is an anti-idiotype single-chain Fv (scFv) antibody specific for the murine (RFB4), chimeric (SM03) and humanized (SM06) versions of an anti-CD22 antibody that has the characteristics of a Type 1 anti-idiotypic antibody, that is, it binds specifically to the CDRs of the named anti-CD22 antibodies and inhibits the binding of the named antibodies to human CD22 protein. A Type 2 idiotypic antibody that specifically recognizes rituximab (a mouse-derived antibody to human CD20) has also been described (see Cragg et al. (2004) An anti-idiotype antibody capable of binding rituximab on the surface of lymphoma cells. Blood 104: 2540-2542).

Other examples include anti-idiotypic antibodies described by Dunn & Kehry in US 2013/0330323 A1. Other examples include myriad anti-idiotypic antibodies published and described. Other examples include novel anti-idiotypic antibodies as discovered in directed screening campaigns using the target antibody or scFv protein as immunogen or screening reagent.

In some embodiments, the tumor and/or tumor microenvironment is transduced with a fusion protein comprising an antibody (or antigen-binding fragment thereof, e.g., secreted scFv or other antibody formats) and a target for one or more additional cellular therapeutics (e.g., a CAR-T target). An antibody (or fragment) can be selected to bind, e.g., to a tumor antigen (e.g., a tumor antigen described herein), and its fusion partner can include a target for one or more additional cellular therapeutics. Such antibodies (or antigen-binding fragments) include, e.g., a monoclonal antibody (mAb), including, for example, scFv and full length mAbs, a VHH domain, a diabody, a nanobody, etc. In one example, a construct encodes a secreted fusion protein consisting of a mAb (e.g., an anti-tumor associated antigen mAb or antigen-binding fragment) and CD19 or a fragment thereof (e.g., a CD19 Ig domain). In some embodiments, an antibody (or fragment) binds to an antigen expressed on several types of cells. In some embodiments, an antibody (or fragment) binds to a tumor-selective antigen. In some embodiments, an antibody (or fragment) binds to a tumor-selective, but not specific, antigen. In some embodiments, an antibody (or fragment) binds to a tumor antigen associated with a hematologic malignancy. In some embodiments, an antibody (or fragment) binds to a tumor antigen associated with a solid tumor. In some embodiments, an antibody (or fragment) binds to one or more of BCMA, CD19, CD20, CD22, ROR1, mesothelin, CD33/IL3Ra, c-Met, PSMA, Glycolipid F77, EGFRvIII, GD-2, NY-ESO-1 TCR, and MAGE A3 TCR.

The target, which is placed on the tumor cells, can be CD19. Other B cell targets can be used, including but not limited to CD20, CD21, CD22, CD23, CD24, CD72, CD79a, CD79b, and BCMA. These B cell targets have particular advantages as CAR T cell targets, along with the list of other targets. In addition, the target can include CD30, Her 2, a target for ADC's or radioimmunotherapy (e.g., a monoclonal antibody carrying a radioisotope).

Peptides that Inhibit Local (e.g., Tumor Microenvironment) Factors

In some embodiments, peptides (e.g., polypeptides and fragments thereof) that inhibit local factors can be expressed as a fusion protein by the tumor cell. Nucleic acids coding for these peptides can be engineered as described herein and/or by any method known to one of skill in the art such that the peptide(s) are expressed. Non-limiting examples include TGFbeta, adenosine receptor 2, vascular endothelial growth factor (VEGF), platelet-derived growth factor (PDGF), indoleamine 2,3-dioxygenase 1 (ID01), and matrix metalloproteinases (MMPs)).

### CD19 as a Scaffold for Inducible CD19 Variant Proteins and CD19 Variant Fusion Proteins

CD19 is a 95 kd transmembrane glycoprotein belonging to the Ig superfamily and includes two extracellular C2-type Ig domains (see, e.g., Tedder Nature Rev. Rheum. 5:572-577 (2009); Wang et al., Exp. Hematol. Oncol. 2012 Nov 29;1(1):36. doi: 10.1186/2162-3619-1-36.)). In some embodiments, one or both of the C2-type Ig domains are used as scaffolds for mutagenesis, and CD19 variants (e.g., CD19 or a portion thereof that include one or more mutations within one or both C2-type Ig domains) can be screened and selected for binding to a target antigen described herein.

The nucleotide sequence of human CD19 is known (see Genbank Accession No. M84371.1). To provide variant nucleic acid sequences that encode CD19 variants that bind a particular antigen, a number of methods known in the art may be utilized. In some embodiments, a screening procedure is used that enables identification and/or isolation of nucleic acids that encode CD19 variants that bind a particular antigen. Exemplary methods include a so-called biopanning step, known from technologies such as phage display (Kang, A. S. et al. 1991. Proc Natl Acad Sci USA 88, 4363-4366), ribosome display (Schaffitzel, C. et al. 1999. J. Immunol. Methods 231, 119-135), DNA display (Cull, M. G. et al. 1992. Proc Natl Acad Sci USA 89, 1865-1869), RNA-peptide display (Roberts, R. W., Szostak, J. W., 1997. Proc Natl Acad Sci USA 94, 12297-12302), covalent display (WO 98/37186), bacterial surface display (Fuchs, P. et al. 1991. Biotechnology 9, 1369-1372), yeast surface display (Boder, E. T., Wittrup, K. D., 1997. Nat Biotechnol 15, 553-557) and eukaryotic virus display (Grabherr, R., Ernst, W., 2001. Comb. Chem. High Throughput. Screen. 4, 185-192). FACS and magnetic bead sorting are also applicable for enrichment (panning) purposes using labeled antigen. Immunodetection assays such as ELISA (Dreher, M. L. et al. 1991. J. Immunol. Methods 139, 197-205) and ELISPOT (Czerkinsky, C. C. et. al. 1983. J Immunol Methods. 65, 109-21) can also be used either following a biopanning step or alone.

Thus, in some embodiments, an inducible construct described herein encodes a CD19 variant (or fragment), either alone or as part of a fusion protein described herein. For example, an inducible construct described herein can encode a CD19 variant (or fragment) selected to bind to a tumor agent and which, upon expression, can bind to the tumor antigen and that itself can be a target for an additional cellular therapeutic (e.g., a CAR-T cell that binds CD19). In some embodiments, an inducible construct described herein encodes a CD19 variant that includes a C2-type Ig domain variant selected to bind a tumor antigen. Upon expression of the CD19 variant, the C2-type Ig domain binds to the tumor antigen on a tumor cell. Subsequently, treatment with (e.g., administration to a subject of) a CAR-T cell that recognizes CD19 kills the tumor cell to which the CD19 variant is bound. In some embodiments, an inducible construct described herein encodes a CD19 variant that includes variants of both C2-type Ig domains, each of which is selected to bind a tumor antigen (e.g., different epitopes of the tumor antigen). Upon expression of the CD19 variant, the C2-type Ig domains bind to the tumor antigen on a tumor cell. Subsequently, treatment with (e.g., administration to a subject of) a CAR-T cell that recognizes CD19 kills the tumor cell to which the CD19 variant is bound.

In some embodiments, a CD19 variant selected for binding to a target antigen is included in a fusion protein. For example, a CD19 variant that includes a C2-type Ig domain variant selected to bind a tumor antigen can be fused to an antibody or fragment thereof that also binds to the tumor antigen (e.g., to a different epitope on the tumor antigen). Exemplary fusion proteins include, e.g., CD19 variant/scFv fusion proteins and CD19 variant/VHH fusion proteins. An inducible construct described herein can encode such a CD19 variant/antibody fusion protein and upon expression, the CD19 variant and the antibody of the fusion protein bind to the tumor antigen on a tumor cell. Subsequently, treatment with (e.g., administration to a subject of) a CAR-T cell that recognizes CD19 kills the tumor cell to which the CD19 variant/antibody fusion protein is bound. In some embodiments, as described herein, the CD19 scaffold genes that are useful in the context of inducible expression will also be useful when modified for the production in vitro of soluble, purified fusion proteins. Additional, non-limiting examples of fusion proteins that include CD19 variants (or fragment) as a scaffold include, e.g., CD19 variant /cytokine fusion proteins and CD19 variant/TLR agonist fusion proteins.

Additional, non-limiting examples of fusion proteins that include CD19 (or fragment) as a scaffold include, e.g., CD19-cytokine fusion proteins, CD19-TLR agonist fusion proteins. Other B cell restricted cell surface markers which contain immunoglobulin-like domains include CD22, CD79a and CD79b. These Ig domains can also be mutagenized to generate variants binding TAA's and TSA's.

In some embodiments, a CD19 variant selected for binding to a target antigen is included in a fusion protein with an anti-idiotype antibody or fragment described herein. For example, a CD19 variant that includes an ECD variant or C2-type Ig domain variant selected to bind a tumor antigen can be fused to an anti-idiotype antibody or fragment thereof that binds to an antibody or portion on a cellular therapeutic, e.g., CAR-T cell. An expression construct described herein can encode such a CD19 variant/anti-idiotype antibody fusion protein and upon expression, the CD19 variant of the fusion protein binds to the tumor antigen on a tumor cell. Subsequently, treatment with (e.g., administration to a subject of) a CAR-T cell that expresses an antibody or fragment recognized by the anti-idiotype antibody or fragment kills the tumor cell to which the CD19 variant/anti-idiotype antibody fusion protein is bound. In some embodiments, an expression construct described herein can encode one or more CD19 variants. For example, in some embodiments, a first CD19 variant that includes an ECD variant or C2-type Ig domain variant selected to bind a tumor antigen can be fused to a second CD19 variant that includes an ECD variant or C2-type Ig domain variant selected to bind an antibody or fragment expressed on a cellular therapeutic (e.g., CAR-T cell).

In some embodiments, a CD19 variant selected for binding to a target antigen is included in a fusion protein with an anti-idiotype peptide that binds an antigen binding receptor of one or more additional cellular therapeutics as described herein. For example, a CD19 variant that includes an ECD variant or C2-type Ig domain variant selected to bind a tumor antigen can be fused to an anti-idiotype peptide that binds to an antibody or portion on a cellular therapeutic, e.g., CAR-T cell. An expression construct described herein can encode such a CD19 variant/anti-idiotype peptide fusion protein and upon expression, the CD19 variant of the fusion protein binds to the tumor antigen on a tumor cell. Subsequently, treatment with (e.g., administration to a subject of) a CAR-T cell that expresses an antibody or fragment recognized by the anti-idiotype peptide kills the tumor cell to which the CD19 variant/anti-idiotype peptide fusion protein is bound.

### Polypeptide Antigens and Antibodies

The below Table 1 presents a non-comprehensive list of certain human polypeptide antigens targeted by known, available antibody agents, and notes certain cancer indications for which the antibody agents have been proposed to be useful:

TABLE 1

| | Antibody (commercial or | |
|---|---|---|
| Human Antigen | scientific name) | Cancer indication |
| CD2 | Siplizumab | Non-Hodgkin's Lymphoma |
| CD3 | UCHT1 | Peripheral or Cutaneous T-cell Lymphoma |
| CD4 | HuMax-CD4 | |
| CD19 | SAR3419, MEDI-551 | Diffuse Large B-cell Lymphoma |
| CD19 and CD3 or | Bispecific antibodies such as | Non-Hodgkin's Lymphoma |
| CD22 | Blinatumomab, DT2219ARL | |
| CD20 | Rituximab, Veltuzumab, | B cell malignancies (Non-Hodgkin's |
| | Tositumomab, Ofatumumab, | lymphoma, Chronic lymphocytic leukemia) |
| | Ibritumomab, Obinutuzumab, | |
| CD22 (SIGLEC2) | Inotuzumab, tetraxetan, CAT- | Chemotherapy-resistant hairy cell |
| | 8015, DCDT29805, Bectumomab | leukemia, Hodgkin's lymphoma |
| CD30 | Brentuximab vedotin | |
| CD33 | Gemtuzumab ozogamicin | Acute myeloid leukemia |
| | (Mylotarg) | |
| CD37 | TRU-016 | Chronic lymphocytic leukemia |
| CD38 | Daratumumab | Multiple myeloma, hematological tumors |
| CD40 | Lucatumumab | Non-Hodgkin's lymphoma |
| CD52 | Alemtuzumab (Campath) | Chronic lymphocytic leukemia |
| CD56 (NCAM1) | Lorvotuzumab | Small Cell Lung Cancer |
| CD66e (CEA) | Labetuzumab | Breast, colon and lung tumors |
| CD70 | SGN-75 | Non-Hodgkin's lymphoma |
| CD74 | Milatuzumab | Non-Hodgkin's lymphoma |
| CD138 (SYND1) | BT062 | Multiple Myeloma |
| CD152 (CTLA-4) | Ipilimumab | Metastatic melanoma |
| CD221 (IGF1R) | AVE1642, IMC-A12, MK-0646, | Glioma, lung, breast, head and neck, |
| | R150, CP 751871 | prostate and thyroid cancer |
| CD254 (RANKL) | Denosumab | Breast and prostate carcinoma |
| CD261 (TRAILR1) | Mapatumumab | Colon, lung and pancreas tumors and |
| CD262 (TRAILR2) | HGS-ETR2, CS-1008 | haematological malignancies |
| CD326 (Epcam) | Edrecolomab, 17-1A, IGN101, | Colon and rectal cancer, malignant ascites, |
| | Catumaxomab, Adecatumumab | epithelial tumors (breast, colon, lung) |
| CD309 (VEGFR2) | IM-2C6, CDP791 | Epithelium-derived solid tumors |
| CD319 (SLAMF7) | HuLuc63 | Multiple myeloma |
| CD340 (HER2) | Trastuzumab, Pertuzumab, Ado- | Breast cancer |
| | trastuzumab emtansine | |
| CAIX (CA9) | cG250 | Renal cell carcinoma |
| EGFR (c-erbB) | Cetuximab, Panitumumab, | Solid tumors including glioma, lung, breast, |
| | nimotuzumab and 806 | colon, and head and neck tumors |
| EPHA3 (HEK) | KB004, IIIA4 | Lung, kidney and colon tumors, melanoma, |
| | | glioma and haematological malignancies |
| Episialin | Epitumomab | Epithelial ovarian tumors |
| FAP | Sibrotuzumab and F19 | Colon, breast, lung, pancreas, and head and |
| | | neck tumors |
| HLA-DR beta | Apolizumab | Chronic lymphocytic leukemia, non- |
| | | Hodkin's lymphoma |
| FOLR-1 | Farletuzumab | Ovarian tumors |
| 5T4 | Anatumomab | Non-small cell lung cancer |
| GD3/GD2 | 3F8, ch14.18, KW-2871 | Neuroectodermal and epithelial tumors |
| gpA33 | huA33 | Colorectal carcinoma |
| GPNMB | Glembatumumab | Breast cancer |
| HER3 (ERBB3) | MM-121 | Breast, colon, lung, ovarian, and prostate |
| | | tumors |
| Integrin αVβ3 | Etaracizumab | Tumor vasculature |
| Integrin α5β1 | Volociximab | Tumor vasculature |
| Lewis-Y antigen | hu3S193, IgN311 | Breast, colon, lung and prostate tumors |
| MET (HGFR) | AMG 102, METMAB, SCH900105 | Breast, ovary and lung tumors |
| Mucin-1/CanAg | Pemtumomab, oregovomab, | Breast, colon, lung and ovarian tumors |
| | Cantuzumab | |
| PSMA | ADC, J591 | Prostate Cancer |
| Phosphatidylserine | Bavituximab | Solid tumors |
| TAG-72 | Minretumomab | Breast, colon and lung tumors |
| Tenascin | 81C6 | Glioma, breast and prostate tumours |
| VEGF | Bevacizumab | Tumour vasculature |

Accordingly, a cellular therapeutic that targets an expressed construct encoding a polypeptide antigen can be used in combination with one or more of these (or other) known antibodies.

Exemplary amino acid and nucleotide sequences of the disclosure are listed in the following Table:

| Amino acid SEQ ID NO: | Nucleotide SEQ ID NO: | Name |
|---|---|---|
| 1 | 201 | Panitumumab Heavy Chain (HC) |
| 2 | 202 | CD19-D1-Panitumumab Light Chain (LC) |
| 3 | 203 | CD19-D1-Panitumumab HC |
| 4 | 204 | Panitumumab LC |
| 5 | 205 | Panitumumab LC-CD19-D1 |
| 6 | 206 | Panitumumab HC-CD19-D1 |
| 7 | 207 | LY2875358 HC |
| 8 | 208 | CD19-D1-LY2875358 LC |
| 9 | 209 | CD19-D1-LY2875358 HC |
| 10 | 210 | LY2875358 LC |
| 11 | 211 | LY2875358 LC-CD19-D1 |
| 12 | 212 | LY2875358 HC-CD19-D1 |
| 13 | 213 | FMC63 CAR-19 construct |
| 14 | 214 | CD19-D1 |
| 15 | 215 | CD19-D1-hulgGFc |
| 16 | 216 | Trastuzumab scFv (VH/VL) |
| 17 | 217 | MOC31 scFv (VH/VL) |
| 18 | 218 | MOC31 scFv (VL/VH) |
| 19 | 219 | LY2875358 scFv (VH/VL) |
| 20 | 220 | LY2875358 scFv (VL/VH) |
| 21 | 221 | Panitumumab scFv (VH/VL) |
| 22 | 222 | Panitumumab scFv (VL/VH) |
| 23 | 223 | CD19-D1+2-soluble TNF |
| 24 | 224 | Trastuzumab scFv (VH/VL)-CD19-D1 |
| 25 | 225 | Trastuzumab scFv (VH/VL)-CD19-D1- |
| | | hulgGFc |
| 26 | 226 | CD19-D1-Trastuzumab scFv (VH/VL) |
| 27 | 227 | CD19-D1-Trastuzumab scFv (VH/VL)- |
| | | hulgGFc |
| 28 | 228 | CD19-D1+2 |
| 29 | 229 | CD19-D1+D2-hulgGFc |
| 30 | 230 | CD19-D2 |
| 31 | 231 | CD19-D2-hulgGFc |
| 32 | 232 | CD19-D1+2-Panitumumab LC |
| 33 | 233 | CD19-D1+2-Panitumumab HC |
| 34 | 234 | Panitumumab LC-CD19-D1+2 |
| 35 | 235 | Panitumumab HC-CD19-D1+2 |
| 36 | 236 | CD19-D1+2-LY2875358 LC |
| 37 | 237 | CD19-D1+2-LY2875358 HC |
| 38 | 238 | LY2875358 LC-CD19-D1+2 |
| 39 | 239 | LY2875358 HC-CD19-D1+2 |
| 40 | 240 | Trastuzumab scFv (VH/VL)-CD19-D1+2 |
| 41 | 241 | Trastuzumab scFv (VH/VL)-CD19-D1+2- |
| | | hulgGFc |
| 42 | 242 | CD19-D1+2-Trastuzumab scFv (VH/VL) |
| 43 | 243 | CD19-D1+2-Trastuzumab scFv (VH/VL)- |
| | | hulgGFc |
| 46 | 246 | human CD69 promoter-tGFP |
| 47 | 247 | human TNFalpha promoter-tGFP |
| 48 | 248 | mouse CD25 promoter-tGFP |
| 49 | 249 | NFAT element × 6 promoter-tGFP |
| 50 | 250 | CD19-ECD-Panitumumab HC |
| 51 | 251 | CD19-ECD-LY2875358 HC |
| 52 | 252 | CD19-ECD-MOC31 scFv (VH/VL) |
| 53 | 253 | CD19-ECD-LY2875358-scFv (VH/VL) |
| 54 | 254 | CD19-ECD-Panitumumab scFv (VH/VL) |
| 55 | 255 | CD19-ECD-Trastuzumab scFv (VH/VL) |
| 56 | 256 | CD19-ECD-hulgGFc-Trastuzumab scFv |
| | | (VH/VL) |
| 57 | 257 | Her2-ECD-Panitumumab scFv (VH/VL) |
| 58 | 258 | Her2-D4-Panitumumab scFv (VH/VL) |
| 63 | 263 | CD19-ECD-Leu16 scFv (VH/VL) |
| 64 | 264 | CD22-D123-FMC63 scFv (VH/VL) |
| 65 | 265 | CD22-D123-Leu16 scFv (VH/VL) |
| 66 | 266 | CMV promoter-tGFP |
| 67 | 267 | CD19-ECD-anti-EGFRvIII scFv (VL/VH) |
| 68 | 268 | CD22-D123-anti-EGFRvIII scFv (VH/VL) |
| 71 | 271 | FMC63 CAR-19 construct Flag-tagged-1 |
| 72 | 272 | FMC63 CAR-19 construct Flag-tagged-2 |
| 73 | 273 | CD19 FMC63 CAR and CMV-#42 |
| 74 | 274 | CD19 FMC63 CAR and CD25 promoter- |
| | | #42 |
| 75 | 275 | CD19 FMC63 CAR and CD69 promoter- |
| | | #42 |
| 76 | 276 | CD19 FMC63 CAR and TNF promoter-#42 |
| 77 | 277 | CD19 FMC63 CAR and NFAT promoter- |
| | | #42 |
| 78 | 278 | Leu16 scFv (VH/VL)-hulgGFc |
| 79 | 279 | Leu16 scFv (VH/VL) |
| 80 | 280 | Leu16 scFv (VL/VH)-hulgGFc |
| 81 | 281 | Leu16 scFv (VL/VH) |
| 82 | 282 | CD19-D1+2-Leu16 scFv (VH/VL)-hulgGFc |
| 83 | 283 | CD19-D1+2-Leu16 scFv (VH/VL) |
| 84 | 284 | CD19-D1+2-Leu16 scFv (VL/VH)-hulgGFc |
| 85 | 285 | CD19-D1+2-Leu16 scFv (VL/VH) |
| 86 | 286 | CD19-D1+2-MOC31 scFv (VH/VL) |
| 87 | 287 | CD19-D1+2-Ly2875358 scFv (VH/VL) |
| 88 | 288 | CD19 D1+2-Panitumumab scFv (VH/VL) |
| 89 | 289 | C11D5.3 scFv (VL/VH) |
| 90 | 290 | C11D5.3 scFv (VH/VL) |
| 91 | 291 | CD19-D1+2-C11D5.3 scFv (VL/VH) |
| 92 | 292 | CD19-D1+2-C11D5.3 scFv (VH/VL) |
| 93 | 293 | CD19-D1+2-hulgGFc-Trastuzumab |
| | | (VH/VL) |
| 94 | 294 | Bispecific CD19-D1+D2-Trastuzumab scFv |
| | | (VH/VL)-Panitumumab scFv (VH/VL) |
| 95 | 295 | Bispecific CD19-D1+D2-Trastuzumab scFv |
| | | (VH/VL)-Panitumumab scFv (VL/VH) |
| 96 | 296 | Bispecific Trastuzumab scFv-Panitumumab scFv (VH/VL) |
| 97 | 297 | Bispecific Trastuzumab scFv- |
| | | Panitumumab scFv (VL/VH) |
| 98 | 298 | lentiviral CMV promoter-#42 |
| 99 | 299 | lentiviral CD25 promoter-#42 |
| 100 | 300 | lentiviral CD69 promoter-#42 |
| 101 | 301 | lentiviral TNFa promoter-#42 |
| 102 | 302 | lentiviral NFATx6 promoter-#42 |
| 103 | 303 | Trastuzumab scFv (VH/VL)-hulgGFc |
| 104 | 304 | CD19-D1+2-extended linker-Leu16 scFv |
| | | (VH/VL)-hulgGFc |
| 105 | 305 | CD19-D1+2-extended linker-Leu16 scFv |
| | | (VH/VL) |
| 106 | 306 | CD19-D1+2-hulgGFc-Leu16 scFv (VH/VL) |
| 107 | 307 | Leu16 scFv (VH/VL)-CD19-D1+2-hulgGFc |
| 108 | 308 | EF1a-#72-T2A-#42 |
| 109 | 309 | EF1a-#42-T2A-#72 |
| 110 | 310 | #98 EF1a promoter and CMV (variant) |
| 111 | 311 | #42 EF1a promoter (pCDH-EF1a) |
| 112 | 312 | CD19 full ECD |
| 113 | 313 | CD19-D1+2-Trastuzumab scFv (VH/VL) |
| 114 | 314 | CD19 D1+D2-MOC31 scFv (VH/VL) |
| 115 | 315 | CD19 D1+D2-LY2875358 scFv (VH/VL) |
| 116 | 316 | CD19 D1+D2-Panitumumab scFv |
| | | (VH/VL) |
| 117 | 317 | FMC63 CAR-Flag |
| 118 | 318 | FMC63 CAR |
| 119 | 319 | anti-CD19 FMC63 CAR heavy chain |
| 120 | 320 | anti-CD19 FMC63 CAR light chain |
| 121 | 321 | anti-FMC63 (anti-Id) scFv VH-VL- |
| | | Trastuzumab scFv-His |
| 122 | 322 | anti-FMC63 (anti-Id) scFv VL-VH- |
| | | Trastuzumab scFv-His |

The following examples are provided for illustrative purposes only and are not intended to limit the invention in any manner.

### EXEMPLIFICATION

### Example 1

### An Antigen-Activation Controlled Promoter Promotes Cytokine Release after Cellular Therapeutic Cells Encounter an Antigen (e.g., Tumor Cells or their Local Environment)

Cytokine support for cancer therapeutics has a long history (e.g. the systemic use of TNF, LTa, IFNs and IL-2). Inherent problems with systemic cytokine therapy include high dosage requirement, minimal efficacy, and toxicity (lethal in the case of TNF and LTa). Such toxicity limits the use of IL-12 and IL-15.

For example, systemic recombinant IL-12 induced multiple serious adverse effects, including renal and systemic toxicity. High-dose levels were linked to temporary immune suppression, which would be unfavorable for effective immunotherapy. (See, Leonard et al., Blood 1997;90:2541-8, and Colombo, MP et al., CytokineGrowth Factor Rev 2002;13:155-68, incorporated herein by reference. The majority of systemic IL-12 trials were associated with toxic adverse events and limited efficacy, since the cytokine did not reach the tumor site(s) in sufficient concentration (S Tugues, et al., 2015 Cell Death Differ 22: 237-246, incorporated herein by reference.

Recombinant IL-15 induces NK and CD8 cell mediated toxicities. Dose-limiting toxicities observed in patients receiving rIL-15 included grade 3 hypotension, thrombocytopenia, and elevations of ALT and AST, resulting in a suboptimal maximum-tolerated dose, having minimal clinical efficacy. (See, Conlon, K., et al., J. of Clinical Oncology Jan 1, 2015: 74-82, incorporated herein by reference).

These and similar studies illustrate the dose-limiting toxicity and minimal efficacy associated with the uncontrolled administration and systemic distribution of cytokines. Accordingly, an individual with cancer or suspected of having cancer (e.g., tumor) is administered with a composition that allows for the direct transduction of the tumor cells so that a fusion protein of a cytokines and a target. The administration can be intravenously or intra-tumorally.

### Example 2

### Release of Cytokines from Transduced Tumor Cells

CART cells, CAR NK cells, TCR T cells, TIL, allogeneic NK cells and autologous NK cells are engineered for prolonged release of IL-21 as a cytokine fusion protein with TAA. Expansion and effector differentiation is induced in CD8+ T cells, and NK cell activation and cytolytic activity is supported.

Engagement of activation signals rapidly secretes IL-15 under the control of the TNF promoter, which supports NK cell expansion (e.g., CAR NK, allogeneic NK cells, and autologous NK cells) or recruits NK cell promulgation in an anti-tumor response (e.g., CAR T, TCR, TIL). Alternatively, various combinations of cytokines expressed and promoters are used, and are expressed in a suitable manner (IL-2, IL-12, IL-36g, IFNg) as a fusion protein.

Sustained local production of an anti-TAA scFv antibody fused to IL-12 recruits immune cells to support and expand the anti-tumor immune response triggered by engagement with the CAR T cell or other cellular therapeutic.

### Example 3

### The targeting Method Utilizes a Single Chain Variable Fragment (scFv) Antibody (or Fragment thereof), or Secreted Heterodimer TCR Alpha/Beta or Gamma/Delta Chains are Fused to an Antibody-Drug Conjugate Target

The target is the polypeptide sequence, protein domain or domains recognized by the ADC, which is an antibody coupled to a toxin. ADC targets are the HER2 receptor, the CD30 cell surface protein, folate receptor alpha, and CD19, among many others.

The targeting method utilizes an scFv antibody or fragment thereof, and is fused to an antibody-drug conjugate target. Alternatively, a secreted heterodimer TCR alpha/beta or gamma/delta chain targeting method is used. Example antibody-drug conjugate target includes CD30, HER2, or ADCC antibody targets.

### Example 4

### The Targeting Method Utilizes a Single Chain Variable Fragment (scFv) Antibody (or Fragment thereof), or Secreted Heterodimer TCR Alpha/Beta or Gamma/Delta Chains are Fused to a Pro-Immune Response Agent

A “pro-immune response agent” is any agent capable of stimulating the immune response by stimulation cells comprising the immune response cell populations. Agents that stimulate immune responses directly include cytokines, the “danger signals” (DAMPs, PAMPs, TLR agonists etc.), agonist antibodies or ligands (e.g. anti-4-1BB, CD4OL, B7-1, and many others), inhibitors of immunosuppressive signals (antagonists of PD-1, PD-L1, CTLA4, Lag-3, TIM-3, ID01, adenosine receptor, TGFbeta, and many others)

The targeting method utilizes a scFv antibody (or fragment thereof), alternatively, the targeting method is a secreted heterodimer TCR alpha/beta or gamma/delta chain. In both targeting methods, the pro-immune response agent includes IL-2, IFNalpha, IL-12, IL-15, IL-21, any TLR agonist and any immune checkpoint antibody (or fragment thereof).

### Example 5

### The Targeting Method Utilizes a Single Chain Variable Fragment (scFv) Antibody (or Fragment thereof), or Secreted Heterodimer TCR Alpha/Beta or Gamma/Belta Chains Fused to Cell Recruiting Moieties

Another method for recruitment of immune effector cells is the development of bispecific T-cell Engagers (e.g. BiTEs) which consist of two scFvs connected by a linker. One arm is an anti-TAA scFv, and the second scFv binds to the CD3 subunit of the TCR complex and therefore triggers T cell activation. Importantly, BiTEs and related molecules (DARTS, diabodies, fCabs etc) elicit repeated rounds of tumor cell lysis by T cells at very low effector/ target (E/T) cell ratios. The cytotoxicity is mediated by membrane perforation and subsequent induction of granzymes and apoptosis, exactly the type of killing that one wished to elicit: in the anti-tumor setting. Normally, the affinity of the anti-TAA scFv tumor-associated antigen is much higher than that directed against CD3—this enforces specificity for the targeted tumor. Many BiTEs have been generated, including BiTEs that target CD19, EpCAM, HER2, carcinoembryonic antigen (CEA), ephrin A2 (EphA2), CD33, and melanoma-associated chondroitin sulfate proteoglycan (MCSP). Other BiTEs that are in clinical studies are composed of EpCAM, prostate-specific membrane antigen (PSNIA), or CEA-binding molecules combined with CD3-binding modules.

In some examples, the targeting method utilizes an scFv antibody (or fragment thereof), alternatively, the targeting method includes a secreted heterodimer TCR alpha/beta or gamma/delta chain. In both targeting methods, the cell recruiting moiety is fused to anti-CD16. In alternative examples, the cell recruiting moiety is fused to an anti-CD3 moiety, and utilizes the BITE® technology described herein.

### Example 6

### Transfection of a Tumor In Vivo using an HPV Pseudovirus with an ScFv-CD19 Fusion Protein in which the ScFv Binds the TAA Her2, and the Tumor is Killed by CD19-Directed CAR T Cells

HPV pseudovirus comprising an ScFv-CD19 fusion protein is injected IV to transduce tumor cells in a xenograft mouse model of breast cancer expressing Her2. The transduced tumors secrete the ScFv-CD19 fusion protein, resulting in coating of tumor cells with the ScFv-Cd19 fusion protein via ScFv binding to Her2. Addition of CD19-directed CAR T cells kills the tumor cells coated with CD19. CAR T cells directed to CD19 are made by standard methods.

### Example 7

### Transfection of a Tumor In Vivo using an AAV-Phage Chimeric Virus with an ScFv-CD19 Fusion Protein in which the ScFv Binds the TAA Her2, and the Tumor is Killed by CD19-Directed CAR T Cells

A chimeric AAV/phage virus is injected IV to transduce tumor cells in a xenograft mouse model of breast cancer expressing Her2. The transduced tumors secrete the ScFv-CD19 fusion protein, resulting in coating of tumor cells with the ScFv-CD19 fusion protein via ScFv binding to Her2. Addition of CD19-directed CAR T cells kills the tumor cells coated with CD19. The generation of CD19 directed CAR T cells is well established.

### Example 8

### Transfection of a Tumor In Vivo using an HPV Pseudovirus with an ScFv-CD30 Fusion Protein in which the ScFv Binds the TAA Her2, and the Tumor is Killed by a CD30-Targeted ADC

HPV pseudovirus comprising an ScFv-CD30 fusion protein is injected IV to transduce tumor cells in a xenograft mouse model of breast cancer expressing Her2. The transduced tumors secrete the ScFv-CD30 fusion protein, resulting in coating of tumor cells with the ScFv-CD30 fusion protein via ScFv binding to Her2. Addition of CD30-directed antibody-drug conjugate (ADC) kills the tumor cells coated with CD30.Adcetris is a commercially available ADC from Seattle Genetics.

### Example 9

### Transduction of Cells with AAV Viral Particles Encoding Fusion Proteins Results in Secreted Functional Fusion Proteins Capable of Directing CAR19 T Cell Targeting and Activation

The present Example demonstrates expression of fusion proteins from cells transduced with AAV viral particles encoding fusion proteins. Further, the present example demonstrates that the expressed fusion proteins are capable of being bound by the anti-CD19 antibody FMC63 and detected by an anti-His antibody binding to the C-terminal His tag. Once expressed the fusion proteins are able to activate CAR19 T cells in the presence of cells that are CD19 negative.

The following table lists the various constructs tested in this example:

| | | Amino Acid SEQ | Nucleotide SEQ ID |
|---|---|---|---|
| Construct # | Description | ID NO: | NO: |
| 113 | CD19-D1+2-Trastuzumab scFv | 113 | 313 |
| | (VH/VL) | | |
| 114 | CD19 D1+D2-MOC31 scFv | 114 | 314 |
| | (VH/VL) | | |
| 115 | CD19 D1+D2-LY2875358 scFv | 115 | 315 |
| | (VH/VL) | | |
| 116 | CD19 D1+D2-Panitumumab | 116 | 316 |
| | scFv (VH/VL) | | |

### Methods

A 12 well plate was seeded with either A431 or 293T cells around 4×10e5 cells/well in DMEM+10% FBS media. The following day, one well was counted to get an accurate count for the viral infections. Cells were infected with a multiplicity of infection (MOI) of 1×10⁶ or 5×10⁶. AAV2 viral particles were made by Vigene (Rockville, Md.). The viral particles were generated from plasmids where the inserts containing the CD19 D1+D2-scFv-His sequences were cloned into pAV-FH. The viral particles AAV-1 (CD19-D1+2-Trastuzumab scFv (VH/VL), SEQ ID NO:113), AAV-3 (CD19 D1+D2-MOC31 scFv (VH/VL), SEQ ID NO:114), AAV-5 (CD19 D1+D2-LY2875358 scFv (VH/VL), SEQ ID NO:115) and AAV-7 (CD19 D1+D2-Panitumumab scFv (VH/VL), SEQ ID NO:116) had titers of 8.39×10¹³, 1.51×10¹⁴, 3.03×10¹⁴ and 2.13×10¹⁴ GC/ml, respectively. Infections were done in 0.6ml/well DMEM+2% FBS where the virus was added directly into the media. Different concentrations of virus were used from 10 μl (aka “104” or “10e4”) to 5×10⁶ (aka “5×106” or “5×10e6”). The following day, the media was changed to DMEM+10%FBS and the incubation continued for 3 or 6 days.

### Elisa

Expression analysis was examined in the supernatant using an ELISA where anti-CD19 FMC63 was used for capture and anti-His used for detection. Briefly, 96 well plates (Pierce, Cat# 15041) were coated with 1.0 μg/ml reagent in 0.1 M carbonate, pH 9.5 for O/N at 4C. The plates were then blocked with 0.3% nonfat dry milk (NFD) in TBS (200 μl/well) for 1 hr at RT. Plates were then washed 3× with wash buffer (1× TBST: 0.1 M Tris, 0.5 M NaCl, 0.05% Tween20). Titrations were performed from undiluted cell culture supernatant or purified protein at 1.0 μg/ml with serial 3× dilutions, 100 μl per well and incubate for 1h at RT. Dilution buffer is 1% BSA in 1× TBS (0.1 M Tris, 0.5 M NaCl) followed by washing 3× with wash buffer. Secondary reagents were added (if needed) such as Biotinylated-reagents at 1 μg/ml concentration at RT for 1 hour. HRP-conjugated reagents were added at 1:2000, applied 100 μl per well, incubated at RT in dark for 1 hr. 100 μl 1-Step Ultra TMB-ELISA (Thermo Fisher, Prod#34028) was added per well. Plates were read at 405 nm when color had developed.

### XTT Cell Proliferation Assay (ATCC, Cat# 30-1011K)

An aliquot of the XTT reagent and the activation reagent was rapidly thawed at 37oC prior to use. 0.1 ml of activation reagent was then added to 5.0 ml of the XTT reagent. 50 μl of the activated—XTT solution was then added to each well. The plate was placed in the cell culture incubator for 2-4 hours and monitored for color development. The absorbance of the plate was read at wavelength 450 nm. The % cell death (aka cytotoxicity) was calculated as follows:

killing=[1-OD(experimental wells-corresponding number of T cells)/OD (tumor cells without T cell-medium)]×100

### Interferon Gamma Concentration Assay by ELISA

A 96 well plate (Pierce, product #15041) was coated with 1.0 μg/ml mouse anti-human IFNγ (BD Pharmingen, Cat# 551221) in 0.1 M carbonate buffer, pH 9.5, overnight at 4° C. The plate was blocked with 0.3% non-fat dry milk solution in tris-buffered saline (TBS) using 200 μl/well for 1 hour at room temperature. The plate was washed x3 with wash buffer (1× TBS/Tween: 0.1 M Tris, 0.5 M NaCl, 0.05% Tween20). 100 μl culture supernatant from the 24 hour or 48 hour culture plates (see above) were added to the ELISA plate. A titration of recombinant human IFNγ (Thermo Fisher, Cat# RIFNG100) was also performed in the same plate from 300 ng/ml with serial 3× dilutions to 2 pg/ml to generate a standard curve. The plate was then incubated for 1 hour at room temperature. The dilution buffer was 1× TBS (0.1 M Tris, 0.5 M NaCl) plus 1% BSA. The plate was washed x3 with wash buffer. Biotinylated mouse anti-human IFNγ (BD Pharmingen, Cat# 554550) was added at 1 μg/ml concentration and the plate was incubated at room temperature for 1 hour. The plate was washed again x3 with wash buffer. HRP-conjugated Streptavidin (Thermo Fisher, Cat# 21130) was added at a 1:2000 dilution from the stock, with 100 μl added per well. The plate was then incubated at room temperature for 1 hour in the dark. The plate was washed again x3 with wash buffer. 100 μl per well of 1-Step Ultra TMB-ELISA development solution (Thermo Fisher, Cat #34028) was added per well. The plate was read at wavelength 405 nm when color developed sufficiently.

### Results

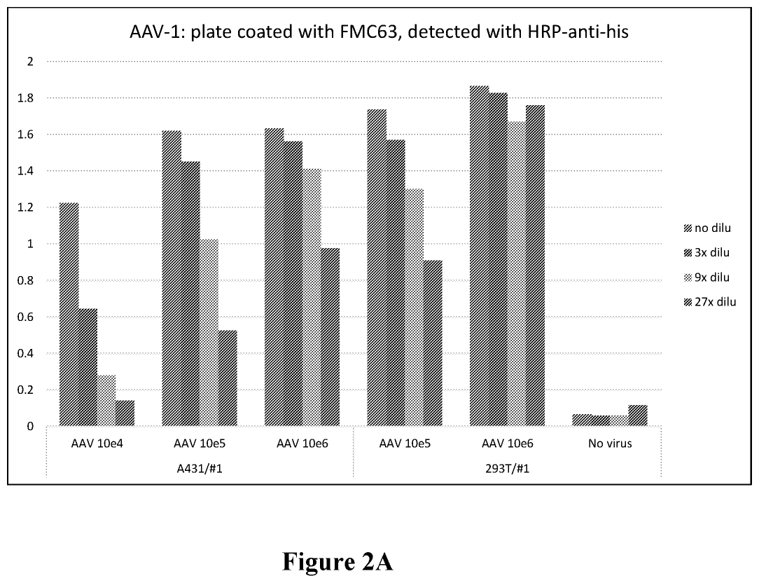

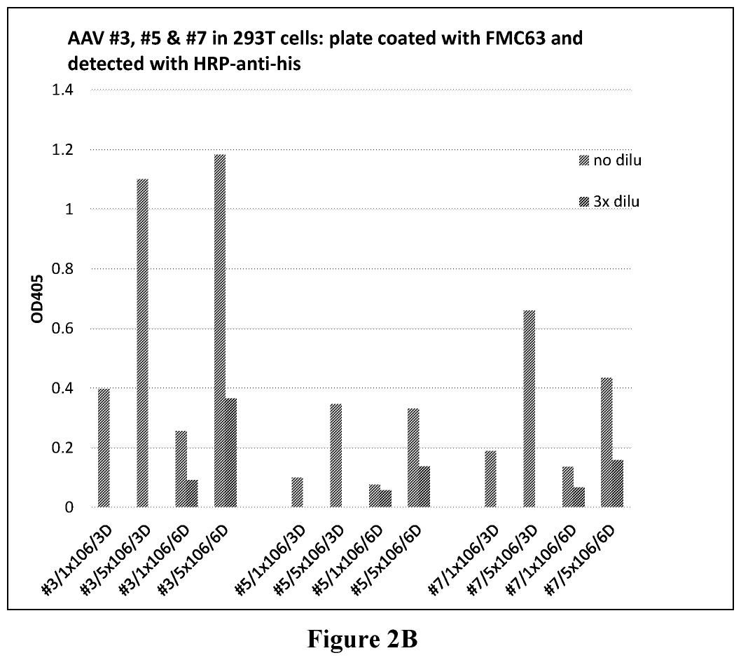

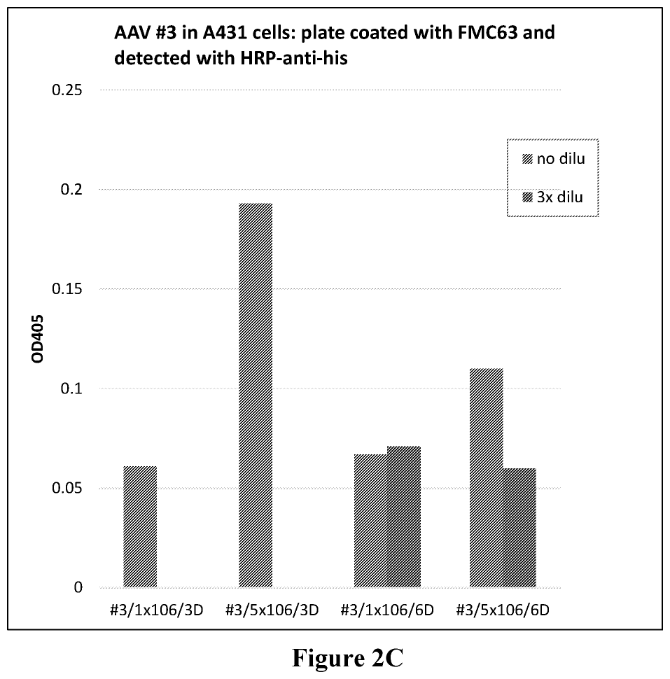

FIGS. 2A-2C demonstrate expression of fusion proteins from transduced cells. FIG. 2A shows detection of fusion protein CD19-D1+2-Trastuzumab scFv (VH/VL) (AAV#1) is expressed after transduction of A431 or 293T cells with AAV viral particles encoding the fusion protein. The expressed fusion protein is capable of being bound by the anti-CD19 antibody FMC63 and detected by an anti-His antibody binding to the C-terminal His tag. FIG. 2B demonstrates detection of expression of fusion proteins AAV-3 (CD19 D1+D2-MOC31 scFv (VH/VL), SEQ ID NO:114), AAV-5 (CD19 D1+D2-LY2875358 scFv (VH/VL), SEQ ID NO:115) and AAV-7 (CD19 D1+D2-Panitumumab scFv (VH/VL), SEQ ID NO:116) resulting from transduction of 293T cells with AAV particles encoding the indicated fusion protein. FIG. 2C demonstrates detection of expression of fusion protein AAV-3 (CD19 D1+D2-MOC31 scFv (VH/VL) resulting from transduction of A431 cells with AAV particles encoding the fusion protein.

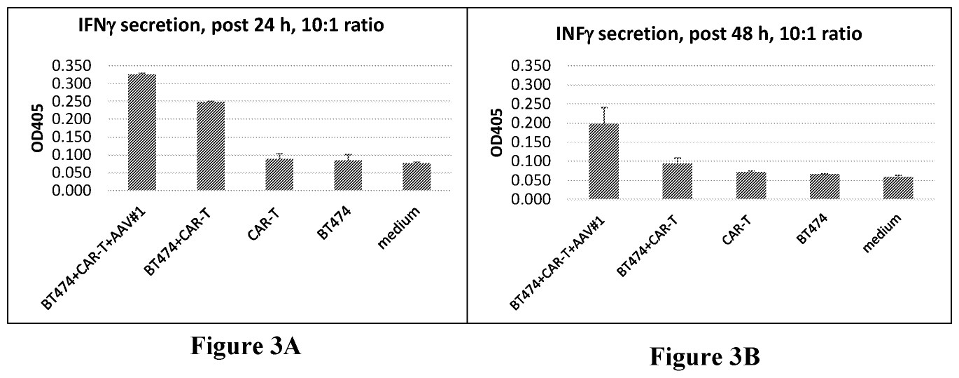

FIGS. 3A and 3B show a summary of results of the IFNγ ELISA measuring induction of IFNγ upon incubation of CAR19 T cells (Promab) with AAV-1-expressed supernatant and BT474 cells. FIG. 3A shows the results of the IFNγ ELISA at 24 hrs at a 10:1 effector:target ratio. FIG. 3B shows the results of the IFNγ ELISA at 48 hrs at a 10:1 effector:target ratio.

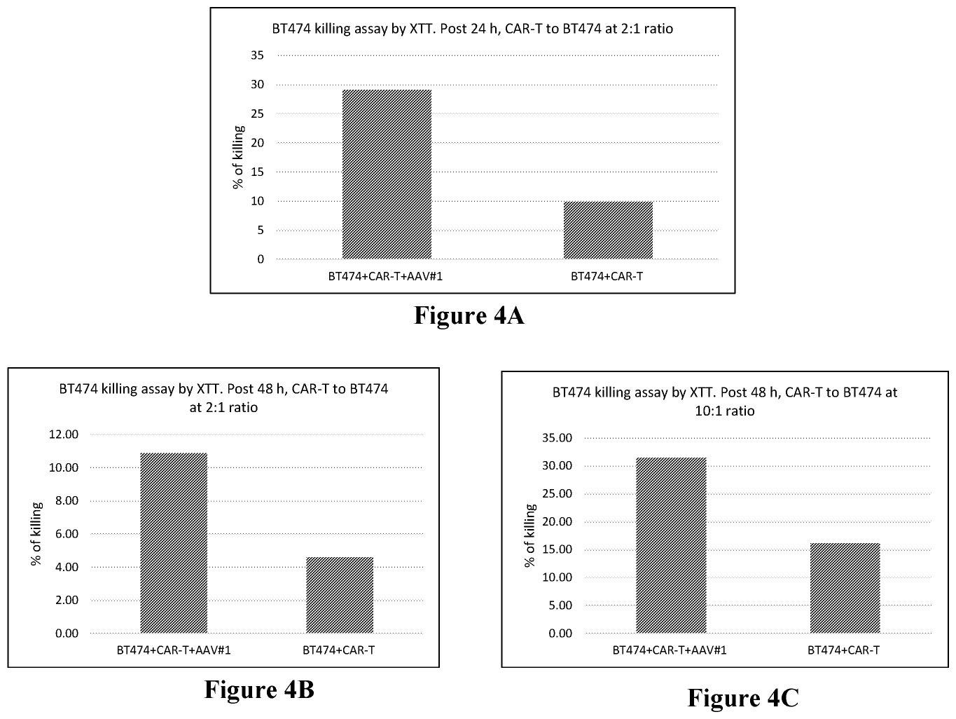

FIGS. 4A-4C show induction of cytotoxicity upon incubation of CAR19 T cells (Promab) with AAV-1-expressed supernatant and BT474 cells. FIG. 4A shows summary XTT-cytotoxicity results for 2:1 effector:target ratio after 24 hours. FIG. 4B shows summary XTT-cytotoxicity results for 2:1 effector:target ratio after 48 hours. FIG. 4C shows summary XTT-cytotoxicity results for 10 :1 effector:target ratio after 48 hours. These results demonstrate that AAV transduction of cells (e.g. tumor cells) can result in expression of functional fusion proteins which can direct CAR19 T cell activation and cytotoxicity.

### Example 10

### Transduction of Cells with AAV Viral Particles Encoding Anti-Idiotype (Anti-Id) scFv/scFv Fusion Proteins Results in Secreted Functional Fusion Proteins Capable of Directing CAR19 T Cell Targeting and Activation

Construction and Expression of Trastuzumab scFv-Anti-Id Fusion Proteins

This example illustrates that an anti-idiotype scFv (136.20.1 scFv, which recognizes the scFv domain of the mouse anti-human antibody FMC63 (see, e.g., Jena B, et al. (2013) Chimeric Antigen Receptor (CAR)-Specific Monoclonal Antibody to Detect CD19-Specific T Cells in Clinical Trials. PLoS ONE 8(3): e57838; US 2016/0096902))can be fused to scFvs that bind to HER2, an antigen expressed on solid tumors and their metastases, e.g., the anti-Her2 scFv that is derived from trastuzumab/hu4D5v8 (e.g. from PDB database (1N8Z) and the Drug Bank database (AC. DB00072), see also Cho HS, Mason K, Ramyar KX, Stanley AM, Gabelli SB, Denney DW, Jr, et al. Structure of the extracellular region of HER2 alone and in complex with the Herceptin Fab. Nature. 2003;421:756-760), or of other anti-Her2 scFvs whether known in the art or newly discovered.

Trastuzumab scFv/anti-Id scFv fusion proteins containing 136.20.1 anti-idiotype scFv and trastuzumab scFv is created using the coding sequences for each scFv, with an appropriate signal sequence, linked together using G4S or other robust linker sequences as needed to allow each VH/VL pair to fold, and also to retain the structural integrity of each scFv by preventing interaction between the two scFv, using methods well known in the art.

The trastuzumab scFv-anti-Id scFv fusion proteins are produced in a variety of configurations, where each scFv is provided in tandem as a VH/VL pair, and in the N- or C-terminal position. For example, trastuzumab scFv-anti-Id scFv fusion proteins include N-terminal 136.20.1 scFv (VH/VL or VL/VH) and C-terminal trastuzumab scFv (VH/VL or VL-VH) and also N-terminal trastuzumab scFv (VH/VL or VL/VH) and C-terminal 136.20.1 scFv (VH/VL or VL/VH).

A His-tag is used to monitor protein expression. The His-tag is N-terminal or C-terminal in placement. A FLAG-tag is used as needed. A biotin label is used as needed. Other tags and labels are used as needed.

The assembled sequences are cloned into expression systems for analysis. For example, the sequences are cloned into the pcDNA-1 vector. The cloned sequences are expressed in mammalian cells. For example, 293T cells are transfected using vector DNA and Lipofectamine 2000® (Invitrogen). Some transfections are for temporary protein production (transient) while some are for cell line development (stable). Optimized sequences are cloned into retroviral, lentiviral or mRNA systems suitable for large scale transduction of human T cells. Protein expression level and quality is determined by Western blot analyses, immunoprecipitation, ELISA analyses, chromatography and/or additional methods as needed.

Trastuzumab scFv-Anti-Id Fusion Proteins are Recognized by FMC63 and by HER2

The ability of the trastuzumab scFv-anti-Id scFv fusion proteins to bind to the distinct ligands is determined using a variety of methods to demonstrate specific binding. ELISA plates are coated with FMC63 antibody or with streptavidin/biotinylated-HER2 to bind to 136.20.1 scFv and trastuzumab scFv, respectively. After binding is allowed to occur, the plates are gently washed to remove unbound materials. Anti-HIS-antibody coupled to horseradish peroxidase (HRP) is used to detect the bound fusion protein. In another iteration, an ELISA plate is coated with anti-HIS antibody to capture the fusion protein, and biotinylated HER2/streptavidin-HRP is used to detect. In another iteration, the ELISA plates is coated with FMC63 antibody and biotinylated HER2/streptavidin-HRP is used to detect. Other iterations are utilized as needed. The ELISAs are used to monitor expression of transient transfections, stable transfections, and cell transductions.

Trastuzumab scFv-Anti-Id Fusion Proteins Bind to HER2 Positive BT474 Cells

The trastuzumab scFv-anti-Id scFv fusion proteins are shown to bind to target (HER2 positive) tumor cells using standard techniques known in the art, e.g. flow cytometry, ELISA, etc. Trastuzumab scFv-anti-Id scFv fusion proteins are incubated with BT474 cells or other human tumor cells or cell lines that are HER2-positive. After incubation the cells are gently washed to remove unbound materials. The bound trastuzumab scFv-anti-Id scFv fusion proteins are detected using fluorescently labeled anti-HIS antibody or FMC63 antibody.

CAR19 T Cells are Redirected to HER2 Positive Tumor Cells via the Trastuzumab scFv-Anti-Id Fusion Protein, and in a Manner that Successfully Activates the CAR19 T Cells so that they lyse the Tumor Cells

Trastuzumab scFv-anti-Id scFv fusion proteins are shown to induce CAR T cell activity using cytokine release and cytotoxicity assays. Trastuzumab scFv-anti-Id scFv fusion proteins are incubated with BT474 cells or other human tumor cells or cell lines that are HER2-positive. Trastuzumab scFv-anti-Id scFv fusion proteins are in the form of a soluble purified protein, or are in a cell culture supernatant, or are secreted from a cell in the culture, for example a CAR T cell that has an FMC63-based CAR domain. FMC63-based CAR T cells are added to the culture if they are not already present. The coculture is allowed to incubate, for example between 4 hours and 72 hours. At an optimal time, supernatants are collected for ELISA analyses, for example, for IL-2 and IFN-gamma. At an optimal time the cells are analyzed using a cytotoxicity assay, for example an XTT assay. The assays demonstrate that trastuzumab scFv-anti-Id scFv fusion proteins redirect FMC63 based CART cells to lyse target HER2-positive tumors cells, causing their cytotoxicity.

Construction and Expression of Various scFv-Anti-Id Fusion Proteins

The scFv from the 136.20.1 anti-idiotype antibody recognizing FMC63 can be fused to many other scFv directed to diverse tumor antigens and investigated for functionality in the same manner as the trastuzumab scFv fusion. In another example, the 136.20.1 scFv is fused to a tumor targeting scFv, for example an scFv that targets CD20, an antigen that is expressed on B cell malignancies, e.g., the anti-CD20 scFv as is disclosed as a component of SEQ ID NO: 65, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 125, or SEQ ID NO: 126 or other variations of the anti-CD20 scFv or of other anti-CD20 scFvs whether known in the art or newly discovered. In a further example, the 136.20.1 anti-idiotype scFv is fused to an scFv that targets BCMA, an antigen that is expressed on plasma cell malignancies including multiple myeloma, e.g. the anti-BCMA scFv as is disclosed as a component of SEQ ID NO: 89, SEQ ID NO: 90, SEQ ID NO: 91, SEQ ID NO: 92, SEQ ID NO: 119, or SEQ ID NO: 120 or other variations of this anti-BCMA scFv or of other anti-BCMA scFvs whether known in the art or newly discovered. In another example, the 136.20.1 anti-idiotype scFv is fused to a tumor targeting scFv, for example, an scFv that targets EGFR, an antigen expressed on solid tumors and their metastases, e.g., the anti-EGFR scFv that is disclosed within SEQ ID NO: 21, SEQ ID NO: 22, SEQ ID NO: 54, SEQ ID NO: 57, SEQ ID NO: 58, SEQ ID NO: 88, SEQ ID NO: 94, SEQ ID NO: 95, SEQ ID NO: 96, SEQ ID NO: 97, or other variations of the anti-EGFR scFv or of other anti-EGFR scFvs whether known in the art or newly discovered.

### Cell Transduction

A 12 well plate is seeded with either A431 or 293T cells around 4×10e5 cells/well in DMEM+10% FBS media. The following day, one well is counted to get an accurate count for the viral infections. Cells are infected with a multiplicity of infection (MOI) of 1×10⁶ or 5×10⁶. AAV2 viral particles are made by Vigene (Rockville, MD). The viral particles are generated from plasmids where the inserts containing sequences encoding the scFv-anti-Id fusion proteins (that include a HIS tag on the scFv) are cloned into pAV-FH. The viral particles are titered using standard methods. Infections are done in 0.6ml/well DMEM+2% FBS where the virus is added directly into the media. Different concentrations of virus are used from approx 10⁴ (aka “104” or “10e4”) to 5×10⁶ (aka “5×106” or “5×10e6”). The following day, the media is changed to DM EM+10%FBS and the incubation continued for 3 or 6 days.

### Elisa Analysis

Expression analysis is examined in the supernatant using an ELISA where anti-CD19 FMC63 is used for capture and anti-His used for detection. Briefly, 96 well plates (Pierce, Cat# 15041) are coated with 1.0 μg/ml reagent in 0.1 M carbonate, pH 9.5 for O/N at 4C. The plates are then blocked with 0.3% nonfat dry milk (NFD) in TBS (200 μl/well) for 1 hr at RT. Plates are then washed 3× with wash buffer (1× TBST: 0.1 M Tris, 0.5 M NaCl, 0.05% Tween20). Titrations are performed from undiluted cell culture supernatant or purified protein at 1.0 μg/ml with serial 3× dilutions, 100 μl per well and incubated for 1h at RT. Dilution buffer is 1% BSA in 1× TBS (0.1 M Tris, 0.5 M NaCl) followed by washing 3× with wash buffer. Secondary reagents are added (if needed) such as Biotinylated-reagents at 1 μg/ml concentration at RT for 1 hour. HRP-conjugated reagents are added at 1:2000, applied 100 μl per well, incubated at RT in dark for 1 hr. 100 μl 1-Step Ultra TMB-ELISA (Thermo Fisher, Prod#34028) is added per well. Plates are read at 405 nm when color has developed. The expressed fusion proteins are capable of being bound by the anti-CD19 antibody FMC63 and detected by an anti-His antibody.

### XTT Cell Proliferation Assay (ATCC, Cat# 30-1011K)

An aliquot of the XTT reagent and the activation reagent is rapidly thawed at 37° C. prior to use. 0.1 ml of activation reagent is then added to 5.0 ml of the XTT reagent. 50 μl of the activated—XTT solution is then added to each well. The plate is placed in the cell culture incubator for 2-4 hours and monitored for color development. The absorbance of the plate is read at wavelength 450 nm. The % cell death (aka cytotoxicity) is calculated as follows:

% killing=[1-OD(experimental wells-corresponding number of T cells)/OD (tumor cells without T cell-medium)]×100

Expressed fusion proteins are capable of directing CAR19 T cell activation and cytotoxicity upon incubation of supernatant with CAR19 T cells and BT474 cells.

### Interferon Gamma Concentration Assay by ELISA

A 96 well plate (Pierce, product #15041) is coated with 1.0 μg/ml mouse anti-human IFNγ (BD Pharmingen, Cat# 551221) in 0.1 M carbonate buffer, pH 9.5, overnight at 4° C. The plate is blocked with 0.3% non-fat dry milk solution in tris-buffered saline (TBS) using 200 μl/well for 1 hour at room temperature. The plate is washed x3 with wash buffer (1× TBS/Tween: 0.1 M Tris, 0.5 M NaCl, 0.05% Tween20). 100 μl culture supernatant from the 24 hour or 48 hour culture plates (see above) are added to the ELISA plate. A titration of recombinant human IFNγ (Thermo Fisher, Cat# RIFNG100) is also performed in the same plate from 300 ng/ml with serial 3× dilutions to 2 pg/ml to generate a standard curve. The plate is then incubated for 1 hour at room temperature. The dilution buffer is 1× TBS (0.1 M Tris, 0.5 M NaCl) plus 1% BSA. The plate is washed x3 with wash buffer. Biotinylated mouse anti-human IFNγ (BD Pharmingen, Cat# 554550) is added at 1 μg/ml concentration and the plate is incubated at room temperature for 1 hour. The plate is washed again x3 with wash buffer. HRP-conjugated Streptavidin (Thermo Fisher, Cat# 21130) is added at a 1:2000 dilution from the stock, with 100 μl added per well. The plate is then incubated at room temperature for 1 hour in the dark. The plate is washed again x3 with wash buffer. 100 μl per well of 1-Step Ultra TMB-ELISA development solution (Thermo Fisher, Cat #34028) is added per well. The plate is read at wavelength 405 nm when color has developed sufficiently. Expressed fusion proteins are capable of inducing IFNγ upon incubation of supernatant with CAR19 T cells and BT474 cells.

### Example 11

### Expression and Functional Testing of anti-FMC63 anti-Is scFv

The following table lists the various constructs tested in this example:

| | | Amino Acid | Nucleotide |
|---|---|---|---|
| | | SEQ ID | SEQ ID |
| Construct # | Description | NO: | NO: |
| 140 | FMC63 CAR-Flag | 117 | 317 |
| 150 | FMC63 CAR | 118 | 318 |
| 151 | anti-CD19 FMC63 | 119 | 319 |
| | CAR heavy chain | | |
| 152 | anti-CD19 FMC63 | 120 | 320 |
| | CAR light chain | | |
| 171 | anti-FMC63 (anti-Id) | 121 | 321 |
| | scFv VH-VL- | | |
| | Trastuzumab scFv- | | |
| | His | | |
| 172 | anti-FMC63 (anti-Id) | 122 | 322 |
| | scFv VL-VH- | | |
| | Trastuzumab scFv- | | |
| | His | | |

### Cloning and Expression of the Anti-FMC63 Antibody

The amino sequence for the variable heavy and light chains for the anti-FMC63 antibody, 136.20.1, was obtained from Cooper et al. patent WO2014190273 Al. The sequences were back translated and used to generate the full antibody chains. For the heavy chain, the sequence of the leader and constant domains were obtained from a murine IgG2a antibody (UniProt P01863). For the kappa light chain, the signal sequence and constant domains were obtained from Uniprot P01863. The nucleotide sequence of the anti-CD19 FMC63 CAR heavy chain (SEQ ID NO: 319; construct #151) and light chain (SEQ ID NO: 320; construct #152) were chemically synthesized by GenScript and cloned into the vector pcDNA3.1(+) (#151) or pcDNA3.1(+)hygro (#152). Equal amounts of the plasmids were co-transfected into 293T cells using lipofectamine 2000 (Invitrogen/Thermo Fisher Prod#11668019) following the manufacturers instructions; supernatants were harvested after 48hr. For the large-scale transfections, 293T cells were seeded into T175 flasks and transfected with lipofectamine 2000, as above, when the cells reached about 80% confluency. Cells were cultured in bovine FBS with low serum IgG (VWR). Supernatants were harvested every 3-4 days.

### Elisa Method

A 96 well plate (Pierce, Cat# 15041) was coated with 1.0 ug/ml goat anti-mlgG in 0.1 M carbonate, pH 9.5 for O/N at 4C. The plate was blocked with 0.3% non-fat milk in Tris buffered saline (TBS 0.1 M Tris, 0.5 M NaCl) (200 ul/well) for 1 hr at RT. Then washed 3× with wash buffer (1× TBST: 0.1 M Tris, 0.5 M NaCl, 0.05% Tween20). The cell culture sup was titrated from 50× dilution down with 3× dilutions,100 ul was added per well and incubated for 1h at RT. Dilution buffer is 1% BSA in 1× TBS. The plate was washed 3× with wash buffer then 100 ul per well HRP-goat anti-mlgG at 1:2000 was applied and incubated at RT in dark for 1 hr. Then 100 ul of 1-Step Ultra TMB-ELISA from Thermo Fisher, Prod#34028 was added per well and the plate was read at 405 nm when color developed.

CAR expression

293T cells were transfected with an anti-CD19 FMC63 CAR vector (SEQ ID NO: 317; construct #140). The CAR sequence (FMC63 VL-VH-Flag-CD28 linker/transmembrane/intracellular domain (ICD)-4-1BB ICD-CD3z ICD) was synthesized by ProMab Biotechnologies. The CAR insert was then cloned into a modified form of the System Biosciences vector pCDH-EF1a to generate construct #140 (SEQ ID NO: 317). The vector were transiently transfected into 293T cells using 2.5ug of DNA and 10 ul of lipofectamine 2000 (Invitrogen/Thermo Fisher). After ˜48hrs, the cells were harvested and resuspended in FACS buffer (1%I3SA, 0.1% sodium azide in PBS). CAR transfected cells (2.5×10\hat{ }5) were incubated with anti-Flag (1 ug/test) for 30 min at 4° C., spun and washed twice with FACS buffer, then followed by incubation with anti-rabbit IgG-APC for 30 min at 4° C. Cells were spun and washed as above, then fixed with 1% PFA in PBS. Fixed cells were analyzed on Accuri 6 for CAR expression (Flag positive).

### Cell Binding

Cells transfected with construct #140 (SEQ ID NO: 317) (2.5×10\hat{ }5 in 50 ul) were incubated with 50 u1 sup or purified (5 ug/ml as final conc.) protein of constructs#151/#152 (SEQ ID NO: 319/ SEQ ID NO: 320) for 30 min at 4° C., spun and washed twice with FACS buffer. This was followed by another incubation with anti-mouse Fc gamma-PE for 30 min at 4° C. Cells were spun and washed as above, and fixed with 1% PFA in PBS. Fixed cells were analyzed on Accuri 6 for CAR expression binding (PE positive).

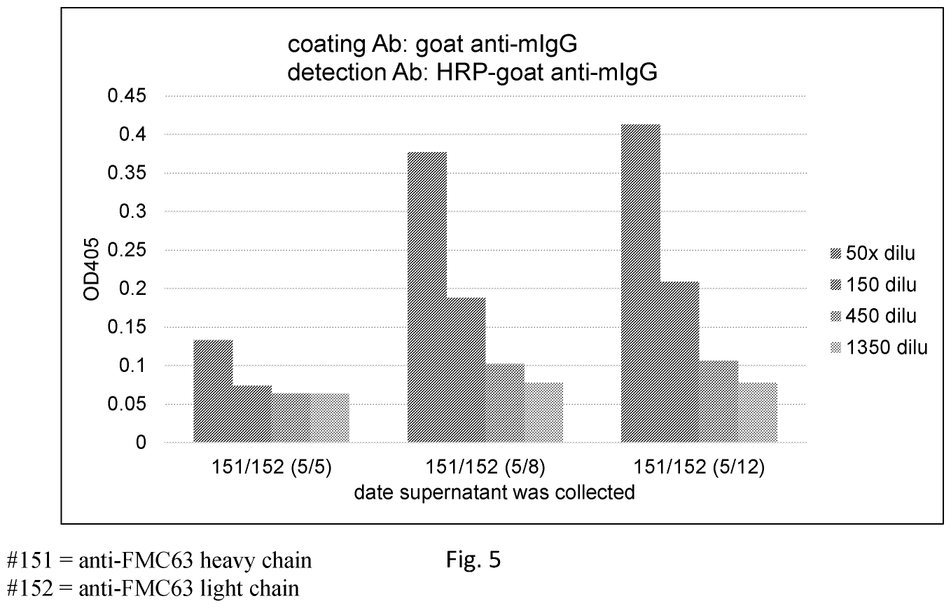

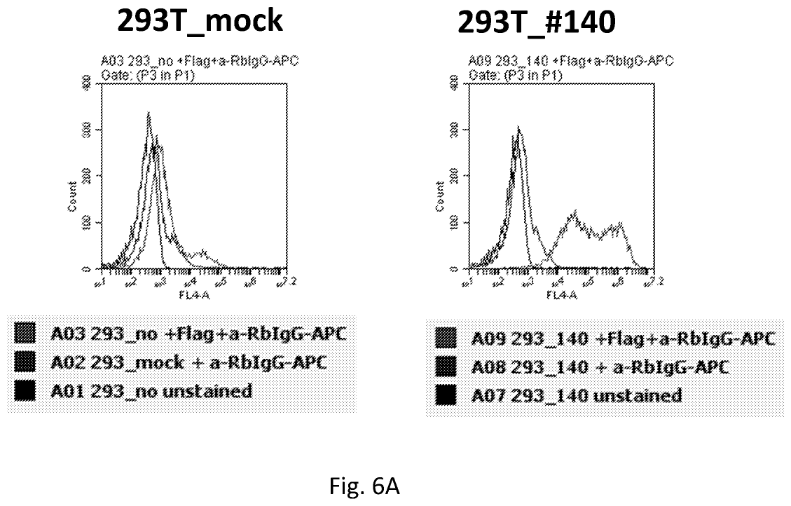

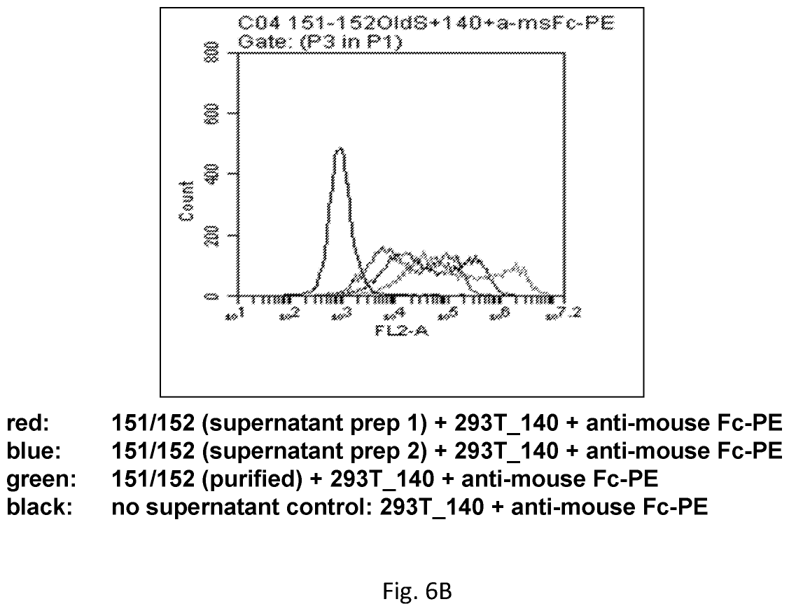

FIG. 5 confirms the secretion of anti-FMC63 antibody by detection of the presence of anti-FMC63 heavy and light chain in supernatnats of transfected 293T cells. Further, the secreted anti-FMC63 antibody binds CAR19 containing an FMC63 domain. FIG. 6A and demonstrates that the CD19 CAR construct is expressed on the surface of transfected 293T cells (#140). FIG. 6B demonstrates binding of the anti-FMC63 anti-Id scFv to the CD19 CAR construst.

### Example 12

### Expression and Functional Testing of Trastuzumab scFv-Anti-Id scFv Fusion Proteins

### Cloning and Expression of Constructs #171 and #172 (SEQ ID NO: 321 and SEQ ID NO: 322, Respectively)

Constructs were generated to express the anti-FMC63 scFv-Trastuzumab scFv fusion proteins with two orientations of the heavy and light variable domains of anti-FMC63. Construct #171 (SEQ ID NO: 121) contains anti-FMC63 VH-linker-VL-linker-Trastuzumab scFv-His and construct #172 (SEQ ID NO: 122) contains the anti-FMC63 in the VL-linker-VH arrangement. The sequences were chemically synthesized by GenScript and cloned into pcDNA3.1(+) hygro. Supernatants containing the bispecific scFvs were produced by transfecting 293T cells using lipofectamine 2000 (Invitrogen/Thermo Fisher). The supernatants were harvested after 72hrs by spinning for 3 min at 12k rpm at 4C.

### Elisa Method

A 96 well plate (Pierce, Cat# 15041) was coated with 1.0 ug/ml Her2-hFc (plate#1) (Acrobiosystems, Cat#HE2-H5253) or FMC63 (plate#2) (NOVUS, Cat#NBP2-527160) in 0.1 M carbonate, pH 9.5 for O/N at 4C. The plates were blocked with 0.3% non-fat milk in TBS (200 ul/well) for 1 hr at RT. The plate was washed 3× with wash buffer (1× TBST: 0.1 M Tris, 0.5 M NaCl, 0.05% Tween20). The cell culture sup was titrated from no dilution down with 3 fold dilutions; the purified construct #42 protein (LakePharma) was started with 1 ug/ml down with 3 fold dilutions. Next, 100 ul was added per well and incubated for 1h at RT. Dilution buffer is 1% BSA in 1× TBS (0.1 M Tris, 0.5 M NaCl). The plates were washed 3× with wash buffer. For the plate#1, 100 ul of 1 ug/ml FMC63 was added to each well for 1 hr at RT and followed by 100 ul HRP-anti-mlgG at 1:2000. For plate#2, 100 ul HRP-anti-his at 1:2000 was applied per well and incubated for 1 hr at RT. For the final step, 100 ul 1-Step Ultra TMB-ELISA from Thermo Fisher, Prod#34028 was added per well and plates were read at 405 nm when color developed.

### Detection of Binding to Her2-Positive Target Cells

Supernatant (100u1) from 293T cells, transfected with constructs #171 or #172, was incubated with 50u1 (2×10e5) SKOV-3 luciferase cells (Cell Biolabs, Inc. #AKR232) on ice for 30 min. The samples were spun, washed 2× with FACS buffer (1%BSA, 0.1% sodium azide in PBS) and then incubated with anti-His tag-PE (5ul/sample, R&D systems #IC050P) on ice for 30 min. The cells were spun, washed 2× with FACS buffer and fixed with 1% PFA in PBS. Fixed cells were analyzed on Accuri 6 for anti-id-Trastuzumab scFv-His binding. The presence of Her2 on the SKOV-3 cells was determined by staining 2×10e5 cells with 1u1/sample anti-Her2 (Novus #NBP2-33064PE) for 30 min on ice, spun, washed 2× with FACS buffer, fixed and read as above. An anti-murine IgG2a-PE antibody 1u1/test (Thermo Fisher #SA1-120-82) was used as a control.

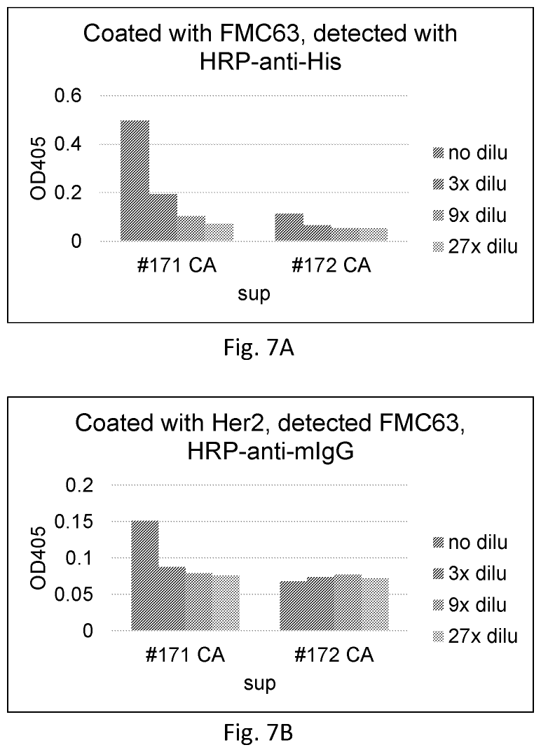

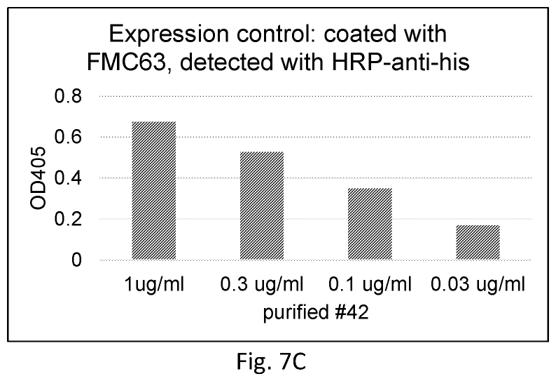

FIGS. 7A-C further demonstrate Trastuzumab scFv/anti-ld scFv fusion proteins containing 136.20.1 anti-idiotype scFv and trastuzumab scFv secreted from transfected 293T cells bind both FMC63 (FIG. 7A) and Her2 (FIG. 7B). Additionally, Trastuzumab scFv/anti-ld scFv fusion proteins, constructs #171 and #172, are capable of recognizing Her2 expressed on SKOV3 cells. FIG. 7C demonstrates binding of a CD19 expressing construct (#42) with the FMC63 coated plate as a control.

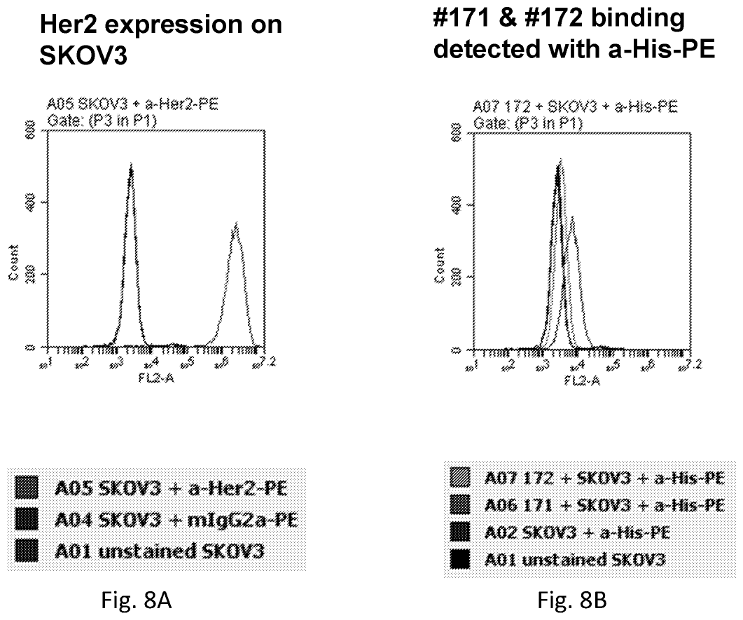

FIG. 8A demonstrates Her2 expression on SKOV3 cells and FIG. 8B demonstrates binding to the SKOV3-Her2 cells by the Trastuzumab scFv/anti-ld scFv fusion protein.

### Example 13

### CAR19 T Cell Targeting and Activation by Trastuzumab scFv-Anti-Id scFv Fusion Proteins

### SKOV3-Her2-Luc Killing Assay

Supernatant from cells transfected with #171 (concentration=0.15ug/ml), starting at 0.075ug/ml was titrated down in 3× serial dilution in RPMI +10%FBS (no antibiotics) medium for 8 points. Five replicates were done per dilution. SKOV3-Her2-Luc cells were seeded at 1×10e4/100u1 RPMI +10% FBS (no antibiotics)/well in a solid white plate. The cells were allowed to settle for ˜2hrs and then spun out and the supernatant removed. Next 50 ul of the 3× serial diluted #171 containing supernatant was added to SKOV3 cells. Then, 50 ul of CAR T cells #150 (SEQ ID NO:318) (5×10e4) was added (5 CART: 1 SKOV3 ratio) and the plate was incubated at 37° C. for 48hr. The sup was collected and the cells washed 2× with PBS. 20 ul 1× lysis buffer from Luciferase assay system kit, Promega, Fisher CAT# PR-E1500 was added into plate. The plate was placed into the luminometer with injector (Glomax Multi Detection System form Promega). The injector adds 100 ul of Luciferase assay reagent per well, then the well is read immediately. The plate is advanced to the next well for a repeat of the inject-then-read process.The % killing was determined for each concentration by dividing it by the RLU (relative luciferase units) of the target cells only using the equation 1-RLU sample/RLU target cell x 100%.

### IFN Gamma ELISA Method

A 96 well plate (Pierce, Cat# 15041) was coated with 2.0 ug/ml anti-INFg NlB42 (BD Pharmingen, cat#551221 through Fisher) at 100 ul in 0.1 M carbonate, pH9.5 for O/N at 4C. The plate was then blocked with 0.3% NF milk in TBS (200 ul/well) for 1 hr at RT and wash 3× with wash buffer 200 ul/well (1× TBST: 0.1 M Tris, 0.5 M NaCl, 0.05% Tween20). Next, 100 ul cell culture sup were transferred on the plate from the killing assay post 24 h and 48 h on to the plate and incubated for 1 h at RT. The interferon gamma (INFg) standard (recombinant human interferon gamma from Thermo Fisher, cat#RIFNG100) was prepared at starting concentration at 0.1 ug/ml and 3× series dilution to 1pg/ml and then 100 ul was added per well and incubated for 1h at RT. The dilution buffer is 1% BSA in 1× TBS (0.1 M Tris, 0.5 M NaCl). The plate was then washed 3× with wash buffer and biotinylated-mouse anti-human INFg (BD Pharmingen, cat# 554550 through Fisher) was added at 1 ug/ml concentration at RT for 1 hour. The plate was washed 3× with wash buffer and HRP-conjugated SA (Pierce high sensitivity Streptavidin-HRP: Thermo Fisher, Cat# 21130) was added at 1:2000; it was applied at 100 ul per well and incubated at RT in the dark for 1 hr. The plate was washed again 3× with wash buffer and 100 ul 1-Step Ultra TMB-ELISA from Thermo Fisher, Prod#34028 was added per well. The plate was read at 405 nm when the color developed.

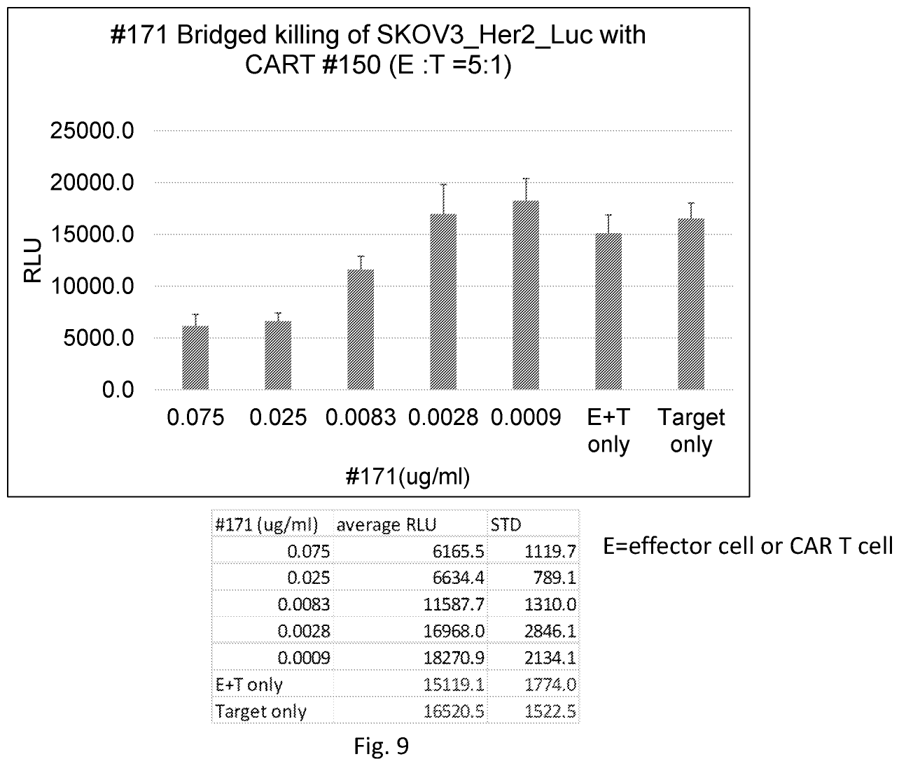

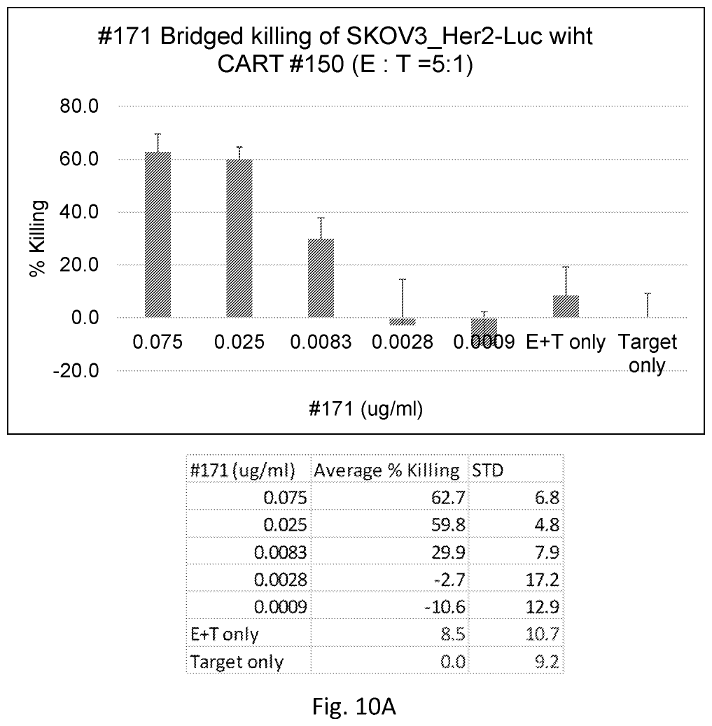

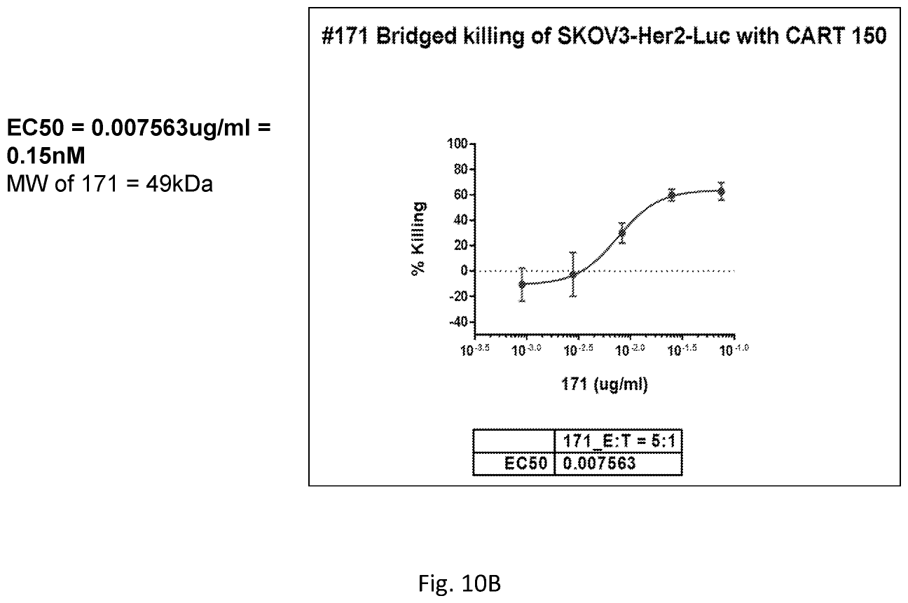

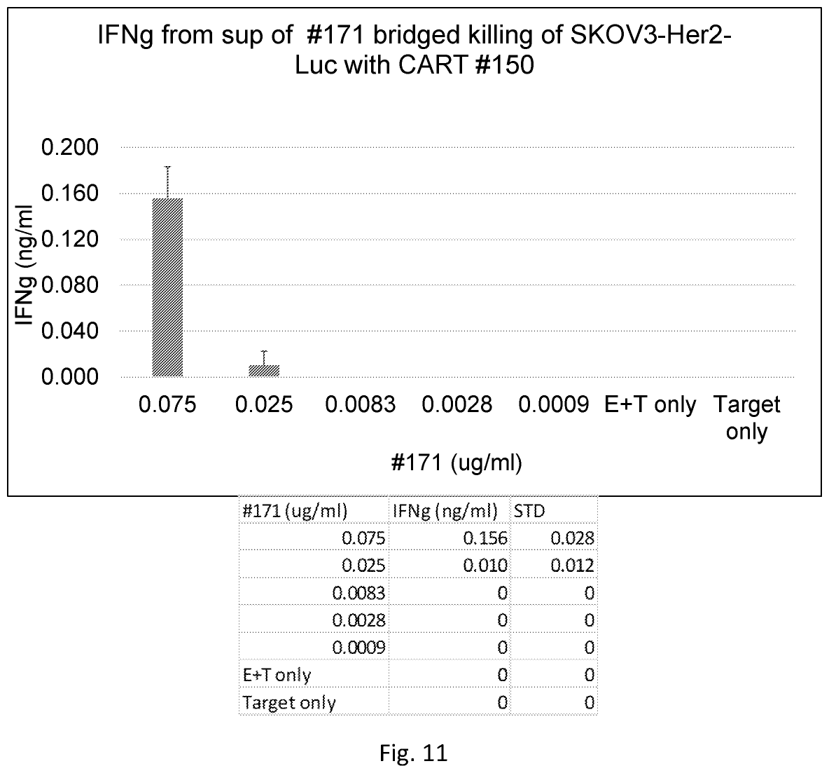

The results of the luciferase release killing assay are shown in FIG. 9. The results demonstrate that Trastuzumab scFv/anti-ld scFv fusion proteins successfully redirected the targeting activity of the CAR19 T cell to kill a Her2-positive (and CD19-negative) cell in a fusion protein dose dependent manner. FIGS. 10A and 10B demonstrate the calculated cytotoxicity and EC50, respectively, of the CAR19 T cell killing as redirected by the Trastuzumab scFv/anti-ld scFv fusion proteins. A summary of the IFNg ELISA results is shown in FIG. 11.

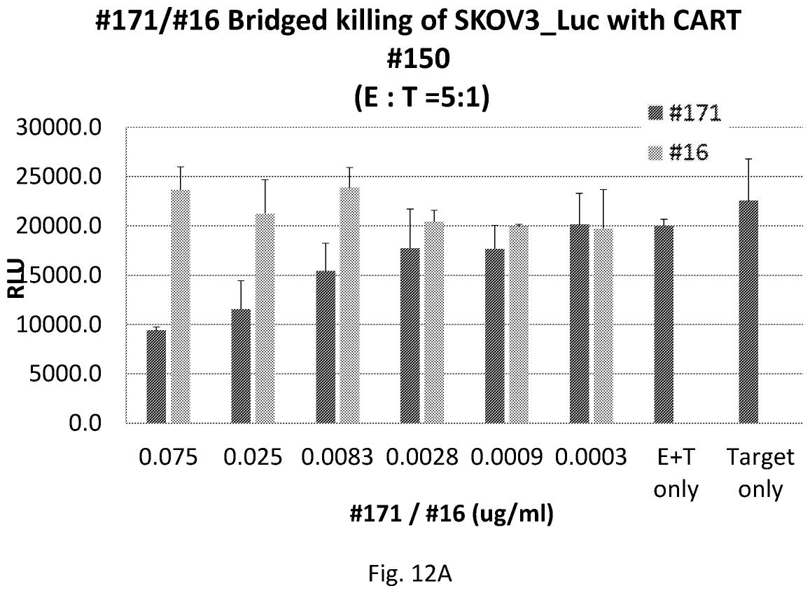

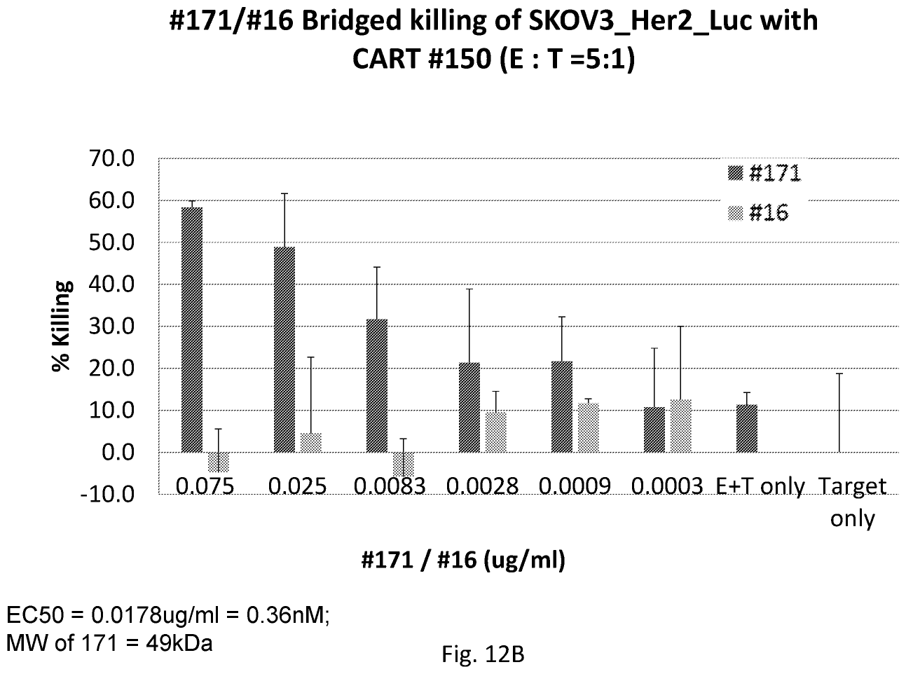

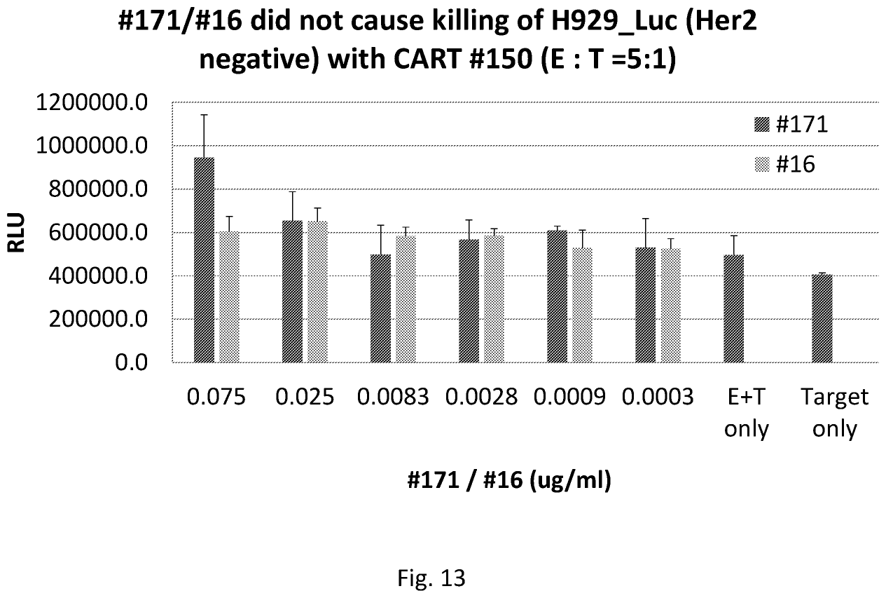

The specificity of redirected killing by Trastuzumab scFv/anti-ld scFv fusion proteins is further demonstrated by comparison to control protiens lacking an anti-Id scFv portion. FIGS. 12A and 12B demonstrate that incubation of CAR19 T cells with a a Her2-positive (and CD19-negative) cell and an anti-Her2 protein (construct #16) using assays as described above does not result in killing whereas a Trastuzumab scFv/anti-ld scFv fusion protein does redirect killing of the Her2 positive cell by a CAR19 T cell. Further, FIG. 13 demonstrates that when the target cell (H929) lacks Her2 no redirected killing by the CAR19 T cell occurs.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

### Listing of Amino Acid Sequences

SEQ ID NO. 1
MEFGLSWVFLVALFRGVQCQVQLQESGPGLVKPSETLSLTCTVSGGSVSSGDYYWTWIRQSPGKGLEWIGHIYYSGNTNYNPSLK
SRLTISIDTSKTQFSLKLSSVTAADTAIYYCVRDRVTGAFDIWGQGTMVTVSSASTKGPSVFPLAPCSRSTSESTAALGCLVKDY
FPEPVTVSWNSGALTSGVHTFPAVLQSSGLYSLSSVVTVPSSNFGTQTYTCNVDHKPSNTKVDKTVERKCCVECPPCPAPPVAGP
SVFLFPPKPKDTLMISRTPEVTCVVVDVSHEDPEVQFNWYVDGVEVHNAKTKPREEQFQSTFRVVSVLTVVHQDWLNGKEYKCKV
SNKGLPAPIEKTISKTKGQPREPQVYTLPPSREEMTKNQVSLTCLVKGFYPSDIAVEWESNGQPENNYKTTPPMLDSDGSFFLYS
KLTVDKSRWQQGNVFSCSVMHEALHNHYTQKSLSLSPG
SEQ ID NO. 2
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGGGGSGGGGSGGGGSDIQMTQSPSSLSASVGDRVTITCQASQDISNYLNWYQ
QKPGKAPKLLIYDASNLETGVPSRFSGSGSGTDFTFTISSLQPEDIATYFCQHFDHLPLAFGGGTKVEIKRTVAAPSVFIFPPSD
EQLKSGTASVVCLLNNFYPREAKVQWKVDNALQSGNSQESVTEQDSKDSTYSLSSTLTLSKADYEKHKVYACEVTHQGLSSPVTK
SFNRGEC
SEQ ID NO. 3
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGGGGSGGGGSGGGGSQVQLQESGPGLVKPSETLSLTCTVSGGSVSSGDYYWT
WIRQSPGKGLEWIGHIYYSGNTNYNPSLKSRLTISIDTSKTQFSLKLSSVTAADTAIYYCVRDRVTGAFDIWGQGTMVTVSSAST
KGPSVFPLAPCSRSTSESTAALGCLVKDYFPEPVTVSWNSGALTSGVHTFPAVLQSSGLYSLSSVVTVPSSNFGTQTYTCNVDHK
PSNTKVDKTVERKCCVECPPCPAPPVAGPSVFLFPPKPKDTLMISRTPEVTCVVVDVSHEDPEVQFNWYVDGVEVHNAKTKPREE
QFQSTFRVVSVLTVVHQDWLNGKEYKCKVSNKGLPAPIEKTISKTKGQPREPQVYTLPPSREEMTKNQVSLTCLVKGFYPSDIAV
EWESNGQPENNYKTTPPMLDSDGSFFLYSKLTVDKSRWQQGNVFSCSVMHEALHNHYTQKSLSLSPG
SEQ ID NO. 4
METDTLLLWVLLLWVPGSTGDIQMTQSPSSLSASVGDRVTITCQASQDISNYLNWYQQKPGKAPKLLIYDASNLETGVPSRFSGS
GSGTDFTFTISSLQPEDIATYFCQHFDHLPLAFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
SEQ ID NO. 5
METDTLLLWVLLLWVPGSTGDIQMTQSPSSLSASVGDRVTITCQASQDISNYLNWYQQKPGKAPKLLIYDASNLETGVPSRFSGS
GSGTDFTFTISSLQPEDIATYFCQHFDHLPLAFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECDKTHTSPPSPAPEAAGGPSEV
RPEEPLVVKVEEGDNAVLQCLKGTSDGPTQQLTWSRESPLKPFLKLSLGLPGLGIHMRPLAIWLFIFNVSQQMGGFYLCQPGPPS
EKAWQPGWTVNVEGSG
SEQ ID NO. 6
MEFGLSWVFLVALFRGVQCQVQLQESGPGLVKPSETLSLTCTVSGGSVSSGDYYWTWIRQSPGKGLEWIGHIYYSGNTNYNPSLK
SRLTISIDTSKTQFSLKLSSVTAADTAIYYCVRDRVTGAFDIWGQGTMVTVSSASTKGPSVFPLAPCSRSTSESTAALGCLVKDY
FPEPVTVSWNSGALTSGVHTFPAVLQSSGLYSLSSVVTVPSSNFGTQTYTCNVDHKPSNTKVDKTVERKCCVECPPCPAPPVAGP
SVFLFPPKPKDTLMISRTPEVTCVVVDVSHEDPEVQFNWYVDGVEVHNAKTKPREEQFQSTFRVVSVLTVVHQDWLNGKEYKCKV
SNKGLPAPIEKTISKTKGQPREPQVYTLPPSREEMTKNQVSLTCLVKGFYPSDIAVEWESNGQPENNYKTTPPMLDSDGSFFLYS
KLTVDKSRWQQGNVFSCSVMHEALHNHYTQKSLSLSPGGGGGSGGGGSGGGGSEVRPEEPLVVKVEEGDNAVLQCLKGTSDGPTQ
QLTWSRESPLKPFLKLSLGLPGLGIHMRPLAIWLFIFNVSQQMGGFYLCQPGPPSEKAWQPGWTVNVEGSG
SEQ ID NO. 7
MEFGLSWVFLVALFRGVQCQVQLVQSGAEVKKPGASVKVSCKASGYTFTDYYMHWVRQAPGQGLEWMGRVNPNRRGTTYNQKFEG
RVTMTTDTSTSTAYMELRSLRSDDTAVYYCARANWLDYWGQGTTVTVSSASTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVDKRVESKYGPPCPPCPAPEAAGGPSVF
LFPPKPKDTLMISRTPEVTCVVVDVSQEDPEVQFNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLG
SEQ ID NO. 8
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGGGGSGGGGSGGGGSDIQMTQSPSSLSASVGDRVTITCSVSSSVSSIYLHWY
QQKPGKAPKLLIYSTSNLASGVPSRFSGSGSGTDFTLTISSLQPEDFATYYCQVYSGYPLTFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTHQGLSSPVT
KSFNRGEC
SEQ ID NO. 9
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGGGGSGGGGSGGGGSQVQLVQSGAEVKKPGASVKVSCKASGYTFTDYYMHWV
RQAPGQGLEWMGRVNPNRRGTTYNQKFEGRVTMTTDTSTSTAYMELRSLRSDDTAVYYCARANWLDYWGQGTTVTVSSASTKGPS
VFPLAPCSRSTSESTAALGCLVKDYFPEPVTVSWNSGALTSGVHTFPAVLQSSGLYSLSSVVTVPSSSLGTKTYTCNVDHKPSNT
KVDKRVESKYGPPCPPCPAPEAAGGPSVFLFPPKPKDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHYTQKSLSLSL
SEQ ID NO. 10
METDTLLLWVLLLWVPGSTGDIQMTQSPSSLSASVGDRVTITCSVSSSVSSIYLHWYQQKPGKAPKLLIYSTSNLASGVPSRFSG
SGSGTDFTLTISSLQPEDFATYYCQVYSGYPLTFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
SEQ ID NO. 11
METDTLLLWVLLLWVPGSTGDIQMTQSPSSLSASVGDRVTITCSVSSSVSSIYLHWYQQKPGKAPKLLIYSTSNLASGVPSRFSG
SGSGTDFTLTISSLQPEDFATYYCQVYSGYPLTFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECDKTHTSPPSPAPEAAGGPSE
VRPEEPLVVKVEEGDNAVLQCLKGTSDGPTQQLTWSRESPLKPFLKLSLGLPGLGIHMRPLAIWLFIFNVSQQMGGFYLCQPGPP
SEKAWQPGWTVNVEGSG
SEQ ID NO. 12
MEFGLSWVFLVALFRGVQCQVQLVQSGAEVKKPGASVKVSCKASGYTFTDYYMHWVRQAPGQGLEWMGRVNPNRRGTTYNQKFEG
RVTMTTDTSTSTAYMELRSLRSDDTAVYYCARANWLDYWGQGTTVTVSSASTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVDKRVESKYGPPCPPCPAPEAAGGPSVF
LFPPKPKDTLMISRTPEVTCVVVDVSQEDPEVQFNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGGGGGSGGGGSGGGGSEVRPEEPLVVKVEEGDNAVLQCLKGTSDGPTQQLT
WSRESPLKPFLKLSLGLPGLGIHMRPLAIWLFIFNVSQQMGGFYLCQPGPPSEKAWQPGWTVNVEGSG
SEQ ID NO. 13
METDTLLLWVLLLWVPGSTGDIQMTQTTSSLSASLGDRVTISCRASQDISKYLNWYQQKPDGTVKLLIYHTSRLHSGVPSRFSGS
GSGTDYSLTISNLEQEDIATYFCQQGNTLPYTFGGGTKLEITGGGGSGGGGSGGGGSGGGGSEVKLQESGPGLVAPSQSLSVTCT
VSGVSLPDYGVSWIRQPPRKGLEWLGVIWGSETTYYNSALKSRLTIIKDNSKSQVFLKMNSLQTDDTAIYYCAKHYYYGGSYAMD
YWGQGTSVTVSSPSQFRVSPLDRTWNLGETVELKCQVLLSNPTSGCSWLFQPRGAAASPTFLLYLSQNKPKAAEGLDTQRFSGKR
LGDTFVLTLSDFRRENEGYYFCSALSNSIMYFSHFVPVFLPAKPTTTPAPRPPTPAPTIASQPLSLRPEACRPAAGGAVHTRGLD
FACDIYIWAPLAGTCGVLLLSLVITRSKRSRLLHSDYMNMTPRRPGPTRKHYQPYAPPRDFAAYRSKRGRKKLLYIFKQPFMRPV
QTTQEEDGCSCRFPEEEEGGCELRVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPR
SEQ ID NO. 14
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGHHHHHH
SEQ ID NO. 15
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQGNVFSCSVMHEALHNHYTQKSL
SLSPG
SEQ ID NO. 16
MEFGLSWVFLVALFRGVQCEVQLVESGGGLVQPGGSLRLSCAASGFNIKDTYIHWVRQAPGKGLEWVARIYPTNGYTRYADSVKG
RFTISADTSKNTAYLQMNSLRAEDTAVYYCSRWGGDGFYAMDYWGQGTLVTVSSASTGGGGSGGGGSGGGGSDIQMTQSPSSLSA
SVGDRVTITCRASQDVNTAVAWYQQKPGKAPKLLIYSASFLYSGVPSRFSGSGSGTDFTLTISSLQPEDFATYYCQQHYTTPPTF
GQGTKVEIKRTGPHHHHHH
SEQ ID NO. 17
MEFGLSWVFLVALFRGVQCQVQLVQSGAEDKKPGESVKISCKASGYTFTNYGMNWVRQAPGQGLKWMGWINTYTGESTYADDFKG
RFAFSLDTSASTAYLQLSSLRGEDTAVYFCARFAIKGDYWGQGTTVTVSSASTGGGGSGGGGSGGGGSGGGGSDIVMTQSPLSLE
VSPGEPASISCRSTKSLLHSDGITYLYWYLQKPGQSPQLLIYQLSNLASGVPDRFSSSGSGTDFTLKISRVEAEDEGTYYCAQNL
EIPRTFGQGTKLEIKRTGPHHHHHH
SEQ ID NO. 18
METDTLLLWVLLLWVPGSTGDIVMTQSPLSLPVTPGEPASISCRSTKSLLHSDGITYLYWYLQKPGQSPQLLIYQLSNLASGVPD
RFSSSGSGTDFTLKISRVEAEDEGVYYCAQNLEIPRTFGCGTKLEIKRTGGGGSGGGGSGGGGSGGGGSQVQLVQSGAEVKKPGE
SVKISCKASGYTFTNYGMNWVRQAPGQCLKWMGWINTYTGESTYADDFKGRFAFSLDTSASTAYLQLSSLRSEDTAVYFCARFAI
KGDYWGQGTLVTVSSGPHHHHHH
SEQ ID NO. 19
MEFGLSWVFLVALFRGVQCQVQLVQSGAEDVKPDASVKLSCKASGYTFTDYYMHWVRQAPGQGLEWMGRVNPNRRGTTYNQKFEG
RVTMTTDTSTSTAYMQLSSLRGEDTAVYYCARANWLDYWGQGTTVTVSSASTGGGGSGGGGSGGGGSGGGGSDIQMTQSPSSLEA
SVGDRVTITCSVSSSVSSIYLHWYQQKPGKSPKLLIYSTSNLASGVPDRFSGSGSGTDFTLTISSLQAEDEGTYYCQVYSGYPLT
FGGGTKLEIKRTGPHHHHHH
SEQ ID NO. 20
METDTLLLWVLLLWVPGSTGDIQMTQSPSSLSASVGDRVTITCSVSSSVSSIYLHWYQQKPGKAPKLLIYSTSNLASGVPSRFSG
SGSGTDFTLTISSLQPEDEATYYCQVYSGYPLTFGCGTKVEIKRTGGGGSGGGGSGGGGSGGGGSQVQLVQSGAEDKKPGASVKV
SCKASGYTFTDYYMHWVRQAPGQCLEWMGRVNPNRRGTTYNQKFEGRVTMTTDTSTSTAYMELRSLRSDDTAVYYCARANWLDYW
GQGTTVTVSSGPHHHHHH
SEQ ID NO. 21
MEFGLSWVFLVALFRGVQCQVQLQESGPGDVKPSETLSLTCTVSGGSVSSGDYYWTWIRQSPGKGLEWIGHIYYSGNTNYNPSLK
SRLTISIDTSKTTFSLQLSSVTGEDTAIYYCVRDRVTGAFDIWGQGTTVTVSSASTGGGGSGGGGSGGGGSGGGGSDIQMTQSPS
SLEASVGDRVTITCQASQDISNYLNWYQQKPGKSPKLLIYDASNLETGVPDRFSGSGSGTDFTFTISSLQAEDEGTYFCQHFDHL
PLAFGGGTKLEIKRTGPHHHHHH
SEQ ID NO. 22
METDTLLLWVLLLWVPGSTGDIQMTQSPSSLSASVGDRVTITCQASQDISNYLNWYQQKPGKAPKLLIYDASNLETGVPSRFSGS
GSGTDFTFTISSLQPEDEATYFCQHFDHLPLAFGCGTKVEIKRTGGGGSGGGGSGGGGSGGGGSQVQLQESGPGDVKPSETLSLT
CTVSGGSVSSGDYYWTWIRQSPGKCLEWIGHIYYSGNTNYNPSLKSRLTISIDTSKTQFSLKLSSVTAADTAIYYCVRDRVTGAF
DIWGQGTTVTVSSGPHHHHHH
SEQ ID NO. 23
MPPPRLLFFLLFLTPMEVRHHHHHHPEEPLVVKVEEGDNAVLQCLKGTSDGPTQQLTWSRESPLKPFLKLSLGLPGLGIHMRPLA
IWLFIFNVSQQMGGFYLCQPGPPSEKAWQPGWTVNVEGSGELFRWNVSDLGGLGCGLKNRSSEGPSSPSGKLMSPKLYVWAKDRP
EIWEGEPPCLPPRDSLNQSLSQDLTMAPGSTLWLSCGVPPDSVSRGPLSWTHVHPKGPKSLLSLELKDDRPARDMWVMETGLLLP
RATAQDAGKYYCHRGNLTMSFHLEITARPGGGGSGGGGSGGGGSVRSSSRTPSDKPVAHVVANPQAEGQLQWLNRRANALLANGV
ELRDNQLVVPSEGLYLIYSQVLFKGQGCPSTHVLLTHTISRIAVSYQTKVNLLSAIKSPCQRETPEGAEAKPWYEPIYLGGVFQL
EKGDRLSAEINRPDYLDFAESGQVYFGIIAL
SEQ ID NO. 24
MEFGLSWVFLVALFRGVQCEVQLVESGGGLVQPGGSLRLSCAASGFNIKDTYIHWVRQAPGKGLEWVARIYPTNGYTRYADSVKG
RFTISADTSKNTAYLQMNSLRAEDTAVYYCSRWGGDGFYAMDYWGQGTLVTVSSASTGGGGSGGGGSGGGGSDIQMTQSPSSLSA
SVGDRVTITCRASQDVNTAVAWYQQKPGKAPKLLIYSASFLYSGVPSRFSGSRSGTDFTLTISSLQPEDFATYYCQQHYTTPPTF
GQGTKVEIKRTDKTHTSPPSPAPEAAGGPSEVRPEEPLVVKVEEGDNAVLQCLKGTSDGPTQQLTWSRESPLKPFLKLSLGLPGL
GIHMRPLAIWLFIFNVSQQMGGFYLCQPGPPSEKAWQPGWTVNVEGSGSRGPHHHHHH
SEQ ID NO. 25
MEFGLSWVFLVALFRGVQCEVQLVESGGGLVQPGGSLRLSCAASGFNIKDTYIHWVRQAPGKGLEWVARIYPTNGYTRYADSVKG
RFTISADTSKNTAYLQMNSLRAEDTAVYYCSRWGGDGFYAMDYWGQGTLVTVSSASTGGGGSGGGGSGGGGSDIQMTQSPSSLSA
SVGDRVTITCRASQDVNTAVAWYQQKPGKAPKLLIYSASFLYSGVPSRFSGSRSGTDFTLTISSLQPEDFATYYCQQHYTTPPTF
GQGTKVEIKRTDKTHTSPPSPAPEAAGGPSEVRPEEPLVVKVEEGDNAVLQCLKGTSDGPTQQLTWSRESPLKPFLKLSLGLPGL
GIHMRPLAIWLFIFNVSQQMGGFYLCQPGPPSEKAWQPGWTVNVEGSGDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLTVDKSRWQQGNVFSCSV
MHEALHNHYTQKSLSLSPG
SEQ ID NO. 26
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGGGGGSGGGGSGGGGSGGGGSEVQLVESGGGLVQPGGSLRLSCAASGFNIKD
TYIHWVRQAPGKGLEWVARIYPTNGYTRYADSVKGRFTISADTSKNTAYLQMNSLRAEDTAVYYCSRWGGDGFYAMDYWGQGTLV
TVSSASTGGGGSGGGGSGGGGSDIQMTQSPSSLSASVGDRVTITCRASQDVNTAVAWYQQKPGKAPKLLIYSASFLYSGVPSRFS
GSRSGTDFTLTISSLQPEDFATYYCQQHYTTPPTFGQGTKVEIKRTSRGPHHHHHH
SEQ ID NO. 27
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGGGGGSGGGGSGGGGSGGGGSEVQLVESGGGLVQPGGSLRLSCAASGFNIKD
TYIHWVRQAPGKGLEWVARIYPTNGYTRYADSVKGRFTISADTSKNTAYLQMNSLRAEDTAVYYCSRWGGDGFYAMDYWGQGTLV
TVSSASTGGGGSGGGGSGGGGSDIQMTQSPSSLSASVGDRVTITCRASQDVNTAVAWYQQKPGKAPKLLIYSASFLYSGVPSRFS
GSRSGTDFTLTISSLQPEDFATYYCQQHYTTPPTFGQGTKVEIKRTDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDKSRWQQGNVFSCSVMH
EALHNHYTQKSLSLSPG
SEQ ID NO. 28
MPPPRLLFFLLFLTPMVERPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDDLTMAPGSTLWLSCGVPPDSVSRGPLSWTHVHPKGPKSLLSLELKDDRPARDMWVMETGLLLPRATAQ
DAGKYYCHRGNLTMSFHLEITARPHHHHHH
SEQ ID NO. 29
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMHEALHNHYTQKSLSLSPG
SEQ ID NO. 30
MPPPRLLFFLFLTPMEVRPRDSLNQSLSQDLTMAPGSTLWLSCGVPPDSVSRGPLSWTHVHPKGPKSLLSLELKDDRPARDMWVM
ETGLLLPRATAQDAGKYYCHRGNLTMSFHLEITARPHHHHHH
SEQ ID NO. 31
MPPPRLLFFLLFLTPMEVRPRDSLNQSLSQDLTMAPGSTLWLSCGVPPDSVSRGPLSWTHVHPKGPKSLLSLELKDDRPARDMWV
METGLLPRATAQDAGKYYCHRGNLTMSFHLEITARPDKTHTCPPCPAPELLGGSPVFLFPPKPKDTLMISRTPEVTCVVVDVSHE
DPEVKFNWYVDGHEVHNAKTKPREEQYNSTYRVVSVLTVLHQDWLNGKEYKCKVSNKALPAPIEKTISKAKGQPREPQVYTLPPS
REEMTKNQVSLTCLVKGFYPSDIAVEWESNGQEPNNYKTTPPVLDSDGSFFLYSKLTVDKSRWQQGNVFSCSVMHEALHNHYTQK
SLSLSPG
SEQ ID NO. 32
MPPPRLLFFLLFLTPMEVRPEEPLVVKVEEGDVANLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWADKRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSDIQMTQSPSSLSASVGDRVTITCQASQDISNYLNWYQQKPGKAPKLL
IYDASNLETGVPSRFSGSGSGTDFTFTISSLQPEDIATYFCQHFDHLPLAFGGGTKVEIKRTVAAPSVFIFPPSDEQLKSGTASV
VCLLNNFYPREAKVQWKVDNALQSGNSQESVTEQDSKDSTYSLSSTLTLSKADYEKHKVYACEVTHQGLSSPVTKSFNRGEC
SEQ ID NO. 33
MPPPRLLFFLLFLTPMEVRPEEPLVVKVEEGDVANLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSQVQLQESGPGLVKPSETLSLTCTVSGGSVSSGDYYWTWIRQSPGKGL
EWIGHIYYSGNTNYNPSLKSRLTISIDTSKTQFSLKLSSVTAADTAIYYCVRDRVTGAFDIWGQGTMVTVSSASTKGPSVFPLAP
CSRSTSESTAALGCLVKDYFPEPVTVSWNSGALTSGVHTFPAVLQSSLGYSLSSVVTVPSSNFGTQTYTCNVDHKPSNTKVDKTV
ERKCCVECPPCPAPPVAGPSVFLFPPKPKDTLMISRPTEVTCVVVDVSHEDPEVQFNWYVDGVEVHNAKTKPREEQFQSTFRVVS
VLTVVHQDWLNGKEYKCKVSNKGLPAPIEKTISKTKGQPREPQVYTLPPSREEMTKNQVSLTCLVKGFYPSDIAVEWESNGQPEN
NYKTTPPMLDSDGSFFLYSKLTVDKSRWQQGNVFSCSVMHEALHNHYTQKSLSLSPG
SEQ ID NO. 34
METDTLLLWVLLLWVPGSTGDIQMTQSPSSLSASVGDRVTITCQASQDISNYLNWYQQKPGKAPKLLIYDASNLETGVPSRFSGS
GSGTDFTFTISSLQPEDIATYFCQHFDHLPLAFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECDKTHTSPPSPAPEAAGGPSPE
EPLVVKVEEGDNAVLQCLKGTSDGPTQQLTWSRESPLKPFLKLSLGLPGLGIHMRPLAIWLFIFNVSQQMGGFYLCQPGPPSEKA
WQPGWTVNVEGSGELFRWNVSDLGGLGCGLKNRSSEGPSSPSGKLMSPKLYVWAKDRPEIWEGEPPCLPPRDSLNQSLSQDLTMA
PGSTLWLSCGVPPDSVSRGPLSWTHVHPKGPKSLLSLELKDDRPARDMWVMETGLLLPRATAQDAGKYYCHRGNLTMSFHLEITA
RP
SEQ ID NO. 35
MEFGLSWVFLVALFRGVQCQVQLQESGPGLVKPSETLSLTCTVSGGSVSSGDYYWTWIRQSPGKGLEWIGHIYYSGNTNYNPSLK
SRLTISIDTSKTQFSLKLSSVTAADTAIYYCVRDRVTGAFDIWGQGTMVTVSSASTKGPSVFPLAPCSRSTSESTAALGCLVKDY
FPEPVTVSWNSGALTSGVHTFPAVLQSSGLYSLSSVVTVPSSNFGTQTYTCNVDHKPSNTKVDKTVERKCCVECPPCPAPPVAGP
SVFLFPPKPKDTLMISRTPEVTCVVVDVSHEDPEVQFNWYVDGVEVHNAKTKPREEQFQSTFRVVSVLTVVHQDWLNGKEYKCKV
SNKGLPAPIEKTISKTKGQPREPQVYTLPPSREEMTKNQVSLTCLVKGFYPSDIAVEWESNGQEPNNYKTTPPMLDSDGSFFLYS
KLTVDKSRWQQGNVFSCSVMHEALHNHYTQKSLSLSPGGGGSGGGGSGGGGSPEEPLVVKVEEGDNAVLQCLKGTSDGPTQQLTW
SRESPLKPFLKLSLGLPGLGIHMRPLAIWLFIFNVSQQMGGFLYCQPGPPSEKAWQPGWTVNVEGSGELFRWNVSDLGGLGCGLK
NRSSEGPSSPSGKLMSPKLYVWAKDRPEIWEGEPPCLPPRDSLNQSLSQDLTMAPGSTLWLSCGVPPDSVSRGPLSWTHVHPKGP
KSLLSLELKDDRPARDMWVMETGLLLPRATAQDAGKYYCHRGNLTMSFHLEITARP
SEQ ID NO. 36
MPPPRLLFFLLFTLPVEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSDIQMTQSPSSLSASVGDRVTITCSVSSSVSSIYLHWYQQKPGKAPKL
LIYSTSNLASGVPSRFSGSGSGTDFTLTISSLQPEDFATYYCQVYSGYPLTFGGGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQGLSSPVTKSFNRGEC
SEQ ID NO. 37
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSQVQLVQSGAEVKKPGASVKVSCKASGYTFTDYYMHWVRQAPGKGLEW
MGRVNPNRRGTTYNQKFEGRVTMTTDTSTSTAYMELRSLRSDDTAVYYCARANWLDYWGQGTTVTVSSASTKGPSVFPLAPCSRS
TSESTAALGCLVKDYFPEPVTVSWNSGALTSGVHTFPAVLQSSGLYSLSSVVTVPSSSLGTKTYTCNVDHKPSNTKVDKRVESKY
GPPCPPCPAPEAAGGPSVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSL
SEQ ID NO. 39
METDTLLLWVLLLWVPGSTGDIQMTQSPSSLSASVGDRVTITCSVSSSVSSIYLHWYQQKPGKAPKLLIYSTSNLASGVPSRFSG
SGSGTDFTLTISSLQPEDFATYYCQVYSGYPLTFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECDKTHTSPPSPAPEAAGGPSP
EEPLVVKVEEGDNALQCLKGTSDGPTQQLTWSRESPLKPFLKLSLGLPGLGIHMRPLAIWLFIFNVWQQMGGFYLCQPGPPSEKA
WQPGWTVNVEGSGELFRWNVSDLGGLGCGLKNRSSEGPSSPSGKLMSPKLYVWAKDRPEIWEGEPPCLPPRDSLNQSLSQDLTMA
PGSTLWLSCGVPPDSVSRGPLSWTHVHPKGPKSLLSLELKDDRPARDMWVMETGLLLPRATAQDAGKYYCHRGNLTMSFHLEITA
RP
SEQ ID NO. 39
MEFGLSWVFLVALFRGVQCQVQLVQSGAEVKKPGASVKVSCKASGYTFTDYYMHWVRQAPGQGLEWMGRVNPNRRGTTYNQKFEG
RVTMTTDTSTSTAYMELRSLRSDDTAVYYCARANWLDYWGQGTTVTVSSASTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVDKRVESKYGPPCPPCPAPEAAGGPSVF
LFPPKPKDTLMISRTPEVTCVVVDVSQEDPEVQFNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGGGGSGGGGSGGGGSPEEPLVVKVEEGDNAVLQCLKGTSDGPTQQLTWSRE
SPLKPFLKLSLGLPGLGIHMRPLAIWLFIFNVSQQMGGFYLCQPGPPSEKAWQPGWTVNVEGSGELFRWNVSDLGGLGCGLKNRS
SEGPSSPSGKLMSPKLYVWAKDRPEIWEGEPPCLPPRDSLNQSLSQDLTMAPGSTLWLSCGVPPDSVSRGPLSWTHVHPKGPKSL
LSLELKDDRPARDMWVMETGLLLPRATAQQDAGKYYCHRGNLTMSFHLEITARP
SEQ ID NO. 40
MEFGLSWVFLVALFRGVQCEVQLVESGGGLVQPGGSLRLSCAASGFNIKDTYIHWVRQAPGKGLEWVARIYPTNGYTRYADSVKG
RFTISADTSKNTAYLQMNSLRAEDTAVYYCSRWGGDGFYAMDYWGQGTLVTVSSASTGGGGSGGGGSGGGGSDIQMTQSPSSLSA
SVGDRVTITCRASQDVNTAVAWYQQKPGKAPKLLIYSASFLYSGVPSRFSGSRSGTDFTLTISSLQPEDFATYYCQQHYTTPPTF
GQGTKVEIMKRTDKTHTSPPSPAPEAAGGPSPEEPLVVKVEEGDNAVLQCLKGTSDGPTQQLTWSRESPLKPFLKLSLGLPGLGI
HMRPLAIWLFIFNVSQQMGGFYLCQPGPPSEKAWQPGWTVNVEGSGELFRWNVSDLGGLGCGLKNRSSEGPSSPSGKLMSPKLYV
WAKDRPEIWEGEPPCLPPRDSLNQSLSQDLTMAPGSTLWLSCGVPPDSVSRGPLSWTHVHPKGPKSLLSLELKDDRPARDMWVME
TGLLLPRATAQDAGKYYCHRGNLTMSFHLEITARPSRGPHHHHHH
SEQ ID NO. 41
MEFGLSWVFLVALFRGVQVEVQLVESGGGLVQPGGSLRLSCAASGFNIKDTYIHWVRQAPGKGLEWVARIYPTNYGYTRYADSVK
GRFTISADTSKNTAYLQMNSLRAEDTAVYYCSRWGGDGFYAMDYWGQGTLVTVSSASTGGGGSGGGGSGGGGSDIQMTQSPSSLS
ASVGDRVTITCRASQDVNTAVAWYQQKPGKAPKLLIYSASFLYSGVPSRFSGSRSGTDFTLTISSLQPEDFATYYCQQHYTTPPT
FGQGTKVEIKRTDKTHTSPPSPAPEAAGGPSPEEPLVVKVEEGDNAVLQCLKGTSDGPTQQLTWSRESPLKPFLKLSLGLPGLGI
HMRPLAIWLFIFNVSQQMGGFYLCQPGPPSEKAWQPGWTVNVEGSGELFRWNVSDLGGLGCGLKNRSSEGPSSPSGKLMSPKLYV
WAKDRPEIWEGEPPCLPPRDSLNQSLSQDLTMAPGSTLWLSCGVPPDSVSRGPLSWTHVHPKGPKSLLSLELKDDRPARDMWVME
TGLLLPRATAQDAGKYYCHRGNLTMSFHLEITARPDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKSRWQQGNVFSCSVMHEALHNHYTQKS
LSLSPG
SEQ ID NO. 42
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTSRGPHHHHHH
SEQ ID NO. 43
MPPPRLLFFLLFLTPMEVRPEEPLVVKVEEDGNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTDKTHTCPPCPAPELLGGPSVFLFPPKPKDTLMISRTPEVTCVVVDVSHED
PEVKFNWYVDGVEVHNAKTKPREEQYNSTRYVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDKSRWQQGNVFSCSVMHEALHNHYTQKS
LSLSPG
SEQ ID NO. 46
MESDESGLPAMEIECRITGTLNGVEFELVGGGEGTPEQGRMTNKMKSTKGALTFSPYLLSHVMGYGFYHFGTYPSGYENPFLHAI
NNGGYTNTRIEKYEDGGVLHVSFSYRYEAGRVIGDFKVMGTGFPEDSVIFTDKIIRSNATVEHLHPMGDNDLDGSFTRTFSLRDG
GYYSSVVDSHMHFKSAIHPSILQNGGPMFAFRRVEEDHSNTELGIVEYQHAFKTPDADAGEERV
SEQ ID NO. 47
MESDESGLPAMEIECRITGTLNGVEFELVGGGEGTPEQGRMTNKMKSTKGALTFSPYLLSHVMGYGFYHFGTYPSGYENPFLHAI
NNGGYTNTRIEKYEDGGVLHVSFSYRYEAGRVIGDFKVMGTGFPEDSVIFTDKIIRSNATVEHLHPMGDNDLDGSFTRTFSLRDD
GGYYSSVVDSHMHFKSAIHPSILQNGGPMFAFRRVEEDHSNTELGIVEYQHAFKTPDADAGEERV
SEQ ID NO. 48
MESDESGLPAMEIECRITGTLNGVEFELVGGGEGTPEQGRMTNKMKSTKGALTFSPYLLSHVMGYGFYHFGTYPSGYENPFLHAI
NNGGYTNTRIEKEDGGVLHNVSFSYRYEAGRVIGDFNVMGTGFPEDSVIFTDKIIRSNATVEHLHPMGDNDLDGSFTRTFSLRDG
GYYSSVVDSHMHFKSAIHPSILQNGGPMFAFRRVEEDHSNTELGIVEYQHAFKTPDADAGEERV
SEQ ID NO. 49
MESDESGLPAMEIECRITGTLNGVEFELVGGGEGTPEQGRMTNKNKSTKGALTFSPYLLSHVMGYGFYHFGTYPSGYENPFLHAI
NNGGYTNTRIEKYEDGGVLHVSFSYRYEAGRVIGDFKVMGTGFPEDSVIFTDKIIRSNATVEHLHPMGDNDLDGSFTRTFSLRDG
GYYSSVVDSHMHFKSAIHPSILQNGGPMFAFRRVEEDHSNTELGIVEYQHAFKTPDADAGEERV
SEQ ID NO. 50
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFLYCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKGGGGSGGGGSGGGGSGGGGSQVQLQESGPGLVKPSETLSLTCTVSGGSV
SSGDYYWTWIRQSPGKGLEWIGHIYYSGNTNYNPSLKSRLTISIDTSKTQFSLKLSSVTAADTAIYYCVRDRVTGAFDIWGQGTM
VTVSSASTKGPSVFPLAPCSRSTSESTAALGCLVKDYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTLMISRTPEVTCVVVDVSHEDPEVQFNWYVDGVEVHN
AKTKPREEQFQSTFRVVSVLTVVHQDWLNGKEYKCKVSNKGLPAPIEKTISKTKGQPREPQVYTLPPSREEMTKNQVSLTCLVKG
FYPSIDAVEWESNGQPENNYKTTPPMLDSDGSFFLYSKLTVDKSRWQQGNVFSCSVMHEALHNHYTQKSLSLSPG
SEQ ID NO. 51
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKGGGGSGGGGSGGGGSGGGGSQVQLVQSGAEVKKPGASVKVSCKASGYTF
TDYYMHWVRQAPGQGLEWMGRVNPNRRGTTYNQKFEGRVTMTTDTSTSTAYMELRSLRSDDTAVYYCARANWLDYWGQGTTVTVS
SASTKGPSVFPLAPCSRSTSESTAALGCKVKDYFPEPVTVSWNSGALTSGVHTFPAVLQSSGLYSLSSVVTVPSSSLGTKTYTCN
CDHKPSNTKVDKRVESKYYGPPCPPCPAPEAAGGPSVFLFPPKPKDTLMISRPTEVTCVVVDVSQEDPEVQFNWYVDGVEVHNAK
TKPREEQFNSTYRVVSVLTVLHQDWLNGKEYKCKVSNKGLPSSIEKTISKAKGQPREPQVYTLPPSQEEMTKNQVSLTCLVKGFY
PSIDAVEWESNGQEPNNYKTTPPVLDSDGSFFLYSRLTVDKSRWQEGNVFSCSVMHEALHNHYTQKSLSLSL
SEQ ID NO. 52
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSFKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHELITARPVLWHWLLRTGGWKGGGGSGGGGSGGGGSGGGGSQVQLVQSGAEDKKPGESVKISCSASGYTF
TNYGMNWVRQAPGQGLKWMGWINTYTGESTYADDFKGRFAFSLDTSASTAYLQLSSLRGEDTAVYFARFAIKGDYWGQGTTVTVS
SASTGGGGSGGGGSGGGGSGGGGSDIVMTQSPLSLEVSPGEPASISVRSTKSLLHSDGITYLYWYLQKPGQSPQLLIYQLSNLAS
GVPDRFSSSGSGTDFTLKISRVEAEDEGTYYCAQNLEIPRTFGQGTKLEIKRTHHHHHH
SEQ ID NO. 53
MPPPRLLFFLLFLTPMVERPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKGGGGSGGGGSGGGGSGGGGSQVQLVQSGAEDVKPDASVKLSCKASGYTF
TDYYMHWVRQAPGQGLEWMGRVNPNRRGTTYNQKFEGRVTMTTDTSTSTAYMQLSSLRGEDTAVYYCARANWLDYWGQGTTVTVS
SASTGGGGSGGGGSGGGGSGGGGSDIQMTQSPSSLEASVGDRVTITCSVSSSVSSIYLHJWYQQKPGKSPKLLIYSTSNLASGVP
DRFSGSGSGTDFTLTISSLQAEDEGTYYCQVYSGYPLTFGGGTKLEIKRTHHHHHH
SEQ ID NO. 54
MPPPRLLFFLLFLTPMEVRPEEPLVVKVEEDGNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKGGGGSGGGGSGGGGSGGGGSQVQLQESGPGDVKPSETLSLTCTVSGGSV
SSGDYYWTWIRQSPGKGLEWIGHIYYSGNTNYNPSLKSRLTISIDTSKTTFSLQLSSVTGEDTAIYYCVRDRVTGAFDIWGQGTT
VTVSSASTGGGGSGGGGSGGGGSGGGGSDIQMTQSPSSLEASVGDRVTITCQASQDISNYLNWYQQKPGKSPKLLIYDASNLETG
VPDRFSGSGSGTDFTFTISSLQAEDEGTYFCQHFDHLPLAFGGGTKLEIKRTHHHHHH
SEQ ID NO. 55
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKGGGGSGGGGSGGGGSGGGGSEVQLVESGGGLVQPGGSLRLSCAASGFNI
KDTYIHWVRQAPGKGLEWVARIYPTNGYTRYADSVKGRFTISADTSKNTAYLQMNSLRAEDTAVYYCSRWGGDGFYAMDYWGQGT
LVTVSSASTGGGGSGGGGSGGGGSDIQMTQSPSSLSASVGDRVTITCRASQDVNTAVAWYQQKPGKAPKLLIYSASFLYSGVPSR
FSGSRSGTDFTLTISSLQPEDFATYYCQQHYTTPPTFGQGTKVEIKRTHHHHHH
SEQ ID NO. 56
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKDKTHTCPPCPAPELLGGPSVFLFPPKPKDTLMISRTPEVTCVVVDVSHE
PEVKFNWYVDGVEVHNAKTKPREEQYNSTYRVVSVLTVLHQDWLNGKEYKCKVSNKALPAPIEKTISKAKGQPREPQVYTLPPSR
EEMTKNQVSLTCLVKGFYPSDAIVEWESNGQPENNYKTTPPVLDSDGSFFLYSKLTVDKSRWQQGNVFSCSVMHEALHNHYTQKS
LSLSPGGGGSGGGGSGGGGSGGGGSEVQLVESGGGLVQPGGSLRLSCAASGFNIKDTYIHWVRQAPGKGLEWVARIYPTNGYTRY
ADSVKGRFTISADTSKNTAYLQMNSLRAEDTAVYYCSRWGGDGFYAMDYWGQGTLVTVSSASTGGGGSGGGGSGGGGSSDIQMTQ
SPSSLSASVGDRVTITCRASQDVNTAVAWYQQKPGKAPKLLIYSASFLYSGVPSRFSGSRSGFTFTLTISSLQPEDFATYYCQQH
YTTPPTFGQGTKVEIKRT
SEQ ID NO. 57
MELAALCRWGLLLALLPPGAASTQVCTGTDMKLRLPASPETHLDMLRHLYQGCQVVQGNLELTYLPTNASLSFLQDIQEVQGYVL
IAHNQVRQVPLQRLRIVRGTQLFEDNYALAVLDNGDPLNNTTPVTGASPGGLRELQLRSLTEILKGGVLIQRNPQLCYQDTILWK
DIFHKNNQLALTLIDTNRSRACHPCSPMCKGSRCWGESSEDCQSLTRTVCAGGCARCKGPLPTDCCHEQCAAGCTGPKHSDCLAC
LHFNHSGICELHCPALVTYNTDTFESMPNPEGRYTFGASCVTACPYNYLSTDVGSCTLVCPLHNQEVTAEDGTQRCEKCSKPCAR
VCYGLGMEHLREVRAVTSANIQEFAGCKKIFGSLAFLPESFDGDPASNTAPLQPEQLQVFETLEEITGYLYISAWPDSLPDLSVF
QNLQVIRGRILHNGAYSLTLQGLGISWLGLRSLRELGSGLALIHHNTHLCFVHTVPWDQLFRNPHQALLHTANRPEDECVGEGLA
CHQLCARGHCWGPGPTQCVNCSQFLRGQECVEECRVLQGLPREYVNARHCLPCHPECQPQNGSVTCFGPEADQCVACAHYKDPPF
CVARCPSGVKPDLSYMPIWKFPDEEGACQPCPINCTHSCVDLDDKGCPAEQRASPLTGGGGSGGGGSGGGGSGGGGSQVQLQESG
PGDVKPSETLSLTCTVSGGSVSSGDYYWTWIRQSPGKGLEWIGHIYYSGNTNYNPSLKSRLTISIDTSKTTFSLQLSSVTGEDTA
IYYCVRDRVTGAFDIWGQGTTVTVSSASTGGGGSGGGGSGGGGSGGGGSDIQMTQSPSSLEASVGDRVTITCQASQDISNYLNWY
QQKPGKSPKLLIYDASNLETGVPDRFSGSGSGTDFTFTISSLQAEDEGTYFCQHFDHLPLAFGGGTKLEIKRTHHHHHH
SEQ ID NO. 58
MELAALCRWGLLLALLPPGAASNRPEDECVGEGLACHQLCARGHCWGPGPTQCVNCSQFLRGQECVEECRVLQGLPREYVNARHC
LPCHPECQPQNGSVTCFGPEADQCVACAHYKDPPFCVARCPSGVKPDLSYMPIWKFPDEEGACQPCPINCTHSCVDLDDKGCPAE
QRASPLTGGGGSGGGGSGGGGSGGGGSQVQLQESGPGDVKPSETLSLTCTVSGGSVSSGDYYWTWIRQSPGKGLEWIGHIYYSGN
TNYNPSLKSRLTISIDTSKTTFSLQLSSVTGEDTAIYYCVRDRVTGAFDIWGQGTTVTVSSASTGGGGSGGGGSGGGGSGGGGSD
IQMTQSPSSLAEASVGDRVTITCQASQDISNYLNWYQQKPGKSPKLLIYDASNLETGVPDRFSGSGSGTDFTFTISSLQAEDEGT
YFCQHFDHLPLAFGGGTKLEIKRTHHHHHH
SEQ ID NO. 63
MPPPRLLFFLLFLTPMEVRPEEPLVVKVEEDGNAVLQCLKGTSDGPTQQLTWSRESPLKPFLKLSLGLPGLGIHMRPLAIWLFIF
NVSQQMGGFYLCQPGPPSEAKWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKGGGGWGGGGSGGGGSGGGGSEVQLQQSGAELVKPGASVKMSCKASGYTF
TSYNMHWVKQTPGQGLEWIGAIYPGNGDTSYNQKFKGKATLTADKSSSTAYMQLSSLTSEDSADYYCARSNY6YGSSYWFFDVWG
AGTTVTVSSGSTSGGGSGGGSGGGGSSDIVLTQSPAILSASPGEKVTMTCRASSSVNYMDWYQKKPGSSPKPWIYATSNLASGVP
ARFSGSGSGTSYSLTISRVEAEDAATYYCQQWSFNPPTFGGGTKLEIKHHHHHH
SEQ ID NO. 64
MHLLGPWLLLVLEYLAFSDSSKWVFEHPETLYAWEGACVWIPCTYRALDGDLESFILFHNPEYNKNTSKFDGTRLYESTKDGKVP
SEQKRVQFLGDKNKACTLSIHPVHLNDSGQLGLRMESKTEKWMERIHLNVSERPFPPHIQLPPEIQESQEVTLTCLLNFSCYGYP
IQLQWLLEGVPMRQAAVTSTSLTIKSVFTRSELKFSPQWSHHGKIVTCQLQDADGKFLSNDTVQLNVKHTPKLEIKVTPSDIAVR
EGDSVTMTCEVSSSNPEYTTVSWLKDGTSLKKQNTFTLNLREVTKDQSGKYCCQVSNDVGPGRSEEVFLQVQYAPEGGGGSGGGG
SGGGGSGGGGSDIQMTQTTSSLSASLGDRVTISCRASQDISKYLNWYQQKPDGTVKLLIHYTSRLHSGVPSRFSGSGSGTDYSLT
ISNLEQEDIATYFCQQGNTLPYTFGGGTKLEITGGGGSGGGGSGGGGSGGGGSEVKLQESGPGLVAPSQSLSVTCTVSGVSLPDY
GVSWIRQPPRKGLEWLGVIWGSETTYYNSALKSRLTIIKDNSKSQVFLKMNSLQTDDTAIYYCAKHYYYGGSYAMDYWGQGTSVT
VSSHHHHHH
SEQ ID NO. 65
MHLLGPWLLLLVLEYLAFSDSSKWVFEHPETLYAWEGACVWWIPCTYRADLGDLESFILFHNPEYNKNTSKFDGTRLYESTKDGK
VPSEQKRQFLGDKNKACTLSIHPVHLNDSGQLGLRMESKTEKWMERIHLNVSERPFPPHIQLPPEIQESQEVTLTCLLNFSCYGY
PIQLQWLLEGVPMRQAAVTSTSLTIKSVFTRSELKFSPQWSHHGKIVTCQLQDADGKFLSNDTVQLNVKHTPKLEIKVTPSDAIV
REGDSVTMTCEVSSSNPEYTTVSWLKDGTSLKKQNTFTLNLREVTKDQSGKYCCQVSNDVGPGRSEEVFLQVQYAPEGGGGSGGG
GSGGGGSGGGGSEVQLQQSGAELVKPGASVKMSCKASGYTFTSYNMHWVKQTPGQGLEWIGAIYPGNGDTSYNQKFKGKATLTAD
KSSSTAYMQLSSLTSEDSADYYCARSNYYGSSYFFDVWGAGTTVTVSSGSTSGGGSGGSGGGGSDIVLTQSPAILSASPGEKVTM
TCRASSSVNYMDWYQKKPGSSPKPWIYATSNLASGVPARFSGSGSGTSYSLTISRVEAEDAATYYCQQWSFNPPTFGGGTKLEIK
HHHHHH
SEQ ID NO. 67
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKGGGGSGGGGSGGGGSGGGGSDILMTQSPSSMSVSLGDTVSITCHSSQDI
NSNIGWLQQRPGKSFKGLIYHGTNLDDEVPSRFSGSGSGADYSLTISSLESEDFADYYCVQYAQFPWTFGGGTKLEIKRGGGGSG
GGGSGGGGSGGGGSMRVLILLWLFTAFPGVLSDVQLQESGPSLVKPSQSLSLTCTVTGYSITSDFAWNWIRQFPGNKLEWMGYIS
YSGNTRYNPSLKSRISITRDTSKNQFFLQLNSVTIEDTATYYCVTAGRGFPYWGQGTLVTVSAHHHHHH
SEQ ID NO. 68
MHLLGPWLLLVLEYLAFSDSSKWVFEHPETLYAWEGACVWIPCTYRALDGDLESFILFHNPEYNKNTSKFDGTRLYESTKDGKVP
SEQKRVQFLGDKNKACTLSIHPVHLNDSGQLGLRMESKTEKWMERIHLNVSERPFPPHIQLPPEIQESQEVTLTCLLNFSCYGYP
IQLQWLLEGVPMRQAAVTSTSLTIKSVFTRSELKFSPQWSHHGKIVTCQLQDADGKFLSNDTVQLNVKHTPKLEIKVTPSDAIVR
EGDSVTMTCEVSSSNPEYTTVSWLKDGTSLKKQNTFTLNLREVTKDQSGKYCCQVSNDVGPGRSEEVFLQVQYAPEGGGGSGGGG
SGGGGSGGGGSDILMTQSPSSMSVSLGDTVSITCHSSQDINSNIGWLQQRPGKSFKGLIYHGTNLDDEVPSRFSGSGSGADYSLT
ISSLESEDFADYYCVQYAQFPWTFGGGTKLEIKRGGGGSGGGGSGGGGSGGGGSMRVLILLWLFTAFPGVLSDVQLQESGPSLVK
PSQSLSLTCTVTGYSITSDFAWNWIRQFPGNKLEYMGYISYSGNTRYNPSLKSRISITRDTSKNQFFLQLNSVTIEDTATYYCVT
AGRGFPYWGQGTLVTVSAHHHHHH
SEQ ID NO. 71
MLRLLLALNLFPSIQVTGDIQMTQTTSSLSASLGDRVTISCRASQDISKYLNWYQQKPDGTVKLLIYHTSRLHSGVPSRFSGSGS
GTDYSLTISNLEQEDIATYFCQQGNTLPYTFGGGTKLEITGGGGSGGGGSGGGGSGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDTAIYYCAKHYYYGGSYAMDYW
GQGTSVTVSSDYKDDDDKIEVMYPPPYLDNEKSNGTIIHVKGKHLCPSPLFPGPSKPFWVLVVVGGVLACYSLLVTVAFIIFWVR
SKRSRLLHSDYMNMTPRRPGPTRKHYQPYAPPRDFAAYRSKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRKNPQEGLYNELQKDKMAEAYSEIGMKGERRRGKGHD
GLYQGLSTATKDTYDALHMQALPPR
SEQ ID NO. 72
MLRLLLALNLFPSIQVTGDIQMTQTTSSLSASLGDRVTISCRASQDISKYLNWYQQKPDGTVKLLIYHTSRLHSGVPSRFSGSGS
GTDYSLTISNLEQEDIATYFCQQGNTLPYTFGGGTKLEITGGGGSGGGGSGGGGSGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDTAIYYCAKHYYYGGSYAMDYW
GQGTSVTVSSDYKDDDDKIEVMYPPPYLDNEKSNGTIIHVKGKHLCPSPLFPGPSKPFWVLVVVGGVLACYSLLVTVAFIIFWVR
SKRSRLLHSDYMNMTPRRPGPTRKHYQPYAPPRDFAAYRSKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRKNPQEGLYNELQKDKMAEAYSEIGMKGERRRGKGHD
GLYQGLSTATKDTYDALHMQALPPR
SEQ ID NO. 73
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMPAGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTHHHHHH
SEQ ID NO. 74
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTHHHHHH
SEQ ID NO. 75
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTHHHHHH
SEQ ID NO. 76
MPPPRLLFFLLFLTPMEVRPEEPLVVKVEEDGNAVLQCLKGTSDGPTQQLTWSREDPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTHHHHHH
SEQ ID NO. 77
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTHHHHHH
SEQ ID NO. 78
MDFQVQIFSFLLISASVIMSRMQAVKLQESGAELVKGPASVKMSCKASGYTFTSYNMHWVKQTPGQGLEWIGAIYPGNGDTSYNQ
KFKGKATLTADKSSSTAYMQLSSLTSEDSADYYCARSNYYGSSYWFFDVWGQGTTVTVSSGGGGSGGGGSGGGGSDIELTQSPTI
LSASPGEKVTMTCRASSSVNYMDWYQKKPGSSPKPWIYATSNLASGVPARFSGSGSGTSYSLTISRVEAEDAATYYCQQWSFNPP
TFGGGTKLEIKRAAAGDPAEPKSPDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
SEQ ID NO. 78
MDFQVQIFSFLLISASVIMSRMAQVKLQESGAELVKPGASVKMSCKASGYTFTSYNMHWVKQTPGQGLEWIGAIYPGNGDTSYNQ
KFKGKATLTADKSSSTAYMQLSSLTSEDSADYYCARSNYYGSSYWFFDVWGQGTTVTVSSGGGGSGGGGSGGGGSDIELTQSPTI
LSASPGEKVTMTCRASSSVNYMDWYQKKPGSSPKPWIYATSNLASGVPARFSGSGSGTSYSLTISRVEAEDAATYYCQQWSFNPP
TFGGGTKLEIKRAAAHHHHHH
SEQ ID NO. 80
MDFQVQIFSFLLISASVIMSRDIELTQSPTILSASPGGEKVTMTCRASSSVMYMDWYQKKPGSSPKPWIYATSNLASGVPARFSG
SGSGTSYSLTISRVEAEDAATYYCQQWSFNPPTFGGGTKLEIKRGGGGSGGGGSGGGGSMAQVKLQESGAELVKPGASVKMSCKA
SGYTFTSYNMHWVKQTPGQGLEWIGAIYPGNGDTSYNQKFKGKATLTADKSSSTAYMQLSSLTSEDSADYYCARSNYYGSSYWFF
DVWGQGTTVTVSSAAAGDPAEPKSPDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDLWNGKEYKCKVSNKALPAPIEKTISKAKGEPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQKSLSLSPG
SEQ ID NO. 81
MDFQVQIFSFLLISASVIMSRDIELTQSPTILSASPGEKVTMTCRASSSVNYMDWYQKKPGSSPKPWIYATSNLASGVPARFSGS
GSGTSYSLTISRVEAEDAATYYCQQWSFNPPTFGGGTKLEIKRGGGGSGGGGSGGGGSMAQVKLQESGAELVKPGASVKMSCKAS
GYTFTSYNMHWVKQTPGQGLEWIGAIYPGNGDTSYNQKFKGKATLTADKSSSTAYMQLSSLTSEDSADYYCARSNYYGSSYWFFD
VWGQGTTVTVSSAAAHHHHHH
SEQ ID NO. 82
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSMAQVKLQESGAELVKPGASVKMSCKASGYTFTSYNMHWVKQT
PGQGLEWIGAIYPGNGDTSYNQKFKGKATLTADKSSSTAYMQLSSLTSEDSADYYCARSNYYGSSYWFFDVWGQGTTVTVSSGGG
GSGGGGSGGGGSDIELTQSPTILSASPGEKVTMTCRASSSVNYMDWYQKKPGSSPKPWIYATSNLASGVPARFSGSGSGTSYSLT
ISRVEAEDAATYYCQQWSFNPPTFGGGTKLEIKRAAAGDPAEPKSPDKTHTCPPCPAPELLGGPSVFLFPPKPKDTLMISRTPEV
TCVVVDVSHEDPEVKFNWYVDGVEVHNAKTKPREEQYNSTRYVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDKSRWQQGNVFSCSVMH
EALHNHYTQKSLSLSPG
SEQ ID NO. 83
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSMAQVKLQESGAELVKPGASVKMSCKASGYTFTSYNMHWVKQT
PGQGLEWIGAIYPGNGDTSYNQKFKGKATLTADKSSSTAYMQLSSLTSEDSADYYCARSNYYGSSYWFFDVWGQGTTVTVSSGGG
GSGGGGSGGGGSDIELTQSPTILSASPGEKVTMTCRASSSVNYMDWYQKKPGSSPKPWIYATSNLASGVPARFSGSGSGTSYSLT
ISRVEAEDAATYYCQQWSFNPPTFGGGTKLEIKRAAAHHHHHH
SEQ ID NO. 84
MPPPRLLFFLLFLTPMEVRPEEPLVVKVEEDGNAVLQCLKGTSDGPTQQLTWSRESPLKPFLKLSLGLPGLGIHMRPLAIWLFIF
NVSQQMGGFYLCQPGPPSEAKWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSDIELTQSPTILSASPGEKVTVTCRASSSVNYMDWYQKKPGSS
PKPWIYATSNLASGVPARFSGSGSGTSYSLTISVEAEDAATYYCQQWSFNPPTFGGGTKLEIKRGGGGSGGGGSGGGGSMAQVKL
QESGAELVKPGASVKMSCKASGYTFTSYNMHWVKQTPGQGLEWIGAIYPGNGDTSYNQKFKGKATLTADKSSSTAYMQLSSLTSE
DSADYYCARSNYYGSSYWFFDVWGQGTTVTVSSAAAGDPAEPKSPDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKSRWQQGNVFSCSVMHE
ALHNHYTQKSLSLSPG
SEQ ID NO. 85
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSDIELTQSPTILSASPGEKVTMTCRASSSVNYMDWYQKKPGSS
PKPWIYATSNLASGVPARFSGSGSGTSYSLTISRVEAEDAATYYCQQWSFNPPTFGGGTKLEIKRGGGGSGGGGSGGGGSMAQVK
LQESGAELVKPGASVKMSCKASGYTFTSYNMHWVKQTPGQGLEWIGAIYPGNGDTSYNQKFKGKATLTADKSSSTAYMQLSSLTS
EDSADYYCARSNYYGSSYWFFDVWGQGTTVTVSSAAHHHHHH
SEQ ID NO. 86
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSQVQLVQSGAEDKKPGESVKISCKASGYTFTNYGMNWVRQAPG
QGLKWMGWINTYTGESTYADDFKGRFAFSLDTSASTAYLQLSSLRGEDTAVYFCARFAIKGDYWGQGTTVTVSSASTGGGGSGGG
GSGGGGSGGGGSDIVMTQSPLSLEVSPGEPASISCRSTKSLLHSDGITYLYWYLQKPGQSPQLLIYQLSNLASGVPDRFSSSGSG
TDFTLKISRVEAEDEGTYYCAQNLEIPRTFGQGTKLEIKRTHHHHHH
SEQ ID NO. 87
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMPAGSTLWLSCGVPPDSVSRGPLSWTHVHPKGPKSLLSLELKDDRPARDMWVEMTGLLLPRATAQD
AGKYYCHRGNLTMSFHLEITARPGGGGSGGGGSGGGGSGGGGSWVQLVQSGAEDVKPDASVKLSCKASGYTFTDYYMHWVRQAPG
QGLEWMGRVNPNRRGTTYNQKFEGRVTMTTDTSTSTAYMQLSSLRGEDTAVYYCARANWLDYWGQGTTVTVSSASTGGGGSGGGG
SGGGGSGGGGSDIQMTQSPSSLEASVGDRVTITCSVSSSVSSIYLHWYQQKPGKSPKLLIYSTSNLASGVPSRFSGSGSGTDFTL
TISSLQAEDEGTYYCQVYSGYPLTFGGGTKLEIKRTHHHHHH
SEQ ID NO. 88
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSQVQLQESGPGDVKPSETLSLTCTVSGGSVSSGDYYWTWIRQS
PGKGLEWIGHIYYSGNTNYNPSLKSRLTISIDTSKTTFSLQLSSVTGEDTAIYYCVRDRVTGAFDIWGQGTTVTVSSASTGGGGS
GGGGSGGGGSGGGGSDIQMTQSPSSLEASVGDRVTITCQASQDISNYLNWYQQKPGKSPKLLIYDASNLETGVPDRFSGSGSGTD
FTFTISSLQAEDEGTYFCQHFDHLPLAFGGGTKLEIKRTHHHHHH
SEQ ID NO. 89
METDTLLLWVLLLWVPGSTGDIVLTQSPPSLAMSLGKRATISCRASESVTILGSHLIHWYQQKPGQPPTLLIQLASNVQTGVPAR
FSGSGSRTDFTLTIDPVEEDDVAVYYCLQSRTIPRTFGGGTKLEIKGSTSGSGKPGSGEGSTKGQIQLVQSGPELKKPGETVKIS
CKASGYTFTDYSINWVKRAPGKGLKWMGWINTETREPAYAYDFRGRFAFSLETSASTAYLQINNLKYEDTATYFCALDYSYAMDY
WGQGTSVTVSSGGGGSGGGGSGGGGSHHHHHH
SEQ ID NO. 90
MEFGLSWVFLVALFRGVQCQIQLVQSGPELKKPGETVKISCSASGYTFTDYSINWVKRAPGKGLKWMGWINTETREPAYAYDFRG
RFAFSLETSASTAYLQINNLKYEDTATYFCALDYSYAMDYWGQGTSVTVSSGSTSGSGKPGSGEGSTKGDIVLTQSPPSLAMSLG
KRATISCRASESVTILGSHLIHWYQQKPGQPPTLLIQLASNVQTGVPARFSGSGSRTDFTLTIDPVEEDDVAVYYCLQSRTIPRT
FGGGTKLEIKGGGGSGGGGSGGGGSHHHHHH
SEQ ID NO. 91
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGGPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSDIVLTQSPPSLAMSLGKRATISCRASESVTILGSHLIHWYQQ
KPGQPPTLLIQLASNVQTGVPARFSGSGSRTDFTLTIDPVEEDDVAVYYCLQSRTIPRTFGGGTKLEIKGSTSGSGKPGSGEGST
KGQIQLVQSGPELKKPGETVKISCKASGYTFTDYSINWVKRAPGKGLKWMGWINTETREPAYAYDFRGRFAFSLETSASTAYLQI
NNLKYEDTATYFCALDYSYAMDYWGQGTSVTVSSGGGGSGGGGSGGGGSHHHHHH
SEQ ID NO. 92
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDSGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVEMTGLLLPRATAQD
AGKYYCHRGNLTMSFHLEITARPGGGGSGGGGSGGGGSGGGGSQIQLVQSGPELKKPGETVKISCKASGYTFTDYSINWVKRAPG
KGLKWMGWINTETREPAYAYDFRGRFAFSLETSASTAYLQINNLKYEDTATYFCALDYSYAMDYWGQGTSVTVSSGSTSGSGKPG
SGEGSTKGDVILTQSPPSLAMSLGKRATISCRASESVTILGSHLIHWYQQKPGQPPTILLIQLASNVQTGVPARFSGSGSRTDFT
LTIDPVEEDDVAVYYCLQSRTIPRTFGGGTKLEIKGGGGSGGGGSGGGGSHHHHHH
SEQ ID NO. 93
MPPPRLLFFLLFLTPMEVRPEEPLVVKVEEDGDNALQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNAKLPAPIEKTISKAKGQPREPQVYTLPPSREEMTKNQVSLTC
LVKGFYPSDIAVEWESNGQEPNNYKTTPPVLDSDGSFFLYSKLTVDKSRWQQGNVFSCSVMHEALHNHYTQKSLSLSPGGGGGSG
GGGSGGGGSGGGGSEVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTGGGGSGGGGSGGGGSDIQMTQSPSSLSASVGDR
VTITCRASQDVNTAVAWYQQKPGKAPKLLIYSASFLYSGVPSRFSGSRSGTDFTLTISSLQPEDFATYYCQQHYTTPPTFGQGTK
VEIKRT
SEQ ID NO. 94
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRRAEDTAVYYCSRWGGDGFYAMDYWGQGTLVTVSSASTGGG
GSGGGGSGGGGSDIQMTQSPSSLSASVGDRVTITCRASQDVNTAVAWYQQKPGKAPKLLIYSASFLYSGVPSRFSGSRSGTDFTL
TISSLQPEDFATYYCQQHYTTPPTFGQGTKVEIKRTGGGGSGGGGSGGGGSGGGGSQVQLQESGPGDVKPSETLSLTCTVSGGSV
SSGDYYWTWIRQSPGKLGEWIGHIYYSGNTNYNPSLKSRLTISIDTSKTTFSLQLSSVTGEDTAIYYCVRDRVTGAFIDWGQGTT
VTVSSASTGGGGSGGGGSGGGGSGGGGSDIQMTQSPSSLEASVGDRVTITCQASQDISNYLNWYQQKPGKSPKLLIYDASNLETG
VPDRFSGSGSGTDFTFTISSLQAEDEGTYFCQHFDHLPLAFGGGTKLEIKRTGGGGSHHHHHH
SEQ ID NO. 95
MPPPRLLFFLLFLTPMEVRPEEPLVVKVEEDGNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTGGGGSGGGGSGGGGSGGGGSDIQMTQSPSSLEASVGDRVTITCQASQDIS
NYLNWYQQKPGKSPKLLIYDASNLETGVPDRFSGSGSGTDFTFTISSLQAEDEGTYFCQHFDHLPLAFGGGTKLEIKRTGGGGSG
GGGSGGGGSGGGGSQVQLQESGPGDVKPSETLSLTCTVSGGSVSSGDYYWTWIRQSPGKGLEWIGHIYYSGNTNYNPSLKSRLTI
SIDTSKTTFSLQLSSVTGEDTAIYYCVRDRVTGAFDIWGQGTTVTVSSASTGGGGSHHHHHH
SEQ ID NO. 96
MEFGLSWVFLVALFRGVQCEVQLVESGGGLVQPGGSLRLSCAASGFNIKDTYIHWVRQAPGKGLEWVARIYPTNGYTRYADSVKG
RFTISADTSKNTAYLQMNSLRAEDTAVYYCSRWGGDGFYAMDYWGQGTLVTVSSASTGGGGSGGGGSGGGGSDIQMTQSPSSLSA
SVGDRVTITCRASQDVNTAVAWYQQKPGKAPKLLIYSASFLYSGVPSRFSGSRSGTDFTLTISSLQPEDFATYYCQQHYTTPPTF
GQGTKVEIKRTGGGGSGGGGSGGGGSGGGGSQVQLQESGPGDVKPSETLSLTCTVSGGSVSSGDYYWTWIRQSPGKGLEWIGHIY
YSGNTNYNPSLKSRLTISIDTSKTTFSLQLSSVTGEDTAIYYCVRDRVTGAFDIWGQGTTVTVSSASTGGGGSGGGGSGGGGSGG
GGSDIQMTQSPSSLEASVGDRVTITCQASQDISNYLNWYQQKPGKSPKLLIYDADNLETGVPDRFSGSGSGTDFTFTISSLQAED
EGTYFCQHFDHLPLAFGGGTKLEIKRTGGGGSHHHHHH
SEQ ID NO. 97
MEFGLSWVFLFVALFRGVQCEVQLVESGGGLVQPGGSLRLSCAASGFNIKDTYIHWVRQAPGKGLEWVARIYPTNGYTRYADSVK
GRFTISADTSKNTAYLQMNSLRAEDTAVYYCSRWGGDGFYAMDYWGQGTLVTVSSASTGGGGSGGGGSGGGGSDIQMTQSPSSLS
ASVGDRVTITCRASQDVNTAVAWYQQKPGKAPKLLIYSASFLYSGVPRFSGSRSGTDFTLTISSLQPEDFATYYCQQHYTTPPTF
GQGTKVEIKRTGGGGSGGGGSGGGGSGGGGSDIQMTQSPSSLEASVGDRVTITCQASQDISNYLNWYQQKPGKSPKLLIYDASNL
ETGVPDRFSGSGSGTDFTFTISSLQAEDEGTYFCQHFDHLPLAFGGGTKLEIKRTGGGGSGGGGSGGGGSGGGGSQVQLQESGPG
DVKPSETLSLTCTVSGGSVSSGDYYWTWIRQSPGKGLEWIGHIYYSGNTNYNPSLKSRLTISIDTSKTTFSLQLSSVTGEDTAIY
YCVRDRVTGAFDIWGQGTTVTVSSASTGGGGSHHHHHH
SEQ ID NO. 98
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTHHHHHH
SEQ ID NO. 99
MDFQVQIFSFLLISASVIMSRMAQVKLQESGAELVKPGASVKMSCKASGYTFTSYNMHWVKQTPGQGLEWIGAIYPGNGDTSYNQ
KFKGKATLTADKSSSTAYMQLSSLTSEDSADYYCARSNYYGSSYWFFDVWGQGTTVTVSSGGGGSGGGGSGGGGSDIELTQSPTI
LSASPGEKVTMTCRASSSVNYMDWYQKKPGSSPKPWIYATSNLASGVPARFSGSGSGTSYSLTISRVEAEDAATYYCQQWSFNPP
TFGGGTKLEIKRAAAGDPAEPKSPDKTHTCPPCPAPELLGGPSVFLFPPKPKDTLMISRTPECTCVVVDVSHEDPEVKFNWYVDG
VEVHNAKTKPREEQYNSTRYVVSVLTVLHQDWLNGKEYKCKVSNKALPAPIEKTISKAKGQPREPQVYTLPPSREEMTKNQVSLT
CLVKGFYPSDIAVEWESNGQEPNNYKTTPPVLDSDGSFFLYSKLTVDKSRWQQGNVFSCSVMHEALHNHYTQKSLSLSPG
SEQ ID NO. 100
MDFQVQIFSFLLISASVIMSRMAQVKLQESGAELVKPGASVKMSCKASGYTFTSYNMHWVKQTPGQGLEWIGAIYPGNGDTSYNQ
KFKGKATLTADKSSSTAYMQLSSLTSEDSADYYCARSNYYGSSYWFFDVWGQGTTVTVSSGGGGSGGGGSGGGGSDIELTQSPTI
LSASPGEKVTMTCRASSSVNYMDWYQKKPGSSPKPWIYATSNLASGVPARFSGSGSGTSYSLTISRVEAEDAATYYCQQWSFNPP
TFGGGTKLEIKRAAAHHHHHH
SEQ ID NO. 101
MDFQVQIFSFLLISASVIMSRDIELTQSPTILSASPGEKVTMTCRASSSVNYMDWYQKKPGSSPKPWIYATSNALSGVPARFSGS
GSGTSYSLTISRVEAEDAATYYCQQWSFNPPTFGGGTKLEIKRGGGGSGGGGSGGGGSMAQVKLQESGAELVKPGASVKMSCKAS
GYTFTSYNMHWVKQTPGQGLEWIGAIYPGNGDTSYNQKFKGKATLTADKSSSTAYMQLSSLTSEDSADYYCARSNYYGSSYWFFD
VWGQGTTVTVSSAAAGDPAEPKSPDKTHTCPPCPAPELLGGPSVFLFPPKPKDTLMISRTPECTCVVVDVSHEDPEVKFNWYVDG
VEVHNAKTKPREEQYNSTYRVVSVLTVLHQDLWNGKEYKCKVSNKALPAPIEKTISKAKGQPREPQVYTLPPSREEMTKNQVSLT
CLVKGFYPSDAIVEWESNGQPENNYKTTPPVLDSDGSFFLYSKLTVDKSRWQQGNVFSCSVMHEALHNHYTQKSLSLSPG
SEQ ID NO. 102
MDFQVQIFSFLLISASVIMSRDIELTQSPTILSASPGEKVTMTCRASSSVNYMDWYQKKPGSSPKPWIYATSNLASGVPARFSGS
GSGTSYSLTISRCEAEDAATYYCQQWSFNPPTFGGGTKLEIKRGGGGSGGGGSGGGGSMAQVKLQESGAELVKPGASVKMSCKAS
GYTFTSYNMHWVKQTPGQGLEWIGAIYPGNGDTSYNQKFKGKATLTADKSSSTAYMQLSSLTSEDSADYYCARSNYYGSSYWFFD
VWGQGTTVTVSSAAAHHHHHH
SEQ ID NO. 103
MEFGLSWVFLFVALFRGVQCEVQLVESGGGLVQPGGSLRLSCAASGFNIKDTYIHWVRQAPGKGLEWVARIYPTNGYTRYADSVK
GRFTISADTSKNTAYLQMNSLRAEDTAVYYCSRWGGDGFYAMDYWGQGTLVTVSSASTGGGGSGGGGSGGGGSDIQMTQSPSSLS
ASVGDRVTITCRASQDVNTAVAWYQQKPGKAPKLLIYSASFLYSGVPSRFSGSRSGTDFTLTISSLQPEDFATYYCQQHYTTPPT
FGQGTKVEIKRTDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHELAHNHYTQKSLSLSPG
SEQ ID NO. 104
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGVYLCQPGPPSEAKWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSGGGGSGGGGSGGGGSMAQVKLQESGAELVKPGASVKMSCKAS
GYTFTSYNMHWVKQTPGQGLEWIGAIYPGNGDTSYNQKFKGKATLTIADKSSSTAYMQLSSLTSEDSADYYCARSNYYGSSYWFF
DVWGQGTTVTVSSGGGGSGGGGSGGGGSDIELTQSPTILSASPGEKVTMTCRASSSVNYMDWYQKKPGSSPKPWIYATSNLASGV
PARFSGSGSGTSYSLTISRVEAEDAATYYCQQWSFNPPTFGGGTKLEIKRAAAGDPAEPKSPDKTHTCPPCPAPELLGGPSVFLF
PPKPKDTLMISRTPEVTCVVVDVSHEDPEVKFNWYVDGVEHNAKTKPREEQYNSTYRVVSVLTVLHQDWLNGKEYKCKVSNKALP
IEKTISKAKGQPREPQVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPG
SEQ ID NO. 105
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEAKWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSGGGGSGGGGSGGGGSMAQVKLQESGAELVKPGASVKMSCKAS
GYTFTSYNMHWVKQTPGQGLEWIGAIYPGNGDTSYNQKFKGKATLTADKSSSTAYMQLSSLTSEDSADYYCARSNYYGSSYWFFD
VWGQGTTVTVSSGGGGSGGGGSGGGGSDIELTQSPTILSASPGEKVTMTCRASSSVNYMDWYQQKPGSSPKPWIYATSNLASGVP
ARFSGSGSGTSYSLTISRVEAEDAATYYCQQWSFNPPTFGGGTKLEIKRAAAHHHHHH
SEQ ID NO. 106
MPPPRLLFFLLFLTPMEVRPEEPLVVKVEEGDNAVLQCLKGTSDGPTQQLTWSREDPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPDPAEPKSPDKTHTCPPCPAPELLGGPSVFLFPPKPKDTLMISRTPEVCTCVVVDVSHEDPEV
KFNWYVDGVEVHNAKTKPREEQYNSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQGNVFSCSVMHEALHNHYTQKSLSL
SPGGGGGSGGGGSGGGGSGGGGSMAQVKLQESGAELVKPGASVKMSCKASGYTFTSYNMHWVKQTPGQGLEWIGAIYPGNGDTSY
NQKFKGKATLTADKSSSTAYMQLSSLTSEDSADYYCARSNYYGSSYWFFDVWGQGTTVTVSSGGGGSGGGGSGGGGSDIELTQSP
TILSASPGEKVTMTCRASSSVNYMDWYQKKPGSSPKPWIYATSNLASGVPARFSGSGSGTSYSLTISRVEAEDAATYYCQQWSFN
PPTFGGGTKLEIKRA
SEQ ID NO. 107
MDFQVQIFSFLLISASVIMSRMAQVKLQESGAELVKPGASVKMSCKASGYTFTSYNMHWVKQTPGQGLEWIGAIYPGNGDTSYNQ
KFKGKATLTADKSSSTAYMQLSSLTSEDSADYYCARSNYYGSSYWFFDVWGQGTTVTVSSGGGGSGGGSGGGGSDIELTQSPTIL
SASPGEKVTMTCRASSSVNYMDWYQKKPGSSPKPWIYATSNLASGVPARFSGSGSGTSYSLTISRVEAEDAATYYCQQWSFNPPT
FGGGTKLEIKRAAAGGGGSGGGGSGGGGSGGGGSPEEPLVVKVEEGDNAVLQCLKGTSDGPTQQLTWSRESPLKPFLKLSLGLPG
LGIHMRPLAIWLFIFNVSQQMGGFYLCQPGPPSEKAWQPGWTVNVEGSGELFRWNVSDLGGLGCGLKNRSSEGPSSPSGKLMSPK
LYVWAKDRPEIWEGEPPCLPPRDSLNQSLSQDLTMAPGSTLWLSCGVPPDSVSRGPLSWTHVHPGKGPKSLLSLELKDDRPARDM
WVMETGLLLPRATAQDAGKYYCHRGNLTMSFHLEITARPDPAEPKSPDKTHTCPPCPAPELLGGPSVFLFPPKPDKTLMISRTPE
VTCVVVDSHEDPEVKFNWYVDGVEVHNAKTKPREEQYNSTRYVVSVLTVLHQDWLNGKEYKCKVSNAKLPAPIEKTISKAKGQPR
EPQVYTLPPSREEMTKNQVSLTCLVKGFYPSDIAVEWESNGQPENNYKTTPPVLDSDGSFFLYSKLTVDKSRWQQGNVFSCSVMH
EALHNHYTQKSLSLSPG
SEQ ID NO. 108
MLRLLLALNLFPSIQVTGDIQMTQTTSSLSASLGDRVITSCRASQDISKYLNWYQQKPDGTVKLLIYHTSRLHSGVPSRFSGSGS
GTDYSLTISNLEQEDIATYFCQQGNTLPYTFGGGTKLEITGGGGSGGGGSGGGGSGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDTAIYYCAKHYYYGGSYAMDYW
GQGTSVTVSSDYKDDDDKIEVMYPPPYLDNEKSNGTIIHVKGKHLCPSPLFPGPSKPFWVLVVVGGVLACYSLLVTVAFIIFWVR
SKRSRLLHSDYMNMTPRRPGPTRKHYQPYAPPRDFAAYRSKRGRKKLLYIFKQPFMRPVQTTQEEDGCSCRFPEEEEGGCELRVK
FSRSADAPAYQQGQNQLYNELNLGRREEYDVLDKRRGRDPEMGGKPQRRKNPQEGLYNELQKDKMAEAYSEIGMKGERRRGKGHD
GLYQGLSTATKDTYDALHMQALPPREGRGSLLTCGDVEENPGPMPPPRLLFFLLFLTPMEVRPEEPLVVKVEEGDNAVLQCLKGT
SDGPTQQLTWSRESPLKPFLKLSLGLPGLGIHMRPLAIWLFIFNVSQQMGGFYLCQPGPPSEKAWQPGWTVNVEGSGELFRWNVS
DLGGLGCGLKNRSSEGPSSPSGKLMSPKLYVWAKDRPEIWEGECPPCLPPRDSLNQSLSQDLTMAPGSTLWLWCGVPPDSVSRGP
LSWTHVHPKGPKSLLSLELKDDRPARDMWVMETGLLLPRATAQDAGKYYCHRGNLTMSFHLEITARPGGGGSGGGGSGGGGSGGG
GSEVQLVESGGGLVQPGGSLRLSCAASGFNIKDTYIHWVRQAPGKGLEWVARIYPTNGYTRYADSVKGRFTISADTSKNTAYLQM
NSLRAEDTAVYYCSRWGGDGFYAMDYWGQGTLVTVSSASTGGGGSGGGGSGGGGSDIQMTQSPSSLSASVGDRVTITCRASQDVN
TAVAWYQQKPGKAPKLLIYSASFLYSGVPSRFSGSRSGTDFTLTISSLQPEDFATYYCQQHYTTPPTFGQGTKVEIKRTSRHHHH
HH
SEQ ID NO. 109
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTSRHHHHHHEGRGSLLTCGDVEENPGPMLRLLLALNLFPSIQVTGDIQMTQ
TTSSLSASLGDRVTISCRASQDISKYLNWYQQKPDGTVKLLIHYTSRLHSGVPSRFSGSGSGTDYSLTISNLEQEDIATYFCQQG
NTLPYTFGGGTKLEITGGGGSGGGGSGGGGSGGGGSEVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKHYYYGGSYAMDYWGQGTSVTVSSDYKDDDDKIEVMYP
PPYLDNEKSNGTIIHVKGKHLCPSPLFPGPSKPFWVLVVVGGVLACYSLLVTVAFIIFWVRSKRSRLLHSDYMNMTPRRPGPTRK
HYQPYAPPRDFAAYRSKRGRKKLLYIFKQPFMRPVQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMKGERRRGKGHDGLYQGLSTATKDTYDALHMQALPP
R
SEQ ID NO. 110
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTHHHHHH
SEQ ID NO. 111
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSEVQLVESGGGLVQPGGSLRLSCAASGFNIKDTYIHWVRQAPG
KLGEWVARIYPTNGYTRYADSVKGRFTISADTSKNTAYLQMNSLRAEDTAVYYCSRWGGDGFYAMDYWGQGTLVTVSSASTGGGG
SGGGGSGGGGSDIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTHHHHHH
SEQ ID NO. 12
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWK
SEQ ID NO. 113
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMW
VMETGLLLPRATAQDAGKYYCHRGNLTMSFHLEITARPGGGGSGGGGSGGGGSGGGGSEVQLVESGGGLVQPGGSLRLSCAASGF
NIKDTYIHWVRQAPGKGLEWVARIYPTNGYTRYADSVKGRFTISADTSKNTAYLQMNSLRAEDTAVYYCSRWGGDGFYAMDYWGQ
GTLVTVSSASTGGGGSGGGGSGGGGSDIQMTQSPSSLSASVGDRVTITCRASQDVNTAVAWYQQKPGKAPKLLIYSASFLYSGVP
SRFSGSRSGTDFTLTISSLQPEDFATYYCQQHYTTPPTFGQGTKVEIKRTSRHHHHHH
SEQ ID NO. 114
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSQVQLVQSGAEDKKPGESVKISCKASGYTFTNYGMNWVRQAPG
QGLKWMGWINTYTGESTYADDFKGRFAFSLDTSASTAYLQLSSLRGEDTAVYFCARFAIKGDYWGQGTTVTVSSASTGGGGSGGG
GSGGGGSGGGGSDIVMTQSPLSLEVSPGEPASISCRSTKSLLHSDGITYLYWYLQKPGQSPQLLIYQLSNLASGVPDRFSSSGSG
TDFTLKISRVEAEDEGTYYCQANLEIPRTFGQGTKLEIKRTHHHHHH
SEQ ID NO. 115
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSQVQLVQSGAEVKPDASVKLSCKASGYTFTDYYMHWVRQAPGQ
GLEWMGRVNPNRRGTTYNQKFEGRVTMTTDTSTSTAYMQLSSLRGEDTAVYYCARANWLDYWGQGTTVTVSSASTGGGGSGGGGS
GGGGSGGGGSDIQMTQSPSSLEASVGDRVTITCSVSSSVSSIYLHWYQQKPGKSPKLLKIYSTSNLASGVPSRFSGSGSGTDFTL
TISSLQAEDEGTYYCQVYSGYPLTFGGGTKLEIKRTHHHHHH
SEQ ID NO. 116
MPPPRLLFFLLFLTPMEVRPEEPLVVKVEEGDNAVLQCLKGTSDGPTQQLTWSRESPLKPFLKLSLGLPGLGIHMRPLAIWLFIF
NVSQQMGGFYLCQPGPPSEKAWQPGWTVNVEGSGELFRWNVSDLGGLGCGLKNRSSEGPSSPSGKLMSPKLYVWAKDRPEIWEGE
PPCLPPRDSLNQSLSQDLTMAPGSTLWLSCGVPPDSVSRGPLSWTHVHPKGPKSLLSLELKDDRPARDMWVMETGLLLPRATAQD
AGKYYCHRGNLTMSFHLEITARPGGGGSGGGGSGGGGSGGGGSQVQLQESGPGDVKPSETLSLTCTVSGGSVSSGDYYWTWIRQS
PGKGLEWIGHIYYSGNTNYNPSLKSRLTISIDTSKTTFSLQLSSVTGEDTAIYYCVDRVTGAFDIWGQGTTVTVSSASTGGGGSG
GGGSGGGGSGGGGSDIQMTQSPSSLEASVGDRVTITCQASQDISNYLNWYQQKPGKSPKLLIYDASNLETGVPDRFSGSGSGTDF
TFTISSLQAEDEGTYFCQHFDHLPLAFGGGTKLEIKRTHHHHHH
SEQ ID NO. 117
MLRLLLALNLFPSIQVTGDIQMTQTTSSLSASLGDRVTISVRASQDISKYLNWYQQKPDGTVKLLIYHTSRLHSGVPSRFSGSGS
GTDYSLTISNLEQEDIATYFCQQGNTLPYTFGGGTKLEITGGGGSGGGGSGGGGSGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDTAIYYCAKHYYYGGSYAMDYW
GQGTSVTVSSDYKDDDDKIEVMYPPPYLDNEKSNGTIIHVKGKHLCPSPLFGPSKPFWVLVVVGGVLACYSLLVTVAFIIFWVRS
KRSRLLHSDYMNMTPRRPGPTRKHYQPYAPPRDFAAYRSKRGRKKLLYIFKQPFMRPVQTTQEEDGCSCRFPEEEEGGCELRVKF
SRSADAPAYQQGQNQLYNELNLGRREEYDVLDKRRGRDPEMGGKPQRRKNPQEGLYNELQKDKMAEAYSEIGMKGERRRGKGHDG
LYQGLSTATKDTYDALHMQALPPR
SEQ ID NO. 118
MLRLLLALNLFPSIQVTGDIQMTQTTSSLSASLGDRVTISCRASQDISKYLNWYQQKPDGTVKLLIYHTSRLHSGVPSRFSGSGS
GTDYSLTISNLEQEDIATYFCQQGNTLPYTFGGGTKELITGGGGSGGGGSGGGGSGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDTAIYYCAKHYYYGGSYAMDYW
GQGTSVTVSSIEVMYPPPYLDNEKSNGTIIHVKGKHLCPSPLFPGPSKPFWVLVVVGGVLACYSLLVTVAFIIFWVRSKRSRLLH
SDYMNMTPRRPGPTRKHYQPYAPPRDFAAYRSKRGRKKLLYIFKQPFMRPVQTTQEEDGCSCRFPEEEGGCELRVKFSRSADAPA
YQQGQNQLYNELNGRREEYDVLDKRRGRDPEMGGKPQRRKNPQEGLYNELQKDKMAEAYSEIGMKGERRRGKGHDGLYQGLSTAT
KDTYDALHMQALPPR
SEQ ID NO. 119
MEKDTLLLWVLLLWVLPGSTGEVLKLVESGGGLVQPGGSLKLSCAASGFDFSRYWMSWVQAPGKGLEWIGEINLDSSTINYTPSL
KDKFIISRDNAKNTLYLQMNSKVRSEDTALYYCARRYDAMDYWGQGTSVTVSSAKTTAPSVYPLALVCGDTTGSSVTLGCLVKGY
FPEPVTLTWNSGSLSSGVHTFPAVLQSDLYTLSSSVTVTSSTWPSQSITCNVAHPASSTKVDKKIEPRGPTIKPCPPCKCPAPNL
LGGPSVFIFPPKIKDVLMISLSPIVTCVVVDVSEDDPDVQISWFVNNVEVHTQATQTHREDYNSTLORVVSALPIQHQDWMSGKE
FKCKVNNKDLPAPIERTISKPGKSVRAPQVYVLPPPEEEMTKKQVTLTCMVDTDFMPEDIYVEWTNNGKTELNYKNTEPVLDSDG
SYFMYSKLRVEKKNWVERNSYSCSVVHEGLHNHHTTKSFSRTPGK
SEQ ID NO. 120
MDFGLIFFIVALLKGVQCDIVLTQSPASLAVSLGQRATISCRASESVDDYGISFMNWFQQKPGQPPKLLIYAAPONQGSGVPARF
SGSGSGTDFSLNIHPMEEDDTAMYFCQQSKDVRWTFGGGTKLEIKRADAAPTVSIFPPSSEQLTSGGASVVCFLNNFYPKDINVK
WKIDGSERQGNVLNSWTDQDSKDSTYSMSSTLTLTKDEYERHNSYTCEATHKTSTSPIVKSFNRNEC
SEQ ID NO. 121
MEKDTLLLWVLLLWVPGSTGEVKLVESGGGLVQPGGSLKLSCAASGFDFSRYWMSWVRQAGKGLEWIGEINLDSSTINYTPSLKD
KFIISRDNAKNTLYLQMSKVRSEDTALYYCARRYDAMDYWGQGTSVTVSSASTGGGGSGGGGSGGGGSGGGGSDIVLTQSPASLA
VSQGQRATISCRAESEVDDYGISFMNWFQQKPGQPPKLLIYAAPNQGSGVPARFSGSGSGTDFSLNIHPMEEDDTATYFCQQSKD
VRWTFGGGTKLEIKRGGGGSGGGGSGGGGSGGGGSEVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWGGDGFYAMDYWGQGTLVTVSSASTGGGGSGGGGSGG
GGSDIQMTQSPSSLSASVGDRVTITCRASQDVNTAVAWYQQKPGKAPKLLIYSASFLYSGVPSRFSGSRSGTDFTLTISSLQPED
FATYYCQQHYTTPPTFGQGTKVEIKRTSRGPHHHHHH
SEQ ID NO. 122
MFGFLIFFIVALLKGVQCDIVLTQSPASLAVSQGQRATISCRASESVDDYGISFMNWFQQKPGQPPKLLIYAAPNQGSGVPARFS
GSGSGTDFSLNIHPMEEDDTATYFCQQSKDVRWTHGGGTKLEIKRGGGGSGGGGSGGGGSGGGGSEVKLVESGGGLVQPGGSLKL
SCAASGFDFSRYWMSWVRQAPGKGLEWIGEINLDSSTINYTPSLKDKFIISRDNAKNTLYLQMSKVRSEDTALYYCARRYDAMDY
WGQGTSVTVSSGGGGSGGGGSGGGGSGGGGSEVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYWGQGTLVTVSSASTGGGGSGGGGSGGGGSD
IQMTQSPSSLSASVGDRVTITCRASQDVNTAVAWYQQKPGKAPKLLIYSASFLYSGVPSRFSGSRSGTDFTLTISSLQPEDFATY
YCQQHYTTPPTFGQGTKVEIKRTSRGPHHHHHH

### Listing of Nucleotide Sequences

SEQID NO. 201
ATGGAGTTTGGGCTGAGCTGGGTTTTCCTCGTTGCTCTTTTTAGAGGTGTCCAGTGTCAGGTGCAGCTGCAGGAGTCG
GGCCCAGGACTGGTGAAGCCTTCGGAGACCCTGTCCCTCACCTGCACTGTCTCTGGTGGCTCCGTCAGCAGTGGTGAT
TACTACTGGACCTGGATCCGGCAGTCCCCAGGGAAGGGACTGGAGTGGATTGGACACATCTATTACAGTGGGAACACC
AATTATAACCCCTCCCTCAAGAGTCGACTCACCATATCAATTGACACGTCCAAGACTCAGTTCTCCCTGAAGCTGAGT
TCTGTGACCGCTGCGGACACGGCCATTTATTACTGTGTGCGAGATCGAGTGACTGGTGCTTTTGATATCTGGGGCCAA
GGGACAATGGTCACCGTCTCTTCAGCCTCCACCAAGGGCCCATCGGTCTTCCCCCTGGCGCCCTGCTCCAGGAGCACC
TCCGAGAGCACAGCGGCCCTGGGCTGCCTGGTCAAGGACTACTTCCCTGAACCGGTGACGGTGTCGTGGAACTCAGGC
GCTCTGACCAGCGGCGTGCACACCTTCCCGGCTGTCCTACAGTCCTCAGGACTCTACTCCCTCAGCAGCGTGGTGACC
GTGCCCTCCAGCAACTTCGGCACCCAGACCTACACCTGCAACGTAGATCACAAGCCCAGCAATACCAAGGTGGACAAG
ACAGTTGAGCGCAAATGTTGTGTCGAGTGCCCACCGTGCCCAGCACCACCTGTGGCAGGACCGTCAGTCTTCCTCTTC
CCCCCAAAACCCAAGGACACCCTCATGATCTCCCGGACCCCTGAGGTCACGTGCGTGGTGGTGGACGTGAGCCACGAA
GACCCCGAGGTCCAGTTCAACTGGTACGTGGACGGCGTGGAGGTGCATAATGCCAAGACAAAGCCACGGGAGGAGCAG
TTCCAAAGCACGTTCCGTGTGGTCAGCGTCCTCACCGTCGTGCACCAGGACTGGCTGAACGGCAAGGAGTACAAGTGC
AAGGTCTCCAACAAAGGCCTCCCAGCCCCCATCGAGAAAACCATCTCCAAAACCAAAGGGCAGCCCCGAGAACCACAG
GTGTACACCCTGCCCCCATCCCGGGAGGAGATGACCAAGAACCAGGTCAGCCTGACCTGCCTGGTCAAAGGCTTCTAC
CCCAGCGACATCGCCGTGGAGTGGGAGAGCAATGGGCAGCCGGAGAACAACTACAAGACCACGCCTCCCATGCTGGAC
TCCGACGGCTCCTTCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGT
SEQID NO. 202
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGGAGGTGGGTCTGGAGGTGGAGGATCTGGTGGA
GGTGGGTCTGACATCCAGATGACCCAGTCTCCATCCTCCCTGTCTGCATCTGTAGGAGACAGAGTCACCATCACTTGC
CAGGCGAGTCAGGACATCAGCAACTATTTAAATTGGTATCAGCAGAAACCAGGGAAAGCCCCTAAACTCCTGATCTAC
GATGCATCCAATTTGGAAACAGGGGTCCCATCAAGGTTCAGTGGAAGTGGATCTGGGACAGATTTTACTTTCACCATC
AGCAGCCTGCAGCCTGAAGATATTGCAACATATTTCTGTCAACACTTTGATCATCTCCCGCTCGCTTTCGGCGGAGGG
ACCAAGGTGGAGATCAAACGAACTGTGGCTGCACCATCTGTCTTCATCTTCCCGCCATCTGATGAGCAGTTGAAATCT
GGAACTGCCTCTGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTCAGCAGCACCCTG
ACGCTGAGCAAAGCAGACTACGAGAAACACAAAGTCTACGCCTGCGAAGTCACCCATCAGGGCCTGAGCTCGCCCGTC
ACAAAGAGCTTCAACAGGGGAGAGTGT
SEQID NO. 203
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGGAGGTGGGTCTGGAGGTGGAGGATCTGGTGGA
GGTGGGTCTCAGGTGCAGCTGCAGGAGTCGGGCCCAGGACTGGTGAAGCCTTCGGAGACCCTGTCCCTCACCTGCACT
GTCTCTGGTGGCTCCGTCAGCAGTGGTGATTACTACTGGACCTGGATCCGGCAGTCCCCAGGGAAGGGACTGGAGTGG
ATTGGACACATCTATTACAGTGGGAACACCAATTATAACCCCTCCCTCAAGAGTCGACTCACCATATCAATTGACACG
TCCAAGACTCAGTTCTCCCTGAAGCTGAGTTCTGTGACCGCTGCGGACACGGCCATTTATTACTGTGTGCGAGATCGA
GTGACTGGTGCTTTTGATATCTGGGGCCAAGGGACAATGGTCACCGTCTCTTCAGCCTCCACCAAGGGCCCATCGGTC
TTCCCCCTGGCGCCCTGCTCCAGGAGCACCTCCGAGAGCACAGCGGCCCTGGGCTGCCTGGTCAAGGACTACTTCCCT
GAACCGGTGACGGTGTCGTGGAACTCAGGCGCTCTGACCAGCGGCGTGCACACCTTCCCGGCTGTCCTACAGTCCTCA
GGACTCTACTCCCTCAGCAGCGTGGTGACCGTGCCCTCCAGCAACTTCGGCACCCAGACCTACACCTGCAACGTAGAT
CACAAGCCCAGCAATACCAAGGTGGACAAGACAGTTGAGCGCAAATGTTGTGTCGAGTGCCCACCGTGCCCAGCACCA
CCTGTGGCAGGACCGTCAGTCTTCCTCTTCCCCCCAAAACCCAAGGACACCCTCATGATCTCCCGGACCCCTGAGGTC
ACGTGCGTGGTGGTGGACGTGAGCCACGAAGACCCCGAGGTCCAGTTCAACTGGTACGTGGACGGCGTGGAGGTGCAT
AATGCCAAGACAAAGCCACGGGAGGAGCAGTTCCAAAGCACGTTCCGTGTGGTCAGCGTCCTCACCGTCGTGCACCAG
GACTGGCTGAACGGCAAGGAGTACAAGTGCAAGGTCTCCAACAAAGGCCTCCCAGCCCCCATCGAGAAAACCATCTCC
AAAACCAAAGGGCAGCCCCGAGAACCACAGGTGTACACCCTGCCCCCATCCCGGGAGGAGATGACCAAGAACCAGGTC
AGCCTGACCTGCCTGGTCAAAGGCTTCTACCCCAGCGACATCGCCGTGGAGTGGGAGAGCAATGGGCAGCCGGAGAAC
AACTACAAGACCACGCCTCCCATGCTGGACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCTGCACAACCACTACACGCAGAAGAGCCTC
TCCCTGTCTCCGGGT
SEQID NO. 204
ATGGAGACAGACACACTCCTGCTATGGGTACTGCTGCTCTGGGTTCCAGGTTCCACTGGTGACATCCAGATGACCCAG
TCTCCATCCTCCCTGTCTGCATCTGTAGGAGACAGAGTCACCATCACTTGCCAGGCGAGTCAGGACATCAGCAACTAT
TTAAATTGGTATCAGCAGAAACCAGGGAAAGCCCCTAAACTCCTGATCTACGATGCATCCAATTTGGAAACAGGGGTC
CCATCAAGGTTCAGTGGAAGTGGATCTGGGACAGATTTTACTTTCACCATCAGCAGCCTGCAGCCTGAAGATATTGCA
ACATATTTCTGTCAACACTTTGATCATCTCCCGCTCGCTTTCGGCGGAGGGACCAAGGTGGAGATCAAACGAACTGTG
GCTGCACCATCTGTCTTCATCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAATCGGGTAACTCCCAGGAGAGT
GTCACAGAGCAGGACAGCAAGGACAGCACCTACAGCCTCAGCAGCACCCTGACGCTGAGCAAAGCAGACTACGAGAAA
CACAAAGTCTACGCCTGCGAAGTCACCCATCAGGGCCTGAGCTCGCCCGTCACAAAGAGCTTCAACAGGGGAGAGTGT
SEQID NO. 205
ATGGAGACAGACACACTCCTGCTATGGGTACTGCTGCTCTGGGTTCCAGGTTCCACTGGTGACATCCAGATGACCCAG
TCTCCATCCTCCCTGTCTGCATCTGTAGGAGACAGAGTCACCATCACTTGCCAGGCGAGTCAGGACATCAGCAACTAT
TTAAATTGGTATCAGCAGAAACCAGGGAAAGCCCCTAAACTCCTGATCTACGATGCATCCAATTTGGAAACAGGGGTC
CCATCAAGGTTCAGTGGAAGTGGATCTGGGACAGATTTTACTTTCACCATCAGCAGCCTGCAGCCTGAAGATATTGCA
ACATATTTCTGTCAACACTTTGATCATCTCCCGCTCGCTTTCGGCGGAGGGACCAAGGTGGAGATCAAACGAACTGTG
GCTGCACCATCTGTCTTCATCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAATCGGGTAACTCCCAGGAGAGT
GTCACAGAGCAGGACAGCAAGGACAGCACCTACAGCCTCAGCAGCACCCTGACGCTGAGCAAAGCAGACTACGAGAAA
CACAAAGTCTACGCCTGCGAAGTCACCCATCAGGGCCTGAGCTCGCCCGTCACAAAGAGCTTCAACAGGGGAGAGTGT
GACAAAACTCACACATCGCCACCGTCCCCAGCACCTGAAGCCGCGGGGGGACCGTCAGAAGTCAGGCCCGAGGAACCT
CTAGTGGTGAAGGTGGAAGAGGGAGATAACGCTGTGCTGCAGTGCCTCAAGGGGACCTCAGATGGCCCCACTCAGCAG
CTGACCTGGTCTCGGGAGTCCCCGCTTAAACCCTTCTTAAAACTCAGCCTGGGGCTGCCAGGCCTGGGAATCCACATG
AGGCCCCTGGCCATCTGGCTTTTCATCTTCAACGTCTCTCAACAGATGGGGGGCTTCTACCTGTGCCAGCCGGGGCCC
CCCTCTGAGAAGGCCTGGCAGCCTGGCTGGACAGTCAATGTGGAGGGCAGCGGG
SEQID NO. 206
ATGGAGTTTGGGCTGAGCTGGGTTTTCCTCGTTGCTCTTTTTAGAGGTGTCCAGTGTCAGGTGCAGCTGCAGGAGTCG
GGCCCAGGACTGGTGAAGCCTTCGGAGACCCTGTCCCTCACCTGCACTGTCTCTGGTGGCTCCGTCAGCAGTGGTGAT
TACTACTGGACCTGGATCCGGCAGTCCCCAGGGAAGGGACTGGAGTGGATTGGACACATCTATTACAGTGGGAACACC
AATTATAACCCCTCCCTCAAGAGTCGACTCACCATATCAATTGACACGTCCAAGACTCAGTTCTCCCTGAAGCTGAGT
TCTGTGACCGCTGCGGACACGGCCATTTATTACTGTGTGCGAGATCGAGTGACTGGTGCTTTTGATATCTGGGGCCAA
GGGACAATGGTCACCGTCTCTTCAGCCTCCACCAAGGGCCCATCGGTCTTCCCCCTGGCGCCCTGCTCCAGGAGCACC
TCCGAGAGCACAGCGGCCCTGGGCTGCCTGGTCAAGGACTACTTCCCTGAACCGGTGACGGTGTCGTGGAACTCAGGC
GCTCTGACCAGCGGCGTGCACACCTTCCCGGCTGTCCTACAGTCCTCAGGACTCTACTCCCTCAGCAGCGTGGTGACC
GTGCCCTCCAGCAACTTCGGCACCCAGACCTACACCTGCAACGTAGATCACAAGCCCAGCAATACCAAGGTGGACAAG
ACAGTTGAGCGCAAATGTTGTGTCGAGTGCCCACCGTGCCCAGCACCACCTGTGGCAGGACCGTCAGTCTTCCTCTTC
CCCCCAAAACCCAAGGACACCCTCATGATCTCCCGGACCCCTGAGGTCACGTGCGTGGTGGTGGACGTGAGCCACGAA
GACCCCGAGGTCCAGTTCAACTGGTACGTGGACGGCGTGGAGGTGCATAATGCCAAGACAAAGCCACGGGAGGAGCAG
TTCCAAAGCACGTTCCGTGTGGTCAGCGTCCTCACCGTCGTGCACCAGGACTGGCTGAACGGCAAGGAGTACAAGTGC
AAGGTCTCCAACAAAGGCCTCCCAGCCCCCATCGAGAAAACCATCTCCAAAACCAAAGGGCAGCCCCGAGAACCACAG
GTGTACACCCTGCCCCCATCCCGGGAGGAGATGACCAAGAACCAGGTCAGCCTGACCTGCCTGGTCAAAGGCTTCTAC
CCCAGCGACATCGCCGTGGAGTGGGAGAGCAATGGGCAGCCGGAGAACAACTACAAGACCACGCCTCCCATGCTGGAC
TCCGACGGCTCCTTCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTGGTGGAGGTGGGTCT
GGAGGTGGAGGATCTGGTGGAGGTGGGTCTGAAGTCAGGCCCGAGGAACCTCTAGTGGTGAAGGTGGAAGAGGGAGAT
AACGCTGTGCTGCAGTGCCTCAAGGGGACCTCAGATGGCCCCACTCAGCAGCTGACCTGGTCTCGGGAGTCCCCGCTT
AAACCCTTCTTAAAACTCAGCCTGGGGCTGCCAGGCCTGGGAATCCACATGAGGCCCCTGGCCATCTGGCTTTTCATC
TTCAACGTCTCTCAACAGATGGGGGGCTTCTACCTGTGCCAGCCGGGGCCCCCCTCTGAGAAGGCCTGGCAGCCTGGC
TGGACAGTCAATGTGGAGGGCAGCGGG
SEQID NO. 207
ATGGAGTTTGGGCTGAGCTGGGTTTTCCTCGTTGCTCTTTTTAGAGGTGTCCAGTGTCAGGTTCAGCTGGTGCAGTCT
GGTGCTGAGGTGAAGAAGCCTGGTGCCTCAGTGAAGGTCTCCTGCAAGGCTTCTGGTTACACATTCACTGACTACTAC
ATGCACTGGGTGCGTCAGGCCCCTGGTCAAGGTCTTGAGTGGATGGGTCGTGTTAATCCTAACCGGAGGGGTACTACC
TACAACCAGAAATTCGAGGGCCGTGTCACCATGACCACAGACACATCCACGAGCACAGCCTACATGGAGCTGCGTAGC
CTGCGTTCTGACGACACGGCCGTGTATTACTGTGCGCGTGCGAACTGGCTTGACTACTGGGGCCAGGGCACCACCGTC
ACCGTCTCCTCCGCCTCCACCAAGGGCCCATCGGTCTTCCCGCTAGCGCCCTGCTCCAGGAGCACCTCCGAGAGCACA
GCCGCCCTGGGCTGCCTGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGTGACCGTGCCCTCCAGC
AGCTTGGGCACGAAGACCTACACCTGCAACGTAGATCACAAGCCCAGCAACACCAAGGTGGACAAGAGAGTTGAGTCC
AAATATGGTCCCCCATGCCCACCCTGCCCAGCACCTGAGGCCGCCGGGGGACCATCAGTCTTCCTGTTCCCCCCAAAA
CCCAAGGACACTCTCATGATCTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGCGGGAGGAGCAGTTCAACAGC
ACGTACCGTGTGGTCAGCGTCCTCACCGTCCTGCACCAGGACTGGCTGAACGGCAAGGAGTACAAGTGCAAGGTCTCC
AACAAAGGCCTCCCGTCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGCTTCTACCCCAGCGAC
ATCGCCGTGGAGTGGGAAAGCAATGGGCAGCCGGAGAACAACTACAAGACCACGCCTCCCGTGCTGGACTCCGACGGC
TCCTTCTTCCTCTACAGCAGGCTAACCGTGGACAAGAGCAGGTGGCAGGAGGGGAATGTCTTCTCATGCTCCGTGATG
CATGAGGCTCTGCACAACCACTACACACAGAAGAGCCTCTCCCTGTCTCTGGGT
SEQID NO. 208
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGGAGGTGGGTCTGGAGGTGGAGGATCTGGTGGA
GGTGGGTCTGACATCCAGATGACCCAGTCTCCATCCTCCCTGTCTGCATCTGTAGGAGACAGAGTCACCATCACTTGC
AGTGTCAGCTCAAGTGTATCCTCCATTTACTTGCACTGGTATCAGCAGAAACCAGGGAAAGCCCCTAAGCTCCTGATC
TATAGCACATCCAACTTGGCTTCTGGAGTCCCATCAAGGTTCAGTGGCAGTGGATCTGGGACAGATTTCACTCTCACC
ATCAGCAGTCTGCAACCTGAAGATTTTGCAACTTACTACTGTCAAGTCTACAGTGGTTACCCGCTCACGTTCGGCGGA
GGGACCAAGGTGGAGATCAAACGAACTGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAAGTACAGTGGAAGGTGGATAAC
GCCCTCCAATCGGGTAACTCCCAGGAGAGTGTCACAGAGCAGGACAGCAAGGACAGCACCTACAGCCTCAGCAGCACC
CTGACGCTGAGCAAAGCAGACTACGAGAAACACAAAGTCTACGCCTGCGAAGTCACCCATCAGGGCCTGAGCTCGCCC
GTCACAAAGAGCTTCAACAGGGGAGAGTGCTAATA
SEQID NO. 209
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGGAGGTGGGTCTGGAGGTGGAGGATCTGGTGGA
GGTGGGTCTCAGGTTCAGCTGGTGCAGTCTGGTGCTGAGGTGAAGAAGCCTGGTGCCTCAGTGAAGGTCTCCTGCAAG
GCTTCTGGTTACACATTCACTGACTACTACATGCACTGGGTGCGTCAGGCCCCTGGTCAAGGTCTTGAGTGGATGGGT
CGTGTTAATCCTAACCGGAGGGGTACTACCTACAACCAGAAATTCGAGGGCCGTGTCACCATGACCACAGACACATCC
ACGAGCACAGCCTACATGGAGCTGCGTAGCCTGCGTTCTGACGACACGGCCGTGTATTACTGTGCGCGTGCGAACTGG
CTTGACTACTGGGGCCAGGGCACCACCGTCACCGTCTCCTCCGCCTCCACCAAGGGCCCATCGGTCTTCCCGCTAGCG
CCCTGCTCCAGGAGCACCTCCGAGAGCACAGCCGCCCTGGGCTGCCTGGTCAAGGACTACTTCCCCG
AACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACGAAGACCTACACCTGCAACGTAGATC
ACAAGCCCAGCAACACCAAGGTGGACAAGAGAGTTGAGTCCAAATATGGTCCCCCATGCCCACCCTGCCCAGCACCTG
AGGCCGCCGGGGGACCATCAGTCTTCCTGTTCCCCCCAAAACCCAAGGACACTCTCATGATCTCCCGGACCCCTGAGG
TCACGTGCGTGGTGGTGGACGTGAGCCAGGAAGACCCCGAGGTCCAGTTCAACTGGTACGTGGATGGCGTGGAGGTGC
ATAATGCCAAGACAAAGCCGCGGGAGGAGCAGTTCAACAGCACGTACCGTGTGGTCAGCGTCCTCACCGTCCTGCACC
AGGACTGGCTGAACGGCAAGGAGTACAAGTGCAAGGTCTCCAACAAAGGCCTCCCGTCCTCCATCGAGAAAACCATCT
CCAAAGCCAAAGGGCAGCCCCGAGAGCCACAGGTGTACACCCTGCCCCCATCCCAGGAGGAGATGACCAAGAACCAGG
TCAGCCTGACCTGCCTGGTCAAAGGCTTCTACCCCAGCGACATCGCCGTGGAGTGGGAAAGCAATGGGCAGCCGGAGA
ACAACTACAAGACCACGCCTCCCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACAACCACTACACACAGAAGAGCC
TCTCCCTGTCTCTG
SEQID NO. 210
ATGGAGACAGACACACTCCTGCTATGGGTACTGCTGCTCTGGGTTCCAGGTTCCACTGGTGACATCCAGATGACCCAG
TCTCCATCCTCCCTGTCTGCATCTGTAGGAGACAGAGTCACCATCACTTGCAGTGTCAGCTCAAGTGTATCCTCCATT
TACTTGCACTGGTATCAGCAGAAACCAGGGAAAGCCCCTAAGCTCCTGATCTATAGCACATCCAACTTGGCTTCTGGA
GTCCCATCAAGGTTCAGTGGCAGTGGATCTGGGACAGATTTCACTCTCACCATCAGCAGTCTGCAACCTGAAGATTTT
GCAACTTACTACTGTCAAGTCTACAGTGGTTACCCGCTCACGTTCGGCGGAGGGACCAAGGTGGAGATCAAACGAACT
GTGGCTGCACCATCTGTCTTCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCTTCAACAGGGGAGAG
TGC
SEQID NO. 211
ATGGAGACAGACACACTCCTGCTATGGGTACTGCTGCTCTGGGTTCCAGGTTCCACTGGTGACATCCAGATGACCCAG
TCTCCATCCTCCCTGTCTGCATCTGTAGGAGACAGAGTCACCATCACTTGCAGTGTCAGCTCAAGTGTATCCTCCATT
TACTTGCACTGGTATCAGCAGAAACCAGGGAAAGCCCCTAAGCTCCTGATCTATAGCACATCCAACTTGGCTTCTGGA
GTCCCATCAAGGTTCAGTGGCAGTGGATCTGGGACAGATTTCACTCTCACCATCAGCAGTCTGCAACCTGAAGATTTT
GCAACTTACTACTGTCAAGTCTACAGTGGTTACCCGCTCACGTTCGGCGGAGGGACCAAGGTGGAGATCAAACGAACT
GTGGCTGCACCATCTGTCTTCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCTTCAACAGGGGAGAG
TGCGACAAAACTCACACATCGCCACCGTCCCCAGCACCTGAAGCCGCGGGGGGACCGTCAGAAGTCAGGCCCGAGGAA
CCTCTAGTGGTGAAGGTGGAAGAGGGAGATAACGCTGTGCTGCAGTGCCTCAAGGGGACCTCAGATGGCCCCACTCAG
CAGCTGACCTGGTCTCGGGAGTCCCCGCTTAAACCCTTCTTAAAACTCAGCCTGGGGCTGCCAGGCCTGGGAATCCAC
ATGAGGCCCCTGGCCATCTGGCTTTTCATCTTCAACGTCTCTCAACAGATGGGGGGCTTCTACCTGTGCCAGCCGGGG
CCCCCCTCTGAGAAGGCCTGGCAGCCTGGCTGGACAGTCAATGTGGAGGGCAGCGGG
SEQID NO. 212
ATGGAGTTTGGGCTGAGCTGGGTTTTCCTCGTTGCTCTTTTTAGAGGTGTCCAGTGTCAGGTTCAGCTGGTGCAGTCT
GGTGCTGAGGTGAAGAAGCCTGGTGCCTCAGTGAAGGTCTCCTGCAAGGCTTCTGGTTACACATTCACTGACTACTAC
ATGCACTGGGTGCGTCAGGCCCCTGGTCAAGGTCTTGAGTGGATGGGTCGTGTTAATCCTAACCGGAGGGGTACTACC
TACAACCAGAAATTCGAGGGCCGTGTCACCATGACCACAGACACATCCACGAGCACAGCCTACATGGAGCTGCGTAGC
CTGCGTTCTGACGACACGGCCGTGTATTACTGTGCGCGTGCGAACTGGCTTGACTACTGGGGCCAGGGCACCACCGTC
ACCGTCTCCTCCGCCTCCACCAAGGGCCCATCGGTCTTCCCGCTAGCGCCCTGCTCCAGGAGCACCTCCGAGAGCACA
GCCGCCCTGGGCTGCCTGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGTGACCGTGCCCTCCAGC
AGCTTGGGCACGAAGACCTACACCTGCAACGTAGATCACAAGCCCAGCAACACCAAGGTGGACAAGAGAGTTGAGTCC
AAATATGGTCCCCCATGCCCACCCTGCCCAGCACCTGAGGCCGCCGGGGGACCATCAGTCTTCCTGTTCCCCCCAAAA
CCCAAGGACACTCTCATGATCTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGCGGGAGGAGCAGTTCAACAGC
ACGTACCGTGTGGTCAGCGTCCTCACCGTCCTGCACCAGGACTGGCTGAACGGCAAGGAGTACAAGTGCAAGGTCTCC
AACAAAGGCCTCCCGTCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGCTTCTACCCCAGCGAC
ATCGCCGTGGAGTGGGAAAGCAATGGGCAGCCGGAGAACAACTACAAGACCACGCCTCCCGTGCTGGACTCCGACGGC
TCCTTCTTCCTCTACAGCAGGCTAACCGTGGACAAGAGCAGGTGGCAGGAGGGGAATGTCTTCTCATGCTCCGTGATG
CATGAGGCTCTGCACAACCACTACACACAGAAGAGCCTCTCCCTGTCTCTGGGTGGTGGAGGTGGGTCTGGAGGTGGA
GGATCTGGTGGAGGTGGGTCTGAAGTCAGGCCCGAGGAACCTCTAGTGGTGAAGGTGGAAGAGGGAGATAACGCTGTG
CTGCAGTGCCTCAAGGGGACCTCAGATGGCCCCACTCAGCAGCTGACCTGGTCTCGGGAGTCCCCGCTTAAACCCTTC
TTAAAACTCAGCCTGGGGCTGCCAGGCCTGGGAATCCACATGAGGCCCCTGGCCATCTGGCTTTTCATCTTCAACGTC
TCTCAACAGATGGGGGGCTTCTACCTGTGCCAGCCGGGGCCCCCCTCTGAGAAGGCCTGGCAGCCTGGCTGGACAGTC
AATGTGGAGGGCAGCGGG
SEQID NO. 213
ATGGAGACAGACACACTCCTGCTATGGGTACTGCTGCTCTGGGTTCCAGGTTCCACTGGTGACATCCAGATGACACAG
ACTACATCCTCCCTGTCTGCCTCTCTGGGAGACAGAGTCACCATCAGTTGCAGGGCAAGTCAGGACATTAGTAAATAT
TTAAATTGGTATCAGCAGAAACCAGATGGAACTGTTAAACTCCTGATCTACCATACATCAAGATTACACTCAGGAGTC
CCATCAAGGTTCAGTGGCAGTGGGTCTGGAACAGATTATTCTCTCACCATTAGCAACCTGGAGCAAGAAGATATTGCC
ACTTACTTTTGCCAACAGGGTAATACGCTTCCGTACACGTTCGGAGGGGGGACTAAGTTGGAAATAACAGGAGGAGGT
GGGTCTGGAGGTGGAGGATCTGGTGGAGGTGGGTCTGGAGGAGGTGGGTCTGAGGTGAAACTGCAGGAGTCAGGACCT
GGCCTGGTGGCGCCCTCACAGAGCCTGTCCGTCACATGCACTGTCTCAGGGGTCTCATTACCCGACTATGGTGTAAGC
TGGATTCGCCAGCCTCCACGAAAGGGTCTGGAGTGGCTGGGAGTAATATGGGGTAGTGAAACCACATACTATAATTCA
GCTCTCAAATCCAGACTGACCATCATCAAGGACAACTCCAAGAGCCAAGTTTTCTTAAAAATGAACAGTCTGCAAACT
GATGACACAGCCATTTACTACTGTGCCAAACATTATTACTACGGTGGTAGCTATGCTATGGACTACTGGGGTCAAGGA
ACCTCAGTCACCGTCTCCTCACCGAGCCAGTTCCGGGTGTCGCCGCTGGATCGGACCTGGAACCTGGGCGAGACAGTG
GAGCTGAAGTGCCAGGTGCTGCTGTCCAACCCGACGTCGGGCTGCTCGTGGCTCTTCCAGCCGCGCGGCGCCGCCGCC
AGTCCCACCTTCCTCCTATACCTCTCCCAAAACAAGCCCAAGGCGGCCGAGGGGCTGGACACCCAGCGGTTCTCGGGC
AAGAGGTTGGGGGACACCTTCGTCCTCACCCTGAGCGACTTCCGCCGAGAGAACGAGGGCTACTATTTCTGCTCGGCC
CTGAGCAACTCCATCATGTACTTCAGCCACTTCGTGCCGGTCTTCCTGCCAGCGAAGCCCACCACGACGCCAGCGCCG
CGACCACCAACACCGGCGCCCACCATCGCGTCGCAGCCCCTGTCCCTGCGCCCAGAGGCGTGCCGGCCAGCGGCGGGG
GGCGCAGTGCACACGAGGGGGCTGGACTTCGCCTGTGATATCTACATCTGGGCGCCCTTGGCCGGGACTTGTGGGGTC
CTTCTCCTGTCACTGGTTATCACCAGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCGCAGCCTATCGCTCCAAACGGGGC
AGAAAGAAACTCCTGTATATATTCAAACAACCATTTATGAGACCAGTACAAACTACTCAAGAGGAAGATGGCTGTAGC
TGCCGATTTCCAGAAGAAGAAGAAGGAGGATGTGAACTGAGAGTGAAGTTCAGCAGGAGCGCAGACGCCCCCGCGTAC
CAGCAGGGCCAGAACCAGCTCTATAACGAGCTCAATCTAGGACGAAGAGAGGAGTACGATGTTTTGGACAAGAGACGT
GGCCGGGACCCTGAGATGGGGGGAAAGCCGCAGAGAAGGAAGAACCCTCAGGAAGGCCTGTACAATGAACTGCAGAAA
GATAAGATGGCGGAGGCCTACAGTGAGATTGGGATGAAAGGCGAGCGCCGGAGGGGCAAGGGGCACGATGGCCTTTAC
CAGGGTCTCAGTACAGCCACCAAGGACACCTACGACGCCCTTCACATGCAGGCCCTGCCCCCTCGC
SEQID NO. 214
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGCATCATCACCATCACCAT
SEQID NO. 215
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGACAAAACTCACACATGCCCACCGTGCCCAGCA
CCTGAACTCCTGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCCCCATCGAGAAAACC
ATCTCCAAAGCCAAAGGGCAGCCCCGAGAACCACAGGTGTACACCCTGCCCCCATCCCGGGAGGAGATGACCAAGAAC
CAGGTCAGCCTGACCTGCCTGGTCAAAGGCTTCTATCCCAGCGACATCGCCGTGGAGTGGGAGAGCAATGGGCAGCCG
GAGAACAACTACAAGACCACGCCTCCCGTGCTGGACTCCGACGGCTCCTTCTTCCTCTACAGCAAGCTCACCGTGGAC
AAGAGCAGGTGGCAGCAGGGGAACGTCTTCTCATGCTCCGTGATGCACGAGGCTCTGCACAACCACTACACGCAGAAG
AGCCTCTCCCTGTCTCCGGGT
SEQID NO. 216
ATGGAGTTTGGGCTGAGCTGGGTTTTCCTCGTTGCTCTTTTTAGAGGTGTCCAGTGTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGGGGCCCCATCATCACCATCACCAT
SEQID NO. 217
ATGGAGTTTGGGCTGAGCTGGGTTTTCCTCGTTGCTCTTTTTAGAGGTGTCCAGTGTCAGGTGCAGCTGGTGCAGAGC
GGCGCCGAGGACAAGAAGCCCGGCGAGAGCGTGAAGATCAGCTGCAAGGCCAGCGGCTACACCTTCACCAACTACGGC
ATGAACTGGGTGAGGCAGGCCCCCGGCCAGGGCCTGAAGTGGATGGGCTGGATCAACACCTACACCGGCGAGAGCACC
TACGCCGACGACTTCAAGGGCAGGTTCGCCTTCAGCCTGGACACCAGCGCCAGCACCGCCTACCTGCAGCTGAGCAGC
CTGAGGGGCGAGGACACCGCCGTGTACTTCTGCGCCAGGTTCGCCATCAAGGGCGACTACTGGGGCCAGGGCACCACC
GTGACCGTGAGCAGCGCCAGCACCGGCGGCGGCGGCAGCGGCGGCGGCGGCAGCGGCGGCGGCGGCAGCGGCGGCGGC
GGCAGCGACATCGTGATGACCCAGAGCCCCCTGAGCCTGGAGGTGAGCCCCGGCGAGCCCGCCAGCATCAGCTGCAGG
AGCACCAAGAGCCTGCTGCACAGCGACGGCATCACCTACCTGTACTGGTACCTGCAGAAGCCCGGCCAGAGCCCCCAG
CTGCTGATCTACCAGCTGAGCAACCTGGCCAGCGGCGTGCCCGACAGGTTCAGCAGCAGCGGCAGCGGCACCGACTTC
ACCCTGAAGATCAGCAGGGTGGAGGCCGAGGACGAGGGCACCTACTACTGCGCCCAGAACCTGGAGATCCCCAGGACC
TTCGGCCAGGGCACCAAGCTGGAGATCAAGAGGACCGGGCCCCATCATCACCATCACCAT
SEQID NO. 218
ATGGAGACAGACACACTCCTGCTATGGGTACTGCTGCTCTGGGTTCCAGGTTCCACTGGTGACATCGTGATGACCCAG
AGCCCCCTGAGCCTGCCCGTGACCCCCGGCGAGCCCGCCAGCATCAGCTGCAGGAGCACCAAGAGCCTGCTGCACAGC
GACGGCATCACCTACCTGTACTGGTACCTGCAGAAGCCCGGCCAGAGCCCCCAGCTGCTGATCTACCAGCTGAGCAAC
CTGGCCAGCGGCGTGCCCGACAGGTTCAGCAGCAGCGGCAGCGGCACCGACTTCACCCTGAAGATCAGCAGGGTGGAG
GCCGAGGACGAGGGCGTGTACTACTGCGCCCAGAACCTGGAGATCCCCAGGACCTTCGGCTGCGGCACCAAGCTGGAG
ATCAAGAGGACCGGCGGCGGCGGCAGCGGCGGCGGCGGCAGCGGCGGCGGCGGCAGCGGCGGCGGCGGCAGCCAGGTG
CAGCTGGTGCAGAGCGGCGCCGAGGTGAAGAAGCCCGGCGAGAGCGTGAAGATCAGCTGCAAGGCCAGCGGCTACACC
TTCACCAACTACGGCATGAACTGGGTGAGGCAGGCCCCCGGCCAGTGCCTGAAGTGGATGGGCTGGATCAACACCTAC
ACCGGCGAGAGCACCTACGCCGACGACTTCAAGGGCAGGTTCGCCTTCAGCCTGGACACCAGCGCCAGCACCGCCTAC
CTGCAGCTGAGCAGCCTGAGGAGCGAGGACACCGCCGTGTACTTCTGCGCCAGGTTCGCCATCAAGGGCGACTACTGG
GGCCAGGGCACCCTGGTGACCGTGAGCAGCGGGCCCCATCATCACCATCACCAT
SEQID NO. 219
ATGGAGTTTGGGCTGAGCTGGGTTTTCCTCGTTGCTCTTTTTAGAGGTGTCCAGTGTCAGGTTCAGCTGGTGCAGTCT
GGTGCTGAGGATGTGAAGCCTGATGCCTCAGTGAAGCTCTCCTGCAAGGCTTCTGGTTACACATTCACTGACTACTAC
ATGCACTGGGTGCGTCAGGCCCCTGGTCAAGGTCTTGAGTGGATGGGTCGTGTTAATCCTAACCGGAGGGGTACTACC
TACAACCAGAAATTCGAGGGCCGTGTCACCATGACCACAGACACATCCACGAGCACAGCCTACATGCAGCTGAGTAGC
CTGCGTGGTGAAGACACGGCCGTGTATTACTGTGCGCGTGCGAACTGGCTTGACTACTGGGGCCAGGGCACCACCGTC
ACCGTCTCCTCCGCCTCCACCGGGGGAGGTGGGTCTGGAGGTGGAGGATCTGGTGGAGGTGGGTCTGGTGGAGGTGGG
TCTGACATCCAGATGACCCAGTCTCCATCCTCCCTGGAGGCATCTGTAGGAGACAGAGTCACCATCACTTGCAGTGTC
AGCTCAAGTGTATCCTCCATTTACTTGCACTGGTATCAGCAGAAACCAGGGAAAAGCCCTAAGCTCCTGATCTATAGC
ACATCCAACTTGGCTTCTGGAGTCCCAGATAGGTTCAGTGGCAGTGGATCTGGGACAGATTTCACTCTCACCATCAGC
AGTCTGCAAGCCGAAGATGAGGGCACTTACTACTGTCAAGTCTACAGTGGTTACCCGCTCACGTTCGGCGGAGGGACC
AAGCTGGAGATCAAACGAACTGGGCCCCATCATCACCATCACCAT
SEQID NO. 220
ATGGAGACAGACACACTCCTGCTATGGGTACTGCTGCTCTGGGTTCCAGGTTCCACTGGTGACATCCAGATGACCCAG
TCTCCATCCTCCCTGTCTGCATCTGTAGGAGACAGAGTCACCATCACTTGCAGTGTCAGCTCAAGTGTATCCTCCATT
TACTTGCACTGGTATCAGCAGAAACCAGGGAAAGCCCCTAAGCTCCTGATCTATAGCACATCCAACTTGGCTTCTGGA
GTCCCATCAAGGTTCAGTGGCAGTGGATCTGGGACAGATTTCACTCTCACCATCAGCAGTCTGCAACCTGAAGATGAA
GCAACTTACTACTGTCAAGTCTACAGTGGTTACCCGCTCACGTTCGGCTGCGGGACCAAGGTGGAGATCAAACGAACT
GGGGGAGGTGGGTCTGGAGGTGGAGGATCTGGTGGAGGTGGGTCTGGTGGAGGTGGGTCTCAGGTTCAGCTGGTGCAG
TCTGGTGCTGAGGATAAGAAGCCTGGTGCCTCAGTGAAGGTCTCCTGCAAGGCTTCTGGTTACACATTCACTGACTAC
TACATGCACTGGGTGCGTCAGGCCCCTGGTCAATGTCTTGAGTGGATGGGTCGTGTTAATCCTAACCGGAGGGGTACT
ACCTACAACCAGAAATTCGAGGGCCGTGTCACCATGACCACAGACACATCCACGAGCACAGCCTACATGGAGCTGCGT
AGCCTGCGTTCTGACGACACGGCCGTGTATTACTGTGCGCGTGCGAACTGGCTTGACTACTGGGGCCAGGGCACCACC
GTCACCGTCTCCTCCGGGCCCCATCATCACCATCACCAT
SEQID NO. 221
ATGGAGTTTGGGCTGAGCTGGGTTTTCCTCGTTGCTCTTTTTAGAGGTGTCCAGTGTCAGGTGCAGCTGCAGGAGAGC
GGCCCCGGCGACGTGAAGCCCAGCGAGACCCTGAGCCTGACCTGCACCGTGAGCGGCGGCAGCGTGAGCAGCGGCGAC
TACTACTGGACCTGGATCAGGCAGAGCCCCGGCAAGGGCCTGGAGTGGATCGGCCACATCTACTACAGCGGCAACACC
AACTACAACCCCAGCCTGAAGAGCAGGCTGACCATCAGCATCGACACCAGCAAGACCACCTTCAGCCTGCAGCTGAGC
AGCGTGACCGGCGAGGACACCGCCATCTACTACTGCGTGAGGGACAGGGTGACCGGCGCCTTCGACATCTGGGGCCAG
GGCACCACCGTGACCGTGAGCAGCGCCAGCACCGGCGGCGGCGGCAGCGGCGGCGGCGGCAGCGGCGGCGGCGGCAGC
GGCGGCGGCGGCAGCGACATCCAGATGACCCAGAGCCCCAGCAGCCTGGAGGCCAGCGTGGGCGACAGGGTGACCATC
ACCTGCCAGGCCAGCCAGGACATCAGCAACTACCTGAACTGGTACCAGCAGAAGCCCGGCAAGAGCCCCAAGCTGCTG
ATCTACGACGCCAGCAACCTGGAGACCGGCGTGCCCGACAGGTTCAGCGGCAGCGGCAGCGGCACCGACTTCACCTTC
ACCATCAGCAGCCTGCAGGCCGAGGACGAGGGCACCTACTTCTGCCAGCACTTCGACCACCTGCCCCTGGCCTTCGGC
GGCGGCACCAAGCTGGAGATCAAGAGGACCGGGCCCCATCATCACCATCACCAT
SEQID NO. 222
ATGGAGACAGACACACTCCTGCTATGGGTACTGCTGCTCTGGGTTCCAGGTTCCACTGGTGACATCCAGATGACCCAG
AGCCCCAGCAGCCTGAGCGCCAGCGTGGGCGACAGGGTGACCATCACCTGCCAGGCCAGCCAGGACATCAGCAACTAC
CTGAACTGGTACCAGCAGAAGCCCGGCAAGGCCCCCAAGCTGCTGATCTACGACGCCAGCAACCTGGAGACCGGCGTG
CCCAGCAGGTTCAGCGGCAGCGGCAGCGGCACCGACTTCACCTTCACCATCAGCAGCCTGCAGCCCGAGGACGAGGCC
ACCTACTTCTGCCAGCACTTCGACCACCTGCCCCTGGCCTTCGGCTGCGGCACCAAGGTGGAGATCAAGAGGACCGGC
GGCGGCGGCAGCGGCGGCGGCGGCAGCGGCGGCGGCGGCAGCGGCGGCGGCGGCAGCCAGGTGCAGCTGCAGGAGAGC
GGCCCCGGCGACGTGAAGCCCAGCGAGACCCTGAGCCTGACCTGCACCGTGAGCGGCGGCAGCGTGAGCAGCGGCGAC
TACTACTGGACCTGGATCAGGCAGAGCCCCGGCAAGTGCCTGGAGTGGATCGGCCACATCTACTACAGCGGCAACACC
AACTACAACCCCAGCCTGAAGAGCAGGCTGACCATCAGCATCGACACCAGCAAGACCCAGTTCAGCCTGAAGCTGAGC
AGCGTGACCGCCGCCGACACCGCCATCTACTACTGCGTGAGGGACAGGGTGACCGGCGCCTTCGACATCTGGGGCCAG
GGCACCACCGTGACCGTGAGCAGCGGGCCCCATCATCACCATCACCAT
SEQID NO. 223
ATGCCACCTCCTCGCCTCCTCTTCTTCCTCCTCTTCCTCACCCCCATGGAAGTCAGGCATCATCACCATCACCATCCC
GAGGAACCTCTAGTGGTGAAGGTGGAAGAGGGAGATAACGCTGTGCTGCAGTGCCTCAAGGGGACCTCAGATGGCCCC
ACTCAGCAGCTGACCTGGTCTCGGGAGTCCCCGCTTAAACCCTTCTTAAAACTCAGCCTGGGGCTGCCAGGCCTGGGA
ATCCACATGAGGCCCCTGGCCATCTGGCTTTTCATCTTCAACGTCTCTCAACAGATGGGGGGCTTCTACCTGTGCCAG
CCGGGGCCCCCCTCTGAGAAGGCCTGGCAGCCTGGCTGGACAGTCAATGTGGAGGGCAGCGGGGAGCTGTTCCGGTGG
AATGTTTCGGACCTAGGTGGCCTGGGCTGTGGCCTGAAGAACAGGTCCTCAGAGGGCCCCAGCTCCCCTTCCGGGAAG
CTCATGAGCCCCAAGCTGTATGTGTGGGCCAAAGACCGCCCTGAGATCTGGGAGGGAGAGCCTCCGTGTCTCCCACCG
AGGGACAGCCTGAACCAGAGCCTCAGCCAGGACCTCACCATGGCCCCTGGCTCCACACTCTGGCTGTCCTGTGGGGTA
CCCCCTGACTCTGTGTCCAGGGGCCCCCTCTCCTGGACCCATGTGCACCCCAAGGGGCCTAAGTCATTGCTGAGCCTA
GAGCTGAAGGACGATCGCCCGGCCAGAGATATGTGGGTAATGGAGACGGGTCTGTTGTTGCCCCGGGCCACAGCTCAA
GACGCTGGAAAGTATTATTGTCACCGTGGCAACCTGACCATGTCATTCCACCTGGAGATCACTGCTCGGCCAGGGGGA
GGTGGGTCTGGAGGTGGAGGATCTGGTGGAGGTGGGTCTGTCAGATCATCTTCTCGAACCCCGAGTGACAAGCCTGTA
GCCCATGTTGTAGCAAACCCTCAAGCTGAGGGGCAGCTCCAGTGGCTGAACCGCCGGGCCAATGCCCTCCTGGCCAAT
GGCGTGGAGCTGAGAGATAACCAGCTGGTGGTGCCATCAGAGGGCCTGTACCTCATCTACTCCCAGGTCCTCTTCAAG
GGCCAAGGCTGCCCCTCCACCCATGTGCTCCTCACCCACACCATCAGCCGCATCGCCGTCTCCTACCAGACCAAGGTC
AACCTCCTCTCTGCCATCAAGAGCCCCTGCCAGAGGGAGACCCCAGAGGGGGCTGAGGCCAAGCCCTGGTATGAGCCC
ATCTATCTGGGAGGGGTCTTCCAGCTGGAGAAGGGTGACCGACTCAGCGCTGAGATCAATCGGCCCGACTATCTCGAC
TTTGCCGAGTCTGGGCAGGTCTACTTTGGGATCATTGCCCTG
SEQID NO. 224
ATGGAGTTTGGGCTGAGCTGGGTTTTCCTCGTTGCTCTTTTTAGAGGTGTCCAGTGTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGGACAAAACTCACACATCGCCACCGTCCCCAGCACCTGAAGCCGCGGGGGGACCGTCAGAA
GTCAGGCCCGAGGAACCTCTAGTGGTGAAGGTGGAAGAGGGAGATAACGCTGTGCTGCAGTGCCTCAAGGGGACCTCA
GATGGCCCCACTCAGCAGCTGACCTGGTCTCGGGAGTCCCCGCTTAAACCCTTCTTAAAACTCAGCCTGGGGCTGCCA
GGCCTGGGAATCCACATGAGGCCCCTGGCCATCTGGCTTTTCATCTTCAACGTCTCTCAACAGATGGGGGGCTTCTAC
CTGTGCCAGCCGGGGCCCCCCTCTGAGAAGGCCTGGCAGCCTGGCTGGACAGTCAATGTGGAGGGCAGCGGGTCTAGA
GGGCCCCATCATCACCATCACCAT
SEQID NO. 225
ATGGAGTTTGGGCTGAGCTGGGTTTTCCTCGTTGCTCTTTTTAGAGGTGTCCAGTGTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGGACAAAACTCACACATCGCCACCGTCCCCAGCACCTGAAGCCGCGGGGGGACCGTCAGAA
GTCAGGCCCGAGGAACCTCTAGTGGTGAAGGTGGAAGAGGGAGATAACGCTGTGCTGCAGTGCCTCAAGGGGACCTCA
GATGGCCCCACTCAGCAGCTGACCTGGTCTCGGGAGTCCCCGCTTAAACCCTTCTTAAAACTCAGCCTGGGGCTGCCA
GGCCTGGGAATCCACATGAGGCCCCTGGCCATCTGGCTTTTCATCTTCAACGTCTCTCAACAGATGGGGGGCTTCTAC
CTGTGCCAGCCGGGGCCCCCCTCTGAGAAGGCCTGGCAGCCTGGCTGGACAGTCAATGTGGAGGGCAGCGGGGACAAA
ACTCACACATGCCCACCGTGCCCAGCACCTGAACTCCTGGGGGGACCGTCAGTCTTCCTCTTCCCCCCAAAACCCAAG
GACACCCTCATGATCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCC
CCATCCCGGGAGGAGATGACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTGGACTCCGACGGCTCCTTC
TTCCTCTACAGCAAGCTCACCGTGGACAAGAGCAGGTGGCAGCAGGGGAACGTCTTCTCATGCTCCGTGATGCACGAG
GCTCTGCACAACCACTACACGCAGAAGAGCCTCTCCCTGTCTCCGGGT
SEQID NO. 226
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGGGGGAGGTGGGTCTGGAGGTGGAGGATCTGGT
GGAGGTGGGTCTGGTGGAGGTGGGTCTGAGGTGCAGCTGGTGGAGTCTGGTGGTGGTCTTGTTCAACCTGGTGGTTCT
CTTCGTCTTTCTTGTGCTGCTTCTGGTTTTAATATTAAAGATACTTATATTCATTGGGTTCGTCAAGCTCCTGGTAAA
GGTCTTGAATGGGTTGCTCGTATTTATCCTACTAATGGTTATACTCGTTATGCTGATTCTGTTAAAGGTCGTTTTACT
ATTTCTGCTGATACTTCTAAAAATACTGCTTATCTTCAAATGAACTCTCTTCGTGCTGAAGATACTGCTGTTTATTAT
TGTTCTCGTTGGGGTGGTGATGGTTTTTATGCTATGGATTATTGGGGTCAAGGTACTCTTGTCACCGTCTCCTCAGCT
AGCACCGGGGGAGGTGGGTCTGGAGGTGGAGGATCTGGTGGAGGTGGGTCTGACATCCAGATGACCCAGTCTCCTTCT
TCTCTTTCTGCTTCTGTTGGTGATCGTGTTACTATTACTTGTCGTGCTTCTCAAGATGTTAATACTGCTGTTGCTTGG
TATCAACAAAAACCTGGTAAAGCTCCTAAACTTCTTATTTATTCTGCTTCTTTTCTTTATTCTGGTGTTCCTTCTCGT
TTTTCTGGTTCTCGTTCTGGTACTGATTTTACTCTTACTATTTCTTCTCTTCAACCTGAAGATTTTGCTACTTATTAT
TGTCAACAACATTATACTACTCCTCCTACTTTTGGTCAAGGTACCAAGGTGGAGATCAAACGTACGTCTAGAGGGCCC
CATCATCACCATCACCAT
SEQID NO. 227
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGGGGGAGGTGGGTCTGGAGGTGGAGGATCTGGT
GGAGGTGGGTCTGGTGGAGGTGGGTCTGAGGTGCAGCTGGTGGAGTCTGGTGGTGGTCTTGTTCAACCTGGTGGTTCT
CTTCGTCTTTCTTGTGCTGCTTCTGGTTTTAATATTAAAGATACTTATATTCATTGGGTTCGTCAAGCTCCTGGTAAA
GGTCTTGAATGGGTTGCTCGTATTTATCCTACTAATGGTTATACTCGTTATGCTGATTCTGTTAAAGGTCGTTTTACT
ATTTCTGCTGATACTTCTAAAAATACTGCTTATCTTCAAATGAACTCTCTTCGTGCTGAAGATACTGCTGTTTATTAT
TGTTCTCGTTGGGGTGGTGATGGTTTTTATGCTATGGATTATTGGGGTCAAGGTACTCTTGTCACCGTCTCCTCAGCT
AGCACCGGGGGAGGTGGGTCTGGAGGTGGAGGATCTGGTGGAGGTGGGTCTGACATCCAGATGACCCAGTCTCCTTCT
TCTCTTTCTGCTTCTGTTGGTGATCGTGTTACTATTACTTGTCGTGCTTCTCAAGATGTTAATACTGCTGTTGCTTGG
TATCAACAAAAACCTGGTAAAGCTCCTAAACTTCTTATTTATTCTGCTTCTTTTCTTTATTCTGGTGTTCCTTCTCGT
TTTTCTGGTTCTCGTTCTGGTACTGATTTTACTCTTACTATTTCTTCTCTTCAACCTGAAGATTTTGCTACTTATTAT
TGTCAACAACATTATACTACTCCTCCTACTTTTGGTCAAGGTACCAAGGTGGAGATCAAACGTACGGACAAAACTCAC
ACATGCCCACCGTGCCCAGCACCTGAACTCCTGGGGGGACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTGCAAGGTCTCCAACAAAGCCCTC
CCAGCCCCCATCGAGAAAACCATCTCCAAAGCCAAAGGGCAGCCCCGAGAACCACAGGTGTACACCCTGCCCCCATCC
CGGGAGGAGATGACCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACGGCTCCTTCTTCCTC
TACAGCAAGCTCACCGTGGACAAGAGCAGGTGGCAGCAGGGGAACGTCTTCTCATGCTCCGTGATGCACGAGGCTCTG
CACAACCACTACACGCAGAAGAGCCTCTCCCTGTCTCCGGGT
SEQID NO. 228
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCACATCATCACCATCACCAT
SEQID NO. 229
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGACAAAACTCACACATGCCCACCG
TGCCCAGCACCTGAACTCCTGGGGGGACCGTCAGTCTTCCTCTTCCCCCCAAAACCCAAGGACACCCTCATGATCTCC
CGGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCCGGGAGGAGATG
ACCAAGAACCAGGTCAGCCTGACCTGCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCACGAGGCTCTGCACAACCACTAC
ACGCAGAAGAGCCTCTCCCTGTCTCCGGGT
SEQID NO. 230
ATGCCACCTCCTCGCCTCCTCTTCTTCCTCCTCTTCCTCACCCCCATGGAAGTCAGGCCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCACATCATCACCATCACCAT
SEQID NO. 231
ATGCCACCTCCTCGCCTCCTCTTCTTCCTCCTCTTCCTCACCCCCATGGAAGTCAGGCCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGACAAAACTCACACATGCCCACCG
TGCCCAGCACCTGAACTCCTGGGGGGACCGTCAGTCTTCCTCTTCCCCCCAAAACCCAAGGACACCCTCATGATCTCC
CGGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCCGGGAGGAGATG
ACCAAGAACCAGGTCAGCCTGACCTGCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCACGAGGCTCTGCACAACCACTAC
ACGCAGAAGAGCCTCTCCCTGTCTCCGGGT
SEQID NO. 232
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGACATCCAGATGACCCAGTCTCCATCCTCCCTGTCTGCATCTGTAGGAGACAGAGTC
ACCATCACTTGCCAGGCGAGTCAGGACATCAGCAACTATTTAAATTGGTATCAGCAGAAACCAGGGAAAGCCCCTAAA
CTCCTGATCTACGATGCATCCAATTTGGAAACAGGGGTCCCATCAAGGTTCAGTGGAAGTGGATCTGGGACAGATTTT
ACTTTCACCATCAGCAGCCTGCAGCCTGAAGATATTGCAACATATTTCTGTCAACACTTTGATCATCTCCCGCTCGCT
TTCGGCGGAGGGACCAAGGTGGAGATCAAACGAACTGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGT
SEQID NO. 233
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTCAGGTGCAGCTGCAGGAGTCGGGCCCAGGACTGGTGAAGCCTTCGGAGACCCTGTCC
CTCACCTGCACTGTCTCTGGTGGCTCCGTCAGCAGTGGTGATTACTACTGGACCTGGATCCGGCAGTCCCCAGGGAAG
GGACTGGAGTGGATTGGACACATCTATTACAGTGGGAACACCAATTATAACCCCTCCCTCAAGAGTCGACTCACCATA
TCAATTGACACGTCCAAGACTCAGTTCTCCCTGAAGCTGAGTTCTGTGACCGCTGCGGACACGGCCATTTATTACTGT
GTGCGAGATCGAGTGACTGGTGCTTTTGATATCTGGGGCCAAGGGACAATGGTCACCGTCTCTTCAGCCTCCACCAAG
GGCCCATCGGTCTTCCCCCTGGCGCCCTGCTCCAGGAGCACCTCCGAGAGCACAGCGGCCCTGGGCTGCCTGGTCAAG
GACTACTTCCCTGAACCGGTGACGGTGTCGTGGAACTCAGGCGCTCTGACCAGCGGCGTGCACACCTTCCCGGCTGTC
CTACAGTCCTCAGGACTCTACTCCCTCAGCAGCGTGGTGACCGTGCCCTCCAGCAACTTCGGCACCCAGACCTACACC
TGCAACGTAGATCACAAGCCCAGCAATACCAAGGTGGACAAGACAGTTGAGCGCAAATGTTGTGTCGAGTGCCCACCG
TGCCCAGCACCACCTGTGGCAGGACCGTCAGTCTTCCTCTTCCCCCCAAAACCCAAGGACACCCTCATGATCTCCCGG
ACCCCTGAGGTCACGTGCGTGGTGGTGGACGTGAGCCACGAAGACCCCGAGGTCCAGTTCAACTGGTACGTGGACGGC
GTGGAGGTGCATAATGCCAAGACAAAGCCACGGGAGGAGCAGTTCCAAAGCACGTTCCGTGTGGTCAGCGTCCTCACC
GTCGTGCACCAGGACTGGCTGAACGGCAAGGAGTACAAGTGCAAGGTCTCCAACAAAGGCCTCCCAGCCCCCATCGAG
AAAACCATCTCCAAAACCAAAGGGCAGCCCCGAGAACCACAGGTGTACACCCTGCCCCCATCCCGGGAGGAGATGACC
AAGAACCAGGTCAGCCTGACCTGCCTGGTCAAAGGCTTCTACCCCAGCGACATCGCCGTGGAGTGGGAGAGCAATGGG
CAGCCGGAGAACAACTACAAGACCACGCCTCCCATGCTGGACTCCGACGGCTCCTTCTTCCTCTACAGCAAGCTCACC
GTGGACAAGAGCAGGTGGCAGCAGGGGAACGTCTTCTCATGCTCCGTGATGCATGAGGCTCTGCACAACCACTACACG
CAGAAGAGCCTCTCCCTGTCTCCGGGT
SEQID NO. 234
ATGGAGACAGACACACTCCTGCTATGGGTACTGCTGCTCTGGGTTCCAGGTTCCACTGGTGACATCCAGATGACCCAG
TCTCCATCCTCCCTGTCTGCATCTGTAGGAGACAGAGTCACCATCACTTGCCAGGCGAGTCAGGACATCAGCAACTAT
TTAAATTGGTATCAGCAGAAACCAGGGAAAGCCCCTAAACTCCTGATCTACGATGCATCCAATTTGGAAACAGGGGTC
CCATCAAGGTTCAGTGGAAGTGGATCTGGGACAGATTTTACTTTCACCATCAGCAGCCTGCAGCCTGAAGATATTGCA
ACATATTTCTGTCAACACTTTGATCATCTCCCGCTCGCTTTCGGCGGAGGGACCAAGGTGGAGATCAAACGAACTGTG
GCTGCACCATCTGTCTTCATCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAATCGGGTAACTCCCAGGAGAGT
GTCACAGAGCAGGACAGCAAGGACAGCACCTACAGCCTCAGCAGCACCCTGACGCTGAGCAAAGCAGACTACGAGAAA
CACAAAGTCTACGCCTGCGAAGTCACCCATCAGGGCCTGAGCTCGCCCGTCACAAAGAGCTTCAACAGGGGAGAGTGT
GACAAAACTCACACATCGCCACCGTCCCCAGCACCTGAAGCCGCGGGGGGACCGTCACCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCA
SEQID NO. 235
ATGGAGTTTGGGCTGAGCTGGGTTTTCCTCGTTGCTCTTTTTAGAGGTGTCCAGTGTCAGGTGCAGCTGCAGGAGTCG
GGCCCAGGACTGGTGAAGCCTTCGGAGACCCTGTCCCTCACCTGCACTGTCTCTGGTGGCTCCGTCAGCAGTGGTGAT
TACTACTGGACCTGGATCCGGCAGTCCCCAGGGAAGGGACTGGAGTGGATTGGACACATCTATTACAGTGGGAACACC
AATTATAACCCCTCCCTCAAGAGTCGACTCACCATATCAATTGACACGTCCAAGACTCAGTTCTCCCTGAAGCTGAGT
TCTGTGACCGCTGCGGACACGGCCATTTATTACTGTGTGCGAGATCGAGTGACTGGTGCTTTTGATATCTGGGGCCAA
GGGACAATGGTCACCGTCTCTTCAGCCTCCACCAAGGGCCCATCGGTCTTCCCCCTGGCGCCCTGCTCCAGGAGCACC
TCCGAGAGCACAGCGGCCCTGGGCTGCCTGGTCAAGGACTACTTCCCTGAACCGGTGACGGTGTCGTGGAACTCAGGC
GCTCTGACCAGCGGCGTGCACACCTTCCCGGCTGTCCTACAGTCCTCAGGACTCTACTCCCTCAGCAGCGTGGTGACC
GTGCCCTCCAGCAACTTCGGCACCCAGACCTACACCTGCAACGTAGATCACAAGCCCAGCAATACCAAGGTGGACAAG
ACAGTTGAGCGCAAATGTTGTGTCGAGTGCCCACCGTGCCCAGCACCACCTGTGGCAGGACCGTCAGTCTTCCTCTTC
CCCCCAAAACCCAAGGACACCCTCATGATCTCCCGGACCCCTGAGGTCACGTGCGTGGTGGTGGACGTGAGCCACGAA
GACCCCGAGGTCCAGTTCAACTGGTACGTGGACGGCGTGGAGGTGCATAATGCCAAGACAAAGCCACGGGAGGAGCAG
TTCCAAAGCACGTTCCGTGTGGTCAGCGTCCTCACCGTCGTGCACCAGGACTGGCTGAACGGCAAGGAGTACAAGTGC
AAGGTCTCCAACAAAGGCCTCCCAGCCCCCATCGAGAAAACCATCTCCAAAACCAAAGGGCAGCCCCGAGAACCACAG
GTGTACACCCTGCCCCCATCCCGGGAGGAGATGACCAAGAACCAGGTCAGCCTGACCTGCCTGGTCAAAGGCTTCTAC
CCCAGCGACATCGCCGTGGAGTGGGAGAGCAATGGGCAGCCGGAGAACAACTACAAGACCACGCCTCCCATGCTGGAC
TCCGACGGCTCCTTCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTGGTGGAGGTGGGTCT
GGAGGTGGAGGATCTGGTGGAGGTGGGTCTCCCGAGGAACCTCTAGTGGTGAAGGTGGAAGAGGGAGATAACGCTGTG
CTGCAGTGCCTCAAGGGGACCTCAGATGGCCCCACTCAGCAGCTGACCTGGTCTCGGGAGTCCCCGCTTAAACCCTTC
TTAAAACTCAGCCTGGGGCTGCCAGGCCTGGGAATCCACATGAGGCCCCTGGCCATCTGGCTTTTCATCTTCAACGTC
TCTCAACAGATGGGGGGCTTCTACCTGTGCCAGCCGGGGCCCCCCTCTGAGAAGGCCTGGCAGCCTGGCTGGACAGTC
AATGTGGAGGGCAGCGGGGAGCTGTTCCGGTGGAATGTTTCGGACCTAGGTGGCCTGGGCTGTGGCCTGAAGAACAGG
TCCTCAGAGGGCCCCAGCTCCCCTTCCGGGAAGCTCATGAGCCCCAAGCTGTATGTGTGGGCCAAAGACCGCCCTGAG
ATCTGGGAGGGAGAGCCTCCGTGTCTCCCACCGAGGGACAGCCTGAACCAGAGCCTCAGCCAGGACCTCACCATGGCC
CCTGGCTCCACACTCTGGCTGTCCTGTGGGGTACCCCCTGACTCTGTGTCCAGGGGCCCCCTCTCCTGGACCCATGTG
CACCCCAAGGGGCCTAAGTCATTGCTGAGCCTAGAGCTGAAGGACGATCGCCCGGCCAGAGATATGTGGGTAATGGAG
ACGGGTCTGTTGTTGCCCCGGGCCACAGCTCAAGACGCTGGAAAGTATTATTGTCACCGTGGCAACCTGACCATGTCA
TTCCACCTGGAGATCACTGCTCGGCCA
SEQID NO. 236
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGACATCCAGATGACCCAGTCTCCATCCTCCCTGTCTGCATCTGTAGGAGACAGAGTC
ACCATCACTTGCAGTGTCAGCTCAAGTGTATCCTCCATTTACTTGCACTGGTATCAGCAGAAACCAGGGAAAGCCCCT
AAGCTCCTGATCTATAGCACATCCAACTTGGCTTCTGGAGTCCCATCAAGGTTCAGTGGCAGTGGATCTGGGACAGAT
TTCACTCTCACCATCAGCAGTCTGCAACCTGAAGATTTTGCAACTTACTACTGTCAAGTCTACAGTGGTTACCCGCTC
ACGTTCGGCGGAGGGACCAAGGTGGAGATCAAACGAACTGTGGCTGCACCATCTGTCTTCATCTTCCCGCCATCTGAT
GAGCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGCT
SEQID NO. 237
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTCAGGTTCAGCTGGTGCAGTCTGGTGCTGAGGTGAAGAAGCCTGGTGCCTCAGTGAAG
GTCTCCTGCAAGGCTTCTGGTTACACATTCACTGACTACTACATGCACTGGGTGCGTCAGGCCCCTGGTCAAGGTCTT
GAGTGGATGGGTCGTGTTAATCCTAACCGGAGGGGTACTACCTACAACCAGAAATTCGAGGGCCGTGTCACCATGACC
ACAGACACATCCACGAGCACAGCCTACATGGAGCTGCGTAGCCTGCGTTCTGACGACACGGCCGTGTATTACTGTGCG
CGTGCGAACTGGCTTGACTACTGGGGCCAGGGCACCACCGTCACCGTCTCCTCCGCCTCCACCAAGGGCCCATCGGTC
TTCCCGCTAGCGCCCTGCTCCAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACGAAGACCTACACCTGCAACGTAGAT
CACAAGCCCAGCAACACCAAGGTGGACAAGAGAGTTGAGTCCAAATATGGTCCCCCATGCCCACCCTGCCCAGCACCT
GAGGCCGCCGGGGGACCATCAGTCTTCCTGTTCCCCCCAAAACCCAAGGACACTCTCATGATCTCCCGGACCCCTGAG
GTCACGTGCGTGGTGGTGGACGTGAGCCAGGAAGACCCCGAGGTCCAGTTCAACTGGTACGTGGATGGCGTGGAGGTG
CATAATGCCAAGACAAAGCCGCGGGAGGAGCAGTTCAACAGCACGTACCGTGTGGTCAGCGTCCTCACCGTCCTGCAC
CAGGACTGGCTGAACGGCAAGGAGTACAAGTGCAAGGTCTCCAACAAAGGCCTCCCGTCCTCCATCGAGAAAACCATC
TCCAAAGCCAAAGGGCAGCCCCGAGAGCCACAGGTGTACACCCTGCCCCCATCCCAGGAGGAGATGACCAAGAACCAG
GTCAGCCTGACCTGCCTGGTCAAAGGCTTCTACCCCAGCGACATCGCCGTGGAGTGGGAAAGCAATGGGCAGCCGGAG
AACAACTACAAGACCACGCCTCCCGTGCTGGACTCCGACGGCTCCTTCTTCCTCTACAGCAGGCTAACCGTGGACAAG
AGCAGGTGGCAGGAGGGGAATGTCTTCTCATGCTCCGTGATGCATGAGG
CTCTGCACAACCACTACACACAGAAGAGCCTCTCCCTGTCTCTG
SEQID NO. 238
ATGGAGACAGACACACTCCTGCTATGGGTACTGCTGCTCTGGGTTCCAGGTTCCACTGGTGACATCCAGATGACCCAG
TCTCCATCCTCCCTGTCTGCATCTGTAGGAGACAGAGTCACCATCACTTGCAGTGTCAGCTCAAGTGTATCCTCCATT
TACTTGCACTGGTATCAGCAGAAACCAGGGAAAGCCCCTAAGCTCCTGATCTATAGCACATCCAACTTGGCTTCTGGA
GTCCCATCAAGGTTCAGTGGCAGTGGATCTGGGACAGATTTCACTCTCACCATCAGCAGTCTGCAACCTGAAGATTTT
GCAACTTACTACTGTCAAGTCTACAGTGGTTACCCGCTCACGTTCGGCGGAGGGACCAAGGTGGAGATCAAACGAACT
GTGGCTGCACCATCTGTCTTCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCTTCAACAGGGGAGAG
TGCGACAAAACTCACACATCGCCACCGTCCCCAGCACCTGAAGCCGCGGGGGGACCGTCACCCGAGGAACCTCTAGTG
GTGAAGGTGGAAGAGGGAGATAACGCTGTGCTGCAGTGCCTCAAGGGGACCTCAGATGGCCCCACTCAGCAGCTGACC
TGGTCTCGGGAGTCCCCGCTTAAACCCTTCTTAAAACTCAGCCTGGGGCTGCCAGGCCTGGGAATCCACATGAGGCCC
CTGGCCATCTGGCTTTTCATCTTCAACGTCTCTCAACAGATGGGGGGCTTCTACCTGTGCCAGCCGGGGCCCCCCTCT
GAGAAGGCCTGGCAGCCTGGCTGGACAGTCAATGTGGAGGGCAGCGGGGAGCTGTTCCGGTGGAATGTTTCGGACCTA
GGTGGCCTGGGCTGTGGCCTGAAGAACAGGTCCTCAGAGGGCCCCAGCTCCCCTTCCGGGAAGCTCATGAGCCCCAAG
CTGTATGTGTGGGCCAAAGACCGCCCTGAGATCTGGGAGGGAGAGCCTCCGTGTCTCCCACCGAGGGACAGCCTGAAC
CAGAGCCTCAGCCAGGACCTCACCATGGCCCCTGGCTCCACACTCTGGCTGTCCTGTGGGGTACCCCCTGACTCTGTG
TCCAGGGGCCCCCTCTCCTGGACCCATGTGCACCCCAAGGGGCCTAAGTCATTGCTGAGCCTAGAGCTGAAGGACGAT
CGCCCGGCCAGAGATATGTGGGTAATGGAGACGGGTCTGTTGTTGCCCCGGGCCACAGCTCAAGACGCTGGAAAGTAT
TATTGTCACCGTGGCAACCTGACCATGTCATTCCACCTGGAGATCACTGCTCGGCCA
SEQID NO. 239
ATGGAGTTTGGGCTGAGCTGGGTTTTCCTCGTTGCTCTTTTTAGAGGTGTCCAGTGTCAGGTTCAGCTGGTGCAGTCT
GGTGCTGAGGTGAAGAAGCCTGGTGCCTCAGTGAAGGTCTCCTGCAAGGCTTCTGGTTACACATTCACTGACTACTAC
ATGCACTGGGTGCGTCAGGCCCCTGGTCAAGGTCTTGAGTGGATGGGTCGTGTTAATCCTAACCGGAGGGGTACTACC
TACAACCAGAAATTCGAGGGCCGTGTCACCATGACCACAGACACATCCACGAGCACAGCCTACATGGAGCTGCGTAGC
CTGCGTTCTGACGACACGGCCGTGTATTACTGTGCGCGTGCGAACTGGCTTGACTACTGGGGCCAGGGCACCACCGTC
ACCGTCTCCTCCGCCTCCACCAAGGGCCCATCGGTCTTCCCGCTAGCGCCCTGCTCCAGGAGCACCTCCGAGAGCACA
GCCGCCCTGGGCTGCCTGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGTGACCGTGCCCTCCAGC
AGCTTGGGCACGAAGACCTACACCTGCAACGTAGATCACAAGCCCAGCAACACCAAGGTGGACAAGAGAGTTGAGTCC
AAATATGGTCCCCCATGCCCACCCTGCCCAGCACCTGAGGCCGCCGGGGGACCATCAGTCTTCCTGTTCCCCCCAAAA
CCCAAGGACACTCTCATGATCTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGCGGGAGGAGCAGTTCAACAGC
ACGTACCGTGTGGTCAGCGTCCTCACCGTCCTGCACCAGGACTGGCTGAACGGCAAGGAGTACAAGTGCAAGGTCTCC
AACAAAGGCCTCCCGTCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGCTTCTACCCCAGCGAC
ATCGCCGTGGAGTGGGAAAGCAATGGGCAGCCGGAGAACAACTACAAGACCACGCCTCCCGTGCTGGACTCCGACGGC
TCCTTCTTCCTCTACAGCAGGCTAACCGTGGACAAGAGCAGGTGGCAGGAGGGGAATGTCTTCTCATGCTCCGTGATG
CATGAGGCTCTGCACAACCACTACACACAGAAGAGCCTCTCCCTGTCTCTGGGTGGTGGAGGTGGGTCTGGAGGTGGA
GGATCTGGTGGAGGTGGGTCTCCCGAGGAACCTCTAGTGGTGAAGGTGGAAGAGGGAGATAACGCTGTGCTGCAGTGC
CTCAAGGGGACCTCAGATGGCCCCACTCAGCAGCTGACCTGGTCTCGGGAGTCCCCGCTTAAACCCTTCTTAAAACTC
AGCCTGGGGCTGCCAGGCCTGGGAATCCACATGAGGCCCCTGGCCATCTGGCTTTTCATCTTCAACGTCTCTCAACAG
ATGGGGGGCTTCTACCTGTGCCAGCCGGGGCCCCCCTCTGAGAAGGCCTGGCAGCCTGGCTGGACAGTCAATGTGGAG
GGCAGCGGGGAGCTGTTCCGGTGGAATGTTTCGGACCTAGGTGGCCTGGGCTGTGGCCTGAAGAACAGGTCCTCAGAG
GGCCCCAGCTCCCCTTCCGGGAAGCTCATGAGCCCCAAGCTGTATGTGTGGGCCAAAGACCGCCCTGAGATCTGGGAG
GGAGAGCCTCCGTGTCTCCCACCGAGGGACAGCCTGAACCAGAGCCTCAGCCAGGACCTCACCATGGCCCCTGGCTCC
ACACTCTGGCTGTCCTGTGGGGTACCCCCTGACTCTGTGTCCAGGGGCCCCCTCTCCTGGACCCATGTGCACCCCAAG
GGGCCTAAGTCATTGCTGAGCCTAGAGCTGAAGGACGATCGCCCGGCCAGAGATATGTGGGTAATGGAGACGGGTCTG
TTGTTGCCCCGGGCCACAGCTCAAGACGCTGGAAAGTATTATTGTCACCGTGGCAACCTGACCATGTCATTCCACCTG
GAGATCACTGCTCGGCCA
SEQID NO. 240
ATGGAGTTTGGGCTGAGCTGGGTTTTCCTCGTTGCTCTTTTTAGAGGTGTCCAGTGTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGGACAAAACTCACACATCGCCACCGTCCCCAGCACCTGAAGCCGCGGGGGGACCGTCACCC
GAGGAACCTCTAGTGGTGAAGGTGGAAGAGGGAGATAACGCTGTGCTGCAGTGCCTCAAGGGGACCTCAGATGGCCCC
ACTCAGCAGCTGACCTGGTCTCGGGAGTCCCCGCTTAAACCCTTCTTAAAACTCAGCCTGGGGCTGCCAGGCCTGGGA
ATCCACATGAGGCCCCTGGCCATCTGGCTTTTCATCTTCAACGTCTCTCAACAGATGGGGGGCTTCTACCTGTGCCAG
CCGGGGCCCCCCTCTGAGAAGGCCTGGCAGCCTGGCTGGACAGTCAATGTGGAGGGCAGCGGGGAGCTGTTCCGGTGG
AATGTTTCGGACCTAGGTGGCCTGGGCTGTGGCCTGAAGAACAGGTCCTCAGAGGGCCCCAGCTCCCCTTCCGGGAAG
CTCATGAGCCCCAAGCTGTATGTGTGGGCCAAAGACCGCCCTGAGATCTGGGAGGGAGAGCCTCCGTGTCTCCCACCG
AGGGACAGCCTGAACCAGAGCCTCAGCCAGGACCTCACCATGGCCCCTGGCTCCACACTCTGGCTGTCCTGTGGGGTA
CCCCCTGACTCTGTGTCCAGGGGCCCCCTCTCCTGGACCCATGTGCACCCCAAGGGGCCTAAGTCATTGCTGAGCCTA
GAGCTGAAGGACGATCGCCCGGCCAGAGATATGTGGGTAATGGAGACGGGTCTGTTGTTGCCCCGGGCCACAGCTCAA
GACGCTGGAAAGTATTATTGTCACCGTGGCAACCTGACCATGTCATTCCACCTGGAGATCACTGCTCGGCCATCTAGA
GGGCCCCATCATCACCATCACCAT
SEQID NO. 241
ATGGAGTTTGGGCTGAGCTGGGTTTTCCTCGTTGCTCTTTTTAGAGGTGTCCAGTGTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGGACAAAACTCACACATCGCCACCGTCCCCAGCACCTGAAGCCGCGGGGGGACCGTCACCC
GAGGAACCTCTAGTGGTGAAGGTGGAAGAGGGAGATAACGCTGTGCTGCAGTGCCTCAAGGGGACCTCAGATGGCCCC
ACTCAGCAGCTGACCTGGTCTCGGGAGTCCCCGCTTAAACCCTTCTTAAAACTCAGCCTGGGGCTGCCAGGCCTGGGA
ATCCACATGAGGCCCCTGGCCATCTGGCTTTTCATCTTCAACGTCTCTCAACAGATGGGGGGCTTCTACCTGTGCCAG
CCGGGGCCCCCCTCTGAGAAGGCCTGGCAGCCTGGCTGGACAGTCAATGTGGAGGGCAGCGGGGAGCTGTTCCGGTGG
AATGTTTCGGACCTAGGTGGCCTGGGCTGTGGCCTGAAGAACAGGTCCTCAGAGGGCCCCAGCTCCCCTTCCGGGAAG
CTCATGAGCCCCAAGCTGTATGTGTGGGCCAAAGACCGCCCTGAGATCTGGGAGGGAGAGCCTCCGTGTCTCCCACCG
AGGGACAGCCTGAACCAGAGCCTCAGCCAGGACCTCACCATGGCCCCTGGCTCCACACTCTGGCTGTCCTGTGGGGTA
CCCCCTGACTCTGTGTCCAGGGGCCCCCTCTCCTGGACCCATGTGCACCCCAAGGGGCCTAAGTCATTGCTGAGCCTA
GAGCTGAAGGACGATCGCCCGGCCAGAGATATGTGGGTAATGGAGACGGGTCTGTTGTTGCCCCGGGCCACAGCTCAA
GACGCTGGAAAGTATTATTGTCACCGTGGCAACCTGACCATGTCATTCCACCTGGAGATCACTGCTCGGCCAGACAAA
ACTCACACATGCCCACCGTGCCCAGCACCTGAACTCCTGGGGGGACCGTCAGTCTTCCTCTTCCCCCCAAAACCCAAG
GACACCCTCATGATCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCC
CCATCCCGGGAGGAGATGACCAAGAACCAGGTCAGCCTGACCTGCCTGGTCAAAGGCTTCTATCCCAGCGACATCGCC
GTGGAGTGGGAGAGCAATGGGCAGCCGGAGAACAACTACAAGACCACGCCTCCCGTGCTGGACTCCGACGGCTCCTTC
TTCCTCTACAGCAAGCTCACCGTGGACAAGAGCAGGTGGCAGCAGGGGAACGTCTTCTCATGCTCCGTGATGCACGAG
GCTCTGCACAACCACTACACGCAGAAGAGCCTCTCCCTGTCTCCGGGT
SEQID NO. 242
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGGGGAGGTGGGTCTGGAGGTGGA
GGATCTGGTGGAGGTGGGTCTGGTGGAGGTGGGTCTGAGGTGCAGCTGGTGGAGTCTGGTGGTGGTCTTGTTCAACCT
GGTGGTTCTCTTCGTCTTTCTTGTGCTGCTTCTGGTTTTAATATTAAAGATACTTATATTCATTGGGTTCGTCAAGCT
CCTGGTAAAGGTCTTGAATGGGTTGCTCGTATTTATCCTACTAATGGTTATACTCGTTATGCTGATTCTGTTAAAGGT
CGTTTTACTATTTCTGCTGATACTTCTAAAAATACTGCTTATCTTCAAATGAACTCTCTTCGTGCTGAAGATACTGCT
GTTTATTATTGTTCTCGTTGGGGTGGTGATGGTTTTTATGCTATGGATTATTGGGGTCAAGGTACTCTTGTCACCGTC
TCCTCAGCTAGCACCGGGGGAGGTGGGTCTGGAGGTGGAGGATCTGGTGGAGGTGGGTCTGACATCCAGATGACCCAG
TCTCCTTCTTCTCTTTCTGCTTCTGTTGGTGATCGTGTTACTATTACTTGTCGTGCTTCTCAAGATGTTAATACTGCT
GTTGCTTGGTATCAACAAAAACCTGGTAAAGCTCCTAAACTTCTTATTTATTCTGCTTCTTTTCTTTATTCTGGTGTT
CCTTCTCGTTTTTCTGGTTCTCGTTCTGGTACTGATTTTACTCTTACTATTTCTTCTCTTCAACCTGAAGATTTTGCT
ACTTATTATTGTCAACAACATTATACTACTCCTCCTACTTTTGGTCAAGGTACCAAGGTGGAGATCAAACGTACGTCT
AGAGGGCCCCATCATCACCATCACCAT
SEQID NO. 243
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGGGGAGGTGGGTCTGGAGGTGGA
GGATCTGGTGGAGGTGGGTCTGGTGGAGGTGGGTCTGAGGTGCAGCTGGTGGAGTCTGGTGGTGGTCTTGTTCAACCT
GGTGGTTCTCTTCGTCTTTCTTGTGCTGCTTCTGGTTTTAATATTAAAGATACTTATATTCATTGGGTTCGTCAAGCT
CCTGGTAAAGGTCTTGAATGGGTTGCTCGTATTTATCCTACTAATGGTTATACTCGTTATGCTGATTCTGTTAAAGGT
CGTTTTACTATTTCTGCTGATACTTCTAAAAATACTGCTTATCTTCAAATGAACTCTCTTCGTGCTGAAGATACTGCT
GTTTATTATTGTTCTCGTTGGGGTGGTGATGGTTTTTATGCTATGGATTATTGGGGTCAAGGTACTCTTGTCACCGTC
TCCTCAGCTAGCACCGGGGGAGGTGGGTCTGGAGGTGGAGGATCTGGTGGAGGTGGGTCTGACATCCAGATGACCCAG
TCTCCTTCTTCTCTTTCTGCTTCTGTTGGTGATCGTGTTACTATTACTTGTCGTGCTTCTCAAGATGTTAATACTGCT
GTTGCTTGGTATCAACAAAAACCTGGTAAAGCTCCTAAACTTCTTATTTATTCTGCTTCTTTTCTTTATTCTGGTGTT
CCTTCTCGTTTTTCTGGTTCTCGTTCTGGTACTGATTTTACTCTTACTATTTCTTCTCTTCAACCTGAAGATTTTGCT
ACTTATTATTGTCAACAACATTATACTACTCCTCCTACTTTTGGTCAAGGTACCAAGGTGGAGATCAAACGTACGGAC
AAAACTCACACATGCCCACCGTGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGTGCAAGGTCTCCAAC
AAAGCCCTCCCAGCCCCCATCGAGAAAACCATCTCCAAAGCCAAAGGGCAGCCCCGAGAACCACAGGTGTACACCCTG
CCCCCATCCCGGGAGGAGATGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATGCTCCGTGATGCAC
GAGGCTCTGCACAACCACTACACGCAGAAGAGCCTCTCCCTGTCTCCGGGT
SEQID NO. 246
AAGCTTAATTTAAGGCAGGATGTCTCAGAGTCTGGGAAAATCCCACTTTCCTCCTGCTACACCTTACAGTTGTGAGAA
AGCACATTTCAGACAACAGGGAAAACCCATACTTCACCACAACAACACACTATACATTGTCTGGTCCACTGGAGCATA
AATTAAAGAGAAACAATGTAGTCAAGCAAGTAGGCGGCAAGAGGAAGGGGGCGGAGACATCATCAGGGAGTATAAACT
CTGAGATGCCTCAGAGCCTCACAGACTCAACAAGAGCTCCAGCAAAGACTTTCACTGTAGCTTGACTTGACCTGAGAT
TAACTAGGGAATCTTGAGAATAAAGAAGCTTAACTAGTTAGCGGACCGACGCGTACGCGGCCGCTCGAGATGGAGAGC
GACGAGAGCGGCCTGCCCGCCATGGAGATCGAGTGCCGCATCACCGGCACCCTGAACGGCGTGGAGTTCGAGCTGGTG
GGCGGCGGAGAGGGCACCCCCGAGCAGGGCCGCATGACCAACAAGATGAAGAGCACCAAAGGCGCCCTGACCTTCAGC
CCCTACCTGCTGAGCCACGTGATGGGCTACGGCTTCTACCACTTCGGCACCTACCCCAGCGGCTACGAGAACCCCTTC
CTGCACGCCATCAACAACGGCGGCTACACCAACACCCGCATCGAGAAGTACGAGGACGGCGGCGTGCTGCACGTGAGC
TTCAGCTACCGCTACGAGGCCGGCCGCGTGATCGGCGACTTCAAGGTGATGGGCACCGGCTTCCCCGAGGACAGCGTG
ATCTTCACCGACAAGATCATCCGCAGCAACGCCACCGTGGAGCACCTGCACCCCATGGGCGATAACGATCTGGATGGC
AGCTTCACCCGCACCTTCAGCCTGCGCGACGGCGGCTACTACAGCTCCGTGGTGGACAGCCACATGCACTTCAAGAGC
GCCATCCACCCCAGCATCCTGCAGAACGGGGGCCCCATGTTCGCCTTCCGCCGCGTGGAGGAGGATCACAGCAACACC
GAGCTGGGCATCGTGGAGTACCAGCACGCCTTCAAGACCCCGGATGCAGATGCCGGTGAAGAAAGAGTT
SEQID NO. 247
AAGCTTTAACGAAGACAGGGCCATGTAGAGGGCCCCAGGGAGTGAAAGGGCCTCCAGGACCTCCAGGTATGGAATACA
GGGGACGTTTAAGAAGATATGGCCACACACTGGGGCCCTGAGAAGTGAGAGCTTCATGAAAAAAATCAGGGACCCCAG
AGTTCCTTGGAAGCCAAGACTGAAACCAGCATTATGAGTCTCCGGGTCAGAATGAAAGAAGAAGGCCTGCCCCAGTGG
GGTCTGTGAATTCCCGGGGGTGATTTCACTCCCCGGGGCTGTCCCAGGCTTGTCCCTGCTACCCCCACCCAGCCTTTC
CTGAGGCCTCAAGCCTGCCACCAAGCCCCCAGCTCCTTCTCCCCGCAGGGACCCAAACACAGGCCTCGGGACTCAACA
CAGCTTTTCCCTCCAACCCCGTTTTCTCTCCCTCAAGGACTCAGCTTTCTGAGGCCCCTCCCAGTTCTAGTTCTATCT
TTTTCCTGCATCCTGTCTGGAAGTTAGAAGGAAACAGACCACAGACCTGGTCCCCAAAAGAAATGGAGGCAATAGGTT
TTGAGGGGCATGGGGACGGGGTTCAGCCTCCAGGGTCCTACACACAAATCAGTCAGTGGCCCAGAAGACCCCCTCGGA
ATCGGAGCAGGGAGGATGGGGAGTGTGAGGGGTATCCTTGATGCTTGTGTGTCCCCAACTTTCCAAATCCCCGCCCCC
GCGATGGAGAAGAAACCGAGACAGAAGGTGCAGGGCCCACTACCGCTTCCTCCAGATGAGCTCATGGGTTTCTCCACC
AAGGAAGTTTTCCGCTGGTTGAATGATTCTTTCCCCGCCCTCCTCTCGCCCCAGGGACATATAAAGGCAGTTGTTGGC
ACACCCAGCCAGCAGACGCTCCCTCAGCAAGGACAGCAGAGGACCAGCTAAGAGGGAGAGAAGCAACTACAGACCCCC
CCTGAAAACAACCCTCAGACGCCACATCCCCTGACAAGCTGCCAGGCAGGTTCTCTTCCTCTCACATACTGACCCACG
GCTCCACCCTCTCTCCCCTGGAAAGGACACAAGCTTAACTAGTTAGCGGACCGACGCGTACGCGGCCGCTCGAGATGG
AGAGCGACGAGAGCGGCCTGCCCGCCATGGAGATCGAGTGCCGCATCACCGGCACCCTGAACGGCGTGGAGTTCGAGC
TGGTGGGCGGCGGAGAGGGCACCCCCGAGCAGGGCCGCATGACCAACAAGATGAAGAGCACCAAAGGCGCCCTGACCT
TCAGCCCCTACCTGCTGAGCCACGTGATGGGCTACGGCTTCTACCACTTCGGCACCTACCCCAGCGGCTACGAGAACC
CCTTCCTGCACGCCATCAACAACGGCGGCTACACCAACACCCGCATCGAGAAGTACGAGGACGGCGGCGTGCTGCACG
TGAGCTTCAGCTACCGCTACGAGGCCGGCCGCGTGATCGGCGACTTCAAGGTGATGGGCACCGGCTTCCCCGAGGACA
GCGTGATCTTCACCGACAAGATCATCCGCAGCAACGCCACCGTGGAGCACCTGCACCCCATGGGCGATAACGATCTGG
ATGGCAGCTTCACCCGCACCTTCAGCCTGCGCGACGGCGGCTACTACAGCTCCGTGGTGGACAGCCACATGCACTTCA
AGAGCGCCATCCACCCCAGCATCCTGCAGAACGGGGGCCCCATGTTCGCCTTCCGCCGCGTGGAGGAGGATCACAGCA
ACACCGAGCTGGGCATCGTGGAGTACCAGCACGCCTTCAAGACCCCGGATGCAGATGCCGGTGAAGAAAGAGTT
SEQID NO. 248
AAGCTTGGGAGAAGCTAGACTTAAAATCTTCCATTGCAGCTGTAAACACATCTGGACAATAGTCTGTTTTCTGCATTT
GTGAATCCCACACCCATGGAACTATGAATCGTGCATCAGAGTTATTTAAAACCACCGTGCATGGAGTGAACCAATACC
GAGGTGTTTGCTTATCATTTTCCTTTGAGCACACAGCACAGCCTTGAACTCAGTGACACTCCTAAGAGGGCTCTAGGG
TCAGGCCAACTTAGATGAGATGCTAGTCTTTAGCTAAAGATGCCCTTCCACCCCCGTTGCACGACCTTGCTTCTCAGT
CTTTGTTGAGTCTTCTGGGGGAGAATCCCCCTAGAGGACTCAGTTTACAAAACCCTAAGTGAGACCACTGCCAAGAAG
TGCTTGCTCACCCCTCCTGCCGCGGCAGGGAATCCCCCTTTCCTTGTACAGGCAAAACACAAAAAAGGACTCATAAGT
GAAGCCTGATCCTTCTCACCAAACACTGCCCACACCTCCTAGTAATTGAACTTGAAAAAAAAAACTGGTTTGAAAAAT
TACCGCAAACCATATTGTCATAAAAAAAAAAAAAAACACTTCCTATATGAGATCACAGAACAGAGTAGGCACAAGTTC
CTGCTGAGCAGATCAGCCTAATGCTTAAATAGAACAACTCCTGGCTGTCATTGACATTGTCTAAAAGCCAAGATGACA
GACTGAGAGGCCTGAGCCCTTGTTCTGGCATTCTCCCAGGAAGATGCAGTAAAGGGGTTGACCCAATATACAAGCTTA
ACTAGTTAGCGGACCGACGCGTACGCGGCCGCTCGAGATGGAGAGCGACGAGAGCGGCCTGCCCGCCATGGAGATCGA
GTGCCGCATCACCGGCACCCTGAACGGCGTGGAGTTCGAGCTGGTGGGCGGCGGAGAGGGCACCCCCGAGCAGGGCCG
CATGACCAACAAGATGAAGAGCACCAAAGGCGCCCTGACCTTCAGCCCCTACCTGCTGAGCCACGTGATGGGCTACGG
CTTCTACCACTTCGGCACCTACCCCAGCGGCTACGAGAACCCCTTCCTGCACGCCATCAACAACGGCGGCTACACCAA
CACCCGCATCGAGAAGTACGAGGACGGCGGCGTGCTGCACGTGAGCTTCAGCTACCGCTACGAGGCCGGCCGCGTGAT
CGGCGACTTCAAGGTGATGGGCACCGGCTTCCCCGAGGACAGCGTGATCTTCACCGACAAGATCATCCGCAGCAACGC
CACCGTGGAGCACCTGCACCCCATGGGCGATAACGATCTGGATGGCAGCTTCACCCGCACCTTCAGCCTGCGCGACGG
CGGCTACTACAGCTCCGTGGTGGACAGCCACATGCACTTCAAGAGCGCCATCCACCCCAGCATCCTGCAGAACGGGGG
CCCCATGTTCGCCTTCCGCCGCGTGGAGGAGGATCACAGCAACACCGAGCTGGGCATCGTGGAGTACCAGCACGCCTT
CAAGACCCCGGATGCAGATGCCGGTGAAGAAAGAGTT
SEQID NO. 249
AAGCTTGATATCGAATTAGGAGGAAAACTGTTTCATACAGAAGGCGTCAATTAGGAGGAAAAACTGTTTCATACAGAA
GGCGTCAATTAGGAGGAAAAACTGTTTCATACAGAAGGCGTCAATTGGTCCCATCGAATTAGGAGGAAAAACTGTTTC
ATACAGAAGGCGTCAATTAGGAGGAAAAACTGTTTCATACAGAAGGCGTCAATTAGGAGGAAAAACTGTTTCATACAG
AAGGCGTCAATTGGTCCCGGGACATTTTGACACCCCCATAATATTTTTCCAGAATTAACAGTATAAATTGCATCTCTT
GTTCAAGAGTTCCCTATCACTCTCTTTAATCACTACTCACAGTAACCTCAACTCCTGAAGCTTAACTAGTTAGCGGAC
CGACGCGTACGCGGCCGCTCGAGATGGAGAGCGACGAGAGCGGCCTGCCCGCCATGGAGATCGAGTGCCGCATCACCG
GCACCCTGAACGGCGTGGAGTTCGAGCTGGTGGGCGGCGGAGAGGGCACCCCCGAGCAGGGCCGCATGACCAACAAGA
TGAAGAGCACCAAAGGCGCCCTGACCTTCAGCCCCTACCTGCTGAGCCACGTGATGGGCTACGGCTTCTACCACTTCG
GCACCTACCCCAGCGGCTACGAGAACCCCTTCCTGCACGCCATCAACAACGGCGGCTACACCAACACCCGCATCGAGA
AGTACGAGGACGGCGGCGTGCTGCACGTGAGCTTCAGCTACCGCTACGAGGCCGGCCGCGTGATCGGCGACTTCAAGG
TGATGGGCACCGGCTTCCCCGAGGACAGCGTGATCTTCACCGACAAGATCATCCGCAGCAACGCCACCGTGGAGCACC
TGCACCCCATGGGCGATAACGATCTGGATGGCAGCTTCACCCGCACCTTCAGCCTGCGCGACGGCGGCTACTACAGCT
CCGTGGTGGACAGCCACATGCACTTCAAGAGCGCCATCCACCCCAGCATCCTGCAGAACGGGGGCCCCATGTTCGCCT
TCCGCCGCGTGGAGGAGGATCACAGCAACACCGAGCTGGGCATCGTGGAGTACCAGCACGCCTTCAAGACCCCGGATG
CAGATGCCGGTGAAGAAAGAGTT
SEQID NO. 250
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGGAGGAGGTGGGTCTGGAGGTGGAGGATCTGGTGGAGGTGGGTCTGGAGGAGGTGGGTCTCAG
GTGCAGCTGCAGGAGTCGGGCCCAGGACTGGTGAAGCCTTCGGAGACCCTGTCCCTCACCTGCACTGTCTCTGGTGGC
TCCGTCAGCAGTGGTGATTACTACTGGACCTGGATCCGGCAGTCCCCAGGGAAGGGACTGGAGTGGATTGGACACATC
TATTACAGTGGGAACACCAATTATAACCCCTCCCTCAAGAGTCGACTCACCATATCAATTGACACGTCCAAGACTCAG
TTCTCCCTGAAGCTGAGTTCTGTGACCGCTGCGGACACGGCCATTTATTACTGTGTGCGAGATCGAGTGACTGGTGCT
TTTGATATCTGGGGCCAAGGGACAATGGTCACCGTCTCTTCAGCCTCCACCAAGGGCCCATCGGTCTTCCCCCTGGCG
CCCTGCTCCAGGAGCACCTCCGAGAGCACAGCGGCCCTGGGCTGCCTGGTCAAGGACTACTTCCCTGAACCGGTGACG
GTGTCGTGGAACTCAGGCGCTCTGACCAGCGGCGTGCACACCTTCCCGGCTGTCCTACAGTCCTCAGGACTCTACTCC
CTCAGCAGCGTGGTGACCGTGCCCTCCAGCAACTTCGGCACCCAGACCTACACCTGCAACGTAGATCACAAGCCCAGC
AATACCAAGGTGGACAAGACAGTTGAGCGCAAATGTTGTGTCGAGTGCCCACCGTGCCCAGCACCACCTGTGGCAGGA
CCGTCAGTCTTCCTCTTCCCCCCAAAACCCAAGGACACCCTCATGATCTCCCGGACCCCTGAGGTCACGTGCGTGGTG
GTGGACGTGAGCCACGAAGACCCCGAGGTCCAGTTCAACTGGTACGTGGACGGCGTGGAGGTGCATAATGCCAAGACA
AAGCCACGGGAGGAGCAGTTCCAAAGCACGTTCCGTGTGGTCAGCGTCCTCACCGTCGTGCACCAGGACTGGCTGAAC
GGCAAGGAGTACAAGTGCAAGGTCTCCAACAAAGGCCTCCCAGCCCCCATCGAGAAAACCATCTCCAAAACCAAAGGG
CAGCCCCGAGAACCACAGGTGTACACCCTGCCCCCATCCCGGGAGGAGATGACCAAGAACCAGGTCAGCCTGACCTGC
CTGGTCAAAGGCTTCTACCCCAGCGACATCGCCGTGGAGTGGGAGAGCAATGGGCAGCCGGAGAACAACTACAAGACC
ACGCCTCCCATGCTGGACTCCGACGGCTCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCCTGTCTCCG
GGT
SEQID NO. 251
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGGAGGAGGTGGGTCTGGAGGAGGTGGGTCTGGAGGTGGAGGATCTGGTGGAGGTGGGTCTCAG
GTTCAGCTGGTGCAGTCTGGTGCTGAGGTGAAGAAGCCTGGTGCCTCAGTGAAGGTCTCCTGCAAGGCTTCTGGTTAC
ACATTCACTGACTACTACATGCACTGGGTGCGTCAGGCCCCTGGTCAAGGTCTTGAGTGGATGGGTCGTGTTAATCCT
AACCGGAGGGGTACTACCTACAACCAGAAATTCGAGGGCCGTGTCACCATGACCACAGACACATCCACGAGCACAGCC
TACATGGAGCTGCGTAGCCTGCGTTCTGACGACACGGCCGTGTATTACTGTGCGCGTGCGAACTGGCTTGACTACTGG
GGCCAGGGCACCACCGTCACCGTCTCCTCCGCCTCCACCAAGGGCCCATCGGTCTTCCCGCTAGCGCCCTGCTCCAGG
AGCACCTCCGAGAGCACAGCCGCCCTGGGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCCTCAGGACTCTACTCCCTCAGCAGCGTG
GTGACCGTGCCCTCCAGCAGCTTGGGCACGAAGACCTACACCTGCAACGTAGATCACAAGCCCAGCAACACCAAGGTG
GACAAGAGAGTTGAGTCCAAATATGGTCCCCCATGCCCACCCTGCCCAGCACCTGAGGCCGCCGGGGGACCATCAGTC
TTCCTGTTCCCCCCAAAACCCAAGGACACTCTCATGATCTCCCGGACCCCTGAGGTCACGTGCGTGGTGGTGGACGTG
AGCCAGGAAGACCCCGAGGTCCAGTTCAACTGGTACGTGGATGGCGTGGAGGTGCATAATGCCAAGACAAAGCCGCGG
GAGGAGCAGTTCAACAGCACGTACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCCAAAGCCAAAGGGCAGCCCCGA
GAGCCACAGGTGTACACCCTGCCCCCATCCCAGGAGGAGATGACCAAGAACCAGGTCAGCCTGACCTGCCTGGTCAAA
GGCTTCTACCCCAGCGACATCGCCGTGGAGTGGGAAAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGGCAGGAGGGGAATGTC
TTCTCATGCTCCGTGATGCATGAGGCTCTGCACAACCACTACACACAGAAGAGCCTCTCCCTGTCTCTG
SEQEDNO. 252
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGGAGGAGGTGGGTCTGGAGGTGGAGGATCTGGTGGAGGTGGGTCTGGAGGAGGTGGGTCTCAG
GTGCAGCTGGTGCAGAGCGGCGCCGAGGACAAGAAGCCCGGCGAGAGCGTGAAGATCAGCTGCAAGGCCAGCGGCTAC
ACCTTCACCAACTACGGCATGAACTGGGTGAGGCAGGCCCCCGGCCAGGGCCTGAAGTGGATGGGCTGGATCAACACC
TACACCGGCGAGAGCACCTACGCCGACGACTTCAAGGGCAGGTTCGCCTTCAGCCTGGACACCAGCGCCAGCACCGCC
TACCTGCAGCTGAGCAGCCTGAGGGGCGAGGACACCGCCGTGTACTTCTGCGCCAGGTTCGCCATCAAGGGCGACTAC
TGGGGCCAGGGCACCACCGTGACCGTGAGCAGCGCCAGCACCGGCGGCGGCGGCAGCGGCGGCGGCGGCAGCGGCGGC
GGCGGCAGCGGCGGCGGCGGCAGCGACATCGTGATGACCCAGAGCCCCCTGAGCCTGGAGGTGAGCCCCGGCGAGCCC
GCCAGCATCAGCTGCAGGAGCACCAAGAGCCTGCTGCACAGCGACGGCATCACCTACCTGTACTGGTACCTGCAGAAG
CCCGGCCAGAGCCCCCAGCTGCTGATCTACCAGCTGAGCAACCTGGCCAGCGGCGTGCCCGACAGGTTCAGCAGCAGC
GGCAGCGGCACCGACTTCACCCTGAAGATCAGCAGGGTGGAGGCCGAGGACGAGGGCACCTACTACTGCGCCCAGAAC
CTGGAGATCCCCAGGACCTTCGGCCAGGGCACCAAGCTGGAGATCAAGAGGACCCATCATCACCATCACCAT
SEQID NO. 253
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGGAGGAGGTGGGTCTGGAGGTGGAGGATCTGGTGGAGGTGGGTCTGGAGGAGGTGGGTCTCAG
GTTCAGCTGGTGCAGTCTGGTGCTGAGGATGTGAAGCCTGATGCCTCAGTGAAGCTCTCCTGCAAGGCTTCTGGTTAC
ACATTCACTGACTACTACATGCACTGGGTGCGTCAGGCCCCTGGTCAAGGTCTTGAGTGGATGGGTCGTGTTAATCCT
AACCGGAGGGGTACTACCTACAACCAGAAATTCGAGGGCCGTGTCACCATGACCACAGACACATCCACGAGCACAGCC
TACATGCAGCTGAGTAGCCTGCGTGGTGAAGACACGGCCGTGTATTACTGTGCGCGTGCGAACTGGCTTGACTACTGG
GGCCAGGGCACCACCGTCACCGTCTCCTCCGCCTCCACCGGGGGAGGTGGGTCTGGAGGTGGAGGATCTGGTGGAGGT
GGGTCTGGTGGAGGTGGGTCTGACATCCAGATGACCCAGTCTCCATCCTCCCTGGAGGCATCTGTAGGAGACAGAGTC
ACCATCACTTGCAGTGTCAGCTCAAGTGTATCCTCCATTTACTTGCACTGGTATCAGCAGAAACCAGGGAAAAGCCCT
AAGCTCCTGATCTATAGCACATCCAACTTGGCTTCTGGAGTCCCAGATAGGTTCAGTGGCAGTGGATCTGGGACAGAT
TTCACTCTCACCATCAGCAGTCTGCAAGCCGAAGATGAGGGCACTTACTACTGTCAAGTCTACAGTGGTTACCCGCTC
ACGTTCGGCGGAGGGACCAAGCTGGAGATCAAACGAACTCATCATCACCATCACCAT
SEQEDNO. 254
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGGAGGAGGTGGGTCTGGAGGTGGAGGATCTGGTGGAGGTGGGTCTGGAGGAGGTGGGTCTCAG
GTGCAGCTGCAGGAGAGCGGCCCCGGCGACGTGAAGCCCAGCGAGACCCTGAGCCTGACCTGCACCGTGAGCGGCGGC
AGCGTGAGCAGCGGCGACTACTACTGGACCTGGATCAGGCAGAGCCCCGGCAAGGGCCTGGAGTGGATCGGCCACATC
TACTACAGCGGCAACACCAACTACAACCCCAGCCTGAAGAGCAGGCTGACCATCAGCATCGACACCAGCAAGACCACC
TTCAGCCTGCAGCTGAGCAGCGTGACCGGCGAGGACACCGCCATCTACTACTGCGTGAGGGACAGGGTGACCGGCGCC
TTCGACATCTGGGGCCAGGGCACCACCGTGACCGTGAGCAGCGCCAGCACCGGCGGCGGCGGCAGCGGCGGCGGCGGC
AGCGGCGGCGGCGGCAGCGGCGGCGGCGGCAGCGACATCCAGATGACCCAGAGCCCCAGCAGCCTGGAGGCCAGCGTG
GGCGACAGGGTGACCATCACCTGCCAGGCCAGCCAGGACATCAGCAACTACCTGAACTGGTACCAGCAGAAGCCCGGC
AAGAGCCCCAAGCTGCTGATCTACGACGCCAGCAACCTGGAGACCGGCGTGCCCGACAGGTTCAGCGGCAGCGGCAGC
GGCACCGACTTCACCTTCACCATCAGCAGCCTGCAGGCCGAGGACGAGGGCACCTACTTCTGCCAGCACTTCGACCAC
CTGCCCCTGGCCTTCGGCGGCGGCACCAAGCTGGAGATCAAGAGGACCCATCATCACCATCACCAT
SEQID NO. 255
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGGGGGAGGTGGGTCTGGAGGTGGAGGATCTGGTGGAGGTGGGTCTGGTGGAGGTGGGTCTGAG
GTGCAGCTGGTGGAGTCTGGTGGTGGTCTTGTTCAACCTGGTGGTTCTCTTCGTCTTTCTTGTGCTGCTTCTGGTTTT
AATATTAAAGATACTTATATTCATTGGGTTCGTCAAGCTCCTGGTAAAGGTCTTGAATGGGTTGCTCGTATTTATCCT
ACTAATGGTTATACTCGTTATGCTGATTCTGTTAAAGGTCGTTTTACTATTTCTGCTGATACTTCTAAAAATACTGCT
TATCTTCAAATGAACTCTCTTCGTGCTGAAGATACTGCTGTTTATTATTGTTCTCGTTGGGGTGGTGATGGTTTTTAT
GCTATGGATTATTGGGGTCAAGGTACTCTTGTCACCGTCTCCTCAGCTAGCACCGGGGGAGGTGGGTCTGGAGGTGGA
GGATCTGGTGGAGGTGGGTCTGACATCCAGATGACCCAGTCTCCTTCTTCTCTTTCTGCTTCTGTTGGTGATCGTGTT
ACTATTACTTGTCGTGCTTCTCAAGATGTTAATACTGCTGTTGCTTGGTATCAACAAAAACCTGGTAAAGCTCCTAAA
CTTCTTATTTATTCTGCTTCTTTTCTTTATTCTGGTGTTCCTTCTCGTTTTTCTGGTTCTCGTTCTGGTACTGATTTT
ACTCTTACTATTTCTTCTCTTCAACCTGAAGATTTTGCTACTTATTATTGTCAACAACATTATACTACTCCTCCTACT
TTTGGTCAAGGTACCAAGGTGGAGATCAAACGTACGCATCATCACCATCACCAT
SEQID NO. 256
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGACAAAACTCACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTCACATGCGTGGTGGTGGACGTGAGC
CACGAAGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCATCTCCAAAGCCAAAGGGCAGCCCCGAGAA
CCACAGGTGTACACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACCACGCCTCCCGTG
CTGGACTCCGACGGCTCCTTCTTCCTCTACAGCAAGCTCACCGTGGACAAGAGCAGGTGGCAGCAGGGGAACGTCTTC
TCATGCTCCGTGATGCACGAGGCTCTGCACAACCACTACACGCAGAAGAGCCTCTCCCTGTCTCCGGGTGGAGGAGGT
GGGTCTGGAGGTGGAGGATCTGGTGGAGGTGGGTCTGGAGGAGGTGGGTCTGAGGTGCAGCTGGTGGAGTCTGGTGGT
GGTCTTGTTCAACCTGGTGGTTCTCTTCGTCTTTCTTGTGCTGCTTCTGGTTTTAATATTAAAGATACTTATATTCAT
TGGGTTCGTCAAGCTCCTGGTAAAGGTCTTGAATGGGTTGCTCGTATTTATCCTACTAATGGTTATACTCGTTATGCT
GATTCTGTTAAAGGTCGTTTTACTATTTCTGCTGATACTTCTAAAAATACTGCTTATCTTCAAATGAACTCTCTTCGT
GCTGAAGATACTGCTGTTTATTATTGTTCTCGTTGGGGTGGTGATGGTTTTTATGCTATGGATTATTGGGGTCAAGGT
ACTCTTGTCACCGTCTCCTCAGCTAGCACCGGGGGAGGTGGGTCTGGAGGTGGAGGATCTGGTGGAGGTGGGTCTGAC
ATCCAGATGACCCAGTCTCCTTCTTCTCTTTCTGCTTCTGTTGGTGATCGTGTTACTATTACTTGTCGTGCTTCTCAA
GATGTTAATACTGCTGTTGCTTGGTATCAACAAAAACCTGGTAAAGCTCCTAAACTTCTTATTTATTCTGCTTCTTTT
CTTTATTCTGGTGTTCCTTCTCGTTTTTCTGGTTCTCGTTCTGGTACTGATTTTACTCTTACTATTTCTTCTCTTCAA
CCTGAAGATTTTGCTACTTATTATTGTCAACAACATTATACTACTCCTCCTACTTTTGGTCAAGGTACCAAGGTGGAG
ATCAAACGTACG
SEQID NO. 257
ATGGAGCTGGCGGCCTTGTGCCGCTGGGGGCTCCTCCTCGCCCTCTTGCCCCCCGGAGCCGCGAGCACCCAAGTGTGC
ACCGGCACAGACATGAAGCTGCGGCTCCCTGCCAGTCCCGAGACCCACCTGGACATGCTCCGCCACCTCTACCAGGGC
TGCCAGGTGGTGCAGGGAAACCTGGAACTCACCTACCTGCCCACCAATGCCAGCCTGTCCTTCCTGCAGGATATCCAG
GAGGTGCAGGGCTACGTGCTCATCGCTCACAACCAAGTGAGGCAGGTCCCACTGCAGAGGCTGCGGATTGTGCGAGGC
ACCCAGCTCTTTGAGGACAACTATGCCCTGGCCGTGCTAGACAATGGAGACCCGCTGAACAATACCACCCCTGTCACA
GGGGCCTCCCCAGGAGGCCTGCGGGAGCTGCAGCTTCGAAGCCTCACAGAGATCTTGAAAGGAGGGGTCTTGATCCAG
CGGAACCCCCAGCTCTGCTACCAGGACACGATTTTGTGGAAGGACATCTTCCACAAGAACAACCAGCTGGCTCTCACA
CTGATAGACACCAACCGCTCTCGGGCCTGCCACCCCTGTTCTCCGATGTGTAAGGGCTCCCGCTGCTGGGGAGAGAGT
TCTGAGGATTGTCAGAGCCTGACGCGCACTGTCTGTGCCGGTGGCTGTGCCCGCTGCAAGGGGCCACTGCCCACTGAC
TGCTGCCATGAGCAGTGTGCTGCCGGCTGCACGGGCCCCAAGCACTCTGACTGCCTGGCCTGCCTCCACTTCAACCAC
AGTGGCATCTGTGAGCTGCACTGCCCAGCCCTGGTCACCTACAACACAGACACGTTTGAGTCCATGCCCAATCCCGAG
GGCCGGTATACATTCGGCGCCAGCTGTGTGACTGCCTGTCCCTACAACTACCTTTCTACGGACGTGGGATCCTGCACC
CTCGTCTGCCCCCTGCACAACCAAGAGGTGACAGCAGAGGATGGAACACAGCGGTGTGAGAAGTGCAGCAAGCCCTGT
GCCCGAGTGTGCTATGGTCTGGGCATGGAGCACTTGCGAGAGGTGAGGGCAGTTACCAGTGCCAATATCCAGGAGTTT
GCTGGCTGCAAGAAGATCTTTGGGAGCCTGGCATTTCTGCCGGAGAGCTTTGATGGGGACCCAGCCTCCAACACTGCC
CCGCTCCAGCCAGAGCAGCTCCAAGTGTTTGAGACTCTGGAAGAGATCACAGGTTACCTATACATCTCAGCATGGCCG
GACAGCCTGCCTGACCTCAGCGTCTTCCAGAACCTGCAAGTAATCCGGGGACGAATTCTGCACAATGGCGCCTACTCG
CTGACCCTGCAAGGGCTGGGCATCAGCTGGCTGGGGCTGCGCTCACTGAGGGAACTGGGCAGTGGACTGGCCCTCATC
CACCATAACACCCACCTCTGCTTCGTGCACACGGTGCCCTGGGACCAGCTCTTTCGGAACCCGCACCAAGCTCTGCTC
CACACTGCCAACCGGCCAGAGGACGAGTGTGTGGGCGAGGGCCTGGCCTGCCACCAGCTGTGCGCCCGAGGGCACTGC
TGGGGTCCAGGGCCCACCCAGTGTGTCAACTGCAGCCAGTTCCTTCGGGGCCAGGAGTGCGTGGAGGAATGCCGAGTA
CTGCAGGGGCTCCCCAGGGAGTATGTGAATGCCAGGCACTGTTTGCCGTGCCACCCTGAGTGTCAGCCCCAGAATGGC
TCAGTGACCTGTTTTGGACCGGAGGCTGACCAGTGTGTGGCCTGTGCCCACTATAAGGACCCTCCCTTCTGCGTGGCC
CGCTGCCCCAGCGGTGTGAAACCTGACCTCTCCTACATGCCCATCTGGAAGTTTCCAGATGAGGAGGGCGCATGCCAG
CCTTGCCCCATCAACTGCACCCACTCCTGTGTGGACCTGGATGACAAGGGCTGCCCCGCCGAGCAGAGAGCCAGCCCT
CTGACGGGAGGAGGTGGGTCTGGAGGTGGAGGATCTGGTGGAGGTGGGTCTGGAGGAGGTGGGTCTCAGGTGCAGCTG
CAGGAGAGCGGCCCCGGCGACGTGAAGCCCAGCGAGACCCTGAGCCTGACCTGCACCGTGAGCGGCGGCAGCGTGAGC
AGCGGCGACTACTACTGGACCTGGATCAGGCAGAGCCCCGGCAAGGGCCTGGAGTGGATCGGCCACATCTACTACAGC
GGCAACACCAACTACAACCCCAGCCTGAAGAGCAGGCTGACCATCAGCATCGACACCAGCAAGACCACCTTCAGCCTG
CAGCTGAGCAGCGTGACCGGCGAGGACACCGCCATCTACTACTGCGTGAGGGACAGGGTGACCGGCGCCTTCGACATC
TGGGGCCAGGGCACCACCGTGACCGTGAGCAGCGCCAGCACCGGCGGCGGCGGCAGCGGCGGCGGCGGCAGCGGCGGC
GGCGGCAGCGGCGGCGGCGGCAGCGACATCCAGATGACCCAGAGCCCCAGCAGCCTGGAGGCCAGCGTGGGCGACAGG
GTGACCATCACCTGCCAGGCCAGCCAGGACATCAGCAACTACCTGAACTGGTACCAGCAGAAGCCCGGCAAGAGCCCC
AAGCTGCTGATCTACGACGCCAGCAACCTGGAGACCGGCGTGCCCGACAGGTTCAGCGGCAGCGGCAGCGGCACCGAC
TTCACCTTCACCATCAGCAGCCTGCAGGCCGAGGACGAGGGCACCTACTTCTGCCAGCACTTCGACCACCTGCCCCTG
GCCTTCGGCGGCGGCACCAAGCTGGAGATCAAGAGGACCCATCATCACCATCACCAT
SEQID NO. 258
ATGGAGCTGGCGGCCTTGTGCCGCTGGGGGCTCCTCCTCGCCCTCTTGCCCCCCGGAGCCGCGAGCAACCGGCCAGAG
GACGAGTGTGTGGGCGAGGGCCTGGCCTGCCACCAGCTGTGCGCCCGAGGGCACTGCTGGGGTCCAGGGCCCACCCAG
TGTGTCAACTGCAGCCAGTTCCTTCGGGGCCAGGAGTGCGTGGAGGAATGCCGAGTACTGCAGGGGCTCCCCAGGGAG
TATGTGAATGCCAGGCACTGTTTGCCGTGCCACCCTGAGTGTCAGCCCCAGAATGGCTCAGTGACCTGTTTTGGACCG
GAGGCTGACCAGTGTGTGGCCTGTGCCCACTATAAGGACCCTCCCTTCTGCGTGGCCCGCTGCCCCAGCGGTGTGAAA
CCTGACCTCTCCTACATGCCCATCTGGAAGTTTCCAGATGAGGAGGGCGCATGCCAGCCTTGCCCCATCAACTGCACC
CACTCCTGTGTGGACCTGGATGACAAGGGCTGCCCCGCCGAGCAGAGAGCCAGCCCTCTGACGGGAGGAGGTGGGTCT
GGAGGTGGAGGATCTGGTGGAGGTGGGTCTGGAGGAGGTGGGTCTCAGGTGCAGCTGCAGGAGAGCGGCCCCGGCGAC
GTGAAGCCCAGCGAGACCCTGAGCCTGACCTGCACCGTGAGCGGCGGCAGCGTGAGCAGCGGCGACTACTACTGGACC
TGGATCAGGCAGAGCCCCGGCAAGGGCCTGGAGTGGATCGGCCACATCTACTACAGCGGCAACACCAACTACAACCCC
AGCCTGAAGAGCAGGCTGACCATCAGCATCGACACCAGCAAGACCACCTTCAGCCTGCAGCTGAGCAGCGTGACCGGC
GAGGACACCGCCATCTACTACTGCGTGAGGGACAGGGTGACCGGCGCCTTCGACATCTGGGGCCAGGGCACCACCGTG
ACCGTGAGCAGCGCCAGCACCGGCGGCGGCGGCAGCGGCGGCGGCGGCAGCGGCGGCGGCGGCAGCGGCGGCGGCGGC
AGCGACATCCAGATGACCCAGAGCCCCAGCAGCCTGGAGGCCAGCGTGGGCGACAGGGTGACCATCACCTGCCAGGCC
AGCCAGGACATCAGCAACTACCTGAACTGGTACCAGCAGAAGCCCGGCAAGAGCCCCAAGCTGCTGATCTACGACGCC
AGCAACCTGGAGACCGGCGTGCCCGACAGGTTCAGCGGCAGCGGCAGCGGCACCGACTTCACCTTCACCATCAGCAGC
CTGCAGGCCGAGGACGAGGGCACCTACTTCTGCCAGCACTTCGACCACCTGCCCCTGGCCTTCGGCGGCGGCACCAAG
CTGGAGATCAAGAGGACCCATCATCACCATCACCAT
SEQID NO. 263
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGGAGGAGGTGGGTCTGGAGGTGGAGGATCTGGTGGAGGTGGGTCTGGAGGAGGTGGGTCTGAG
GTGCAGCTGCAGCAGTCTGGGGCTGAGCTGGTGAAGCCTGGGGCCTCAGTGAAGATGTCCTGCAAGGCTTCTGGCTAC
ACATTTACCAGTTACAATATGCACTGGGTAAAGCAGACACCTGGACAGGGCCTGGAATGGATTGGAGCTATTTATCCA
GGAAATGGTGATACTTCCTACAATCAGAAGTTCAAAGGCAAGGCCACATTGACTGCAGACAAATCCTCCAGCACAGCC
TACATGCAGCTCAGCAGCCTGACATCTGAGGACTCTGCGGACTATTACTGTGCAAGATCTAATTATTACGGTAGTAGC
TACTGGTTCTTCGATGTCTGGGGCGCAGGGACCACGGTCACCGTCTCCTCAGGCAGTACTAGCGGTGGTGGCTCCGGG
GGCGGTTCCGGTGGGGGCGGCAGCAGCGACATTGTGCTGACCCAATCTCCAGCTATCCTGTCTGCATCTCCAGGGGAG
AAGGTCACAATGACTTGCAGGGCCAGCTCAAGTGTAAATTACATGGACTGGTACCAGAAGAAGCCAGGATCCTCCCCC
AAACCCTGGATTTATGCCACATCCAACCTGGCTTCTGGAGTCCCTGCTCGCTTCAGTGGCAGTGGGTCTGGGACCTCT
TACTCTCTCACAATCAGCAGAGTGGAGGCTGAAGATGCTGCCACTTATTACTGCCAGCAGTGGAGTTTTAATCCACCC
ACGTTCGGAGGGGGGACCAAGCTGGAAATAAAACATCATCACCATCACCAT
SEQID NO. 264
ATGCATCTCCTCGGCCCCTGGCTCCTGCTCCTGGTTCTAGAATACTTGGCTTTCTCTGACTCAAGTAAATGGGTTTTT
GAGCACCCTGAAACCCTCTACGCCTGGGAGGGGGCCTGCGTCTGGATCCCCTGCACCTACAGAGCCCTAGATGGTGAC
CTGGAAAGCTTCATCCTGTTCCACAATCCTGAGTATAACAAGAACACCTCGAAGTTTGATGGGACAAGACTCTATGAA
AGCACAAAGGATGGGAAGGTTCCTTCTGAGCAGAAAAGGGTGCAATTCCTGGGAGACAAGAATAAGGCCTGCACACTG
AGTATCCACCCGGTGCACCTCAATGACAGTGGTCAGCTGGGGCTGAGGATGGAGTCCAAGACTGAGAAATGGATGGAA
CGAATACACCTCAATGTCTCTGAAAGGCCTTTTCCACCTCATATCCAGCTCCCTCCAGAAATTCAAGAGTCCCAGGAA
GTCACTCTGACCTGCTTGCTGAATTTCTCCTGCTATGGGTATCCGATCCAATTGCAGTGGCTCCTAGAGGGGGTTCCA
ATGAGGCAGGCTGCTGTCACCTCGACCTCCTTGACCATCAAGTCTGTCTTCACCCGGAGCGAGCTCAAGTTCTCCCCA
CAGTGGAGTCACCATGGGAAGATTGTGACCTGCCAGCTTCAGGATGCAGATGGGAAGTTCCTCTCCAATGACACGGTG
CAGCTGAACGTGAAGCACACCCCGAAGTTGGAGATCAAGGTCACTCCCAGTGATGCCATAGTGAGGGAGGGGGACTCT
GTGACCATGACCTGCGAGGTCAGCAGCAGCAACCCGGAGTACACGACGGTATCCTGGCTCAAGGATGGGACCTCGCTG
AAGAAGCAGAATACATTCACGCTAAACCTGCGCGAAGTGACCAAGGACCAGAGTGGGAAGTACTGCTGTCAGGTCTCC
AATGACGTGGGCCCGGGAAGGTCGGAAGAAGTGTTCCTGCAAGTGCAGTATGCCCCGGAAGGAGGAGGTGGGTCTGGA
GGTGGAGGATCTGGTGGAGGTGGGTCTGGAGGAGGTGGGTCTGACATCCAGATGACACAGACTACATCCTCCCTGTCT
GCCTCTCTGGGAGACAGAGTCACCATCAGTTGCAGGGCAAGTCAGGACATTAGTAAATATTTAAATTGGTATCAGCAG
AAACCAGATGGAACTGTTAAACTCCTGATCTACCATACATCAAGATTACACTCAGGAGTCCCATCAAGGTTCAGTGGC
AGTGGGTCTGGAACAGATTATTCTCTCACCATTAGCAACCTGGAGCAAGAAGATATTGCCACTTACTTTTGCCAACAG
GGTAATACGCTTCCGTACACGTTCGGAGGGGGGACTAAGTTGGAAATAACAGGAGGAGGTGGGTCTGGAGGTGGAGGA
TCTGGTGGAGGTGGGTCTGGAGGAGGTGGGTCTGAGGTGAAACTGCAGGAGTCAGGACCTGGCCTGGTGGCGCCCTCA
CAGAGCCTGTCCGTCACATGCACTGTCTCAGGGGTCTCATTACCCGACTATGGTGTAAGCTGGATTCGCCAGCCTCCA
CGAAAGGGTCTGGAGTGGCTGGGAGTAATATGGGGTAGTGAAACCACATACTATAATTCAGCTCTCAAATCCAGACTG
ACCATCATCAAGGACAACTCCAAGAGCCAAGTTTTCTTAAAAATGAACAGTCTGCAAACTGATGACACAGCCATTTAC
TACTGTGCCAAACATTATTACTACGGTGGTAGCTATGCTATGGACTACTGGGGTCAAGGAACCTCAGTCACCGTCTCC
TCACATCATCACCATCACCAT
SEQID NO. 265
ATGCATCTCCTCGGCCCCTGGCTCCTGCTCCTGGTTCTAGAATACTTGGCTTTCTCTGACTCAAGTAAATGGGTTTTT
GAGCACCCTGAAACCCTCTACGCCTGGGAGGGGGCCTGCGTCTGGATCCCCTGCACCTACAGAGCCCTAGATGGTGAC
CTGGAAAGCTTCATCCTGTTCCACAATCCTGAGTATAACAAGAACACCTCGAAGTTTGATGGGACAAGACTCTATGAA
AGCACAAAGGATGGGAAGGTTCCTTCTGAGCAGAAAAGGGTGCAATTCCTGGGAGACAAGAATAAGGCCTGCACACTG
AGTATCCACCCGGTGCACCTCAATGACAGTGGTCAGCTGGGGCTGAGGATGGAGTCCAAGACTGAGAAATGGATGGAA
CGAATACACCTCAATGTCTCTGAAAGGCCTTTTCCACCTCATATCCAGCTCCCTCCAGAAATTCAAGAGTCCCAGGAA
GTCACTCTGACCTGCTTGCTGAATTTCTCCTGCTATGGGTATCCGATCCAATTGCAGTGGCTCCTAGAGGGGGTTCCA
ATGAGGCAGGCTGCTGTCACCTCGACCTCCTTGACCATCAAGTCTGTCTTCACCCGGAGCGAGCTCAAGTTCTCCCCA
CAGTGGAGTCACCATGGGAAGATTGTGACCTGCCAGCTTCAGGATGCAGATGGGAAGTTCCTCTCCAATGACACGGTG
CAGCTGAACGTGAAGCACACCCCGAAGTTGGAGATCAAGGTCACTCCCAGTGATGCCATAGTGAGGGAGGGGGACTCT
GTGACCATGACCTGCGAGGTCAGCAGCAGCAACCCGGAGTACACGACGGTATCCTGGCTCAAGGATGGGACCTCGCTG
AAGAAGCAGAATACATTCACGCTAAACCTGCGCGAAGTGACCAAGGACCAGAGTGGGAAGTACTGCTGTCAGGTCTCC
AATGACGTGGGCCCGGGAAGGTCGGAAGAAGTGTTCCTGCAAGTGCAGTATGCCCCGGAAGGAGGAGGTGGGTCTGGA
GGTGGAGGATCTGGTGGAGGTGGGTCTGGAGGAGGTGGGTCTGAGGTGCAGCTGCAGCAGTCTGGGGCTGAGCTGGTG
AAGCCTGGGGCCTCAGTGAAGATGTCCTGCAAGGCTTCTGGCTACACATTTACCAGTTACAATATGCACTGGGTAAAG
CAGACACCTGGACAGGGCCTGGAATGGATTGGAGCTATTTATCCAGGAAATGGTGATACTTCCTACAATCAGAAGTTC
AAAGGCAAGGCCACATTGACTGCAGACAAATCCTCCAGCACAGCCTACATGCAGCTCAGCAGCCTGACATCTGAGGAC
TCTGCGGACTATTACTGTGCAAGATCTAATTATTACGGTAGTAGCTACTGGTTCTTCGATGTCTGGGGCGCAGGGACC
ACGGTCACCGTCTCCTCAGGCAGTACTAGCGGTGGTGGCTCCGGGGGCGGTTCCGGTGGGGGCGGCAGCAGCGACATT
GTGCTGACCCAATCTCCAGCTATCCTGTCTGCATCTCCAGGGGAGAAGGTCACAATGACTTGCAGGGCCAGCTCAAGT
GTAAATTACATGGACTGGTACCAGAAGAAGCCAGGATCCTCCCCCAAACCCTGGATTTATGCCACATCCAACCTGGCT
TCTGGAGTCCCTGCTCGCTTCAGTGGCAGTGGGTCTGGGACCTCTTACTCTCTCACAATCAGCAGAGTGGAGGCTGAA
GATGCTGCCACTTATTACTGCCAGCAGTGGAGTTTTAATCCACCCACGTTCGGAGGGGGGACCAAGCTGGAAATAAAA
CATCATCACCATCACCAT
SEQID NO. 266
AAGCTTGTTGACATTGATTATTGACTAGTTATTAATAGTAATCAATTACGGGGTCATTAGTTCATAGCCCATATATGG
AGTTCCGCGTTACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTATTACCATGG
TGATGCGGTTTTGGCAGTACATCAATGGGCGTGGATAGCGGTTTGACTCACGGGGATTTCCAAGTCTCCACCCCATTG
ACGTCAATGGGAGTTTGTTTTGGCACCAAAATCAACGGGACTTTCCAAAATGTCGTAACAACTCCGCCCCATTGACGC
AAATGGGCGGTAGGCGTGTACGGTGGGAGGTCTATATAAGCAGAGCTCAAGCTTAACTAGTTAGCGGACCGACGCGTA
CGCGGCCGCTCGAGATGGAGAGCGACGAGAGCGGCCTGCCCGCCATGGAGATCGAGTGCCGCATCACCGGCACCCTGA
ACGGCGTGGAGTTCGAGCTGGTGGGCGGCGGAGAGGGCACCCCCGAGCAGGGCCGCATGACCAACAAGATGAAGAGCA
CCAAAGGCGCCCTGACCTTCAGCCCCTACCTGCTGAGCCACGTGATGGGCTACGGCTTCTACCACTTCGGCACCTACC
CCAGCGGCTACGAGAACCCCTTCCTGCACGCCATCAACAACGGCGGCTACACCAACACCCGCATCGAGAAGTACGAGG
ACGGCGGCGTGCTGCACGTGAGCTTCAGCTACCGCTACGAGGCCGGCCGCGTGATCGGCGACTTCAAGGTGATGGGCA
CCGGCTTCCCCGAGGACAGCGTGATCTTCACCGACAAGATCATCCGCAGCAACGCCACCGTGGAGCACCTGCACCCCA
TGGGCGATAACGATCTGGATGGCAGCTTCACCCGCACCTTCAGCCTGCGCGACGGCGGCTACTACAGCTCCGTGGTGG
ACAGCCACATGCACTTCAAGAGCGCCATCCACCCCAGCATCCTGCAGAACGGGGGCCCCATGTTCGCCTTCCGCCGCG
TGGAGGAGGATCACAGCAACACCGAGCTGGGCATCGTGGAGTACCAGCACGCCTTCAAGACCCCGGATGCAGATGCCG
GTGAAGAAAGAGTT
SEQID NO. 267
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAGGGAGGAGGTGGGTCTGGAGGTGGAGGATCTGGTGGAGGTGGGTCTGGAGGAGGTGGGTCTGAC
ATCCTGATGACCCAATCTCCATCCTCCATGTCTGTATCTCTGGGAGACACAGTCAGCATCACTTGCCATTCAAGTCAG
GACATTAACAGTAATATAGGGTGGTTGCAGCAGAGACCAGGGAAATCATTTAAGGGCCTGATCTATCATGGAACCAAC
TTGGACGATGAAGTTCCATCAAGGTTCAGTGGCAGTGGATCTGGAGCCGATTATTCTCTCACCATCAGCAGCCTGGAA
TCTGAAGATTTTGCAGACTATTACTGTGTACAGTATGCTCAGTTTCCGTGGACGTTCGGTGGAGGCACCAAGCTCGAA
ATCAAACGTGGAGGAGGTGGGTCTGGAGGTGGAGGATCTGGTGGAGGTGGGTCTGGAGGAGGTGGGTCTATGAGAGTG
CTGATTCTTTTGTGGCTGTTCACAGCCTTTCCTGGTGTCCTGTCTGATGTGCAGCTTCAGGAGTCGGGACCTAGCCTG
GTGAAACCTTCTCAGTCTCTGTCCCTCACCTGCACTGTCACTGGCTACTCAATCACCAGTGATTTTGCCTGGAACTGG
ATCCGGCAGTTTCCAGGAAACAAGCTGGAGTGGATGGGCTACATAAGTTATAGTGGTAACACTAGGTACAACCCATCT
CTCAAAAGTCGAATCTCTATCACTCGAGACACATCCAAGAACCAATTCTTCCTGCAGTTGAATTCTGTGACTATTGAG
GACACAGCCACATATTACTGTGTAACGGCGGGACGCGGGTTTCCTTATTGGGGCCAAGGGACTCTGGTCACTGTCTCT
GCACATCATCACCATCACCAT
SEQID NO. 268
ATGCATCTCCTCGGCCCCTGGCTCCTGCTCCTGGTTCTAGAATACTTGGCTTTCTCTGACTCAAGTAAATGGGTTTTT
GAGCACCCTGAAACCCTCTACGCCTGGGAGGGGGCCTGCGTCTGGATCCCCTGCACCTACAGAGCCCTAGATGGTGAC
CTGGAAAGCTTCATCCTGTTCCACAATCCTGAGTATAACAAGAACACCTCGAAGTTTGATGGGACAAGACTCTATGAA
AGCACAAAGGATGGGAAGGTTCCTTCTGAGCAGAAAAGGGTGCAATTCCTGGGAGACAAGAATAAGGCCTGCACACTG
AGTATCCACCCGGTGCACCTCAATGACAGTGGTCAGCTGGGGCTGAGGATGGAGTCCAAGACTGAGAAATGGATGGAA
CGAATACACCTCAATGTCTCTGAAAGGCCTTTTCCACCTCATATCCAGCTCCCTCCAGAAATTCAAGAGTCCCAGGAA
GTCACTCTGACCTGCTTGCTGAATTTCTCCTGCTATGGGTATCCGATCCAATTGCAGTGGCTCCTAGAGGGGGTTCCA
ATGAGGCAGGCTGCTGTCACCTCGACCTCCTTGACCATCAAGTCTGTCTTCACCCGGAGCGAGCTCAAGTTCTCCCCA
CAGTGGAGTCACCATGGGAAGATTGTGACCTGCCAGCTTCAGGATGCAGATGGGAAGTTCCTCTCCAATGACACGGTG
CAGCTGAACGTGAAGCACACCCCGAAGTTGGAGATCAAGGTCACTCCCAGTGATGCCATAGTGAGGGAGGGGGACTCT
GTGACCATGACCTGCGAGGTCAGCAGCAGCAACCCGGAGTACACGACGGTATCCTGGCTCAAGGATGGGACCTCGCTG
AAGAAGCAGAATACATTCACGCTAAACCTGCGCGAAGTGACCAAGGACCAGAGTGGGAAGTACTGCTGTCAGGTCTCC
AATGACGTGGGCCCGGGAAGGTCGGAAGAAGTGTTCCTGCAAGTGCAGTATGCCCCGGAAGGAGGAGGTGGGTCTGGA
GGTGGAGGATCTGGTGGAGGTGGGTCTGGAGGAGGTGGGTCTGACATCCTGATGACCCAATCTCCATCCTCCATGTCT
GTATCTCTGGGAGACACAGTCAGCATCACTTGCCATTCAAGTCAGGACATTAACAGTAATATAGGGTGGTTGCAGCAG
AGACCAGGGAAATCATTTAAGGGCCTGATCTATCATGGAACCAACTTGGACGATGAAGTTCCATCAAGGTTCAGTGGC
AGTGGATCTGGAGCCGATTATTCTCTCACCATCAGCAGCCTGGAATCTGAAGATTTTGCAGACTATTACTGTGTACAG
TATGCTCAGTTTCCGTGGACGTTCGGTGGAGGCACCAAGCTCGAAATCAAACGTGGAGGAGGTGGGTCTGGAGGTGGA
GGATCTGGTGGAGGTGGGTCTGGAGGAGGTGGGTCTATGAGAGTGCTGATTCTTTTGTGGCTGTTCACAGCCTTTCCT
GGTGTCCTGTCTGATGTGCAGCTTCAGGAGTCGGGACCTAGCCTGGTGAAACCTTCTCAGTCTCTGTCCCTCACCTGC
ACTGTCACTGGCTACTCAATCACCAGTGATTTTGCCTGGAACTGGATCCGGCAGTTTCCAGGAAACAAGCTGGAGTGG
ATGGGCTACATAAGTTATAGTGGTAACACTAGGTACAACCCATCTCTCAAAAGTCGAATCTCTATCACTCGAGACACA
TCCAAGAACCAATTCTTCCTGCAGTTGAATTCTGTGACTATTGAGGACACAGCCACATATTACTGTGTAACGGCGGGA
CGCGGGTTTCCTTATTGGGGCCAAGGGACTCTGGTCACTGTCTCTGCACATCATCACCATCACCAT
SEQID NO. 271
ATGCTCAGGCTGCTCTTGGCTCTCAACTTATTCCCTTCAATTCAAGTAACAGGAGACATCCAGATGACACAGACTACA
TCCTCCCTGTCTGCCTCTCTGGGAGACAGAGTCACCATCAGTTGCAGGGCAAGTCAGGACATTAGTAAATATTTAAAT
TGGTATCAGCAGAAACCAGATGGAACTGTTAAACTCCTGATCTACCATACATCAAGATTACACTCAGGAGTCCCATCA
AGGTTCAGTGGCAGTGGGTCTGGAACAGATTATTCTCTCACCATTAGCAACCTGGAGCAAGAAGATATTGCCACTTAC
TTTTGCCAACAGGGTAATACGCTTCCGTACACGTTCGGAGGGGGGACTAAGTTGGAAATAACAGGAGGAGGTGGGTCT
GGAGGTGGAGGATCTGGTGGAGGTGGGTCTGGAGGAGGTGGGTCTGAGGTGAAACTGCAGGAGTCAGGACCTGGCCTG
GTGGCGCCCTCACAGAGCCTGTCCGTCACATGCACTGTCTCAGGGGTCTCATTACCCGACTATGGTGTAAGCTGGATT
CGCCAGCCTCCACGAAAGGGTCTGGAGTGGCTGGGAGTAATATGGGGTAGTGAAACCACATACTATAATTCAGCTCTC
AAATCCAGACTGACCATCATCAAGGACAACTCCAAGAGCCAAGTTTTCTTAAAAATGAACAGTCTGCAAACTGATGAC
ACAGCCATTTACTACTGTGCCAAACATTATTACTACGGTGGTAGCTATGCTATGGACTACTGGGGTCAAGGAACCTCA
GTCACCGTCTCCTCAGACTACAAAGACGATGACGACAAGATTGAAGTTATGTATCCTCCTCCTTACCTAGACAATGAG
AAGAGCAATGGAACCATTATCCATGTGAAAGGGAAACACCTTTGTCCAAGTCCCCTATTTCCCGGACCTTCTAAGCCC
TTTTGGGTGCTGGTGGTGGTTGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCGCCGCCCCGGGCCCACCCGCAAG
CATTACCAGCCCTATGCCCCACCACGCGACTTCGCAGCCTATCGCTCCAAACGGGGCAGAAAGAAACTCCTGTATATA
TTCAAACAACCATTTATGAGACCAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGAGACGTGGCCGGGACCCTGAGATGGGG
GGAAAGCCGCAGAGAAGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGTCTCAGTACAGCCACC
AAGGACACCTACGACGCCCTTCACATGCAGGCCCTGCCCCCTCGC
SEQID NO. 272
ATGCTCAGGCTGCTCTTGGCTCTCAACTTATTCCCTTCAATTCAAGTAACAGGAGACATCCAGATGACACAGACTACA
TCCTCCCTGTCTGCCTCTCTGGGAGACAGAGTCACCATCAGTTGCAGGGCAAGTCAGGACATTAGTAAATATTTAAAT
TGGTATCAGCAGAAACCAGATGGAACTGTTAAACTCCTGATCTACCATACATCAAGATTACACTCAGGAGTCCCATCA
AGGTTCAGTGGCAGTGGGTCTGGAACAGATTATTCTCTCACCATTAGCAACCTGGAGCAAGAAGATATTGCCACTTAC
TTTTGCCAACAGGGTAATACGCTTCCGTACACGTTCGGAGGGGGGACTAAGTTGGAAATAACAGGAGGAGGTGGGTCT
GGAGGTGGAGGATCTGGTGGAGGTGGGTCTGGAGGAGGTGGGTCTGAGGTGAAACTGCAGGAGTCAGGACCTGGCCTG
GTGGCGCCCTCACAGAGCCTGTCCGTCACATGCACTGTCTCAGGGGTCTCATTACCCGACTATGGTGTAAGCTGGATT
CGCCAGCCTCCACGAAAGGGTCTGGAGTGGCTGGGAGTAATATGGGGTAGTGAAACCACATACTATAATTCAGCTCTC
AAATCCAGACTGACCATCATCAAGGACAACTCCAAGAGCCAAGTTTTCTTAAAAATGAACAGTCTGCAAACTGATGAC
ACAGCCATTTACTACTGTGCCAAACATTATTACTACGGTGGTAGCTATGCTATGGACTACTGGGGTCAAGGAACCTCA
GTCACCGTCTCCTCAGACTACAAAGACGATGACGACAAGATTGAAGTTATGTATCCTCCTCCTTACCTAGACAATGAG
AAGAGCAATGGAACCATTATCCATGTGAAAGGGAAACACCTTTGTCCAAGTCCCCTATTTCCCGGACCTTCTAAGCCC
TTTTGGGTGCTGGTGGTGGTTGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCGCCGCCCCGGGCCCACCCGCAAG
CATTACCAGCCCTATGCCCCACCACGCGACTTCGCAGCCTATCGCTCCAAACGGGGCAGAAAGAAACTCCTGTATATA
TTCAAACAACCATTTATGAGACCAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGAGACGTGGCCGGGACCCTGAGATGGGG
GGAAAGCCGCAGAGAAGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGTCTCAGTACAGCCACC
AAGGACACCTACGACGCCCTTCACATGCAGGCCCTGCCCCCTCGC
SEQID NO. 273
TATTAATAGTAATCAATTACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTTCCCATAGTAACGCCAATA
GGGACTTTCCATTGACGTCAATGGGTGGAGTATTTACGGTAAACTGCCCACTTGGCAGTACATCAAGTGTATCATATG
CCAAGTACGCCCCCTATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCGGTTTTGGCAGTACATCAATGGG
CGTGGATAGCGGTTTGACTCACGGGGATTTCCAAGTCTCCACCCCATTGACGTCAATGGGAGTTTGTTTTGGCACCAA
AATCAACGGGACTTTCCAAAATGTCGTAACAACTCCGCCCCATTGACGCAAATGGGCGGTAGGCGTGTACGGTGGGAG
GTCTATATAAGCAGAGCTCGCGGCCGCGCCACCATGCCACCTCCTCGCCTCCTCTTCTTCCTCCTCTTCCTCACCCCC
ATGGAAGTCAGGCCCGAGGAACCTCTAGTGGTGAAGGTGGAAGAGGGAGATAACGCTGTGCTGCAGTGCCTCAAGGGG
ACCTCAGATGGCCCCACTCAGCAGCTGACTTGGTCTCGGGAGTCCCCGCTTAAACCCTTCTTAAAACTCAGCCTGGGG
CTGCCAGGCCTGGGAATCCACATGAGGCCCCTGGCCATCTGGCTTTTCATCTTCAACGTCTCTCAACAGATGGGGGGC
TTCTACCTGTGCCAGCCGGGGCCCCCCTCTGAGAAGGCCTGGCAGCCTGGCTGGACAGTCAATGTGGAGGGCAGCGGG
GAGCTGTTCCGGTGGAATGTTTCGGACCTAGGTGGCCTGGGCTGTGGCCTGAAGAACAGGTCCTCAGAGGGCCCCAGC
TCCCCTTCCGGGAAGCTCATGAGCCCCAAGCTGTATGTGTGGGCCAAAGACCGCCCTGAGATCTGGGAGGGAGAGCCT
CCGTGTCTCCCACCGAGGGACAGCCTGAACCAGAGCCTCAGCCAGGACCTCACCATGGCCCCTGGCTCCACACTCTGG
CTGTCCTGTGGGGTACCCCCTGACTCTGTGTCCAGGGGCCCCCTCTCCTGGACCCATGTGCACCCCAAGGGGCCTAAG
TCATTGCTGAGCCTAGAGCTGAAGGACGATCGCCCGGCCAGAGATATGTGGGTAATGGAGACGGGTCTGTTGTTGCCC
CGGGCCACAGCTCAAGACGCTGGAAAGTATTATTGTCACCGTGGCAACCTGACCATGTCATTCCACCTGGAGATCACT
GCTCGGCCAGGGGGAGGTGGGTCTGGAGGTGGAGGATCTGGTGGAGGTGGGTCTGGTGGAGGTGGGTCTGAGGTGCAG
CTGGTGGAGTCTGGTGGTGGTCTTGTTCAACCCGGTGGTTCTCTTCGTCTTTCTTGTGCTGCTTCTGGTTTTAATATT
AAAGATACTTATATTCATTGGGTTCGTCAAGCTCCTGGTAAAGGTCTTGAATGGGTTGCTCGTATTTATCCTACTAAT
GGTTATACTCGTTATGCTGATTCTGTTAAAGGTCGTTTTACTATTTCTGCTGATACTTCTAAAAATACTGCTTATCTT
CAAATGAACTCTCTTCGTGCTGAAGATACTGCTGTTTATTATTGTTCTCGTTGGGGTGGTGATGGTTTTTATGCTATG
GATTATTGGGGTCAAGGTACTCTTGTCACCGTCTCCTCAGCTAGCACCGGGGGAGGTGGGTCTGGAGGTGGAGGATCT
GGTGGAGGTGGGTCTGACATCCAGATGACCCAGTCTCCTTCTTCTCTTTCTGCTTCTGTTGGTGATCGTGTTACTATT
ACTTGTCGTGCTTCTCAAGATGTTAATACTGCTGTTGCTTGGTATCAACAAAAACCCGGTAAAGCTCCTAAACTTCTT
ATTTATTCTGCTTCTTTTCTTTATTCTGGTGTTCCTTCTCGTTTTTCTGGTTCTCGTTCTGGTACTGATTTTACTCTT
ACTATTTCTTCTCTTCAACCTGAAGATTTTGCTACTTATTATTGTCAACAACATTATACTACTCCTCCTACTTTTGGT
CAAGGTACCAAGGTGGAGATCAAACGTACGCATCATCACCATCACCAT
SEQID NO. 274
GGGAGAAGCTAGACTTAAAATCTTCCATTGCAGCTGTAAACACATCTGGACAATAGTCTGTTTTCTGCATTTGTGAAT
CCCACACCCATGGAACTATGAATCGTGCATCAGAGTTATTTAAAACCACCGTGCATGGAGTGAACCAATACCGAGGTG
TTTGCTTATCATTTTCCTTTGAGCACACAGCACAGCCTTGAACTCAGTGACACTCCTAAGAGGGCTCTAGGGTCAGGC
CAACTTAGATGAGATGCTAGTCTTTAGCTAAAGATGCCCTTCCACCCCCGTTGCACGACCTTGCTTCTCAGTCTTTGT
TGAGTCTTCTGGGGGAGAATCCCCCTAGAGGACTCAGTTTACAAAACCCTAAGTGAGACCACTGCCAAGAAGTGCTTG
CTCACCCCTCCTGCCGCGGCAGGGAATCCCCCTTTCCTTGTACAGGCAAAACACAAAAAAGGACTCATAAGTGAAGCC
TGATCCTTCTCACCAAACACTGCCCACACCTCCTAGTAATTGAACTTGAAAAAAAAAACTGGTTTGAAAAATTACCGC
AAACCATATTGTCATAAAAAAAAAAAAAAACACTTCCTATATGAGATCACAGAACAGAGTAGGCACAAGTTCCTGCTG
AGCAGATCAGCCTAATGCTTAAATAGAACAACTCCTGGCTGTCATTGACATTGTCTAAAAGCCAAGATGACAGACTGA
GAGGCCTGAGCCCTTGTTCTGGCATTCTCCCAGGAAGATGCAGTAAAGGGGTTGACCCAATATACGCGGCCGCGCCAC
CATGCCACCTCCTCGCCTCCTCTTCTTCCTCCTCTTCCTCACCCCCATGGAAGTCAGGCCCGAGGAACCTCTAGTGGT
GAAGGTGGAAGAGGGAGATAACGCTGTGCTGCAGTGCCTCAAGGGGACCTCAGATGGCCCCACTCAGCAGCTGACTTG
GTCTCGGGAGTCCCCGCTTAAACCCTTCTTAAAACTCAGCCTGGGGCTGCCAGGCCTGGGAATCCACATGAGGCCCCT
GGCCATCTGGCTTTTCATCTTCAACGTCTCTCAACAGATGGGGGGCTTCTACCTGTGCCAGCCGGGGCCCCCCTCTGA
GAAGGCCTGGCAGCCTGGCTGGACAGTCAATGTGGAGGGCAGCGGGGAGCTGTTCCGGTGGAATGTTTCGGACCTAGG
TGGCCTGGGCTGTGGCCTGAAGAACAGGTCCTCAGAGGGCCCCAGCTCCCCTTCCGGGAAGCTCATGAGCCCCAAGCT
GTATGTGTGGGCCAAAGACCGCCCTGAGATCTGGGAGGGAGAGCCTCCGTGTCTCCCACCGAGGGACAGCCTGAACCA
GAGCCTCAGCCAGGACCTCACCATGGCCCCTGGCTCCACACTCTGGCTGTCCTGTGGGGTACCCCCTGACTCTGTGTC
CAGGGGCCCCCTCTCCTGGACCCATGTGCACCCCAAGGGGCCTAAGTCATTGCTGAGCCTAGAGCTGAAGGACGATCG
CCCGGCCAGAGATATGTGGGTAATGGAGACGGGTCTGTTGTTGCCCCGGGCCACAGCTCAAGACGCTGGAAAGTATTA
TTGTCACCGTGGCAACCTGACCATGTCATTCCACCTGGAGATCACTGCTCGGCCAGGGGGAGGTGGGTCTGGAGGTGG
AGGATCTGGTGGAGGTGGGTCTGGTGGAGGTGGGTCTGAGGTGCAGCTGGTGGAGTCTGGTGGTGGTCTTGTTCAACC
CGGTGGTTCTCTTCGTCTTTCTTGTGCTGCTTCTGGTTTTAATATTAAAGATACTTATATTCATTGGGTTCGTCAAGC
TCCTGGTAAAGGTCTTGAATGGGTTGCTCGTATTTATCCTACTAATGGTTATACTCGTTATGCTGATTCTGTTAAAGG
TCGTTTTACTATTTCTGCTGATACTTCTAAAAATACTGCTTATCTTCAAATGAACTCTCTTCGTGCTGAAGATACTGC
TGTTTATTATTGTTCTCGTTGGGGTGGTGATGGTTTTTATGCTATGGATTATTGGGGTCAAGGTACTCTTGTCACCGT
CTCCTCAGCTAGCACCGGGGGAGGTGGGTCTGGAGGTGGAGGATCTGGTGGAGGTGGGTCTGACATCCAGATGACCCA
GTCTCCTTCTTCTCTTTCTGCTTCTGTTGGTGATCGTGTTACTATTACTTGTCGTGCTTCTCAAGATGTTAATACTGC
TGTTGCTTGGTATCAACAAAAACCCGGTAAAGCTCCTAAACTTCTTATTTATTCTGCTTCTTTTCTTTATTCTGGTGT
TCCTTCTCGTTTTTCTGGTTCTCGTTCTGGTACTGATTTTACTCTTACTATTTCTTCTCTTCAACCTGAAGATTTTGC
TACTTATTATTGTCAACAACATTATACTACTCCTCCTACTTTTGGTCAAGGTACCAAGGTGGAGATCAAACGTACGCA
TCATCACCATCACCAT
SEQID NO. 275
AATTTAAGGCAGGATGTCTCAGAGTCTGGGAAAATCCCACTTTCCTCCTGCTACACCTTACAGTTGTGAGAAAGCACA
TTTCAGACAACAGGGAAAACCCATACTTCACCACAACAACACACTATACATTGTCTGGTCCACTGGAGCATAAATTAA
AGAGAAACAATGTAGTCAAGCAAGTAGGCGGCAAGAGGAAGGGGGCGGAGACATCATCAGGGAGTATAAACTCTGAGA
TGCCTCAGAGCCTCACAGACTCAACAAGAGCTCCAGCAAAGACTTTCACTGTAGCTTGACTTGACCTGAGATTAACTA
GGGAATCTTGAGAATAAAGGCGGCCGCGCCACCATGCCACCTCCTCGCCTCCTCTTCTTCCTCCTCTTCCTCACCCCC
ATGGAAGTCAGGCCCGAGGAACCTCTAGTGGTGAAGGTGGAAGAGGGAGATAACGCTGTGCTGCAGTGCCTCAAGGGG
ACCTCAGATGGCCCCACTCAGCAGCTGACTTGGTCTCGGGAGTCCCCGCTTAAACCCTTCTTAAAACTCAGCCTGGGG
CTGCCAGGCCTGGGAATCCACATGAGGCCCCTGGCCATCTGGCTTTTCATCTTCAACGTCTCTCAACAGATGGGGGGC
TTCTACCTGTGCCAGCCGGGGCCCCCCTCTGAGAAGGCCTGGCAGCCTGGCTGGACAGTCAATGTGGAGGGCAGCGGG
GAGCTGTTCCGGTGGAATGTTTCGGACCTAGGTGGCCTGGGCTGTGGCCTGAAGAACAGGTCCTCAGAGGGCCCCAGC
TCCCCTTCCGGGAAGCTCATGAGCCCCAAGCTGTATGTGTGGGCCAAAGACCGCCCTGAGATCTGGGAGGGAGAGCCT
CCGTGTCTCCCACCGAGGGACAGCCTGAACCAGAGCCTCAGCCAGGACCTCACCATGGCCCCTGGCTCCACACTCTGG
CTGTCCTGTGGGGTACCCCCTGACTCTGTGTCCAGGGGCCCCCTCTCCTGGACCCATGTGCACCCCAAGGGGCCTAAG
TCATTGCTGAGCCTAGAGCTGAAGGACGATCGCCCGGCCAGAGATATGTGGGTAATGGAGACGGGTCTGTTGTTGCCC
CGGGCCACAGCTCAAGACGCTGGAAAGTATTATTGTCACCGTGGCAACCTGACCATGTCATTCCACCTGGAGATCACT
GCTCGGCCAGGGGGAGGTGGGTCTGGAGGTGGAGGATCTGGTGGAGGTGGGTCTGGTGGAGGTGGGTCTGAGGTGCAG
CTGGTGGAGTCTGGTGGTGGTCTTGTTCAACCCGGTGGTTCTCTTCGTCTTTCTTGTGCTGCTTCTGGTTTTAATATT
AAAGATACTTATATTCATTGGGTTCGTCAAGCTCCTGGTAAAGGTCTTGAATGGGTTGCTCGTATTTATCCTACTAAT
GGTTATACTCGTTATGCTGATTCTGTTAAAGGTCGTTTTACTATTTCTGCTGATACTTCTAAAAATACTGCTTATCTT
CAAATGAACTCTCTTCGTGCTGAAGATACTGCTGTTTATTATTGTTCTCGTTGGGGTGGTGATGGTTTTTATGCTATG
GATTATTGGGGTCAAGGTACTCTTGTCACCGTCTCCTCAGCTAGCACCGGGGGAGGTGGGTCTGGAGGTGGAGGATCT
GGTGGAGGTGGGTCTGACATCCAGATGACCCAGTCTCCTTCTTCTCTTTCTGCTTCTGTTGGTGATCGTGTTACTATT
ACTTGTCGTGCTTCTCAAGATGTTAATACTGCTGTTGCTTGGTATCAACAAAAACCCGGTAAAGCTCCTAAACTTCTT
ATTTATTCTGCTTCTTTTCTTTATTCTGGTGTTCCTTCTCGTTTTTCTGGTTCTCGTTCTGGTACTGATTTTACTCTT
ACTATTTCTTCTCTTCAACCTGAAGATTTTGCTACTTATTATTGTCAACAACATTATACTACTCCTCCTACTTTTGGT
CAAGGTACCAAGGTGGAGATCAAACGTACGCATCATCACCATCACCAT
SEQID NO. 276
TAACGAAGACAGGGCCATGTAGAGGGCCCCAGGGAGTGAAAGGGCCTCCAGGACCTCCAGGTATGGAATACAGGGGAC
GTTTAAGAAGATATGGCCACACACTGGGGCCCTGAGAAGTGAGAGCTTCATGAAAAAAATCAGGGACCCCAGAGTTCC
TTGGAAGCCAAGACTGAAACCAGCATTATGAGTCTCCGGGTCAGAATGAAAGAAGAAGGCCTGCCCCAGTGGGGTCTG
TGAATTCCCGGGGGTGATTTCACTCCCCGGGGCTGTCCCAGGCTTGTCCCTGCTACCCCCACCCAGCCTTTCCTGAGG
CCTCAAGCCTGCCACCAAGCCCCCAGCTCCTTCTCCCCGCAGGGACCCAAACACAGGCCTCGGGACTCAACACAGCTT
TTCCCTCCAACCCCGTTTTCTCTCCCTCAAGGACTCAGCTTTCTGAGGCCCCTCCCAGTTCTAGTTCTATCTTTTTCC
TGCATCCTGTCTGGAAGTTAGAAGGAAACAGACCACAGACCCGGTCCCCAAAAGAAATGGAGGCAATAGGTTTTGAGG
GGCATGGGGACGGGGTTCAGCCTCCAGGGTCCTACACACAAATCAGTCAGTGGCCCAGAAGACCCCCTCGGAATCGGA
GCAGGGAGGATGGGGAGTGTGAGGGGTATCCTTGATGCTTGTGTGTCCCCAACTTTCCAAATCCCCGCCCCCGCGATG
GAGAAGAAACCGAGACAGAAGGTGCAGGGCCCACTACCGCTTCCTCCAGATGAGCTCATGGGTTTCTCCACCAAGGAA
GTTTTCCGCTGGTTGAATGATTCTTTCCCCGCCCTCCTCTCGCCCCAGGGACATATAAAGGCAGTTGTTGGCACACCC
AGCCAGCAGACGCTCCCTCAGCAAGGACAGCAGAGGACCAGCTAAGAGGGAGAGAAGCAACTACAGACCCCCCCTGAA
AACAACCCTCAGACGCCACATCCCCTGACAAGCTGCCAGGCAGGTTCTCTTCCTCTCACATACTGACCCACGGCTCCA
CCCTCTCTCCCCTGGAAAGGACACGCGGCCGCGCCACCATGCCACCTCCTCGCCTCCTCTTCTTCCTCCTCTTCCTCA
CCCCCATGGAAGTCAGGCCCGAGGAACCTCTAGTGGTGAAGGTGGAAGAGGGAGATAACGCTGTGCTGCAGTGCCTCA
AGGGGACCTCAGATGGCCCCACTCAGCAGCTGACTTGGTCTCGGGAGTCCCCGCTTAAACCCTTCTTAAAACTCAGCC
TGGGGCTGCCAGGCCTGGGAATCCACATGAGGCCCCTGGCCATCTGGCTTTTCATCTTCAACGTCTCTCAACAGATGG
GGGGCTTCTACCTGTGCCAGCCGGGGCCCCCCTCTGAGAAGGCCTGGCAGCCTGGCTGGACAGTCAATGTGGAGGGCA
GCGGGGAGCTGTTCCGGTGGAATGTTTCGGACCTAGGTGGCCTGGGCTGTGGCCTGAAGAACAGGTCCTCAGAGGGCC
CCAGCTCCCCTTCCGGGAAGCTCATGAGCCCCAAGCTGTATGTGTGGGCCAAAGACCGCCCTGAGATCTGGGAGGGAG
AGCCTCCGTGTCTCCCACCGAGGGACAGCCTGAACCAGAGCCTCAGCCAGGACCTCACCATGGCCCCTGGCTCCACAC
TCTGGCTGTCCTGTGGGGTACCCCCTGACTCTGTGTCCAGGGGCCCCCTCTCCTGGACCCATGTGCACCCCAAGGGGC
CTAAGTCATTGCTGAGCCTAGAGCTGAAGGACGATCGCCCGGCCAGAGATATGTGGGTAATGGAGACGGGTCTGTTGT
TGCCCCGGGCCACAGCTCAAGACGCTGGAAAGTATTATTGTCACCGTGGCAACCTGACCATGTCATTCCACCTGGAGA
TCACTGCTCGGCCAGGGGGAGGTGGGTCTGGAGGTGGAGGATCTGGTGGAGGTGGGTCTGGTGGAGGTGGGTCTGAGG
TGCAGCTGGTGGAGTCTGGTGGTGGTCTTGTTCAACCCGGTGGTTCTCTTCGTCTTTCTTGTGCTGCTTCTGGTTTTA
ATATTAAAGATACTTATATTCATTGGGTTCGTCAAGCTCCTGGTAAAGGTCTTGAATGGGTTGCTCGTATTTATCCTA
CTAATGGTTATACTCGTTATGCTGATTCTGTTAAAGGTCGTTTTACTATTTCTGCTGATACTTCTAAAAATACTGCTT
ATCTTCAAATGAACTCTCTTCGTGCTGAAGATACTGCTGTTTATTATTGTTCTCGTTGGGGTGGTGATGGTTTTTATG
CTATGGATTATTGGGGTCAAGGTACTCTTGTCACCGTCTCCTCAGCTAGCACCGGGGGAGGTGGGTCTGGAGGTGGAG
GATCTGGTGGAGGTGGGTCTGACATCCAGATGACCCAGTCTCCTTCTTCTCTTTCTGCTTCTGTTGGTGATCGTGTTA
CTATTACTTGTCGTGCTTCTCAAGATGTTAATACTGCTGTTGCTTGGTATCAACAAAAACCCGGTAAAGCTCCTAAAC
TTCTTATTTATTCTGCTTCTTTTCTTTATTCTGGTGTTCCTTCTCGTTTTTCTGGTTCTCGTTCTGGTACTGATTTTA
CTCTTACTATTTCTTCTCTTCAACCTGAAGATTTTGCTACTTATTATTGTCAACAACATTATACTACTCCTCCTACTT
TTGGTCAAGGTACCAAGGTGGAGATCAAACGTACGCATCATCACCATCACCAT
SEQID NO. 277
GATATCGAATTAGGAGGAAAACTGTTTCATACAGAAGGCGTCAATTAGGAGGAAAAACTGTTTCATACAGAAGGCGTC
AATTAGGAGGAAAAACTGTTTCATACAGAAGGCGTCAATTGGTCCCATCGAATTAGGAGGAAAAACTGTTTCATACAG
AAGGCGTCAATTAGGAGGAAAAACTGTTTCATACAGAAGGCGTCAATTAGGAGGAAAAACTGTTTCATACAGAAGGCG
TCAATTGGTCCCGGGACATTTTGACACCCCCATAATATTTTTCCAGAATTAACAGTATAAATTGCATCTCTTGTTCAA
GAGTTCCCTATCACTCTCTTTAATCACTACTCACAGTAACCTCAACTCCTGGCGGCCGCGCCACCATGCCACCTCCTC
GCCTCCTCTTCTTCCTCCTCTTCCTCACCCCCATGGAAGTCAGGCCCGAGGAACCTCTAGTGGTGAAGGTGGAAGAGG
GAGATAACGCTGTGCTGCAGTGCCTCAAGGGGACCTCAGATGGCCCCACTCAGCAGCTGACTTGGTCTCGGGAGTCCC
CGCTTAAACCCTTCTTAAAACTCAGCCTGGGGCTGCCAGGCCTGGGAATCCACATGAGGCCCCTGGCCATCTGGCTTT
TCATCTTCAACGTCTCTCAACAGATGGGGGGCTTCTACCTGTGCCAGCCGGGGCCCCCCTCTGAGAAGGCCTGGCAGC
CTGGCTGGACAGTCAATGTGGAGGGCAGCGGGGAGCTGTTCCGGTGGAATGTTTCGGACCTAGGTGGCCTGGGCTGTG
GCCTGAAGAACAGGTCCTCAGAGGGCCCCAGCTCCCCTTCCGGGAAGCTCATGAGCCCCAAGCTGTATGTGTGGGCCA
AAGACCGCCCTGAGATCTGGGAGGGAGAGCCTCCGTGTCTCCCACCGAGGGACAGCCTGAACCAGAGCCTCAGCCAGG
ACCTCACCATGGCCCCTGGCTCCACACTCTGGCTGTCCTGTGGGGTACCCCCTGACTCTGTGTCCAGGGGCCCCCTCT
CCTGGACCCATGTGCACCCCAAGGGGCCTAAGTCATTGCTGAGCCTAGAGCTGAAGGACGATCGCCCGGCCAGAGATA
TGTGGGTAATGGAGACGGGTCTGTTGTTGCCCCGGGCCACAGCTCAAGACGCTGGAAAGTATTATTGTCACCGTGGCA
ACCTGACCATGTCATTCCACCTGGAGATCACTGCTCGGCCAGGGGGAGGTGGGTCTGGAGGTGGAGGATCTGGTGGAG
GTGGGTCTGGTGGAGGTGGGTCTGAGGTGCAGCTGGTGGAGTCTGGTGGTGGTCTTGTTCAACCCGGTGGTTCTCTTC
GTCTTTCTTGTGCTGCTTCTGGTTTTAATATTAAAGATACTTATATTCATTGGGTTCGTCAAGCTCCTGGTAAAGGTC
TTGAATGGGTTGCTCGTATTTATCCTACTAATGGTTATACTCGTTATGCTGATTCTGTTAAAGGTCGTTTTACTATTT
CTGCTGATACTTCTAAAAATACTGCTTATCTTCAAATGAACTCTCTTCGTGCTGAAGATACTGCTGTTTATTATTGTT
CTCGTTGGGGTGGTGATGGTTTTTATGCTATGGATTATTGGGGTCAAGGTACTCTTGTCACCGTCTCCTCAGCTAGCA
CCGGGGGAGGTGGGTCTGGAGGTGGAGGATCTGGTGGAGGTGGGTCTGACATCCAGATGACCCAGTCTCCTTCTTCTC
TTTCTGCTTCTGTTGGTGATCGTGTTACTATTACTTGTCGTGCTTCTCAAGATGTTAATACTGCTGTTGCTTGGTATC
AACAAAAACCCGGTAAAGCTCCTAAACTTCTTATTTATTCTGCTTCTTTTCTTTATTCTGGTGTTCCTTCTCGTTTTT
CTGGTTCTCGTTCTGGTACTGATTTTACTCTTACTATTTCTTCTCTTCAACCTGAAGATTTTGCTACTTATTATTGTC
AACAACATTATACTACTCCTCCTACTTTTGGTCAAGGTACCAAGGTGGAGATCAAACGTACGCATCATCACCATCACC
AT
SEQID NO. 278
ATGGATTTTCAGGTGCAGATTTTCAGCTTCCTGCTAATCAGTGCCTCAGTCATAATGTCTAGAATGGCCCAGGTCAAA
CTACAGGAGTCAGGGGCTGAGCTGGTGAAGCCTGGGGCCTCAGTGAAGATGTCCTGCAAGGCTTCTGGCTACACATTT
ACCAGTTACAATATGCACTGGGTAAAGCAGACACCTGGACAGGGCCTGGAATGGATTGGAGCTATTTATCCAGGAAAT
GGTGATACTTCCTACAATCAGAAGTTCAAAGGCAAGGCCACATTGACTGCAGACAAATCCTCCAGCACAGCCTACATG
CAGCTCAGCAGCCTGACATCTGAGGACTCTGCGGACTATTACTGTGCAAGATCTAATTATTACGGTAGTAGCTACTGG
TTCTTCGATGTCTGGGGCCAAGGGACCACGGTCACCGTCTCCTCAGGTGGAGGCGGTTCAGGCGGAGGTGGCTCTGGC
GGTGGCGGATCGGACATCGAGCTCACTCAGTCTCCAACAATCCTGTCTGCATCTCCAGGGGAGAAGGTCACAATGACT
TGCAGGGCCAGCTCAAGTGTAAATTACATGGACTGGTACCAGAAGAAGCCAGGATCCTCCCCCAAACCCTGGATTTAT
GCCACATCCAACCTGGCTTCTGGAGTCCCTGCTCGCTTCAGTGGCAGTGGGTCTGGGACCTCTTACTCTCTCACAATC
AGCAGAGTGGAGGCTGAAGATGCTGCCACTTATTACTGCCAGCAGTGGAGTTTTAATCCACCCACGTTCGGAGGGGGG
ACAAAGTTGGAAATAAAACGGGCCGCCGCTGGTGATCCCGCCGAGCCCAAATCTCCTGACAAAACTCACACATGCCCA
CCGTGCCCAGCACCTGAACTCCTGGGGGGACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCATCCCGGGAGGAG
ATGACCAAGAACCAGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTTCCTCTACAGCAAG
CTCACCGTGGACAAGAGCAGGTGGCAGCAGGGGAACGTCTTCTCATGCTCCGTGATGCACGAGGCTCTGCACAACCAC
TACACGCAGAAGAGCCTCTCCCTGTCTCCGGGT
SEQID NO. 279
ATGGATTTTCAGGTGCAGATTTTCAGCTTCCTGCTAATCAGTGCCTCAGTCATAATGTCTAGAATGGCCCAGGTCAAA
CTACAGGAGTCAGGGGCTGAGCTGGTGAAGCCTGGGGCCTCAGTGAAGATGTCCTGCAAGGCTTCTGGCTACACATTT
ACCAGTTACAATATGCACTGGGTAAAGCAGACACCTGGACAGGGCCTGGAATGGATTGGAGCTATTTATCCAGGAAAT
GGTGATACTTCCTACAATCAGAAGTTCAAAGGCAAGGCCACATTGACTGCAGACAAATCCTCCAGCACAGCCTACATG
CAGCTCAGCAGCCTGACATCTGAGGACTCTGCGGACTATTACTGTGCAAGATCTAATTATTACGGTAGTAGCTACTGG
TTCTTCGATGTCTGGGGCCAAGGGACCACGGTCACCGTCTCCTCAGGTGGAGGCGGTTCAGGCGGAGGTGGCTCTGGC
GGTGGCGGATCGGACATCGAGCTCACTCAGTCTCCAACAATCCTGTCTGCATCTCCAGGGGAGAAGGTCACAATGACT
TGCAGGGCCAGCTCAAGTGTAAATTACATGGACTGGTACCAGAAGAAGCCAGGATCCTCCCCCAAACCCTGGATTTAT
GCCACATCCAACCTGGCTTCTGGAGTCCCTGCTCGCTTCAGTGGCAGTGGGTCTGGGACCTCTTACTCTCTCACAATC
AGCAGAGTGGAGGCTGAAGATGCTGCCACTTATTACTGCCAGCAGTGGAGTTTTAATCCACCCACGTTCGGAGGGGGG
ACAAAGTTGGAAATAAAACGGGCCGCCGCTCATCATCACCATCACCAT
SEQID NO. 280
ATGGATTTTCAGGTGCAGATTTTCAGCTTCCTGCTAATCAGTGCCTCAGTCATAATGTCTAGAGACATCGAGCTCACT
CAGTCTCCAACAATCCTGTCTGCATCTCCAGGGGAGAAGGTCACAATGACTTGCAGGGCCAGCTCAAGTGTAAATTAC
ATGGACTGGTACCAGAAGAAGCCAGGATCCTCCCCCAAACCCTGGATTTATGCCACATCCAACCTGGCTTCTGGAGTC
CCTGCTCGCTTCAGTGGCAGTGGGTCTGGGACCTCTTACTCTCTCACAATCAGCAGAGTGGAGGCTGAAGATGCTGCC
ACTTATTACTGCCAGCAGTGGAGTTTTAATCCACCCACGTTCGGAGGGGGGACAAAGTTGGAAATAAAACGGGGTGGA
GGCGGTTCAGGCGGAGGTGGCTCTGGCGGTGGCGGATCGATGGCCCAGGTCAAACTACAGGAGTCAGGGGCTGAGCTG
GTGAAGCCTGGGGCCTCAGTGAAGATGTCCTGCAAGGCTTCTGGCTACACATTTACCAGTTACAATATGCACTGGGTA
AAGCAGACACCTGGACAGGGCCTGGAATGGATTGGAGCTATTTATCCAGGAAATGGTGATACTTCCTACAATCAGAAG
TTCAAAGGCAAGGCCACATTGACTGCAGACAAATCCTCCAGCACAGCCTACATGCAGCTCAGCAGCCTGACATCTGAG
GACTCTGCGGACTATTACTGTGCAAGATCTAATTATTACGGTAGTAGCTACTGGTTCTTCGATGTCTGGGGCCAAGGG
ACCACGGTCACCGTCTCCTCAGCCGCCGCTGGTGATCCCGCCGAGCCCAAATCTCCTGACAAAACTCACACATGCCCA
CCGTGCCCAGCACCTGAACTCCTGGGGGGACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCATCCCGGGAGGAG
ATGACCAAGAACCAGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTTCCTCTACAGCAAG
CTCACCGTGGACAAGAGCAGGTGGCAGCAGGGGAACGTCTTCTCATGCTCCGTGATGCACGAGGCTCTGCACAACCAC
TACACGCAGAAGAGCCTCTCCCTGTCTCCGGGT
SEQID NO. 281
ATGGATTTTCAGGTGCAGATTTTCAGCTTCCTGCTAATCAGTGCCTCAGTCATAATGTCTAGAGACATCGAGCTCACT
CAGTCTCCAACAATCCTGTCTGCATCTCCAGGGGAGAAGGTCACAATGACTTGCAGGGCCAGCTCAAGTGTAAATTAC
ATGGACTGGTACCAGAAGAAGCCAGGATCCTCCCCCAAACCCTGGATTTATGCCACATCCAACCTGGCTTCTGGAGTC
CCTGCTCGCTTCAGTGGCAGTGGGTCTGGGACCTCTTACTCTCTCACAATCAGCAGAGTGGAGGCTGAAGATGCTGCC
ACTTATTACTGCCAGCAGTGGAGTTTTAATCCACCCACGTTCGGAGGGGGGACAAAGTTGGAAATAAAACGGGGTGGA
GGCGGTTCAGGCGGAGGTGGCTCTGGCGGTGGCGGATCGATGGCCCAGGTCAAACTACAGGAGTCAGGGGCTGAGCTG
GTGAAGCCTGGGGCCTCAGTGAAGATGTCCTGCAAGGCTTCTGGCTACACATTTACCAGTTACAATATGCACTGGGTA
AAGCAGACACCTGGACAGGGCCTGGAATGGATTGGAGCTATTTATCCAGGAAATGGTGATACTTCCTACAATCAGAAG
TTCAAAGGCAAGGCCACATTGACTGCAGACAAATCCTCCAGCACAGCCTACATGCAGCTCAGCAGCCTGACATCTGAG
GACTCTGCGGACTATTACTGTGCAAGATCTAATTATTACGGTAGTAGCTACTGGTTCTTCGATGTCTGGGGCCAAGGG
ACCACGGTCACCGTCTCCTCAGCCGCCGCTCATCATCACCATCACCAT
SEQID NO. 282
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTATGGCCCAGGTCAAACTACAGGAGTCAGGGGCTGAGCTGGTG
AAGCCTGGGGCCTCAGTGAAGATGTCCTGCAAGGCTTCTGGCTACACATTTACCAGTTACAATATGCACTGGGTAAAG
CAGACACCTGGACAGGGCCTGGAATGGATTGGAGCTATTTATCCAGGAAATGGTGATACTTCCTACAATCAGAAGTTC
AAAGGCAAGGCCACATTGACTGCAGACAAATCCTCCAGCACAGCCTACATGCAGCTCAGCAGCCTGACATCTGAGGAC
TCTGCGGACTATTACTGTGCAAGATCTAATTATTACGGTAGTAGCTACTGGTTCTTCGATGTCTGGGGCCAAGGGACC
ACGGTCACCGTCTCCTCAGGTGGAGGCGGTTCAGGCGGAGGTGGCTCTGGCGGTGGCGGATCGGACATCGAGCTCACT
CAGTCTCCAACAATCCTGTCTGCATCTCCAGGGGAGAAGGTCACAATGACTTGCAGGGCCAGCTCAAGTGTAAATTAC
ATGGACTGGTACCAGAAGAAGCCAGGATCCTCCCCCAAACCCTGGATTTATGCCACATCCAACCTGGCTTCTGGAGTC
CCTGCTCGCTTCAGTGGCAGTGGGTCTGGGACCTCTTACTCTCTCACAATCAGCAGAGTGGAGGCTGAAGATGCTGCC
ACTTATTACTGCCAGCAGTGGAGTTTTAATCCACCCACGTTCGGAGGGGGGACAAAGTTGGAAATAAAACGGGCCGCC
GCTGGTGATCCCGCCGAGCCCAAATCTCCTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAGGTCACATGCGTG
GTGGTGGACGTGAGCCACGAAGACCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCCCATCGAGAAAACCATCTCCAAAGCCAAA
GGGCAGCCCCGAGAACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAAGCTCACCGTGGACAAGAGCAGGTGGCAG
CAGGGGAACGTCTTCTCATGCTCCGTGATGCACGAGGCTCTGCACAACCACTACACGCAGAAGAGCCTCTCCCTGTCT
CCGGGT
SEQID NO. 283
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTATGGCCCAGGTCAAACTACAGGAGTCAGGGGCTGAGCTGGTG
AAGCCTGGGGCCTCAGTGAAGATGTCCTGCAAGGCTTCTGGCTACACATTTACCAGTTACAATATGCACTGGGTAAAG
CAGACACCTGGACAGGGCCTGGAATGGATTGGAGCTATTTATCCAGGAAATGGTGATACTTCCTACAATCAGAAGTTC
AAAGGCAAGGCCACATTGACTGCAGACAAATCCTCCAGCACAGCCTACATGCAGCTCAGCAGCCTGACATCTGAGGAC
TCTGCGGACTATTACTGTGCAAGATCTAATTATTACGGTAGTAGCTACTGGTTCTTCGATGTCTGGGGCCAAGGGACC
ACGGTCACCGTCTCCTCAGGTGGAGGCGGTTCAGGCGGAGGTGGCTCTGGCGGTGGCGGATCGGACATCGAGCTCACT
CAGTCTCCAACAATCCTGTCTGCATCTCCAGGGGAGAAGGTCACAATGACTTGCAGGGCCAGCTCAAGTGTAAATTAC
ATGGACTGGTACCAGAAGAAGCCAGGATCCTCCCCCAAACCCTGGATTTATGCCACATCCAACCTGGCTTCTGGAGTC
CCTGCTCGCTTCAGTGGCAGTGGGTCTGGGACCTCTTACTCTCTCACAATCAGCAGAGTGGAGGCTGAAGATGCTGCC
ACTTATTACTGCCAGCAGTGGAGTTTTAATCCACCCACGTTCGGAGGGGGGACAAAGTTGGAAATAAAACGGGCCGCC
GCTCATCATCACCATCACCAT
SEQID NO. 284
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTGACATCGAGCTCACTCAGTCTCCAACAATCCTGTCTGCATCT
CCAGGGGAGAAGGTCACAATGACTTGCAGGGCCAGCTCAAGTGTAAATTACATGGACTGGTACCAGAAGAAGCCAGGA
TCCTCCCCCAAACCCTGGATTTATGCCACATCCAACCTGGCTTCTGGAGTCCCTGCTCGCTTCAGTGGCAGTGGGTCT
GGGACCTCTTACTCTCTCACAATCAGCAGAGTGGAGGCTGAAGATGCTGCCACTTATTACTGCCAGCAGTGGAGTTTT
AATCCACCCACGTTCGGAGGGGGGACAAAGTTGGAAATAAAACGGGGTGGAGGCGGTTCAGGCGGAGGTGGCTCTGGC
GGTGGCGGATCGATGGCCCAGGTCAAACTACAGGAGTCAGGGGCTGAGCTGGTGAAGCCTGGGGCCTCAGTGAAGATG
TCCTGCAAGGCTTCTGGCTACACATTTACCAGTTACAATATGCACTGGGTAAAGCAGACACCTGGACAGGGCCTGGAA
TGGATTGGAGCTATTTATCCAGGAAATGGTGATACTTCCTACAATCAGAAGTTCAAAGGCAAGGCCACATTGACTGCA
GACAAATCCTCCAGCACAGCCTACATGCAGCTCAGCAGCCTGACATCTGAGGACTCTGCGGACTATTACTGTGCAAGA
TCTAATTATTACGGTAGTAGCTACTGGTTCTTCGATGTCTGGGGCCAAGGGACCACGGTCACCGTCTCCTCAGCCGCC
GCTGGTGATCCCGCCGAGCCCAAATCTCCTGACAAAACTCACACATGCCCACCGTGCCCAGCACCTGAACTCCTGGGG
GGACCGTCAGTCTTCCTCTTCCCCCCAAAACCCAAGGACACCCTCATGATCTCCCGGACCCCTGAGGTCACATGCGTG
GTGGTGGACGTGAGCCACGAAGACCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCCCATCGAGAAAACCATCTCCAAAGCCAAA
GGGCAGCCCCGAGAACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAAGCTCACCGTGGACAAGAGCAGGTGGCAG
CAGGGGAACGTCTTCTCATGCTCCGTGATGCACGAGGCTCTGCACAACCACTACACGCAGAAGAGCCTCTCCCTGTCT
CCGGGT
SEQID NO. 285
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTGACATCGAGCTCACTCAGTCTCCAACAATCCTGTCTGCATCT
CCAGGGGAGAAGGTCACAATGACTTGCAGGGCCAGCTCAAGTGTAAATTACATGGACTGGTACCAGAAGAAGCCAGGA
TCCTCCCCCAAACCCTGGATTTATGCCACATCCAACCTGGCTTCTGGAGTCCCTGCTCGCTTCAGTGGCAGTGGGTCT
GGGACCTCTTACTCTCTCACAATCAGCAGAGTGGAGGCTGAAGATGCTGCCACTTATTACTGCCAGCAGTGGAGTTTT
AATCCACCCACGTTCGGAGGGGGGACAAAGTTGGAAATAAAACGGGGTGGAGGCGGTTCAGGCGGAGGTGGCTCTGGC
GGTGGCGGATCGATGGCCCAGGTCAAACTACAGGAGTCAGGGGCTGAGCTGGTGAAGCCTGGGGCCTCAGTGAAGATG
TCCTGCAAGGCTTCTGGCTACACATTTACCAGTTACAATATGCACTGGGTAAAGCAGACACCTGGACAGGGCCTGGAA
TGGATTGGAGCTATTTATCCAGGAAATGGTGATACTTCCTACAATCAGAAGTTCAAAGGCAAGGCCACATTGACTGCA
GACAAATCCTCCAGCACAGCCTACATGCAGCTCAGCAGCCTGACATCTGAGGACTCTGCGGACTATTACTGTGCAAGA
TCTAATTATTACGGTAGTAGCTACTGGTTCTTCGATGTCTGGGGCCAAGGGACCACGGTCACCGTCTCCTCAGCCGCC
GCTCATCATCACCATCACCAT
SEQID NO. 286
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTCAGGTGCAGCTGGTGCAGAGCGGCGCCGAGGACAAGAAGCCC
GGCGAGAGCGTGAAGATCAGCTGCAAGGCCAGCGGCTACACCTTCACCAACTACGGCATGAACTGGGTGAGGCAGGCC
CCCGGCCAGGGCCTGAAGTGGATGGGCTGGATCAACACCTACACCGGCGAGAGCACCTACGCCGACGACTTCAAGGGC
AGGTTCGCCTTCAGCCTGGACACCAGCGCCAGCACCGCCTACCTGCAGCTGAGCAGCCTGAGGGGCGAGGACACCGCC
GTGTACTTCTGCGCCAGGTTCGCCATCAAGGGCGACTACTGGGGCCAGGGCACCACCGTGACCGTGAGCAGCGCCAGC
ACCGGCGGCGGCGGCAGCGGCGGCGGCGGCAGCGGCGGCGGCGGCAGCGGCGGCGGCGGCAGCGACATCGTGATGACC
CAGAGCCCCCTGAGCCTGGAGGTGAGCCCCGGCGAGCCCGCCAGCATCAGCTGCAGGAGCACCAAGAGCCTGCTGCAC
AGCGACGGCATCACCTACCTGTACTGGTACCTGCAGAAGCCCGGCCAGAGCCCCCAGCTGCTGATCTACCAGCTGAGC
AACCTGGCCAGCGGCGTGCCCGACAGGTTCAGCAGCAGCGGCAGCGGCACCGACTTCACCCTGAAGATCAGCAGGGTG
GAGGCCGAGGACGAGGGCACCTACTACTGCGCCCAGAACCTGGAGATCCCCAGGACCTTCGGCCAGGGCACCAAGCTG
GAGATCAAGAGGACCCATCATCACCATCACCAT
SEQID NO. 287
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTCAGGTTCAGCTGGTGCAGTCTGGTGCTGAGGATGTGAAGCCT
GATGCCTCAGTGAAGCTCTCCTGCAAGGCTTCTGGTTACACATTCACTGACTACTACATGCACTGGGTGCGTCAGGCC
CCTGGTCAAGGTCTTGAGTGGATGGGTCGTGTTAATCCTAACCGGAGGGGTACTACCTACAACCAGAAATTCGAGGGC
CGTGTCACCATGACCACAGACACATCCACGAGCACAGCCTACATGCAGCTGAGTAGCCTGCGTGGTGAAGACACGGCC
GTGTATTACTGTGCGCGTGCGAACTGGCTTGACTACTGGGGCCAGGGCACCACCGTCACCGTCTCCTCCGCCTCCACC
GGGGGAGGTGGGTCTGGAGGTGGAGGATCTGGTGGAGGTGGGTCTGGTGGAGGTGGGTCTGACATCCAGATGACCCAG
TCTCCATCCTCCCTGGAGGCATCTGTAGGAGACAGAGTCACCATCACTTGCAGTGTCAGCTCAAGTGTATCCTCCATT
TACTTGCACTGGTATCAGCAGAAACCAGGGAAAAGCCCTAAGCTCCTGATCTATAGCACATCCAACTTGGCTTCTGGA
GTCCCAGATAGGTTCAGTGGCAGTGGATCTGGGACAGATTTCACTCTCACCATCAGCAGTCTGCAAGCCGAAGATGAG
GGCACTTACTACTGTCAAGTCTACAGTGGTTACCCGCTCACGTTCGGCGGAGGGACCAAGCTGGAGATCAAACGAACT
CATCATCACCATCACCAT
SEQID NO. 288
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTCAGGTGCAGCTGCAGGAGAGCGGCCCCGGCGACGTGAAGCCC
AGCGAGACCCTGAGCCTGACCTGCACCGTGAGCGGCGGCAGCGTGAGCAGCGGCGACTACTACTGGACCTGGATCAGG
CAGAGCCCCGGCAAGGGCCTGGAGTGGATCGGCCACATCTACTACAGCGGCAACACCAACTACAACCCCAGCCTGAAG
AGCAGGCTGACCATCAGCATCGACACCAGCAAGACCACCTTCAGCCTGCAGCTGAGCAGCGTGACCGGCGAGGACACC
GCCATCTACTACTGCGTGAGGGACAGGGTGACCGGCGCCTTCGACATCTGGGGCCAGGGCACCACCGTGACCGTGAGC
AGCGCCAGCACCGGCGGCGGCGGCAGCGGCGGCGGCGGCAGCGGCGGCGGCGGCAGCGGCGGCGGCGGCAGCGACATC
CAGATGACCCAGAGCCCCAGCAGCCTGGAGGCCAGCGTGGGCGACAGGGTGACCATCACCTGCCAGGCCAGCCAGGAC
ATCAGCAACTACCTGAACTGGTACCAGCAGAAGCCCGGCAAGAGCCCCAAGCTGCTGATCTACGACGCCAGCAACCTG
GAGACCGGCGTGCCCGACAGGTTCAGCGGCAGCGGCAGCGGCACCGACTTCACCTTCACCATCAGCAGCCTGCAGGCC
GAGGACGAGGGCACCTACTTCTGCCAGCACTTCGACCACCTGCCCCTGGCCTTCGGCGGCGGCACCAAGCTGGAGATC
AAGAGGACCCATCATCACCATCACCAT
SEQID NO. 289
ATGGAGACAGACACACTCCTGCTATGGGTACTGCTGCTCTGGGTTCCAGGTTCCACTGGTGACATCGTGCTGACCCAG
AGCCCCCCCAGCCTGGCCATGAGCCTGGGCAAGAGGGCCACCATCAGCTGCAGGGCCAGCGAGAGCGTGACCATCCTG
GGCAGCCACCTGATCCACTGGTACCAGCAGAAGCCCGGCCAGCCCCCCACCCTGCTGATCCAGCTGGCCAGCAACGTG
CAGACCGGCGTGCCCGCCAGGTTCAGCGGCAGCGGCAGCAGGACCGACTTCACCCTGACCATCGACCCCGTGGAGGAG
GACGACGTGGCCGTGTACTACTGCCTGCAGAGCAGGACCATCCCCAGGACCTTCGGCGGCGGCACCAAGCTGGAGATC
AAGGGCAGCACCAGCGGCAGCGGCAAGCCCGGCAGCGGCGAGGGCAGCACCAAGGGCCAGATCCAGCTGGTGCAGAGC
GGCCCCGAGCTGAAGAAGCCCGGCGAGACCGTGAAGATCAGCTGCAAGGCCAGCGGCTACACCTTCACCGACTACAGC
ATCAACTGGGTGAAGAGGGCCCCCGGCAAGGGCCTGAAGTGGATGGGCTGGATCAACACCGAGACCAGGGAGCCCGCC
TACGCCTACGACTTCAGGGGCAGGTTCGCCTTCAGCCTGGAGACCAGCGCCAGCACCGCCTACCTGCAGATCAACAAC
CTGAAGTACGAGGACACCGCCACCTACTTCTGCGCCCTGGACTACAGCTACGCCATGGACTACTGGGGCCAGGGCACC
AGCGTGACCGTGAGCAGCGGAGGAGGTGGGTCTGGAGGTGGAGGATCTGGTGGAGGTGGGTCTCATCATCACCATCAC
CAT
SEQID NO. 290
ATGGAGTTTGGGCTGAGCTGGGTTTTCCTCGTTGCTCTTTTTAGAGGTGTCCAGTGTCAGATCCAGCTGGTGCAGAGC
GGCCCCGAGCTGAAGAAGCCCGGCGAGACCGTGAAGATCAGCTGCAAGGCCAGCGGCTACACCTTCACCGACTACAGC
ATCAACTGGGTGAAGAGGGCCCCCGGCAAGGGCCTGAAGTGGATGGGCTGGATCAACACCGAGACCAGGGAGCCCGCC
TACGCCTACGACTTCAGGGGCAGGTTCGCCTTCAGCCTGGAGACCAGCGCCAGCACCGCCTACCTGCAGATCAACAAC
CTGAAGTACGAGGACACCGCCACCTACTTCTGCGCCCTGGACTACAGCTACGCCATGGACTACTGGGGCCAGGGCACC
AGCGTGACCGTGAGCAGCGGCAGCACCAGCGGCAGCGGCAAGCCCGGCAGCGGCGAGGGCAGCACCAAGGGC
GACATCGTGCTGACCCAGAGCCCCCCCAGCCTGGCCATGAGCCTGGGCAAGAGGGCCACCATCAGCTGCAGGGCCAGC
GAGAGCGTGACCATCCTGGGCAGCCACCTGATCCACTGGTACCAGCAGAAGCCCGGCCAGCCCCCCACCCTGCTGATC
CAGCTGGCCAGCAACGTGCAGACCGGCGTGCCCGCCAGGTTCAGCGGCAGCGGCAGCAGGACCGACTTCACCCTGACC
ATCGACCCCGTGGAGGAGGACGACGTGGCCGTGTACTACTGCCTGCAGAGCAGGACCATCCCCAGGACCTTCGGCGGC
GGCACCAAGCTGGAGATCAAGGGAGGAGGTGGGTCTGGAGGTGGAGGATCTGGTGGAGGTGGGTCTCATCATCACCAT
CACCAT
SEQID NO. 291
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTGACATCGTGCTGACCCAGAGCCCCCCCAGCCTGGCCATGAGC
CTGGGCAAGAGGGCCACCATCAGCTGCAGGGCCAGCGAGAGCGTGACCATCCTGGGCAGCCACCTGATCCACTGGTAC
CAGCAGAAGCCCGGCCAGCCCCCCACCCTGCTGATCCAGCTGGCCAGCAACGTGCAGACCGGCGTGCCCGCCAGGTTC
AGCGGCAGCGGCAGCAGGACCGACTTCACCCTGACCATCGACCCCGTGGAGGAGGACGACGTGGCCGTGTACTACTGC
CTGCAGAGCAGGACCATCCCCAGGACCTTCGGCGGCGGCACCAAGCTGGAGATCAAGGGCAGCACCAGCGGCAGCGGC
AAGCCCGGCAGCGGCGAGGGCAGCACCAAGGGCCAGATCCAGCTGGTGCAGAGCGGCCCCGAGCTGAAGAAGCCCGGC
GAGACCGTGAAGATCAGCTGCAAGGCCAGCGGCTACACCTTCACCGACTACAGCATCAACTGGGTGAAGAGGGCCCCC
GGCAAGGGCCTGAAGTGGATGGGCTGGATCAACACCGAGACCAGGGAGCCCGCCTACGCCTACGACTTCAGGGGCAGG
TTCGCCTTCAGCCTGGAGACCAGCGCCAGCACCGCCTACCTGCAGATCAACAACCTGAAGTACGAGGACACCGCCACC
TACTTCTGCGCCCTGGACTACAGCTACGCCATGGACTACTGGGGCCAGGGCACCAGCGTGACCGTGAGCAGCGGAGGA
GGTGGGTCTGGAGGTGGAGGATCTGGTGGAGGTGGGTCTCATCATCACCATCACCAT
SEQID NO. 292
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTCAGATCCAGCTGGTGCAGAGCGGCCCCGAGCTGAAGAAGCCC
GGCGAGACCGTGAAGATCAGCTGCAAGGCCAGCGGCTACACCTTCACCGACTACAGCATCAACTGGGTGAAGAGGGCC
CCCGGCAAGGGCCTGAAGTGGATGGGCTGGATCAACACCGAGACCAGGGAGCCCGCCTACGCCTACGACTTCAGGGGC
AGGTTCGCCTTCAGCCTGGAGACCAGCGCCAGCACCGCCTACCTGCAGATCAACAACCTGAAGTACGAGGACACCGCC
ACCTACTTCTGCGCCCTGGACTACAGCTACGCCATGGACTACTGGGGCCAGGGCACCAGCGTGACCGTGAGCAGCGGC
AGCACCAGCGGCAGCGGCAAGCCCGGCAGCGGCGAGGGCAGCACCAAGGGCGACATCGTGCTGACCCAGAGCCCCCCC
AGCCTGGCCATGAGCCTGGGCAAGAGGGCCACCATCAGCTGCAGGGCCAGCGAGAGCGTGACCATCCTGGGCAGCCAC
CTGATCCACTGGTACCAGCAGAAGCCCGGCCAGCCCCCCACCCTGCTGATCCAGCTGGCCAGCAACGTGCAGACCGGC
GTGCCCGCCAGGTTCAGCGGCAGCGGCAGCAGGACCGACTTCACCCTGACCATCGACCCCGTGGAGGAGGACGACGTG
GCCGTGTACTACTGCCTGCAGAGCAGGACCATCCCCAGGACCTTCGGCGGCGGCACCAAGCTGGAGATCAAGGGAGGA
GGTGGGTCTGGAGGTGGAGGATCTGGTGGAGGTGGGTCTCATCATCACCATCACCAT
SEQID NO. 293
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGACAAAACTCACACATGCCCACCG
TGCCCAGCACCTGAACTCCTGGGGGGACCGTCAGTCTTCCTCTTCCCCCCAAAACCCAAGGACACCCTCATGATCTCC
CGGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCCGGGAGGAGATG
ACCAAGAACCAGGTCAGCCTGACCTGCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTGATGCACGAGGCTCTGCACAACCACTAC
ACGCAGAAGAGCCTCTCCCTGTCTCCGGGTGGAGGAGGTGGGTCTGGAGGTGGAGGATCTGGTGGAGGTGGGTCTGGA
GGAGGTGGGTCTGAGGTGCAGCTGGTGGAGTCTGGTGGTGGTCTTGTTCAACCTGGTGGTTCTCTTCGTCTTTCTTGT
GCTGCTTCTGGTTTTAATATTAAAGATACTTATATTCATTGGGTTCGTCAAGCTCCTGGTAAAGGTCTTGAATGGGTT
GCTCGTATTTATCCTACTAATGGTTATACTCGTTATGCTGATTCTGTTAAAGGTCGTTTTACTATTTCTGCTGATACT
TCTAAAAATACTGCTTATCTTCAAATGAACTCTCTTCGTGCTGAAGATACTGCTGTTTATTATTGTTCTCGTTGGGGT
GGTGATGGTTTTTATGCTATGGATTATTGGGGTCAAGGTACTCTTGTCACCGTCTCCTCAGCTAGCACCGGGGGAGGT
GGGTCTGGAGGTGGAGGATCTGGTGGAGGTGGGTCTGACATCCAGATGACCCAGTCTCCTTCTTCTCTTTCTGCTTCT
GTTGGTGATCGTGTTACTATTACTTGTCGTGCTTCTCAAGATGTTAATACTGCTGTTGCTTGGTATCAACAAAAACCT
GGTAAAGCTCCTAAACTTCTTATTTATTCTGCTTCTTTTCTTTATTCTGGTGTTCCTTCTCGTTTTTCTGGTTCTCGT
TCTGGTACTGATTTTACTCTTACTATTTCTTCTCTTCAACCTGAAGATTTTGCTACTTATTATTGTCAACAACATTAT
ACTACTCCTCCTACTTTTGGTCAAGGTACCAAGGTGGAGATCAAACGTACG
SEQID NO. 294
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGGGGAGGTGGGTCTGGAGGTGGA
GGATCTGGTGGAGGTGGGTCTGGTGGAGGTGGGTCTGAGGTGCAGCTGGTGGAGTCTGGTGGTGGTCTTGTTCAACCT
GGTGGTTCTCTTCGTCTTTCTTGTGCTGCTTCTGGTTTTAATATTAAAGATACTTATATTCATTGGGTTCGTCAAGCT
CCTGGTAAAGGTCTTGAATGGGTTGCTCGTATTTATCCTACTAATGGTTATACTCGTTATGCTGATTCTGTTAAAGGT
CGTTTTACTATTTCTGCTGATACTTCTAAAAATACTGCTTATCTTCAAATGAACTCTCTTCGTGCTGAAGATACTGCT
GTTTATTATTGTTCTCGTTGGGGTGGTGATGGTTTTTATGCTATGGATTATTGGGGTCAAGGTACTCTTGTCACCGTC
TCCTCAGCTAGCACCGGGGGAGGTGGGTCTGGAGGTGGAGGATCTGGTGGAGGTGGGTCTGACATCCAGATGACCCAG
TCTCCTTCTTCTCTTTCTGCTTCTGTTGGTGATCGTGTTACTATTACTTGTCGTGCTTCTCAAGATGTTAATACTGCT
GTTGCTTGGTATCAACAAAAACCTGGTAAAGCTCCTAAACTTCTTATTTATTCTGCTTCTTTTCTTTATTCTGGTGTT
CCTTCTCGTTTTTCTGGTTCTCGTTCTGGTACTGATTTTACTCTTACTATTTCTTCTCTTCAACCTGAAGATTTTGCT
ACTTATTATTGTCAACAACATTATACTACTCCTCCTACTTTTGGTCAAGGTACCAAGGTGGAGATCAAACGTACGGGA
GGAGGTGGGTCTGGAGGTGGAGGATCTGGTGGAGGTGGGTCTGGAGGAGGTGGGTCTCAGGTGCAGCTGCAGGAGAGC
GGCCCCGGCGACGTGAAGCCCAGCGAGACCCTGAGCCTGACCTGCACCGTGAGCGGCGGCAGCGTGAGCAGCGGCGAC
TACTACTGGACCTGGATCAGGCAGAGCCCCGGCAAGGGCCTGGAGTGGATCGGCCACATCTACTACAGCGGCAACACC
AACTACAACCCCAGCCTGAAGAGCAGGCTGACCATCAGCATCGACACCAGCAAGACCACCTTCAGCCTGCAGCTGAGC
AGCGTGACCGGCGAGGACACCGCCATCTACTACTGCGTGAGGGACAGGGTGACCGGCGCCTTCGACATCTGGGGCCAG
GGCACCACCGTGACCGTGAGCAGCGCCAGCACCGGCGGAGGTGGCAGCGGCGGAGGTGGCAGCGGCGGAGGTGGCAGC
GGCGGAGGTGGCAGCGACATCCAGATGACCCAGAGCCCCAGCAGCCTGGAGGCCAGCGTGGGCGACAGGGTGACCATC
ACCTGCCAGGCCAGCCAGGACATCAGCAACTACCTGAACTGGTACCAGCAGAAGCCCGGCAAGAGCCCCAAGCTGCTG
ATCTACGACGCCAGCAACCTGGAGACCGGCGTGCCCGACAGGTTCAGCGGCAGCGGCAGCGGCACCGACTTCACCTTC
ACCATCAGCAGCCTGCAGGCCGAGGACGAGGGCACCTACTTCTGCCAGCACTTCGACCACCTGCCCCTGGCCTTCGGC
GGCGGCACCAAGCTGGAGATCAAGAGGACCGGAGGAGGTGGGTCTCATCATCACCATCACCAT
SEQID NO. 295
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGGGGAGGTGGGTCTGGAGGTGGA
GGATCTGGTGGAGGTGGGTCTGGTGGAGGTGGGTCTGAGGTGCAGCTGGTGGAGTCTGGTGGTGGTCTTGTTCAACCT
GGTGGTTCTCTTCGTCTTTCTTGTGCTGCTTCTGGTTTTAATATTAAAGATACTTATATTCATTGGGTTCGTCAAGCT
CCTGGTAAAGGTCTTGAATGGGTTGCTCGTATTTATCCTACTAATGGTTATACTCGTTATGCTGATTCTGTTAAAGGT
CGTTTTACTATTTCTGCTGATACTTCTAAAAATACTGCTTATCTTCAAATGAACTCTCTTCGTGCTGAAGATACTGCT
GTTTATTATTGTTCTCGTTGGGGTGGTGATGGTTTTTATGCTATGGATTATTGGGGTCAAGGTACTCTTGTCACCGTC
TCCTCAGCTAGCACCGGGGGAGGTGGGTCTGGAGGTGGAGGATCTGGTGGAGGTGGGTCTGACATCCAGATGACCCAG
TCTCCTTCTTCTCTTTCTGCTTCTGTTGGTGATCGTGTTACTATTACTTGTCGTGCTTCTCAAGATGTTAATACTGCT
GTTGCTTGGTATCAACAAAAACCTGGTAAAGCTCCTAAACTTCTTATTTATTCTGCTTCTTTTCTTTATTCTGGTGTT
CCTTCTCGTTTTTCTGGTTCTCGTTCTGGTACTGATTTTACTCTTACTATTTCTTCTCTTCAACCTGAAGATTTTGCT
ACTTATTATTGTCAACAACATTATACTACTCCTCCTACTTTTGGTCAAGGTACCAAGGTGGAGATCAAACGTACGGGA
GGAGGTGGGTCTGGAGGTGGAGGATCTGGTGGAGGTGGGTCTGGAGGAGGTGGGTCTGACATCCAGATGACCCAGAGC
CCCAGCAGCCTGGAGGCCAGCGTGGGCGACAGGGTGACCATCACCTGCCAGGCCAGCCAGGACATCAGCAACTACCTG
AACTGGTACCAGCAGAAGCCCGGCAAGAGCCCCAAGCTGCTGATCTACGACGCCAGCAACCTGGAGACCGGCGTGCCC
GACAGGTTCAGCGGCAGCGGCAGCGGCACCGACTTCACCTTCACCATCAGCAGCCTGCAGGCCGAGGACGAGGGCACC
TACTTCTGCCAGCACTTCGACCACCTGCCCCTGGCCTTCGGCGGCGGCACCAAGCTGGAGATCAAGAGGACCGGCGGA
GGTGGCAGCGGCGGAGGTGGCAGCGGCGGAGGTGGCAGCGGCGGAGGTGGCAGCCAGGTGCAGCTGCAGGAGAGCGGC
CCCGGCGACGTGAAGCCCAGCGAGACCCTGAGCCTGACCTGCACCGTGAGCGGCGGCAGCGTGAGCAGCGGCGACTAC
TACTGGACCTGGATCAGGCAGAGCCCCGGCAAGGGCCTGGAGTGGATCGGCCACATCTACTACAGCGGCAACACCAAC
TACAACCCCAGCCTGAAGAGCAGGCTGACCATCAGCATCGACACCAGCAAGACCACCTTCAGCCTGCAGCTGAGCAGC
GTGACCGGCGAGGACACCGCCATCTACTACTGCGTGAGGGACAGGGTGACCGGCGCCTTCGACATCTGGGGCCAGGGC
ACCACCGTGACCGTGAGCAGCGCCAGCACCGGAGGAGGTGGGTCTCATCATCACCATCACCAT
SEQID NO. 296
ATGGAGTTTGGGCTGAGCTGGGTTTTCCTCGTTGCTCTTTTTAGAGGTGTCCAGTGTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGGGAGGAGGTGGGTCTGGAGGTGGAGGATCTGGTGGAGGTGGGTCTGGAGGAGGTGGGTCT
CAGGTGCAGCTGCAGGAGAGCGGCCCCGGCGACGTGAAGCCCAGCGAGACCCTGAGCCTGACCTGCACCGTGAGCGGC
GGCAGCGTGAGCAGCGGCGACTACTACTGGACCTGGATCAGGCAGAGCCCCGGCAAGGGCCTGGAGTGGATCGGCCAC
ATCTACTACAGCGGCAACACCAACTACAACCCCAGCCTGAAGAGCAGGCTGACCATCAGCATCGACACCAGCAAGACC
ACCTTCAGCCTGCAGCTGAGCAGCGTGACCGGCGAGGACACCGCCATCTACTACTGCGTGAGGGACAGGGTGACCGGC
GCCTTCGACATCTGGGGCCAGGGCACCACCGTGACCGTGAGCAGCGCCAGCACCGGCGGAGGTGGCAGCGGCGGAGGT
GGCAGCGGCGGAGGTGGCAGCGGCGGAGGTGGCAGCGACATCCAGATGACCCAGAGCCCCAGCAGCCTGGAGGCCAGC
GTGGGCGACAGGGTGACCATCACCTGCCAGGCCAGCCAGGACATCAGCAACTACCTGAACTGGTACCAGCAGAAGCCC
GGCAAGAGCCCCAAGCTGCTGATCTACGACGCCAGCAACCTGGAGACCGGCGTGCCCGACAGGTTCAGCGGCAGCGGC
AGCGGCACCGACTTCACCTTCACCATCAGCAGCCTGCAGGCCGAGGACGAGGGCACCTACTTCTGCCAGCACTTCGAC
CACCTGCCCCTGGCCTTCGGCGGCGGCACCAAGCTGGAGATCAAGAGGACCGGAGGAGGTGGGTCTCATCATCACCAT
CACCAT
SEQID NO. 297
ATGGAGTTTGGGCTGAGCTGGGTTTTCCTCGTTGCTCTTTTTAGAGGTGTCCAGTGTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGGGAGGAGGTGGGTCTGGAGGTGGAGGATCTGGTGGAGGTGGGTCTGGAGGAGGTGGGTCT
GACATCCAGATGACCCAGAGCCCCAGCAGCCTGGAGGCCAGCGTGGGCGACAGGGTGACCATCACCTGCCAGGCCAGC
CAGGACATCAGCAACTACCTGAACTGGTACCAGCAGAAGCCCGGCAAGAGCCCCAAGCTGCTGATCTACGACGCCAGC
AACCTGGAGACCGGCGTGCCCGACAGGTTCAGCGGCAGCGGCAGCGGCACCGACTTCACCTTCACCATCAGCAGCCTG
CAGGCCGAGGACGAGGGCACCTACTTCTGCCAGCACTTCGACCACCTGCCCCTGGCCTTCGGCGGCGGCACCAAGCTG
GAGATCAAGAGGACCGGCGGAGGTGGCAGCGGCGGAGGTGGCAGCGGCGGAGGTGGCAGCGGCGGAGGTGGCAGCCAG
GTGCAGCTGCAGGAGAGCGGCCCCGGCGACGTGAAGCCCAGCGAGACCCTGAGCCTGACCTGCACCGTGAGCGGCGGC
AGCGTGAGCAGCGGCGACTACTACTGGACCTGGATCAGGCAGAGCCCCGGCAAGGGCCTGGAGTGGATCGGCCACATC
TACTACAGCGGCAACACCAACTACAACCCCAGCCTGAAGAGCAGGCTGACCATCAGCATCGACACCAGCAAGACCACC
TTCAGCCTGCAGCTGAGCAGCGTGACCGGCGAGGACACCGCCATCTACTACTGCGTGAGGGACAGGGTGACCGGCGCC
TTCGACATCTGGGGCCAGGGCACCACCGTGACCGTGAGCAGCGCCAGCACCGGAGGAGGTGGGTCTCATCATCACCAT
CACCAT
SEQID NO. 298
GATATCGAATTAGGAGGAAAACTGTTTCATACAGAAGGCGTCAATTAGGAGGAAAAACTGTTTCATACAGAAGGCGTC
AATTAGGAGGAAAAACTGTTTCATACAGAAGGCGTCAATTGGTCCCATCGAATTAGGAGGAAAAACTGTTTCATACAG
AAGGCGTCAATTAGGAGGAAAAACTGTTTCATACAGAAGGCGTCAATTAGGAGGAAAAACTGTTTCATACAGAAGGCG
TCAATTGGTCCCGGGACATTTTGACACCCCCATAATATTTTTCCAGAATTAACAGTATAAATTGCATCTCTTGTTCAA
GAGTTCCCTATCACTCTCTTTAATCACTACTCACAGTAACCTCAACTCCTGGCGGCCGCGCCACCATGCCACCTCCTC
GCCTCCTCTTCTTCCTCCTCTTCCTCACCCCCATGGAAGTCAGGCCCGAGGAACCTCTAGTGGTGAAGGTGGAAGAGG
GAGATAACGCTGTGCTGCAGTGCCTCAAGGGGACCTCAGATGGCCCCACTCAGCAGCTGACTTGGTCTCGGGAGTCCC
CGCTTAAACCCTTCTTAAAACTCAGCCTGGGGCTGCCAGGCCTGGGAATCCACATGAGGCCCCTGGCCATCTGGCTTT
TCATCTTCAACGTCTCTCAACAGATGGGGGGCTTCTACCTGTGCCAGCCGGGGCCCCCCTCTGAGAAGGCCTGGCAGC
CTGGCTGGACAGTCAATGTGGAGGGCAGCGGGGAGCTGTTCCGGTGGAATGTTTCGGACCTAGGTGGCCTGGGCTGTG
GCCTGAAGAACAGGTCCTCAGAGGGCCCCAGCTCCCCTTCCGGGAAGCTCATGAGCCCCAAGCTGTATGTGTGGGCCA
AAGACCGCCCTGAGATCTGGGAGGGAGAGCCTCCGTGTCTCCCACCGAGGGACAGCCTGAACCAGAGCCTCAGCCAGG
ACCTCACCATGGCCCCTGGCTCCACACTCTGGCTGTCCTGTGGGGTACCCCCTGACTCTGTGTCCAGGGGCCCCCTCT
CCTGGACCCATGTGCACCCCAAGGGGCCTAAGTCATTGCTGAGCCTAGAGCTGAAGGACGATCGCCCGGCCAGAGATA
TGTGGGTAATGGAGACGGGTCTGTTGTTGCCCCGGGCCACAGCTCAAGACGCTGGAAAGTATTATTGTCACCGTGGCA
ACCTGACCATGTCATTCCACCTGGAGATCACTGCTCGGCCAGGGGGAGGTGGGTCTGGAGGTGGAGGATCTGGTGGAG
GTGGGTCTGGTGGAGGTGGGTCTGAGGTGCAGCTGGTGGAGTCTGGTGGTGGTCTTGTTCAACCCGGTGGTTCTCTTC
GTCTTTCTTGTGCTGCTTCTGGTTTTAATATTAAAGATACTTATATTCATTGGGTTCGTCAAGCTCCTGGTAAAGGTC
TTGAATGGGTTGCTCGTATTTATCCTACTAATGGTTATACTCGTTATGCTGATTCTGTTAAAGGTCGTTTTACTATTT
CTGCTGATACTTCTAAAAATACTGCTTATCTTCAAATGAACTCTCTTCGTGCTGAAGATACTGCTGTTTATTATTGTT
CTCGTTGGGGTGGTGATGGTTTTTATGCTATGGATTATTGGGGTCAAGGTACTCTTGTCACCGTCTCCTCAGCTAGCA
CCGGGGGAGGTGGGTCTGGAGGTGGAGGATCTGGTGGAGGTGGGTCTGACATCCAGATGACCCAGTCTCCTTCTTCTC
TTTCTGCTTCTGTTGGTGATCGTGTTACTATTACTTGTCGTGCTTCTCAAGATGTTAATACTGCTGTTGCTTGGTATC
AACAAAAACCCGGTAAAGCTCCTAAACTTCTTATTTATTCTGCTTCTTTTCTTTATTCTGGTGTTCCTTCTCGTTTTT
CTGGTTCTCGTTCTGGTACTGATTTTACTCTTACTATTTCTTCTCTTCAACCTGAAGATTTTGCTACTTATTATTGTC
AACAACATTATACTACTCCTCCTACTTTTGGTCAAGGTACCAAGGTGGAGATCAAACGTACGCATCATCACCATCACC
AT
SEQID NO. 299
ATGGATTTTCAGGTGCAGATTTTCAGCTTCCTGCTAATCAGTGCCTCAGTCATAATGTCTAGAATGGCCCAGGTCAAA
CTACAGGAGTCAGGGGCTGAGCTGGTGAAGCCTGGGGCCTCAGTGAAGATGTCCTGCAAGGCTTCTGGCTACACATTT
ACCAGTTACAATATGCACTGGGTAAAGCAGACACCTGGACAGGGCCTGGAATGGATTGGAGCTATTTATCCAGGAAAT
GGTGATACTTCCTACAATCAGAAGTTCAAAGGCAAGGCCACATTGACTGCAGACAAATCCTCCAGCACAGCCTACATG
CAGCTCAGCAGCCTGACATCTGAGGACTCTGCGGACTATTACTGTGCAAGATCTAATTATTACGGTAGTAGCTACTGG
TTCTTCGATGTCTGGGGCCAAGGGACCACGGTCACCGTCTCCTCAGGTGGAGGCGGTTCAGGCGGAGGTGGCTCTGGC
GGTGGCGGATCGGACATCGAGCTCACTCAGTCTCCAACAATCCTGTCTGCATCTCCAGGGGAGAAGGTCACAATGACT
TGCAGGGCCAGCTCAAGTGTAAATTACATGGACTGGTACCAGAAGAAGCCAGGATCCTCCCCCAAACCCTGGATTTAT
GCCACATCCAACCTGGCTTCTGGAGTCCCTGCTCGCTTCAGTGGCAGTGGGTCTGGGACCTCTTACTCTCTCACAATC
AGCAGAGTGGAGGCTGAAGATGCTGCCACTTATTACTGCCAGCAGTGGAGTTTTAATCCACCCACGTTCGGAGGGGGG
ACAAAGTTGGAAATAAAACGGGCCGCCGCTGGTGATCCCGCCGAGCCCAAATCTCCTGACAAAACTCACACATGCCCA
CCGTGCCCAGCACCTGAACTCCTGGGGGGACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCATCCCGGGAGGAG
ATGACCAAGAACCAGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTTCCTCTACAGCAAG
CTCACCGTGGACAAGAGCAGGTGGCAGCAGGGGAACGTCTTCTCATGCTCCGTGATGCACGAGGCTCTGCACAACCAC
TACACGCAGAAGAGCCTCTCCCTGTCTCCGGGT
SEQID NO. 300
ATGGATTTTCAGGTGCAGATTTTCAGCTTCCTGCTAATCAGTGCCTCAGTCATAATGTCTAGAATGGCCCAGGTCAAA
CTACAGGAGTCAGGGGCTGAGCTGGTGAAGCCTGGGGCCTCAGTGAAGATGTCCTGCAAGGCTTCTGGCTACACATTT
ACCAGTTACAATATGCACTGGGTAAAGCAGACACCTGGACAGGGCCTGGAATGGATTGGAGCTATTTATCCAGGAAAT
GGTGATACTTCCTACAATCAGAAGTTCAAAGGCAAGGCCACATTGACTGCAGACAAATCCTCCAGCACAGCCTACATG
CAGCTCAGCAGCCTGACATCTGAGGACTCTGCGGACTATTACTGTGCAAGATCTAATTATTACGGTAGTAGCTACTGG
TTCTTCGATGTCTGGGGCCAAGGGACCACGGTCACCGTCTCCTCAGGTGGAGGCGGTTCAGGCGGAGGTGGCTCTGGC
GGTGGCGGATCGGACATCGAGCTCACTCAGTCTCCAACAATCCTGTCTGCATCTCCAGGGGAGAAGGTCACAATGACT
TGCAGGGCCAGCTCAAGTGTAAATTACATGGACTGGTACCAGAAGAAGCCAGGATCCTCCCCCAAACCCTGGATTTAT
GCCACATCCAACCTGGCTTCTGGAGTCCCTGCTCGCTTCAGTGGCAGTGGGTCTGGGACCTCTTACTCTCTCACAATC
AGCAGAGTGGAGGCTGAAGATGCTGCCACTTATTACTGCCAGCAGTGGAGTTTTAATCCACCCACGTTCGGAGGGGGG
ACAAAGTTGGAAATAAAACGGGCCGCCGCTCATCATCACCATCACCAT
SEQID NO. 301
ATGGATTTTCAGGTGCAGATTTTCAGCTTCCTGCTAATCAGTGCCTCAGTCATAATGTCTAGAGACATCGAGCTCACT
CAGTCTCCAACAATCCTGTCTGCATCTCCAGGGGAGAAGGTCACAATGACTTGCAGGGCCAGCTCAAGTGTAAATTAC
ATGGACTGGTACCAGAAGAAGCCAGGATCCTCCCCCAAACCCTGGATTTATGCCACATCCAACCTGGCTTCTGGAGTC
CCTGCTCGCTTCAGTGGCAGTGGGTCTGGGACCTCTTACTCTCTCACAATCAGCAGAGTGGAGGCTGAAGATGCTGCC
ACTTATTACTGCCAGCAGTGGAGTTTTAATCCACCCACGTTCGGAGGGGGGACAAAGTTGGAAATAAAACGGGGTGGA
GGCGGTTCAGGCGGAGGTGGCTCTGGCGGTGGCGGATCGATGGCCCAGGTCAAACTACAGGAGTCAGGGGCTGAGCTG
GTGAAGCCTGGGGCCTCAGTGAAGATGTCCTGCAAGGCTTCTGGCTACACATTTACCAGTTACAATATGCACTGGGTA
AAGCAGACACCTGGACAGGGCCTGGAATGGATTGGAGCTATTTATCCAGGAAATGGTGATACTTCCTACAATCAGAAG
TTCAAAGGCAAGGCCACATTGACTGCAGACAAATCCTCCAGCACAGCCTACATGCAGCTCAGCAGCCTGACATCTGAG
GACTCTGCGGACTATTACTGTGCAAGATCTAATTATTACGGTAGTAGCTACTGGTTCTTCGATGTCTGGGGCCAAGGG
ACCACGGTCACCGTCTCCTCAGCCGCCGCTGGTGATCCCGCCGAGCCCAAATCTCCTGACAAAACTCACACATGCCCA
CCGTGCCCAGCACCTGAACTCCTGGGGGGACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCATCCCGGGAGGAG
ATGACCAAGAACCAGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTTCCTCTACAGCAAG
CTCACCGTGGACAAGAGCAGGTGGCAGCAGGGGAACGTCTTCTCATGCTCCGTGATGCACGAGGCTCTGCACAACCAC
TACACGCAGAAGAGCCTCTCCCTGTCTCCGGGT
SEQID NO. 302
ATGGATTTTCAGGTGCAGATTTTCAGCTTCCTGCTAATCAGTGCCTCAGTCATAATGTCTAGAGACATCGAGCTCACT
CAGTCTCCAACAATCCTGTCTGCATCTCCAGGGGAGAAGGTCACAATGACTTGCAGGGCCAGCTCAAGTGTAAATTAC
ATGGACTGGTACCAGAAGAAGCCAGGATCCTCCCCCAAACCCTGGATTTATGCCACATCCAACCTGGCTTCTGGAGTC
CCTGCTCGCTTCAGTGGCAGTGGGTCTGGGACCTCTTACTCTCTCACAATCAGCAGAGTGGAGGCTGAAGATGCTGCC
ACTTATTACTGCCAGCAGTGGAGTTTTAATCCACCCACGTTCGGAGGGGGGACAAAGTTGGAAATAAAACGGGGTGGA
GGCGGTTCAGGCGGAGGTGGCTCTGGCGGTGGCGGATCGATGGCCCAGGTCAAACTACAGGAGTCAGGGGCTGAGCTG
GTGAAGCCTGGGGCCTCAGTGAAGATGTCCTGCAAGGCTTCTGGCTACACATTTACCAGTTACAATATGCACTGGGTA
AAGCAGACACCTGGACAGGGCCTGGAATGGATTGGAGCTATTTATCCAGGAAATGGTGATACTTCCTACAATCAGAAG
TTCAAAGGCAAGGCCACATTGACTGCAGACAAATCCTCCAGCACAGCCTACATGCAGCTCAGCAGCCTGACATCTGAG
GACTCTGCGGACTATTACTGTGCAAGATCTAATTATTACGGTAGTAGCTACTGGTTCTTCGATGTCTGGGGCCAAGGG
ACCACGGTCACCGTCTCCTCAGCCGCCGCTCATCATCACCATCACCAT
SEQID NO. 303
ATGGAGTTTGGGCTGAGCTGGGTTTTCCTCGTTGCTCTTTTTAGAGGTGTCCAGTGTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGGACAAAACTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAGGTCACATGCGTGGTGGTGGACGTG
AGCCACGAAGACCCTGAGGTCAAGTTCAACTGGTACGTGGACGGCGTGGAGGTGCATAATGCCAAGACAAAGCCGCGG
GAGGAGCAGTACAACAGCACGTACCGTGTGGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGACCTGCCTGGTCAAA
GGCTTCTATCCCAGCGACATCGCCGTGGAGTGGGAGAGCAATGGGCAGCCGGAGAACAACTACAAGACCACGCCTCCC
GTGCTGGACTCCGACGGCTCCTTCTTCCTCTACAGCAAGCTCACCGTGGACAAGAGCAGGTGGCAGCAGGGGAACGTC
TTCTCATGCTCCGTGATGCACGAGGCTCTGCACAACCACTACACGCAGAAGAGCCTCTCCCTGTCTCCGGGT
SEQID NO. 304
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTGGAGGAGGTGGGTCTGGAGGTGGAGGATCTGGTGGAGGTGGG
TCTATGGCCCAGGTCAAACTACAGGAGTCAGGGGCTGAGCTGGTGAAGCCTGGGGCCTCAGTGAAGATGTCCTGCAAG
GCTTCTGGCTACACATTTACCAGTTACAATATGCACTGGGTAAAGCAGACACCTGGACAGGGCCTGGAATGGATTGGA
GCTATTTATCCAGGAAATGGTGATACTTCCTACAATCAGAAGTTCAAAGGCAAGGCCACATTGACTGCAGACAAATCC
TCCAGCACAGCCTACATGCAGCTCAGCAGCCTGACATCTGAGGACTCTGCGGACTATTACTGTGCAAGATCTAATTAT
TACGGTAGTAGCTACTGGTTCTTCGATGTCTGGGGCCAAGGGACCACGGTCACCGTCTCCTCAGGTGGAGGCGGTTCA
GGCGGAGGTGGCTCTGGCGGTGGCGGATCGGACATCGAGCTCACTCAGTCTCCAACAATCCTGTCTGCATCTCCAGGG
GAGAAGGTCACAATGACTTGCAGGGCCAGCTCAAGTGTAAATTACATGGACTGGTACCAGAAGAAGCCAGGATCCTCC
CCCAAACCCTGGATTTATGCCACATCCAACCTGGCTTCTGGAGTCCCTGCTCGCTTCAGTGGCAGTGGGTCTGGGACC
TCTTACTCTCTCACAATCAGCAGAGTGGAGGCTGAAGATGCTGCCACTTATTACTGCCAGCAGTGGAGTTTTAATCCA
CCCACGTTCGGAGGGGGGACAAAGTTGGAAATAAAACGGGCCGCCGCTGGTGATCCCGCCGAGCCCAAATCTCCTGAC
AAAACTCACACATGCCCACCGTGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGTGCAAGGTCTCCAAC
AAAGCCCTCCCAGCCCCCATCGAGAAAACCATCTCCAAAGCCAAAGGGCAGCCCCGAGAACCACAGGTGTACACCCTG
CCCCCATCCCGGGAGGAGATGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGACTCCGACGGCTCC
TTCTTCCTCTACAGCAAGCTCACCGTGGACAAGAGCAGGTGGCAGCAGGGGAACGTCTTCTCATGCTCCGTGATGCAC
GAGGCTCTGCACAACCACTACACGCAGAAGAGCCTCTCCCTGTCTCCGGGT
SEQID NO. 305
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTGGAGGAGGTGGGTCTGGAGGTGGAGGATCTGGTGGAGGTGGG
TCTATGGCCCAGGTCAAACTACAGGAGTCAGGGGCTGAGCTGGTGAAGCCTGGGGCCTCAGTGAAGATGTCCTGCAAG
GCTTCTGGCTACACATTTACCAGTTACAATATGCACTGGGTAAAGCAGACACCTGGACAGGGCCTGGAATGGATTGGA
GCTATTTATCCAGGAAATGGTGATACTTCCTACAATCAGAAGTTCAAAGGCAAGGCCACATTGACTGCAGACAAATCC
TCCAGCACAGCCTACATGCAGCTCAGCAGCCTGACATCTGAGGACTCTGCGGACTATTACTGTGCAAGATCTAATTAT
TACGGTAGTAGCTACTGGTTCTTCGATGTCTGGGGCCAAGGGACCACGGTCACCGTCTCCTCAGGTGGAGGCGGTTCA
GGCGGAGGTGGCTCTGGCGGTGGCGGATCGGACATCGAGCTCACTCAGTCTCCAACAATCCTGTCTGCATCTCCAGGG
GAGAAGGTCACAATGACTTGCAGGGCCAGCTCAAGTGTAAATTACATGGACTGGTACCAGAAGAAGCCAGGATCCTCC
CCCAAACCCTGGATTTATGCCACATCCAACCTGGCTTCTGGAGTCCCTGCTCGCTTCAGTGGCAGTGGGTCTGGGACC
TCTTACTCTCTCACAATCAGCAGAGTGGAGGCTGAAGATGCTGCCACTTATTACTGCCAGCAGTGGAGTTTTAATCCA
CCCACGTTCGGAGGGGGGACAAAGTTGGAAATAAAACGGGCCGCCGCTCATCATCACCATCACCAT
SEQID NO. 306
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGATCCCGCCGAGCCCAAATCTCCT
GACAAAACTCACACATGCCCACCGTGCCCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAATGGCAAGGAGTACAAGTGCAAGGTCTCC
AACAAAGCCCTCCCAGCCCCCATCGAGAAAACCATCTCCAAAGCCAAAGGGCAGCCCCGAGAACCACAGGTGTACACC
CTGCCCCCATCCCGGGAGGAGATGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTCATGCTCCGTGATG
CACGAGGCTCTGCACAACCACTACACGCAGAAGAGCCTCTCCCTGTCTCCGGGTGGAGGAGGTGGGTCTGGAGGTGGA
GGATCTGGTGGAGGTGGGTCTGGAGGAGGTGGGTCTATGGCCCAGGTCAAACTACAGGAGTCAGGGGCTGAGCTGGTG
AAGCCTGGGGCCTCAGTGAAGATGTCCTGCAAGGCTTCTGGCTACACATTTACCAGTTACAATATGCACTGGGTAAAG
CAGACACCTGGACAGGGCCTGGAATGGATTGGAGCTATTTATCCAGGAAATGGTGATACTTCCTACAATCAGAAGTTC
AAAGGCAAGGCCACATTGACTGCAGACAAATCCTCCAGCACAGCCTACATGCAGCTCAGCAGCCTGACATCTGAGGAC
TCTGCGGACTATTACTGTGCAAGATCTAATTATTACGGTAGTAGCTACTGGTTCTTCGATGTCTGGGGCCAAGGGACC
ACGGTCACCGTCTCCTCAGGTGGAGGCGGTTCAGGCGGAGGTGGCTCTGGCGGTGGCGGATCGGACATCGAGCTCACT
CAGTCTCCAACAATCCTGTCTGCATCTCCAGGGGAGAAGGTCACAATGACTTGCAGGGCCAGCTCAAGTGTAAATTAC
ATGGACTGGTACCAGAAGAAGCCAGGATCCTCCCCCAAACCCTGGATTTATGCCACATCCAACCTGGCTTCTGGAGTC
CCTGCTCGCTTCAGTGGCAGTGGGTCTGGGACCTCTTACTCTCTCACAATCAGCAGAGTGGAGGCTGAAGATGCTGCC
ACTTATTACTGCCAGCAGTGGAGTTTTAATCCACCCACGTTCGGAGGGGGGACAAAGTTGGAAATAAAACGGGCT
SEQID NO. 307
ATGGATTTTCAGGTGCAGATTTTCAGCTTCCTGCTAATCAGTGCCTCAGTCATAATGTCTAGAATGGCCCAGGTCAAA
CTACAGGAGTCAGGGGCTGAGCTGGTGAAGCCTGGGGCCTCAGTGAAGATGTCCTGCAAGGCTTCTGGCTACACATTT
ACCAGTTACAATATGCACTGGGTAAAGCAGACACCTGGACAGGGCCTGGAATGGATTGGAGCTATTTATCCAGGAAAT
GGTGATACTTCCTACAATCAGAAGTTCAAAGGCAAGGCCACATTGACTGCAGACAAATCCTCCAGCACAGCCTACATG
CAGCTCAGCAGCCTGACATCTGAGGACTCTGCGGACTATTACTGTGCAAGATCTAATTATTACGGTAGTAGCTACTGG
TTCTTCGATGTCTGGGGCCAAGGGACCACGGTCACCGTCTCCTCAGGTGGAGGCGGTTCAGGCGGAGGTGGCTCTGGC
GGTGGCGGATCGGACATCGAGCTCACTCAGTCTCCAACAATCCTGTCTGCATCTCCAGGGGAGAAGGTCACAATGACT
TGCAGGGCCAGCTCAAGTGTAAATTACATGGACTGGTACCAGAAGAAGCCAGGATCCTCCCCCAAACCCTGGATTTAT
GCCACATCCAACCTGGCTTCTGGAGTCCCTGCTCGCTTCAGTGGCAGTGGGTCTGGGACCTCTTACTCTCTCACAATC
AGCAGAGTGGAGGCTGAAGATGCTGCCACTTATTACTGCCAGCAGTGGAGTTTTAATCCACCCACGTTCGGAGGGGGG
ACAAAGTTGGAAATAAAACGGGCCGCCGCTGGAGGAGGTGGGTCTGGAGGTGGAGGATCTGGTGGAGGTGGGTCTGGA
GGAGGTGGGTCTCCCGAGGAACCTCTAGTGGTGAAGGTGGAAGAGGGAGATAACGCTGTGCTGCAGTGCCTCAAGGGG
ACCTCAGATGGCCCCACTCAGCAGCTGACCTGGTCTCGGGAGTCCCCGCTTAAACCCTTCTTAAAACTCAGCCTGGGG
CTGCCAGGCCTGGGAATCCACATGAGGCCCCTGGCCATCTGGCTTTTCATCTTCAACGTCTCTCAACAGATGGGGGGC
TTCTACCTGTGCCAGCCGGGGCCCCCCTCTGAGAAGGCCTGGCAGCCTGGCTGGACAGTCAATGTGGAGGGCAGCGGG
GAGCTGTTCCGGTGGAATGTTTCGGACCTAGGTGGCCTGGGCTGTGGCCTGAAGAACAGGTCCTCAGAGGGCCCCAGC
TCCCCTTCCGGGAAGCTCATGAGCCCCAAGCTGTATGTGTGGGCCAAAGACCGCCCTGAGATCTGGGAGGGAGAGCCT
CCGTGTCTCCCACCGAGGGACAGCCTGAACCAGAGCCTCAGCCAGGACCTCACCATGGCCCCTGGCTCCACACTCTGG
CTGTCCTGTGGGGTACCCCCTGACTCTGTGTCCAGGGGCCCCCTCTCCTGGACCCATGTGCACCCCAAGGGGCCTAAG
TCATTGCTGAGCCTAGAGCTGAAGGACGATCGCCCGGCCAGAGATATGTGGGTAATGGAGACGGGTCTGTTGTTGCCC
CGGGCCACAGCTCAAGACGCTGGAAAGTATTATTGTCACCGTGGCAACCTGACCATGTCATTCCACCTGGAGATCACT
GCTCGGCCAGATCCCGCCGAGCCCAAATCTCCTGACAAAACTCACACATGCCCACCGTGCCCAGCACCTGAACTCCTG
GGGGGACCGTCAGTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTGCACCAGGACTGG
CTGAATGGCAAGGAGTACAAGTGCAAGGTCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGAGGAGATGACCAAGAACCAGGTCAGCCTG
ACCTGCCTGGTCAAAGGCTTCTATCCCAGCGACATCGCCGTGGAGTGGGAGAGCAATGGGCAGCCGGAGAACAACTAC
AAGACCACGCCTCCCGTGCTGGACTCCGACGGCTCCTTCTTCCTCTACAGCAAGCTCACCGTGGACAAGAGCAGGTGG
CAGCAGGGGAACGTCTTCTCATGCTCCGTGATGCACGAGGCTCTGCACAACCACTACACGCAGAAGAGCCTCTCCCTG
TCTCCGGGT
SEQID NO. 308
ATGCTCAGGCTGCTCTTGGCTCTCAACTTATTCCCTTCAATTCAAGTAACAGGAGACATCCAGATGACACAGACTACA
TCCTCCCTGTCTGCCTCTCTGGGAGACAGAGTCACCATCAGTTGCAGGGCAAGTCAGGACATTAGTAAATATTTAAAT
TGGTATCAGCAGAAACCAGATGGAACTGTTAAACTCCTGATCTACCATACATCAAGATTACACTCAGGAGTCCCATCA
AGGTTCAGTGGCAGTGGGTCTGGAACAGATTATTCTCTCACCATTAGCAACCTGGAGCAAGAAGATATTGCCACTTAC
TTTTGCCAACAGGGTAATACGCTTCCGTACACGTTCGGAGGGGGGACTAAGTTGGAAATAACAGGAGGAGGTGGGTCT
GGAGGTGGAGGATCTGGTGGAGGTGGGTCTGGAGGAGGTGGGTCTGAGGTGAAACTGCAGGAGTCAGGACCTGGCCTG
GTGGCGCCCTCACAGAGCCTGTCCGTCACATGCACTGTCTCAGGGGTCTCATTACCCGACTATGGTGTAAGCTGGATT
CGCCAGCCTCCACGAAAGGGTCTGGAGTGGCTGGGAGTAATATGGGGTAGTGAAACCACATACTATAATTCAGCTCTC
AAATCCAGACTGACCATCATCAAGGACAACTCCAAGAGCCAAGTTTTCTTAAAAATGAACAGTCTGCAAACTGATGAC
ACAGCCATTTACTACTGTGCCAAACATTATTACTACGGTGGTAGCTATGCTATGGACTACTGGGGTCAAGGAACCTCA
GTCACCGTCTCCTCAGACTACAAAGACGATGACGACAAGATTGAAGTTATGTATCCTCCTCCTTACCTAGACAATGAG
AAGAGCAATGGAACCATTATCCATGTGAAAGGGAAACACCTTTGTCCAAGTCCCCTATTTCCCGGACCTTCTAAGCCC
TTTTGGGTGCTGGTGGTGGTTGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCGCCGCCCCGGGCCCACCCGCAAG
CATTACCAGCCCTATGCCCCACCACGCGACTTCGCAGCCTATCGCTCCAAACGGGGCAGAAAGAAACTCCTGTATATA
TTCAAACAACCATTTATGAGACCAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGAGACGTGGCCGGGACCCTGAGATGGGG
GGAAAGCCGCAGAGAAGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGTCTCAGTACAGCCACC
AAGGACACCTACGACGCCCTTCACATGCAGGCCCTGCCCCCTCGCGAGGGCAGAGGAAGTCTTCTAACATGCGGTGAC
GTGGAGGAGAATCCCGGCCCTATGCCACCTCCTCGCCTCCTCTTCTTCCTCCTCTTCCTCACCCCCATGGAAGTCAGG
CCCGAGGAACCTCTAGTGGTGAAGGTGGAAGAGGGAGATAACGCTGTGCTGCAGTGCCTCAAGGGGACCTCAGATGGC
CCCACTCAGCAGCTGACCTGGTCTCGGGAGTCCCCGCTTAAACCCTTCTTAAAACTCAGCCTGGGGCTGCCAGGCCTG
GGAATCCACATGAGGCCCCTGGCCATCTGGCTTTTCATCTTCAACGTCTCTCAACAGATGGGGGGCTTCTACCTGTGC
CAGCCGGGGCCCCCCTCTGAGAAGGCCTGGCAGCCTGGCTGGACAGTCAATGTGGAGGGCAGCGGGGAGCTGTTCCGG
TGGAATGTTTCGGACCTAGGTGGCCTGGGCTGTGGCCTGAAGAACAGGTCCTCAGAGGGCCCCAGCTCCCCTTCCGGG
AAGCTCATGAGCCCCAAGCTGTATGTGTGGGCCAAAGACCGCCCTGAGATCTGGGAGGGAGAGCCTCCGTGTCTCCCA
CCGAGGGACAGCCTGAACCAGAGCCTCAGCCAGGACCTCACCATGGCCCCTGGCTCCACACTCTGGCTGTCCTGTGGG
GTACCCCCTGACTCTGTGTCCAGGGGCCCCCTCTCCTGGACCCATGTGCACCCCAAGGGGCCTAAGTCATTGCTGAGC
CTAGAGCTGAAGGACGATCGCCCGGCCAGAGATATGTGGGTAATGGAGACGGGTCTGTTGTTGCCCCGGGCCACAGCT
CAAGACGCTGGAAAGTATTATTGTCACCGTGGCAACCTGACCATGTCATTCCACCTGGAGATCACTGCTCGGCCAGGG
GGAGGTGGGTCTGGAGGTGGAGGATCTGGTGGAGGTGGGTCTGGTGGAGGTGGGTCTGAGGTGCAGCTGGTGGAGTCT
GGTGGTGGTCTTGTTCAACCTGGTGGTTCTCTTCGTCTTTCTTGTGCTGCTTCTGGTTTTAATATTAAAGATACTTAT
ATTCATTGGGTTCGTCAAGCTCCTGGTAAAGGTCTTGAATGGGTTGCTCGTATTTATCCTACTAATGGTTATACTCGT
TATGCTGATTCTGTTAAAGGTCGTTTTACTATTTCTGCTGATACTTCTAAAAATACTGCTTATCTTCAAATGAACTCT
CTTCGTGCTGAAGATACTGCTGTTTATTATTGTTCTCGTTGGGGTGGTGATGGTTTTTATGCTATGGATTATTGGGGT
CAAGGTACTCTTGTCACCGTCTCCTCAGCTAGCACCGGGGGAGGTGGGTCTGGAGGTGGAGGATCTGGTGGAGGTGGG
TCTGACATCCAGATGACCCAGTCTCCTTCTTCTCTTTCTGCTTCTGTTGGTGATCGTGTTACTATTACTTGTCGTGCT
TCTCAAGATGTTAATACTGCTGTTGCTTGGTATCAACAAAAACCTGGTAAAGCTCCTAAACTTCTTATTTATTCTGCT
TCTTTTCTTTATTCTGGTGTTCCTTCTCGTTTTTCTGGTTCTCGTTCTGGTACTGATTTTACTCTTACTATTTCTTCT
CTTCAACCTGAAGATTTTGCTACTTATTATTGTCAACAACATTATACTACTCCTCCTACTTTTGGTCAAGGTACCAAG
GTGGAGATCAAACGTACGTCTAGACATCATCACCATCACCAT
SEQID NO. 309
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGGGGAGGTGGGTCTGGAGGTGGA
GGATCTGGTGGAGGTGGGTCTGGTGGAGGTGGGTCTGAGGTGCAGCTGGTGGAGTCTGGTGGTGGTCTTGTTCAACCT
GGTGGTTCTCTTCGTCTTTCTTGTGCTGCTTCTGGTTTTAATATTAAAGATACTTATATTCATTGGGTTCGTCAAGCT
CCTGGTAAAGGTCTTGAATGGGTTGCTCGTATTTATCCTACTAATGGTTATACTCGTTATGCTGATTCTGTTAAAGGT
CGTTTTACTATTTCTGCTGATACTTCTAAAAATACTGCTTATCTTCAAATGAACTCTCTTCGTGCTGAAGATACTGCT
GTTTATTATTGTTCTCGTTGGGGTGGTGATGGTTTTTATGCTATGGATTATTGGGGTCAAGGTACTCTTGTCACCGTC
TCCTCAGCTAGCACCGGGGGAGGTGGGTCTGGAGGTGGAGGATCTGGTGGAGGTGGGTCTGACATCCAGATGACCCAG
TCTCCTTCTTCTCTTTCTGCTTCTGTTGGTGATCGTGTTACTATTACTTGTCGTGCTTCTCAAGATGTTAATACTGCT
GTTGCTTGGTATCAACAAAAACCTGGTAAAGCTCCTAAACTTCTTATTTATTCTGCTTCTTTTCTTTATTCTGGTGTT
CCTTCTCGTTTTTCTGGTTCTCGTTCTGGTACTGATTTTACTCTTACTATTTCTTCTCTTCAACCTGAAGATTTTGCT
ACTTATTATTGTCAACAACATTATACTACTCCTCCTACTTTTGGTCAAGGTACCAAGGTGGAGATCAAACGTACGTCT
AGACATCATCACCATCACCATGAGGGCAGAGGAAGTCTTCTAACATGCGGTGACGTGGAGGAGAATCCCGGCCCTATG
CTCAGGCTGCTCTTGGCTCTCAACTTATTCCCTTCAATTCAAGTAACAGGAGACATCCAGATGACACAGACTACATCC
TCCCTGTCTGCCTCTCTGGGAGACAGAGTCACCATCAGTTGCAGGGCAAGTCAGGACATTAGTAAATATTTAAATTGG
TATCAGCAGAAACCAGATGGAACTGTTAAACTCCTGATCTACCATACATCAAGATTACACTCAGGAGTCCCATCAAGG
TTCAGTGGCAGTGGGTCTGGAACAGATTATTCTCTCACCATTAGCAACCTGGAGCAAGAAGATATTGCCACTTACTTT
TGCCAACAGGGTAATACGCTTCCGTACACGTTCGGAGGGGGGACTAAGTTGGAAATAACAGGAGGAGGTGGGTCTGGA
GGTGGAGGATCTGGTGGAGGTGGGTCTGGAGGAGGTGGGTCTGAGGTGAAACTGCAGGAGTCAGGACCTGGCCTGGTG
GCGCCCTCACAGAGCCTGTCCGTCACATGCACTGTCTCAGGGGTCTCATTACCCGACTATGGTGTAAGCTGGATTCGC
CAGCCTCCACGAAAGGGTCTGGAGTGGCTGGGAGTAATATGGGGTAGTGAAACCACATACTATAATTCAGCTCTCAAA
TCCAGACTGACCATCATCAAGGACAACTCCAAGAGCCAAGTTTTCTTAAAAATGAACAGTCTGCAAACTGATGACACA
GCCATTTACTACTGTGCCAAACATTATTACTACGGTGGTAGCTATGCTATGGACTACTGGGGTCAAGGAACCTCAGTC
ACCGTCTCCTCAGACTACAAAGACGATGACGACAAGATTGAAGTTATGTATCCTCCTCCTTACCTAGACAATGAGAAG
AGCAATGGAACCATTATCCATGTGAAAGGGAAACACCTTTGTCCAAGTCCCCTATTTCCCGGACCTTCTAAGCCCTTT
TGGGTGCTGGTGGTGGTTGGTGGAGTCCTGGCTTGCTATAGCTTGCTAGTAACAGTGGCCTTTATTATTTTCTGGGTG
AGGAGTAAGAGGAGCAGGCTCCTGCACAGTGACTACATGAACATGACTCCCCGCCGCCCCGGGCCCACCCGCAAGCAT
TACCAGCCCTATGCCCCACCACGCGACTTCGCAGCCTATCGCTCCAAACGGGGCAGAAAGAAACTCCTGTATATATTC
AAACAACCATTTATGAGACCAGTACAAACTACTCAAGAGGAAGATGGCTGTAGCTGCCGATTTCCAGAAGAAGAAGAA
GGAGGATGTGAACTGAGAGTGAAGTTCAGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGACGTGGCCGGGACCCTGAGATGGGGGGA
AAGCCGCAGAGAAGGAAGAACCCTCAGGAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTTTACCAGGGTCTCAGTACAGCCACCAAG
GACACCTACGACGCCCTTCACATGCAGGCCCTGCCCCCTCGC
SEQID NO. 310
GATATCGAATTAGGAGGAAAACTGTTTCATACAGAAGGCGTCAATTAGGAGGAAAAACTGTTTCATACAGAAGGCGTC
AATTAGGAGGAAAAACTGTTTCATACAGAAGGCGTCAATTGGTCCCATCGAATTAGGAGGAAAAACTGTTTCATACAG
AAGGCGTCAATTAGGAGGAAAAACTGTTTCATACAGAAGGCGTCAATTAGGAGGAAAAACTGTTTCATACAGAAGGCG
TCAATTGGTCCCGGGACATTTTGACACCCCCATAATATTTTTCCAGAATTAACAGTATAAATTGCATCTCTTGTTCAA
GAGTTCCCTATCACTCTCTTTAATCACTACTCACAGTAACCTCAACTCCTGGCGGCCGCGCCACCATGCCACCTCCTC
GCCTCCTCTTCTTCCTCCTCTTCCTCACCCCCATGGAAGTCAGGCCCGAGGAACCTCTAGTGGTGAAGGTGGAAGAGG
GAGATAACGCTGTGCTGCAGTGCCTCAAGGGGACCTCAGATGGCCCCACTCAGCAGCTGACTTGGTCTCGGGAGTCCC
CGCTTAAACCCTTCTTAAAACTCAGCCTGGGGCTGCCAGGCCTGGGAATCCACATGAGGCCCCTGGCCATCTGGCTTT
TCATCTTCAACGTCTCTCAACAGATGGGGGGCTTCTACCTGTGCCAGCCGGGGCCCCCCTCTGAGAAGGCCTGGCAGC
CTGGCTGGACAGTCAATGTGGAGGGCAGCGGGGAGCTGTTCCGGTGGAATGTTTCGGACCTAGGTGGCCTGGGCTGTG
GCCTGAAGAACAGGTCCTCAGAGGGCCCCAGCTCCCCTTCCGGGAAGCTCATGAGCCCCAAGCTGTATGTGTGGGCCA
AAGACCGCCCTGAGATCTGGGAGGGAGAGCCTCCGTGTCTCCCACCGAGGGACAGCCTGAACCAGAGCCTCAGCCAGG
ACCTCACCATGGCCCCTGGCTCCACACTCTGGCTGTCCTGTGGGGTACCCCCTGACTCTGTGTCCAGGGGCCCCCTCT
CCTGGACCCATGTGCACCCCAAGGGGCCTAAGTCATTGCTGAGCCTAGAGCTGAAGGACGATCGCCCGGCCAGAGATA
TGTGGGTAATGGAGACGGGTCTGTTGTTGCCCCGGGCCACAGCTCAAGACGCTGGAAAGTATTATTGTCACCGTGGCA
ACCTGACCATGTCATTCCACCTGGAGATCACTGCTCGGCCAGGGGGAGGTGGGTCTGGAGGTGGAGGATCTGGTGGAG
GTGGGTCTGGTGGAGGTGGGTCTGAGGTGCAGCTGGTGGAGTCTGGTGGTGGTCTTGTTCAACCCGGTGGTTCTCTTC
GTCTTTCTTGTGCTGCTTCTGGTTTTAATATTAAAGATACTTATATTCATTGGGTTCGTCAAGCTCCTGGTAAAGGTC
TTGAATGGGTTGCTCGTATTTATCCTACTAATGGTTATACTCGTTATGCTGATTCTGTTAAAGGTCGTTTTACTATTT
CTGCTGATACTTCTAAAAATACTGCTTATCTTCAAATGAACTCTCTTCGTGCTGAAGATACTGCTGTTTATTATTGTT
CTCGTTGGGGTGGTGATGGTTTTTATGCTATGGATTATTGGGGTCAAGGTACTCTTGTCACCGTCTCCTCAGCTAGCA
CCGGGGGAGGTGGGTCTGGAGGTGGAGGATCTGGTGGAGGTGGGTCTGACATCCAGATGACCCAGTCTCCTTCTTCTC
TTTCTGCTTCTGTTGGTGATCGTGTTACTATTACTTGTCGTGCTTCTCAAGATGTTAATACTGCTGTTGCTTGGTATC
AACAAAAACCCGGTAAAGCTCCTAAACTTCTTATTTATTCTGCTTCTTTTCTTTATTCTGGTGTTCCTTCTCGTTTTT
CTGGTTCTCGTTCTGGTACTGATTTTACTCTTACTATTTCTTCTCTTCAACCTGAAGATTTTGCTACTTATTATTGTC
AACAACATTATACTACTCCTCCTACTTTTGGTCAAGGTACCAAGGTGGAGATCAAACGTACGCATCATCACCATCACC
AT
SEQID NO. 311
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGGGGAGGTGGGTCTGGAGGTGGA
GGATCTGGTGGAGGTGGGTCTGGTGGAGGTGGGTCTGAGGTGCAGCTGGTGGAGTCTGGTGGTGGTCTTGTTCAACCT
GGTGGTTCTCTTCGTCTTTCTTGTGCTGCTTCTGGTTTTAATATTAAAGATACTTATATTCATTGGGTTCGTCAAGCT
CCTGGTAAAGGTCTTGAATGGGTTGCTCGTATTTATCCTACTAATGGTTATACTCGTTATGCTGATTCTGTTAAAGGT
CGTTTTACTATTTCTGCTGATACTTCTAAAAATACTGCTTATCTTCAAATGAACTCTCTTCGTGCTGAAGATACTGCT
GTTTATTATTGTTCTCGTTGGGGTGGTGATGGTTTTTATGCTATGGATTATTGGGGTCAAGGTACTCTTGTCACCGTC
TCCTCAGCTAGCACCGGGGGAGGTGGGTCTGGAGGTGGAGGATCTGGTGGAGGTGGGTCTGACATCCAGATGACCCAG
TCTCCTTCTTCTCTTTCTGCTTCTGTTGGTGATCGTGTTACTATTACTTGTCGTGCTTCTCAAGATGTTAATACTGCT
GTTGCTTGGTATCAACAAAAACCTGGTAAAGCTCCTAAACTTCTTATTTATTCTGCTTCTTTTCTTTATTCTGGTGTT
CCTTCTCGTTTTTCTGGTTCTCGTTCTGGTACTGATTTTACTCTTACTATTTCTTCTCTTCAACCTGAAGATTTTGCT
ACTTATTATTGTCAACAACATTATACTACTCCTCCTACTTTTGGTCAAGGTACCAAGGTGGAGATCAAACGTACGCAC
CACCATCACCACCAT
SEQID NO. 312
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGGCACTGGCTGCTGAGG
ACTGGTGGCTGGAAG
SEQID NO. 313
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGGGGAGGTGGGTCTGGAGGTGGA
GGATCTGGTGGAGGTGGGTCTGGTGGAGGTGGGTCTGAGGTGCAGCTGGTGGAGTCTGGTGGTGGTCTTGTTCAACCT
GGTGGTTCTCTTCGTCTTTCTTGTGCTGCTTCTGGTTTTAATATTAAAGATACTTATATTCATTGGGTTCGTCAAGCT
CCTGGTAAAGGTCTTGAATGGGTTGCTCGTATTTATCCTACTAATGGTTATACTCGTTATGCTGATTCTGTTAAAGGT
CGTTTTACTATTTCTGCTGATACTTCTAAAAATACTGCTTATCTTCAAATGAACTCTCTTCGTGCTGAAGATACTGCT
GTTTATTATTGTTCTCGTTGGGGTGGTGATGGTTTTTATGCTATGGATTATTGGGGTCAAGGTACTCTTGTCACCGTC
TCCTCAGCTAGCACCGGGGGAGGTGGGTCTGGAGGTGGAGGATCTGGTGGAGGTGGGTCTGACATCCAGATGACCCAG
TCTCCTTCTTCTCTTTCTGCTTCTGTTGGTGATCGTGTTACTATTACTTGTCGTGCTTCTCAAGATGTTAATACTGCT
GTTGCTTGGTATCAACAAAAACCTGGTAAAGCTCCTAAACTTCTTATTTATTCTGCTTCTTTTCTTTATTCTGGTGTT
CCTTCTCGTTTTTCTGGTTCTCGTTCTGGTACTGATTTTACTCTTACTATTTCTTCTCTTCAACCTGAAGATTTTGCT
ACTTATTATTGTCAACAACATTATACTACTCCTCCTACTTTTGGTCAAGGTACCAAGGTGGAGATCAAACGTACGTCT
AGACATCATCACCATCACCAT
SEQID NO. 314
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTCAGGTGCAGCTGGTGCAGAGCGGCGCCGAGGACAAGAAGCCC
GGCGAGAGCGTGAAGATCAGCTGCAAGGCCAGCGGCTACACCTTCACCAACTACGGCATGAACTGGGTGAGGCAGGCC
CCCGGCCAGGGCCTGAAGTGGATGGGCTGGATCAACACCTACACCGGCGAGAGCACCTACGCCGACGACTTCAAGGGC
AGGTTCGCCTTCAGCCTGGACACCAGCGCCAGCACCGCCTACCTGCAGCTGAGCAGCCTGAGGGGCGAGGACACCGCC
GTGTACTTCTGCGCCAGGTTCGCCATCAAGGGCGACTACTGGGGCCAGGGCACCACCGTGACCGTGAGCAGCGCCAGC
ACCGGCGGCGGCGGCAGCGGCGGCGGCGGCAGCGGCGGCGGCGGCAGCGGCGGCGGCGGCAGCGACATCGTGATGACC
CAGAGCCCCCTGAGCCTGGAGGTGAGCCCCGGCGAGCCCGCCAGCATCAGCTGCAGGAGCACCAAGAGCCTGCTGCAC
AGCGACGGCATCACCTACCTGTACTGGTACCTGCAGAAGCCCGGCCAGAGCCCCCAGCTGCTGATCTACCAGCTGAGC
AACCTGGCCAGCGGCGTGCCCGACAGGTTCAGCAGCAGCGGCAGCGGCACCGACTTCACCCTGAAGATCAGCAGGGTG
GAGGCCGAGGACGAGGGCACCTACTACTGCGCCCAGAACCTGGAGATCCCCAGGACCTTCGGCCAGGGCACCAAGCTG
GAGATCAAGAGGACCCATCATCACCATCACCAT
SEQID NO. 315
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTCAGGTTCAGCTGGTGCAGTCTGGTGCTGAGGATGTGAAGCCT
GATGCCTCAGTGAAGCTCTCCTGCAAGGCTTCTGGTTACACATTCACTGACTACTACATGCACTGGGTGCGTCAGGCC
CCTGGTCAAGGTCTTGAGTGGATGGGTCGTGTTAATCCTAACCGGAGGGGTACTACCTACAACCAGAAATTCGAGGGC
CGTGTCACCATGACCACAGACACATCCACGAGCACAGCCTACATGCAGCTGAGTAGCCTGCGTGGTGAAGACACGGCC
GTGTATTACTGTGCGCGTGCGAACTGGCTTGACTACTGGGGCCAGGGCACCACCGTCACCGTCTCCTCCGCCTCCACC
GGGGGAGGTGGGTCTGGAGGTGGAGGATCTGGTGGAGGTGGGTCTGGTGGAGGTGGGTCTGACATCCAGATGACCCAG
TCTCCATCCTCCCTGGAGGCATCTGTAGGAGACAGAGTCACCATCACTTGCAGTGTCAGCTCAAGTGTATCCTCCATT
TACTTGCACTGGTATCAGCAGAAACCAGGGAAAAGCCCTAAGCTCCTGATCTATAGCACATCCAACTTGGCTTCTGGA
GTCCCAGATAGGTTCAGTGGCAGTGGATCTGGGACAGATTTCACTCTCACCATCAGCAGTCTGCAAGCCGAAGATGAG
GGCACTTACTACTGTCAAGTCTACAGTGGTTACCCGCTCACGTTCGGCGGAGGGACCAAGCTGGAGATCAAACGAACT
CATCATCACCATCACCAT
SEQID NO. 316
ATGCCACCTCCTCGCCTCCTCTTCTTCCTCCTCTTCCTCACCCCCATGGAAGTCAGGCCCGAGGAACCTCTAGTGGTG
AAGGTGGAAGAGGGAGATAACGCTGTGCTGCAGTGCCTCAAGGGGACCTCAGATGGCCCCACTCAGCAGCTGACCTGG
TCTCGGGAGTCCCCGCTTAAACCCTTCTTAAAACTCAGCCTGGGGCTGCCAGGCCTGGGAATCCACATGAGGCCCCTG
GCCATCTGGCTTTTCATCTTCAACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCTCATGAGCCCCAAGCTG
TATGTGTGGGCCAAAGACCGCCCTGAGATCTGGGAGGGAGAGCCTCCGTGTCTCCCACCGAGGGACAGCCTGAACCAG
AGCCTCAGCCAGGACCTCACCATGGCCCCTGGCTCCACACTCTGGCTGTCCTGTGGGGTACCCCCTGACTCTGTGTCC
AGGGGCCCCCTCTCCTGGACCCATGTGCACCCCAAGGGGCCTAAGTCATTGCTGAGCCTAGAGCTGAAGGACGATCGC
CCGGCCAGAGATATGTGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGGAGGAGGTGGGTCTGGAGGTGGA
GGATCTGGTGGAGGTGGGTCTGGAGGAGGTGGGTCTCAGGTGCAGCTGCAGGAGAGCGGCCCCGGCGACGTGAAGCCC
AGCGAGACCCTGAGCCTGACCTGCACCGTGAGCGGCGGCAGCGTGAGCAGCGGCGACTACTACTGGACCTGGATCAGG
CAGAGCCCCGGCAAGGGCCTGGAGTGGATCGGCCACATCTACTACAGCGGCAACACCAACTACAACCCCAGCCTGAAG
AGCAGGCTGACCATCAGCATCGACACCAGCAAGACCACCTTCAGCCTGCAGCTGAGCAGCGTGACCGGCGAGGACACC
GCCATCTACTACTGCGTGAGGGACAGGGTGACCGGCGCCTTCGACATCTGGGGCCAGGGCACCACCGTGACCGTGAGC
AGCGCCAGCACCGGCGGCGGCGGCAGCGGCGGCGGCGGCAGCGGCGGCGGCGGCAGCGGCGGCGGCGGCAGCGACATC
CAGATGACCCAGAGCCCCAGCAGCCTGGAGGCCAGCGTGGGCGACAGGGTGACCATCACCTGCCAGGCCAGCCAGGAC
ATCAGCAACTACCTGAACTGGTACCAGCAGAAGCCCGGCAAGAGCCCCAAGCTGCTGATCTACGACGCCAGCAACCTG
GAGACCGGCGTGCCCGACAGGTTCAGCGGCAGCGGCAGCGGCACCGACTTCACCTTCACCATCAGCAGCCTGCAGGCC
GAGGACGAGGGCACCTACTTCTGCCAGCACTTCGACCACCTGCCCCTGGCCTTCGGCGGCGGCACCAAGCTGGAGATC
AAGAGGACCCATCATCACCATCACCAT
SEQID NO. 317
ATGCTCAGGCTGCTCTTGGCTCTCAACTTATTCCCTTCAATTCAAGTAACAGGAGACATCCAGATGACACAGACTACA
TCCTCCCTGTCTGCCTCTCTGGGAGACAGAGTCACCATCAGTTGCAGGGCAAGTCAGGACATTAGTAAATATTTAAAT
TGGTATCAGCAGAAACCAGATGGAACTGTTAAACTCCTGATCTACCATACATCAAGATTACACTCAGGAGTCCCATCA
AGGTTCAGTGGCAGTGGGTCTGGAACAGATTATTCTCTCACCATTAGCAACCTGGAGCAAGAAGATATTGCCACTTAC
TTTTGCCAACAGGGTAATACGCTTCCGTACACGTTCGGAGGGGGGACTAAGTTGGAAATAACAGGAGGAGGTGGGTCT
GGAGGTGGAGGATCTGGTGGAGGTGGGTCTGGAGGAGGTGGGTCTGAGGTGAAACTGCAGGAGTCAGGACCTGGCCTG
GTGGCGCCCTCACAGAGCCTGTCCGTCACATGCACTGTCTCAGGGGTCTCATTACCCGACTATGGTGTAAGCTGGATT
CGCCAGCCTCCACGAAAGGGTCTGGAGTGGCTGGGAGTAATATGGGGTAGTGAAACCACATACTATAATTCAGCTCTC
AAATCCAGACTGACCATCATCAAGGACAACTCCAAGAGCCAAGTTTTCTTAAAAATGAACAGTCTGCAAACTGATGAC
ACAGCCATTTACTACTGTGCCAAACATTATTACTACGGTGGTAGCTATGCTATGGACTACTGGGGTCAAGGAACCTCA
GTCACCGTCTCCTCAGACTACAAAGACGATGACGACAAGATTGAAGTTATGTATCCTCCTCCTTACCTAGACAATGAG
AAGAGCAATGGAACCATTATCCATGTGAAAGGGAAACACCTTTGTCCAAGTCCCCTATTTCCCGGACCTTCTAAGCCC
TTTTGGGTGCTGGTGGTGGTTGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCGCCGCCCCGGGCCCACCCGCAAG
CATTACCAGCCCTATGCCCCACCACGCGACTTCGCAGCCTATCGCTCCAAACGGGGCAGAAAGAAACTCCTGTATATA
TTCAAACAACCATTTATGAGACCAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAGAGACGTGGCCGGGACCCTGAGATGGGG
GGAAAGCCGCAGAGAAGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGTCTCAGTACAGCCACC
AAGGACACCTACGACGCCCTTCACATGCAGGCCCTGCCCCCTCGC
SEQID NO. 318
ATGCTCAGGCTGCTCTTGGCTCTCAACTTATTCCCTTCAATTCAAGTAACAGGAGACATCCAGATGACACAGACTACA
TCCTCCCTGTCTGCCTCTCTGGGAGACAGAGTCACCATCAGTTGCAGGGCAAGTCAGGACATTAGTAAATATTTAAAT
TGGTATCAGCAGAAACCAGATGGAACTGTTAAACTCCTGATCTACCATACATCAAGATTACACTCAGGAGTCCCATCA
AGGTTCAGTGGCAGTGGGTCTGGAACAGATTATTCTCTCACCATTAGCAACCTGGAGCAAGAAGATATTGCCACTTAC
TTTTGCCAACAGGGTAATACGCTTCCGTACACGTTCGGAGGGGGGACTAAGTTGGAAATAACAGGAGGAGGTGGGTCT
GGAGGTGGAGGATCTGGTGGAGGTGGGTCTGGAGGAGGTGGGTCTGAGGTGAAACTGCAGGAGTCAGGACCTGGCCTG
GTGGCGCCCTCACAGAGCCTGTCCGTCACATGCACTGTCTCAGGGGTCTCATTACCCGACTATGGTGTAAGCTGGATT
CGCCAGCCTCCACGAAAGGGTCTGGAGTGGCTGGGAGTAATATGGGGTAGTGAAACCACATACTATAATTCAGCTCTC
AAATCCAGACTGACCATCATCAAGGACAACTCCAAGAGCCAAGTTTTCTTAAAAATGAACAGTCTGCAAACTGATGAC
ACAGCCATTTACTACTGTGCCAAACATTATTACTACGGTGGTAGCTATGCTATGGACTACTGGGGTCAAGGAACCTCA
GTCACCGTCTCCTCAATTGAAGTTATGTATCCTCCTCCTTACCTAGACAATGAGAAGAGCAATGGAACCATTATCCAT
GTGAAAGGGAAACACCTTTGTCCAAGTCCCCTATTTCCCGGACCTTCTAAGCCCTTTTGGGTGCTGGTGGTGGTTGGT
GGAGTCCTGGCTTGCTATAGCTTGCTAGTAACAGTGGCCTTTATTATTTTCTGGGTGAGGAGTAAGAGGAGCAGGCTC
CTGCACAGTGACTACATGAACATGACTCCCCGCCGCCCCGGGCCCACCCGCAAGCATTACCAGCCCTATGCCCCACCA
CGCGACTTCGCAGCCTATCGCTCCAAACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAAGAAGGAGGATGTGAACTGAGAGTG
AAGTTCAGCAGGAGCGCAGACGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGAAAGCCGCAGAGAAGGAAGAAC
CCTCAGGAAGGCCTGTACAATGAACTGCAGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGCCACCAAGGACACCTACGACGCCCTTCAC
ATGCAGGCCCTGCCCCCTCGC
SEQID NO. 319
ATGGAGAAGGACACCCTGCTGCTGTGGGTGCTGCTGCTGTGGGTGCCCGGCAGCACCGGCGAGGTGAAGCTGGTGGAG
AGCGGCGGCGGCCTGGTGCAGCCCGGCGGCAGCCTGAAGCTGAGCTGCGCCGCCAGCGGCTTCGACTTCAGCAGGTAC
TGGATGAGCTGGGTGAGGCAGGCCCCCGGCAAGGGCCTGGAGTGGATCGGCGAGATCAACCTGGACAGCAGCACCATC
AACTACACCCCCAGCCTGAAGGACAAGTTCATCATCAGCAGGGACAACGCCAAGAACACCCTGTACCTGCAGATGAGC
AAGGTGAGGAGCGAGGACACCGCCCTGTACTACTGCGCCAGGAGGTACGACGCCATGGACTACTGGGGCCAGGGCACC
AGCGTGACCGTGAGCAGCGCCAAGACCACCGCCCCCAGCGTGTACCCCCTGGCCCCCGTGTGCGGCGACACCACCGGC
AGCAGCGTGACCCTGGGCTGCCTGGTGAAGGGCTACTTCCCCGAGCCCGTGACCCTGACCTGGAACAGCGGCAGCCTG
AGCAGCGGCGTGCACACCTTCCCCGCCGTGCTGCAGAGCGACCTGTACACCCTGAGCAGCAGCGTGACCGTGACCAGC
AGCACCTGGCCCAGCCAGAGCATCACCTGCAACGTGGCCCACCCCGCCAGCAGCACCAAGGTGGACAAGAAGATCGAG
CCCAGGGGCCCCACCATCAAGCCCTGCCCCCCCTGCAAGTGCCCCGCCCCCAACCTGCTGGGCGGCCCCAGCGTGTTC
ATCTTCCCCCCCAAGATCAAGGACGTGCTGATGATCAGCCTGAGCCCCATCGTGACCTGCGTGGTGGTGGACGTGAGC
GAGGACGACCCCGACGTGCAGATCAGCTGGTTCGTGAACAACGTGGAGGTGCACACCGCCCAGACCCAGACCCACAGG
GAGGACTACAACAGCACCCTGAGGGTGGTGAGCGCCCTGCCCATCCAGCACCAGGACTGGATGAGCGGCAAGGAGTTC
AAGTGCAAGGTGAACAACAAGGACCTGCCCGCCCCCATCGAGAGGACCATCAGCAAGCCCAAGGGCAGCGTGAGGGCC
CCCCAGGTGTACGTGCTGCCCCCCCCCGAGGAGGAGATGACCAAGAAGCAGGTGACCCTGACCTGCATGGTGACCGAC
TTCATGCCCGAGGACATCTACGTGGAGTGGACCAACAACGGCAAGACCGAGCTGAACTACAAGAACACCGAGCCCGTG
CTGGACAGCGACGGCAGCTACTTCATGTACAGCAAGCTGAGGGTGGAGAAGAAGAACTGGGTGGAGAGGAACAGCTAC
AGCTGCAGCGTGGTGCACGAGGGCCTGCACAACCACCACACCACCAAGAGCTTCAGCAGGACCCCCGGCAAG
SEQID NO. 320
ATGGACTTCGGCCTGATCTTCTTCATCGTGGCCCTGCTGAAGGGCGTGCAGTGCGACATCGTGCTGACCCAGAGCCCC
GCCAGCCTGGCCGTGAGCCTGGGCCAGAGGGCCACCATCAGCTGCAGGGCCAGCGAGAGCGTGGACGACTACGGCATC
AGCTTCATGAACTGGTTCCAGCAGAAGCCCGGCCAGCCCCCCAAGCTGCTGATCTACGCCGCCCCCAACCAGGGCAGC
GGCGTGCCCGCCAGGTTCAGCGGCAGCGGCAGCGGCACCGACTTCAGCCTGAACATCCACCCCATGGAGGAGGACGAC
ACCGCCATGTACTTCTGCCAGCAGAGCAAGGACGTGAGGTGGACCTTCGGCGGCGGCACCAAGCTGGAGATCAAGAGG
GCCGACGCCGCCCCCACCGTGAGCATCTTCCCCCCCAGCAGCGAGCAGCTGACCAGCGGCGGCGCCAGCGTGGTGTGC
TTCCTGAACAACTTCTACCCCAAGGACATCAACGTGAAGTGGAAGATCGACGGCAGCGAGAGGCAGAACGGCGTGCTG
AACAGCTGGACCGACCAGGACAGCAAGGACAGCACCTACAGCATGAGCAGCACCCTGACCCTGACCAAGGACGAGTAC
GAGAGGCACAACAGCTACACCTGCGAGGCCACCCACAAGACCAGCACCAGCCCCATCGTGAAGAGCTTCAACAGGAAC
GAGTGC
SEQID NO. 321
ATGGAGAAGGACACCCTGCTGCTGTGGGTGCTGCTGCTGTGGGTGCCCGGCAGCACCGGCGAGGTGAAGCTGGTGGAG
AGCGGCGGCGGCCTGGTGCAGCCCGGCGGCAGCCTGAAGCTGAGCTGCGCCGCCAGCGGCTTCGACTTCAGCAGGTAC
TGGATGAGCTGGGTGAGGCAGGCCCCCGGCAAGGGCCTGGAGTGGATCGGCGAGATCAACCTGGACAGCAGCACCATC
AACTACACCCCCAGCCTGAAGGACAAGTTCATCATCAGCAGGGACAACGCCAAGAACACCCTGTACCTGCAGATGAGC
AAGGTGAGGAGCGAGGACACCGCCCTGTACTACTGCGCCAGGAGGTACGACGCCATGGACTACTGGGGCCAGGGCACC
AGCGTGACCGTGAGCAGCGCCAGCACCGGCGGCGGCGGCAGCGGCGGCGGCGGCAGCGGCGGCGGCGGCAGCGGCGGC
GGCGGCAGCGACATCGTGCTGACCCAGAGCCCCGCCAGCCTGGCCGTGAGCCAGGGCCAGAGGGCCACCATCAGCTGC
AGGGCCAGCGAGAGCGTGGACGACTACGGCATCAGCTTCATGAACTGGTTCCAGCAGAAGCCCGGCCAGCCCCCCAAG
CTGCTGATCTACGCCGCCCCCAACCAGGGCAGCGGCGTGCCCGCCAGGTTCAGCGGCAGCGGCAGCGGCACCGACTTC
AGCCTGAACATCCACCCCATGGAGGAGGACGACACCGCCACCTACTTCTGCCAGCAGAGCAAGGACGTGAGGTGGACC
TTCGGCGGCGGCACCAAGCTGGAGATCAAGAGGGGAGGAGGTGGGTCTGGAGGTGGAGGATCTGGTGGAGGTGGGTCT
GGAGGAGGTGGGTCTGAGGTGCAGCTGGTGGAGTCTGGTGGTGGTCTTGTTCAACCTGGTGGTTCTCTTCGTCTTTCT
TGTGCTGCTTCTGGTTTTAATATTAAAGATACTTATATTCATTGGGTTCGTCAAGCTCCTGGTAAAGGTCTTGAATGG
GTTGCTCGTATTTATCCTACTAATGGTTATACTCGTTATGCTGATTCTGTTAAAGGTCGTTTTACTATTTCTGCTGAT
ACTTCTAAAAATACTGCTTATCTTCAAATGAACTCTCTTCGTGCTGAAGATACTGCTGTTTATTATTGTTCTCGTTGG
GGTGGTGATGGTTTTTATGCTATGGATTATTGGGGTCAAGGTACTCTTGTCACCGTCTCCTCAGCTAGCACCGGGGGA
GGTGGGTCTGGAGGTGGAGGATCTGGTGGAGGTGGGTCTGACATCCAGATGACCCAGTCTCCTTCTTCTCTTTCTGCT
TCTGTTGGTGATCGTGTT ACTATTACTTGTCGTGCTTCTCAAGATGTTAATACTGCTGT
TGCTTGGTATCAACAAAAACCTGGTAAAGCTCCTAAACTTCTTATTTATTCTGCTTCTTTTCTTTATTCTGGTGTTCC
TTCTCGTTTTTCTGGTTCTCGTTCTGGTACTGATTTTACTCTTACTATTTCTTCTCTTCAACCTGAAGATTTTGCTAC
TTATTATTGTCAACAACATTATACTACTCCTCCTACTTTTGGTCAAGGTACCAAGGTGGAGATCAAACGTACGTCTAG
AGGGCCCCATCATCACCATCACCAT
SEQID NO. 322
ATGGACTTCGGCCTGATCTTCTTCATCGTGGCCCTGCTGAAGGGCGTGCAGTGCGACATCGTGCTGACCCAGAGCCCC
GCCAGCCTGGCCGTGAGCCAGGGCCAGAGGGCCACCATCAGCTGCAGGGCCAGCGAGAGCGTGGACGACTACGGCATC
AGCTTCATGAACTGGTTCCAGCAGAAGCCCGGCCAGCCCCCCAAGCTGCTGATCTACGCCGCCCCCAACCAGGGCAGC
GGCGTGCCCGCCAGGTTCAGCGGCAGCGGCAGCGGCACCGACTTCAGCCTGAACATCCACCCCATGGAGGAGGACGAC
ACCGCCACCTACTTCTGCCAGCAGAGCAAGGACGTGAGGTGGACCTTCGGCGGCGGCACCAAGCTGGAGATCAAGAGG
GGCGGCGGCGGCAGCGGCGGCGGCGGCAGCGGCGGCGGCGGCAGCGGCGGCGGCGGCAGCGAGGTGAAGCTGGTGGAG
AGCGGCGGCGGCCTGGTGCAGCCCGGCGGCAGCCTGAAGCTGAGCTGCGCCGCCAGCGGCTTCGACTTCAGCAGGTAC
TGGATGAGCTGGGTGAGGCAGGCCCCCGGCAAGGGCCTGGAGTGGATCGGCGAGATCAACCTGGACAGCAGCACCATC
AACTACACCCCCAGCCTGAAGGACAAGTTCATCATCAGCAGGGACAACGCCAAGAACACCCTGTACCTGCAGATGAGC
AAGGTGAGGAGCGAGGACACCGCCCTGTACTACTGCGCCAGGAGGTACGACGCCATGGACTACTGGGGCCAGGGCACC
AGCGTGACCGTGAGCAGCGGAGGAGGTGGGTCTGGAGGTGGAGGATCTGGTGGAGGTGGGTCTGGAGGAGGTGGGTCT
GAGGTGCAGCTGGTGGAGTCTGGTGGTGGTCTTGTTCAACCTGGTGGTTCTCTTCGTCTTTCTTGTGCTGCTTCTGGT
TTTAATATTAAAGATACTTATATTCATTGGGTTCGTCAAGCTCCTGGTAAAGGTCTTGAATGGGTTGCTCGTATTTAT
CCTACTAATGGTTATACTCGTTATGCTGATTCTGTTAAAGGTCGTTTTACTATTTCTGCTGATACTTCTAAAAATACT
GCTTATCTTCAAATGAACTCTCTTCGTGCTGAAGATACTGCTGTTTATTATTGTTCTCGTTGGGGTGGTGATGGTTTT
TATGCTATGGATTATTGGGGTCAAGGTACTCTTGTCACCGTCTCCTCAGCTAGCACCGGGGGAGGTGGGTCTGGAGGT
GGAGGATCTGGTGGAGGTGGGTCTGACATCCAGATGACCCAGTCTCCTTCTTCTCTTTCTGCTTCTGTTGGTGATCGT
GTTACTATTACTTGTCGTGCTTCTCAAGATGTTAATACTGCTGTTGCTTGGTATCAACAAAAACCTGGTAAAGCTCCT
AAACTTCTTATTTATTCTGCTTCTTTTCTTTATTCTGGTGTTCCTTCTCGTTTTTCTGGTTCTCGTTCTGGTACTGAT
TTTACTCTTACTATTTCTTCTCTTCAACCTGAAGATTTTGCTACTTATTATTGTCAACAACATTATACTACTCCTCCT
ACTTTTGGTCAAGGTACCAAGGTGGAGATCAAACGTACGTCTAGAGGGCCCCATCATCACCATCACCAT

1. A recombinant adenoviral vector (Ad), adeno-associated viral (AAV) vector, oncolytic viral vector, or chimeric AAV/phage (AAVP) vector, encoding a fusion protein comprising (i) an antibody or antibody fragment to a tumor-associated antigens (TAA) or tumor-specific antigens (TSA) and (ii) an anti-idiotype antibody or fragment, or an anti-idiotype peptide.

2. The vector of claim 1, wherein the TAA or TSA is selected from the group consisting of glioma-associated antigen, carcinoembryonic antigen (CEA), β-human chorionic gonadotropin, alphafetoprotein (AFP), lectin-reactive AFP, thyroglobulin, RAGE-1 , MN-CA IX, human telomerase reverse transcriptase, RU1 , RU2 (AS), intestinal carboxyi esterase, mut hsp70-2, M-CSF, prostase, prostate-specific antigen (PSA), PAP, NY-ESO- 1 , LAGE-la, p53, prostein, PSMA, Her2/neu, survivin and telomerase, prostate-carcinoma tumor antigen- 1 (PCTA-1), MAGE, ELF2M, neutrophil elastase, ephrinB2, CD22, insulin growth factor (IGF)-I, IGF-II, IGF-I receptor and mesothelin, EphA2, HER2, GD2, Glypican-3, 5T4, 8H9, αvβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-AI MAGE Al, HLA-A2, ILl IRa, IL13Ra2, KDR, Lambda, Lewis-Y, MCSP, Mesothelin, Mud, Muc16, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, and VEGF receptors.

3. The vector of claim 1, wherein the anti-idiotype antibody or fragment, or the anti-idiotype peptide, binds an anti-CD19, anti-CD20, anti-CD21, anti-CD22, anti-CD24, anti-CD79a, anti-CD79b, anti-ROR1, or anti-BCMA antibody or fragment thereof.

4. The vector of claim 3, wherein the anti-idiotype antibody or fragment, or the anti-idiotype peptide, binds an anti-CD19 antibody or fragment (e.g., an scFv).

5. (canceled)

6. The vector of claim 1, wherein the vector integrates into the genome of a cancer cell.

7. The vector of claim 1, wherein the anti-idiotype antibody or fragment, or the anti-idiotype peptide, binds a chimeric antigen receptor (CAR).

8. The vector of claim 1, wherein the anti-idiotype antibody or fragment, or the anti-idiotype peptide, binds an ADC antibody, ADCC antibody, and/or a radiotherapeutic antibody.

9. A recombinant tumor cell comprising the vector of claim 1.

10. (canceled)

11. A method of producing a recombinant tumor cell capable of expressing a fusion protein comprising (i) an antibody or antibody fragment to a tumor-associated antigens (TAA) or tumor-specific antigens (TSA) and (ii) an anti-idiotype antibody or fragment, or an anti-idiotype peptide, said method comprising (a) introducing the vector of claim 1 into a tumor cell; and (b) culturing the tumor cell such that the vector is transduced into the tumor cell.

12. (canceled)

13. The method of claim 11 wherein the TAA or TSA is selected from the group consisting of glioma-associated antigen, carcinoembryonic antigen (CEA), β-human chorionic gonadotropin, alphafetoprotein (AFP), lectin-reactive AFP, thyroglobulin, RAGE-1 , MN-CA IX, human telomerase reverse transcriptase, RU1 , RU2 (AS), intestinal carboxyi esterase, mut hsp70-2, M-CSF, prostase, prostate-specific antigen (PSA), PAP, NY-ESO- 1 , LAGE-la, p53, prostein, PSMA, Her2/neu, survivin and telomerase, prostate-carcinoma tumor antigen- 1 (PCTA-1), MAGE, ELF2M, neutrophil elastase, ephrinB2, CD22, insulin growth factor (IGF)-I, IGF-II, IGF-I receptor and mesothelin, EphA2, HER2, GD2, Glypican-3, 5T4, 8H9, αvβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-AI MAGE Al, HLA-A2, IL1 IRa, IL13Ra2, KDR, Lambda, Lewis-Y, MCSP, Mesothelin, Mud, Muc16, NCAM, NKG2D ligands, NY-ESO-1, PRAME, PSCA, PSC1, PSMA, ROR1, SURVIVIN, TAG72, TEM1, TEM8, VEGRR2, carcinoembryonic antigen, HMW-MAA, and VEGF receptors.

14. The method of claim 11 wherein the anti-idiotype antibody or fragment, or the anti-idiotype peptide, binds a chimeric antigen receptor (CAR).

15. The method of claim 11 wherein the anti-idiotype antibody or fragment, or the anti-idiotype peptide, binds an ADC antibody, ADCC antibody, and/or a radiotherapeutic antibody.

16. A method of treating cancer in an individual in need thereof comprising administering a composition comprising the vector of claim 1.

17. The method of claim 16 wherein a tumor cell of the individual expresses the fusion protein.

18. (canceled)

19. The method of claim 16 wherein the fusion protein comprises an anti-idiotype antibody or fragment, or the anti-idiotype peptide, that binds a CAR.

20. The method of claim 16, wherein the anti-idiotype antibody or fragment, or the anti-idiotype peptide, binds an anti-CD19, anti-CD20, anti-CD21, anti-CD22, anti-CD24, anti-CD79a, anti-CD79b, anti-ROR1, or anti-BCMA antibody or fragment thereof.

21. The method of claim 20, wherein the anti-idiotype antibody or fragment, or the anti-idiotype peptide, binds an anti-CD19 antibody or fragment (e.g. an scFv).

22. The method of claim claim 16 wherein the fusion protein comprises an anti-idiotype antibody or fragment, or the anti-idiotype peptide, that binds an immune cell in the individual.

23. The method of claim 16 further comprising administration of CAR T cells to the individual.

24.-27. (canceled)
